(19) United States
(12) Patent Application Publication
(10) Pub. No.: US 2026/0033123 A1
(43) Pub. Date: Jan. 29, 2026

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Seonyeong GWAK, Suwon-si (KR); Hanill Lee, Suwon-si (KR); Seungjae Lee, Suwon-si (KR); Jiah Yoon, Suwon-si (KR); Suheon Im, Suwon-si (KR); Mijin Lee, Suwon-si (KR); Hyungyu Lee, Suwon-si (KR)

(21) Appl. No.: 19/259,285

(22) Filed: Jul. 3, 2025

(30) Foreign Application Priority Data
Jul. 23, 2024 (KR) 10-2024-0097291

(51) Int. Cl. H10K 50/11; C07D 307/91; H10K 85/40; H10K 85/60

(52) U.S. Cl. CPC H10K 50/11; C07D 307/91; H10K 85/40; H10K 85/622; H10K 85/626; H10K 85/633; H10K 85/636; H10K 85/654; H10K 85/6572; H10K 85/6574; H10K 85/6576

(57) ABSTRACT

A compound for an organic optoelectronic device a composition for an organic optoelectronic device including the compound for an organic optoelectronic device, an organic optoelectronic device, and a display device including the organic optoelectronic device, the compound being represented by Chemical Formula 1 or Chemical Formula 2:

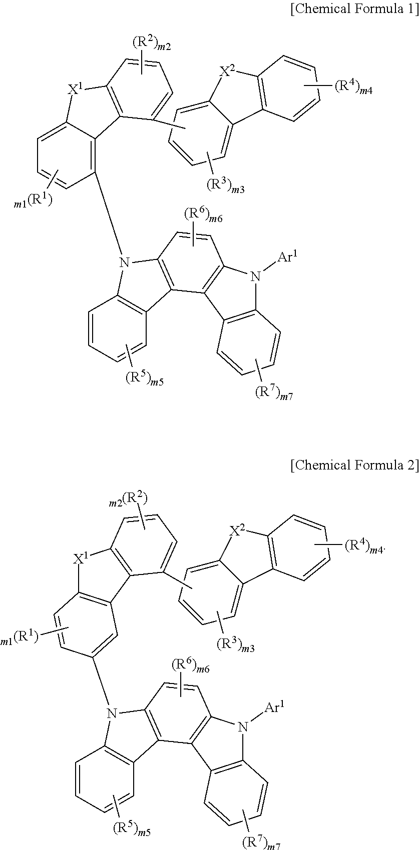

### CROSS-REFERENCE TO RELATED APPLICATION

This application claims priority to and the benefit of Korean Patent Application No. 10-2024-0097291 filed in the Korean Intellectual Property Office on Jul. 23, 2024, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device may include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode may be influenced by an organic material between electrodes.

### SUMMARY

The embodiments may be realized by providing a compound for an organic optoelectronic device, the compound being represented by Chemical Formula 1 or Chemical Formula 2:

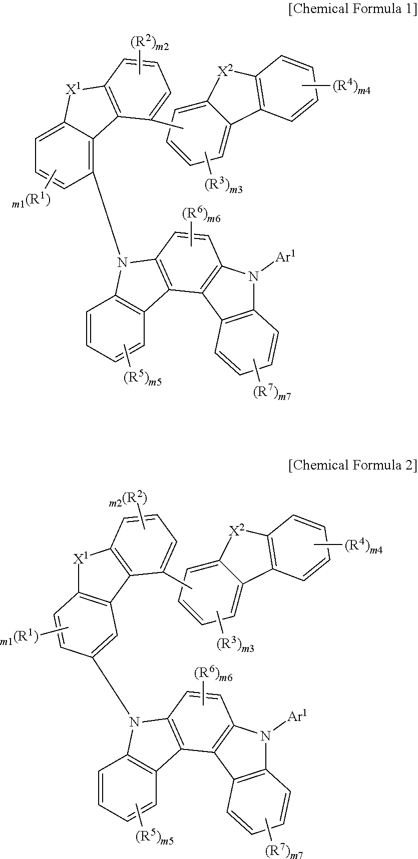

wherein, in Chemical Formula 1 and Chemical Formula 2, X¹ and X² are each independently O or S, R¹ to R⁷ are each independently hydrogen, deuterium, a cyano group, a substituted or unsubstituted C1 to C10 alkyl group, a substituted or unsubstituted C1 to C10 alkylsilyl group, a substituted or unsubstituted C3 to C20 cycloalkyl group, or a substituted or unsubstituted C6 to C20 aryl group, Ar¹ is a substituted or unsubstituted C6 to C20 aryl group, m1 to m3 are each independently an integer of 1 to 3, m4, m5, and m7 are each independently an integer of 1 to 4, and m6 is an integer of 1 or 2.

The embodiments may be realized by providing a composition for an organic optoelectronic device, the composition including a first compound, and a second compound, wherein the first compound may be the compound for an organic optoelectronic device according to an embodiment, and the second compound may be represented by Chemical Formula 3:

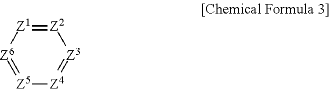

in Chemical Formula 3, Z¹ to Z⁶ may each independently be N or C-L^{a}-R^{a}, provided that at least two of Z¹ to Z⁶ are N, each L^{a} may independently be a single bond, a substituted or unsubstituted C6 to C20 arylene group, a substituted or unsubstituted C2 to C20 heterocyclic group, or a combination thereof, each R^{a} may independently be hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof, and each R^{a} may be separately present or adjacent groups thereof may be linked to each other to form a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring.

The embodiments may be realized by providing an organic optoelectronic device including an anode and a cathode facing each other, and at least one organic layer between the anode and the cathode, wherein the at least one organic layer includes the compound for an organic optoelectronic device according to an embodiment.

The embodiments may be realized by providing an organic optoelectronic device including an anode and a cathode facing each other, and at least one organic layer between the anode and the cathode, wherein the at least one organic layer includes the composition for an organic optoelectronic device according to an embodiment.

The embodiments may be realized by providing a display device including the organic photoelectronic device according to an embodiment.

### BRIEF DESCRIPTION OF THE DRAWING

Features will become apparent to those of skill in the art by describing in detail exemplary embodiments with reference to the attached drawing in which:

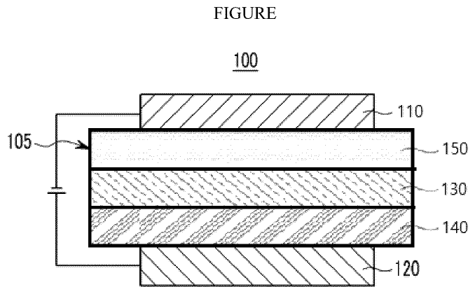

the FIG. 1s a cross-sectional view showing an organic light emitting diode according to some example embodiments.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In a specific example, the “substituted” refers to replacement of at least one hydrogen of a substituent or a compound by deuterium, a C1 to C20 alkyl group, a C6 to C30 aryl group, or a cyano group. In a specific example, the “substituted” refers to replacement of at least one hydrogen of a substituent or a compound by deuterium, a C1 to C5 alkyl group, a C6 to C18 aryl group, or a cyano group. In a specific example, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

As used herein, “unsubstituted” refers to non-replacement of a hydrogen atom by another substituent and remaining of the hydrogen atom. As used herein, the term “or” is not an exclusive term, e.g., “A or B” would include A, B, or A and B.

As used herein, “hydrogen substitution (—H)” may include “deuterium substitution (-D)” or “tritium substitution (-T).” For example, any hydrogen in any compound described herein may be protium, deuterium, or tritium (e.g., based on natural or artificial substitution).

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzooxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to some example embodiments is described.

A compound for an organic optoelectronic device according to some example embodiments is represented by Chemical Formula 1 or Chemical Formula 2.

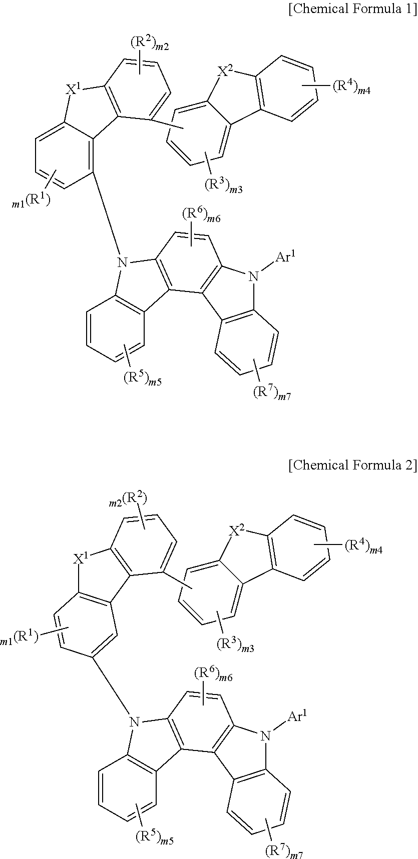

In Chemical Formula 1 and Chemical Formula 2, X¹ and X² may each independently be, e.g., O or S.

R¹ to R⁷ may each independently be or include, e.g., hydrogen, deuterium, a cyano group, a substituted or unsubstituted C1 to C10 alkyl group, a substituted or unsubstituted C1 to C10 alkylsilyl group, a substituted or unsubstituted C3 to C20 cycloalkyl group, or a substituted or unsubstituted C6 to C20 aryl group.

Ar¹ may be, e.g., a substituted or unsubstituted C6 to C20 aryl group.

m1 to m3 may each independently be, e.g., an integer of 1 to 3.

m4, m5, and m7 may each independently be an integer of 1 to 4.

m6 may be, e.g., an integer of 1 or 2.

The compound represented by Chemical Formula 1 or Chemical Formula 2 has an indolocarbazole core fused with benzocarbazole at the 3rd and 4th positions of carbazole, and has a 1-dibenzofuranyl group (or 1-dibenzothiophenyl group) or a 2-dibenzofuranyl group (or 2-dibenzothiophenyl group) as an N-substituent of the indolocarbazole, and another dibenzofuranyl group (or dibenzothiophenyl group) at the 9th position of the 1-dibenzofuranyl group (or 1-dibenzothiophenyl group) or 2-dibenzothiophenyl group.

When the indolocarbazole core is fused with benzocarbazole at 3rd and 4th positions of the carbazole, the driving voltage may be lowered when applied to an organic light emitting diode and by substituting with a 1-dibenzofuranyl group (or a 1-dibenzothiophenyl group) or a 2-dibenzofuranyl group (or a 2-dibenzothiophenyl group), ΔEst may be reduced and high-efficiency characteristics may be exhibited.

In an implementation, when another dibenzofuranyl group (or dibenzothiophenyl group) is present at the 9th position, the stability of the substituted 1-dibenzofuranyl group (or 1-dibenzothiophenyl group) or 2-dibenzofuranyl group (or 2-dibenzothiophenyl group) increases, so that a long life-span characteristic may be implemented.

In an implementation, when another dibenzofuranyl group (or dibenzothiophenyl group) is present at the 9th position, the compound has a lower LUMO energy level compared to when it has another substituent, such as a carbazolyl group, and due to the low LUMO energy level, the LUMO region may be extended to the terminal dibenzofuranyl group (or dibenzothiophenyl group). This may help allow for the implementation of an organic light emitting diode with improved efficiency and life-span characteristics by appropriately maintaining charge balance when used with a host having electron characteristics of a specific structure.

In an implementation, m1 may be 2 or 3, and each R¹ may be the same or different from each other.

In an implementation, m2 may be 2 or 3, and each R² may be the same or different from each other.

In an implementation, m3 may be 2 or 3, and each R³ may be the same or different from each other.

In an implementation, m4 may be 2, 3, or 4, and each R⁴ may be the same or different from each other.

In an implementation, m5 may be 2, 3, or 4, and each R⁵ may be the same or different from each other.

In an implementation, m6 may be 2, and each R⁶ may be the same or different from each other.

In an implementation, m7 may be 2, 3, or 4, and each R⁷ may be the same or different from each other.

In an implementation, Chemical Formula 1 may be, e.g., represented by one of Chemical Formula 1A to Chemical Formula 1D.

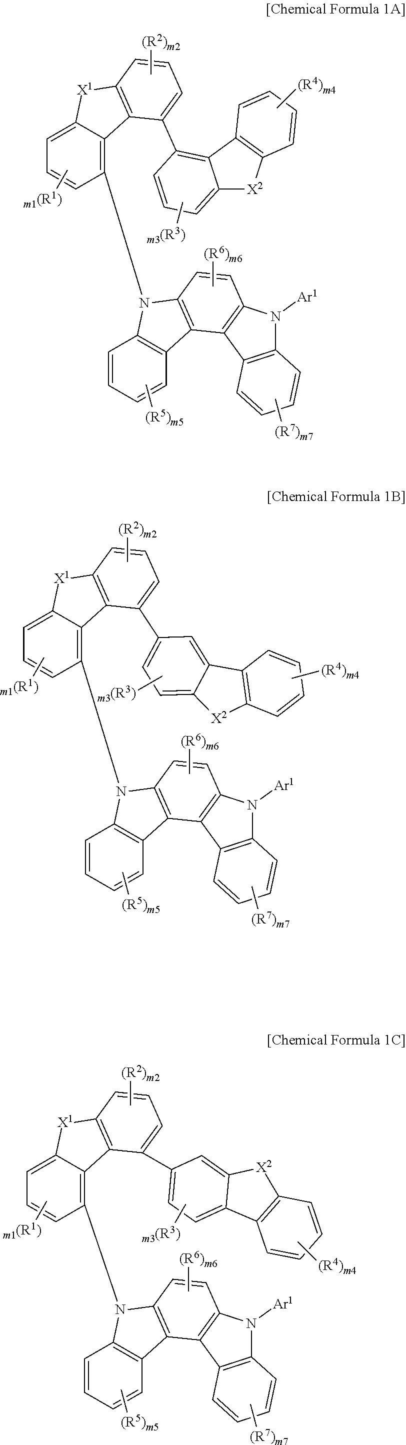

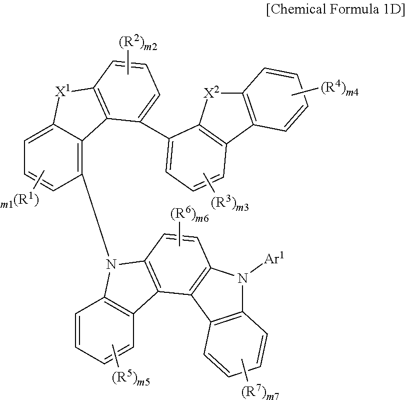

In Chemical Formula 1A to Chemical Formula 1D, X¹ and X², R¹ to R⁷, Ar¹, and m1 to m7 may be defined the same as those described above.

In an implementation, Chemical Formula 1 may be, e.g., represented by one of Chemical Formula 1A, Chemical Formula 1B, and Chemical Formula 1D.

In an implementation, Chemical Formula 2 may be, e.g., represented by one of Chemical Formula 2A to Chemical Formula 2D.

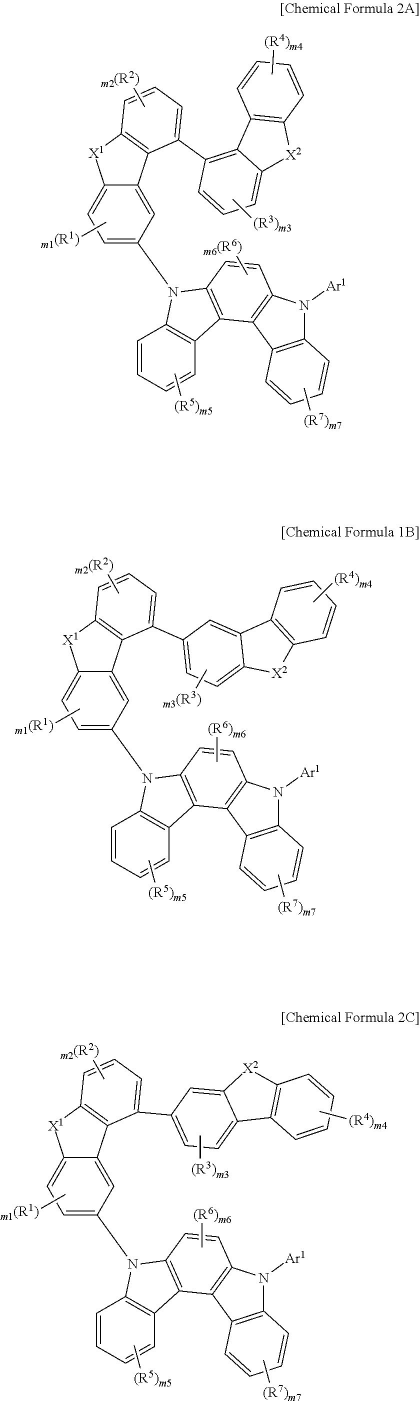

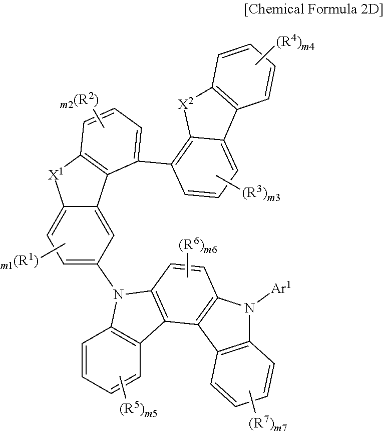

In Chemical Formula 2A to Chemical Formula 2D, X¹ and X², R¹ to R⁷, Ar¹, and m1 to m7 may be defined the same as those described above.

In an implementation, Ar¹ may be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted fluorenyl group, or a substituted or unsubstituted triphenylene group.

In an implementation, R¹ to R⁷ may each independently be, e.g., hydrogen, deuterium, a cyano group, a substituted or unsubstituted C1 to C5 alkyl group, a substituted or unsubstituted C1 to C5 alkylsilyl group, a substituted or unsubstituted C3 to C10 cycloalkyl group, or a substituted or unsubstituted C6 to C12 aryl group.

In an implementation, R¹ to R⁷ may each independently be, e.g., hydrogen, deuterium, a cyano group, a substituted or unsubstituted tert-butyl group, a substituted or unsubstituted trimethylsilyl group, a substituted or unsubstituted cyclopropyl group, a substituted or unsubstituted cyclobutyl group, a substituted or unsubstituted cyclopentyl group, a substituted or unsubstituted cyclohexyl group, a substituted or unsubstituted adamantyl group, a substituted or unsubstituted phenyl group, a substituted or unsubstituted biphenyl group, or a substituted or unsubstituted naphthyl group.

In an implementation, R¹ to R⁷ may each independently be, e.g., hydrogen, deuterium, a cyano group, a substituted or unsubstituted tert-butyl group, a substituted or unsubstituted cyclopropyl group, a substituted or unsubstituted adamantyl group, or a substituted or unsubstituted phenyl group.

In an implementation, the compound represented by Chemical Formula 1 or Chemical Formula 2 may be, e.g., a compound of Group 1, Group 1-1, or Group 1-2.

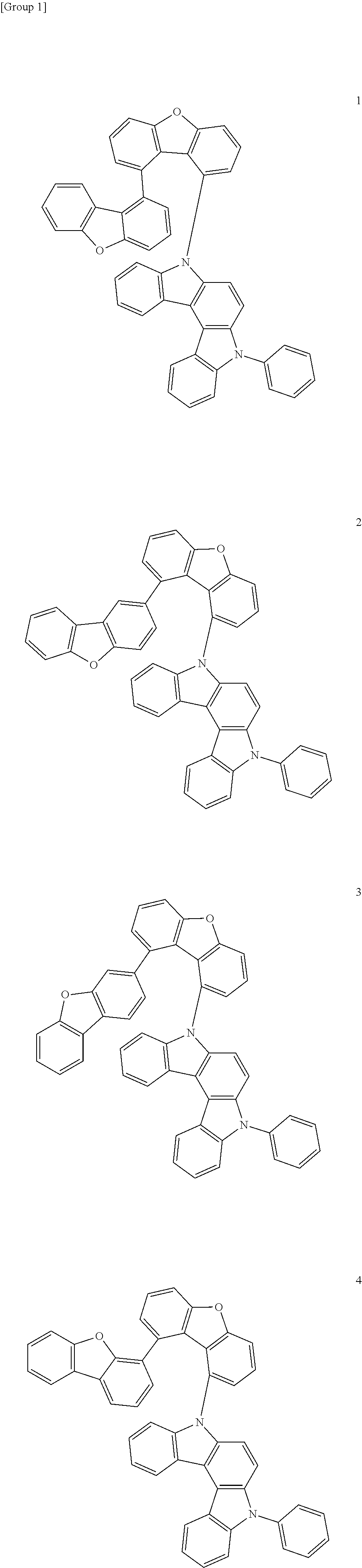 [SMILES: c1ccc(-n2c3ccccc3c3c4c5ccccc5n(-c5cccc6oc7cccc(-c8cccc9oc%10ccccc%10c89)c7c56)c4ccc32)cc1 ; c1ccc(-n2c3ccccc3c3c4c5ccccc5n(-c5cccc6oc7cccc(-c8ccc9oc%10ccccc%10c9c8)c7c56)c4ccc32)cc1 ; c1ccc(-n2c3ccccc3c3c4c5ccccc5n(-c5cccc6oc7cccc(-c8ccc9c(c8)oc8ccccc89)c7c56)c4ccc32)cc1 ; c1ccc(-n2c3ccccc3c3c4c5ccccc5n(-c5cccc6oc7cccc(-c8cccc9c8oc8ccccc89)c7c56)c4ccc32)cc1]

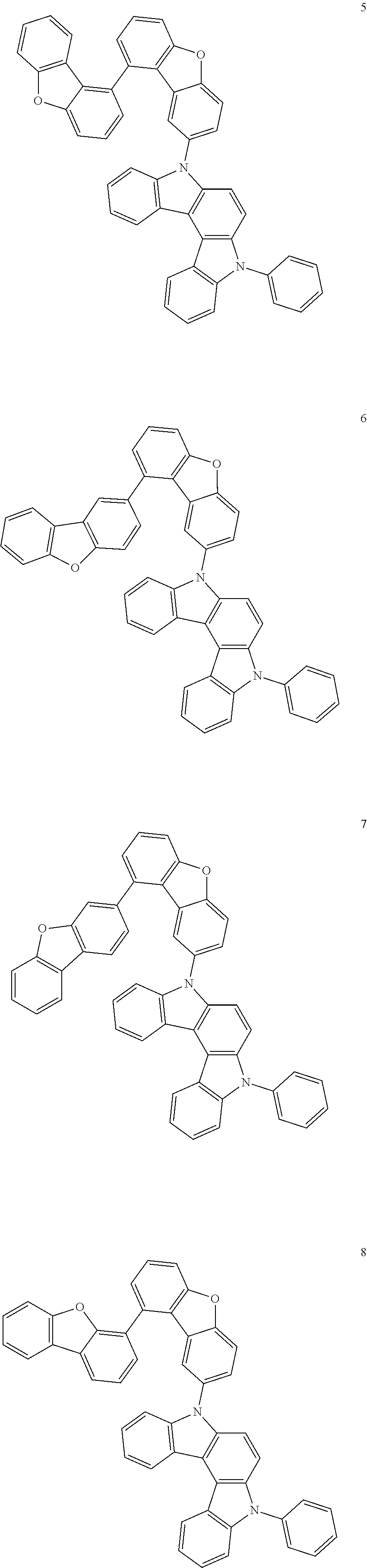 [SMILES: c1ccc(-n2c3ccccc3c3c4c5ccccc5n(-c5ccc6oc7cccc(-c8cccc9oc%10ccccc%10c89)c7c6c5)c4ccc32)cc1 ; c1ccc(-n2c3ccccc3c3c4c5ccccc5n(-c5ccc6oc7cccc(-c8ccc9oc%10ccccc%10c9c8)c7c6c5)c4ccc32)cc1 ; c1ccc(-n2c3ccccc3c3c4c5ccccc5n(-c5ccc6oc7cccc(-c8ccc9c(c8)oc8ccccc89)c7c6c5)c4ccc32)cc1 ; c1ccc(-n2c3ccccc3c3c4c5ccccc5n(-c5ccc6oc7cccc(-c8cccc9c8oc8ccccc89)c7c6c5)c4ccc32)cc1]

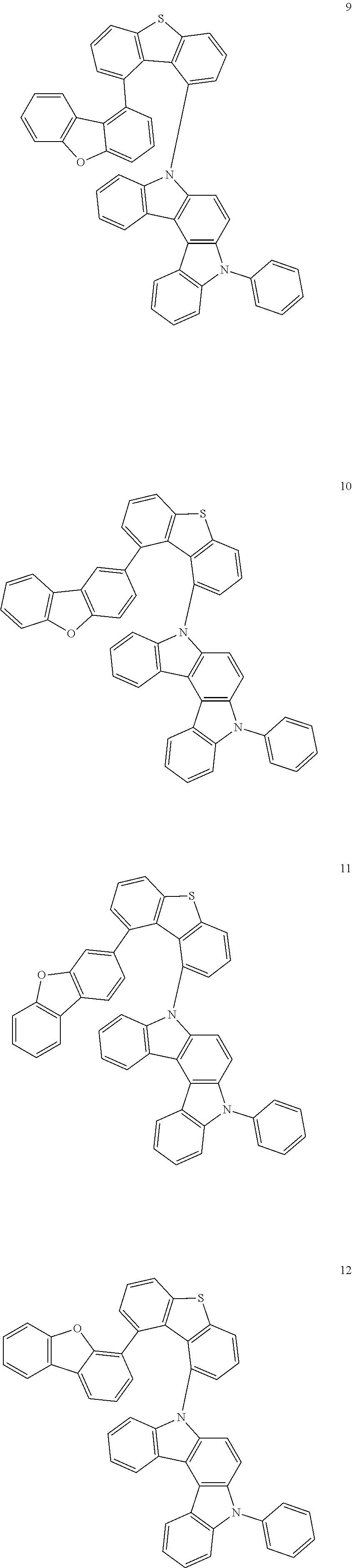 [SMILES: c1ccc(-n2c3ccccc3c3c4c5ccccc5n(-c5cccc6sc7cccc(-c8cccc9oc%10ccccc%10c89)c7c56)c4ccc32)cc1 ; c1ccc(-n2c3ccccc3c3c4c5ccccc5n(-c5cccc6sc7cccc(-c8ccc9oc%10ccccc%10c9c8)c7c56)c4ccc32)cc1 ; c1ccc(-n2c3ccccc3c3c4c5ccccc5n(-c5cccc6sc7cccc(-c8ccc9c(c8)oc8ccccc89)c7c56)c4ccc32)cc1 ; c1ccc(-n2c3ccccc3c3c4c5ccccc5n(-c5cccc6sc7cccc(-c8cccc9c8oc8ccccc89)c7c56)c4ccc32)cc1]

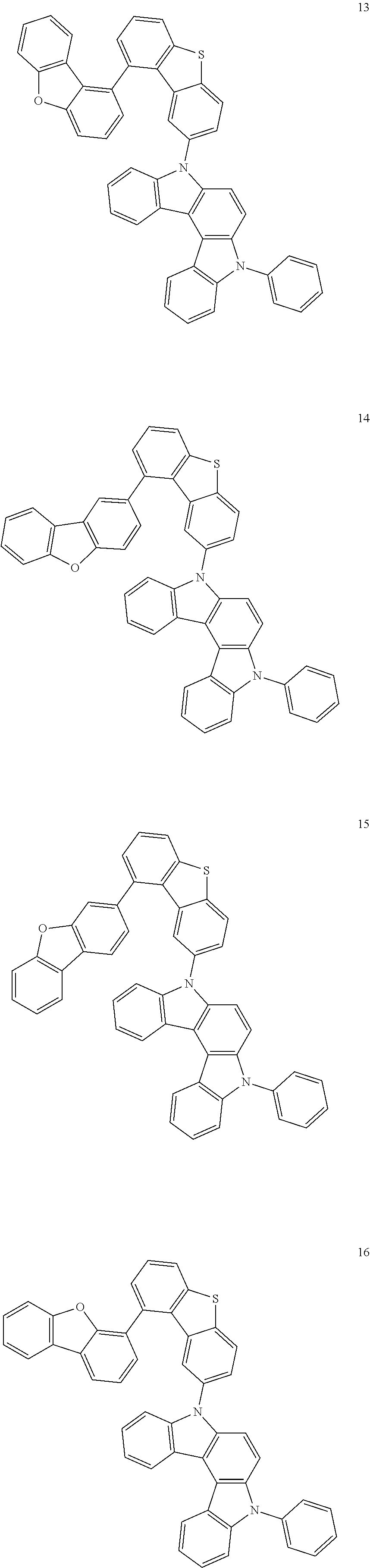 [SMILES: c1ccc(-n2c3ccccc3c3c4c5ccccc5n(-c5ccc6sc7cccc(-c8cccc9oc%10ccccc%10c89)c7c6c5)c4ccc32)cc1 ; c1ccc(-n2c3ccccc3c3c4c5ccccc5n(-c5ccc6sc7cccc(-c8ccc9oc%10ccccc%10c9c8)c7c6c5)c4ccc32)cc1 ; c1ccc(-n2c3ccccc3c3c4c5ccccc5n(-c5ccc6sc7cccc(-c8ccc9c(c8)oc8ccccc89)c7c6c5)c4ccc32)cc1 ; c1ccc(-n2c3ccccc3c3c4c5ccccc5n(-c5ccc6sc7cccc(-c8cccc9c8oc8ccccc89)c7c6c5)c4ccc32)cc1]

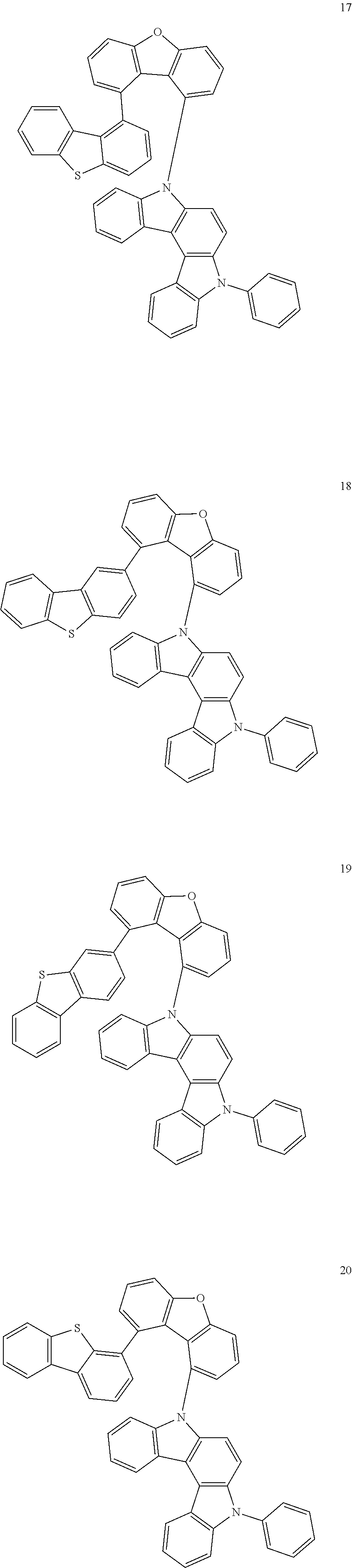 [SMILES: c1ccc(-n2c3ccccc3c3c4c5ccccc5n(-c5cccc6oc7cccc(-c8cccc9sc%10ccccc%10c89)c7c56)c4ccc32)cc1 ; c1ccc(-n2c3ccccc3c3c4c5ccccc5n(-c5cccc6oc7cccc(-c8ccc9sc%10ccccc%10c9c8)c7c56)c4ccc32)cc1 ; c1ccc(-n2c3ccccc3c3c4c5ccccc5n(-c5cccc6oc7cccc(-c8ccc9c(c8)sc8ccccc89)c7c56)c4ccc32)cc1 ; c1ccc(-n2c3ccccc3c3c4c5ccccc5n(-c5cccc6oc7cccc(-c8cccc9c8sc8ccccc89)c7c56)c4ccc32)cc1]

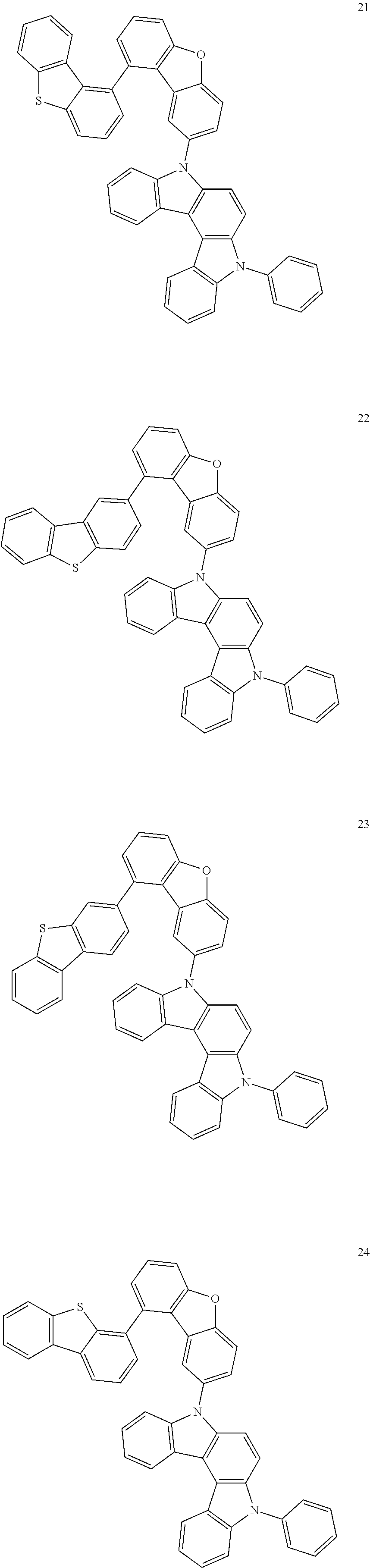 [SMILES: c1ccc(-n2c3ccccc3c3c4c5ccccc5n(-c5ccc6oc7cccc(-c8cccc9sc%10ccccc%10c89)c7c6c5)c4ccc32)cc1 ; c1ccc(-n2c3ccccc3c3c4c5ccccc5n(-c5ccc6oc7cccc(-c8ccc9sc%10ccccc%10c9c8)c7c6c5)c4ccc32)cc1 ; c1ccc(-n2c3ccccc3c3c4c5ccccc5n(-c5ccc6oc7cccc(-c8ccc9c(c8)sc8ccccc89)c7c6c5)c4ccc32)cc1 ; c1ccc(-n2c3ccccc3c3c4c5ccccc5n(-c5ccc6oc7cccc(-c8cccc9c8sc8ccccc89)c7c6c5)c4ccc32)cc1]

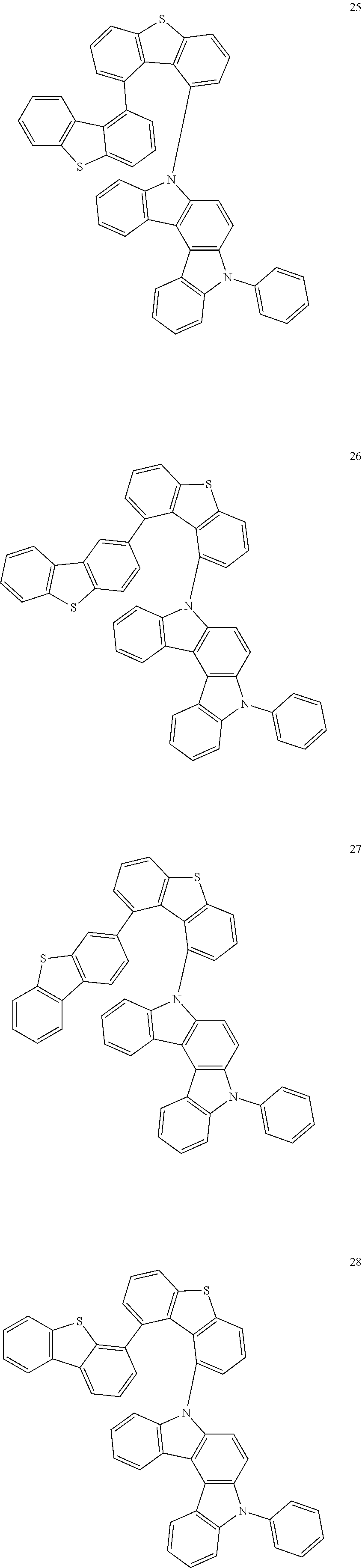 [SMILES: c1ccc(-n2c3ccccc3c3c4c5ccccc5n(-c5cccc6sc7cccc(-c8cccc9sc%10ccccc%10c89)c7c56)c4ccc32)cc1 ; c1ccc(-n2c3ccccc3c3c4c5ccccc5n(-c5cccc6sc7cccc(-c8ccc9sc%10ccccc%10c9c8)c7c56)c4ccc32)cc1 ; c1ccc(-n2c3ccccc3c3c4c5ccccc5n(-c5cccc6sc7cccc(-c8ccc9c(c8)sc8ccccc89)c7c56)c4ccc32)cc1 ; c1ccc(-n2c3ccccc3c3c4c5ccccc5n(-c5cccc6sc7cccc(-c8cccc9c8sc8ccccc89)c7c56)c4ccc32)cc1]

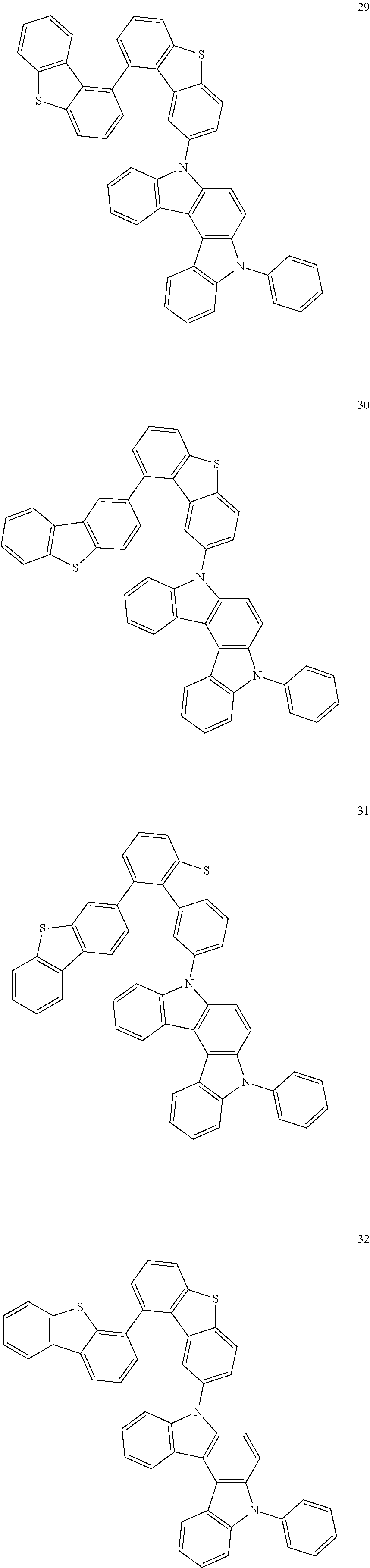 [SMILES: c1ccc(-n2c3ccccc3c3c4c5ccccc5n(-c5ccc6sc7cccc(-c8cccc9sc%10ccccc%10c89)c7c6c5)c4ccc32)cc1 ; c1ccc(-n2c3ccccc3c3c4c5ccccc5n(-c5ccc6sc7cccc(-c8ccc9sc%10ccccc%10c9c8)c7c6c5)c4ccc32)cc1 ; c1ccc(-n2c3ccccc3c3c4c5ccccc5n(-c5ccc6sc7cccc(-c8ccc9c(c8)sc8ccccc89)c7c6c5)c4ccc32)cc1 ; c1ccc(-n2c3ccccc3c3c4c5ccccc5n(-c5ccc6sc7cccc(-c8cccc9c8sc8ccccc89)c7c6c5)c4ccc32)cc1]

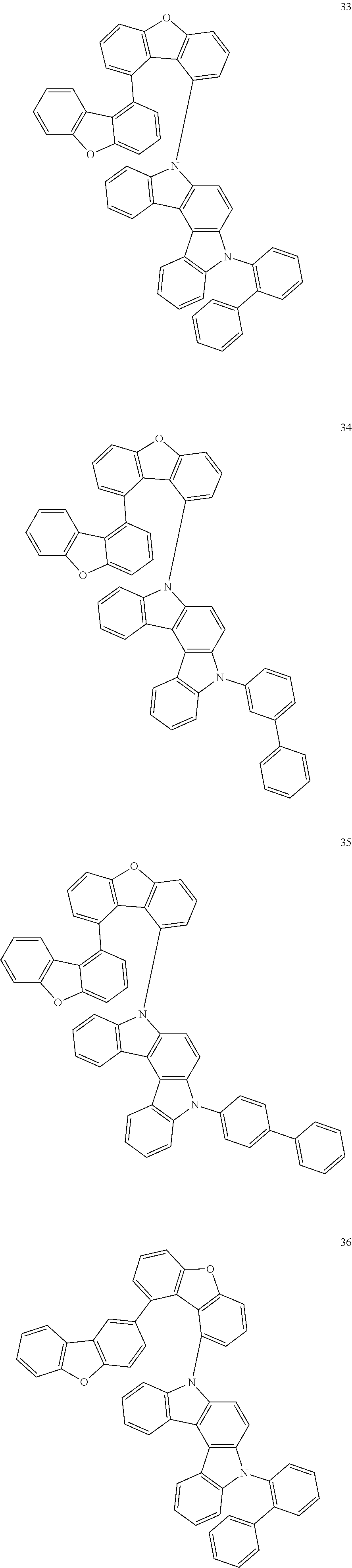 [SMILES: c1ccc(-c2ccccc2-n2c3ccccc3c3c4c5ccccc5n(-c5cccc6oc7cccc(-c8cccc9oc%10ccccc%10c89)c7c56)c4ccc32)cc1 ; c1ccc(-c2cccc(-n3c4ccccc4c4c5c6ccccc6n(-c6cccc7oc8cccc(-c9cccc%10oc%11ccccc%11c9%10)c8c67)c5ccc43)c2)cc1 ; c1ccc(-c2ccc(-n3c4ccccc4c4c5c6ccccc6n(-c6cccc7oc8cccc(-c9cccc%10oc%11ccccc%11c9%10)c8c67)c5ccc43)cc2)cc1 ; c1ccc(-c2ccccc2-n2c3ccccc3c3c4c5ccccc5n(-c5cccc6oc7cccc(-c8ccc9oc%10ccccc%10c9c8)c7c56)c4ccc32)cc1]

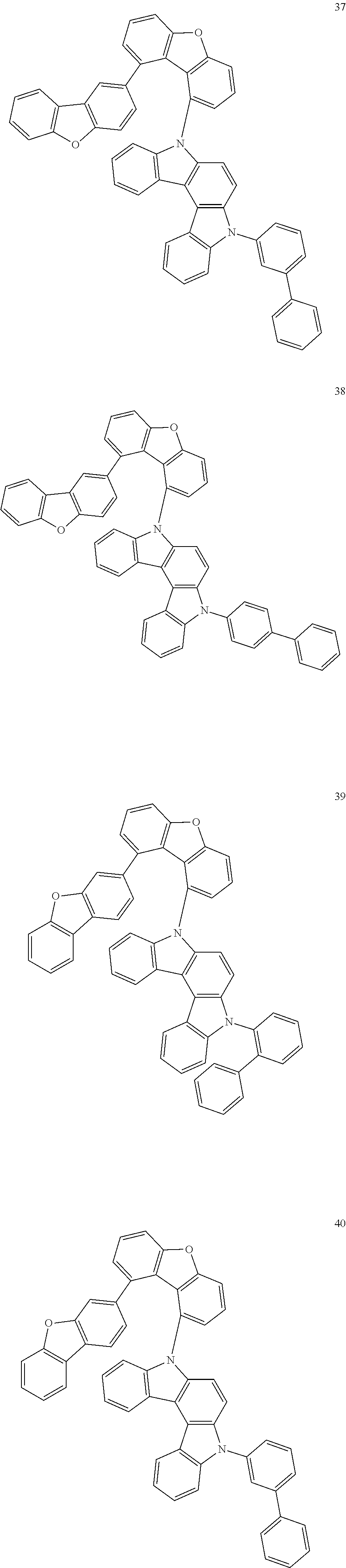 [SMILES: c1ccc(-c2cccc(-n3c4ccccc4c4c5c6ccccc6n(-c6cccc7oc8cccc(-c9ccc%10oc%11ccccc%11c%10c9)c8c67)c5ccc43)c2)cc1 ; c1ccc(-c2ccc(-n3c4ccccc4c4c5c6ccccc6n(-c6cccc7oc8cccc(-c9ccc%10oc%11ccccc%11c%10c9)c8c67)c5ccc43)cc2)cc1 ; c1ccc(-c2ccccc2-n2c3ccccc3c3c4c5ccccc5n(-c5cccc6oc7cccc(-c8ccc9c(c8)oc8ccccc89)c7c56)c4ccc32)cc1 ; c1ccc(-c2cccc(-n3c4ccccc4c4c5c6ccccc6n(-c6cccc7oc8cccc(-c9ccc%10c(c9)oc9ccccc9%10)c8c67)c5ccc43)c2)cc1]

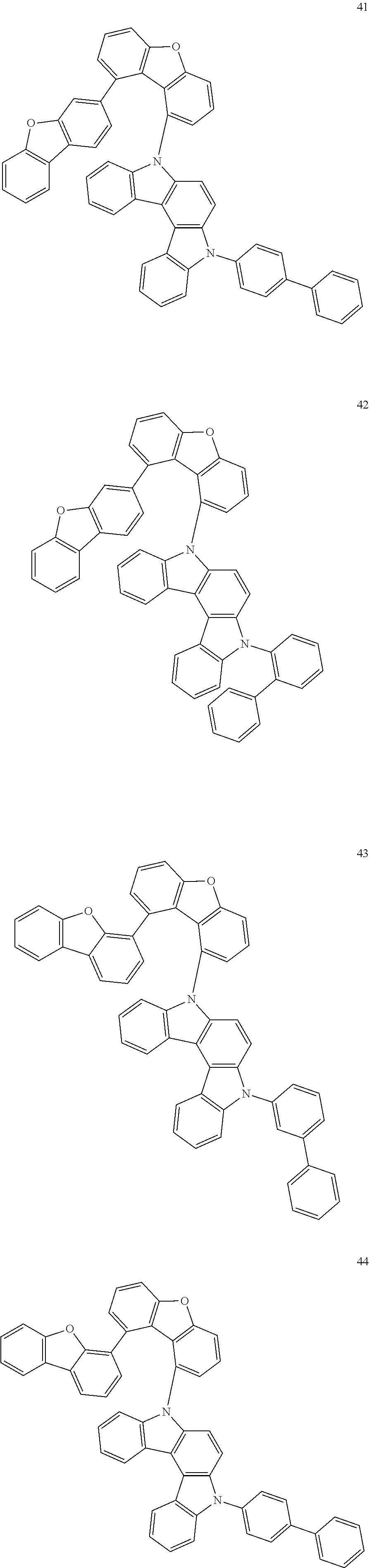 [SMILES: c1ccc(-c2ccc(-n3c4ccccc4c4c5c6ccccc6n(-c6cccc7oc8cccc(-c9ccc%10c(c9)oc9ccccc9%10)c8c67)c5ccc43)cc2)cc1 ; c1ccc(-c2ccccc2-n2c3ccccc3c3c4c5ccccc5n(-c5cccc6oc7cccc(-c8ccc9c(c8)oc8ccccc89)c7c56)c4ccc32)cc1 ; c1ccc(-c2cccc(-n3c4ccccc4c4c5c6ccccc6n(-c6cccc7oc8cccc(-c9cccc%10c9oc9ccccc9%10)c8c67)c5ccc43)c2)cc1 ; c1ccc(-c2ccc(-n3c4ccccc4c4c5c6ccccc6n(-c6cccc7oc8cccc(-c9cccc%10c9oc9ccccc9%10)c8c67)c5ccc43)cc2)cc1]

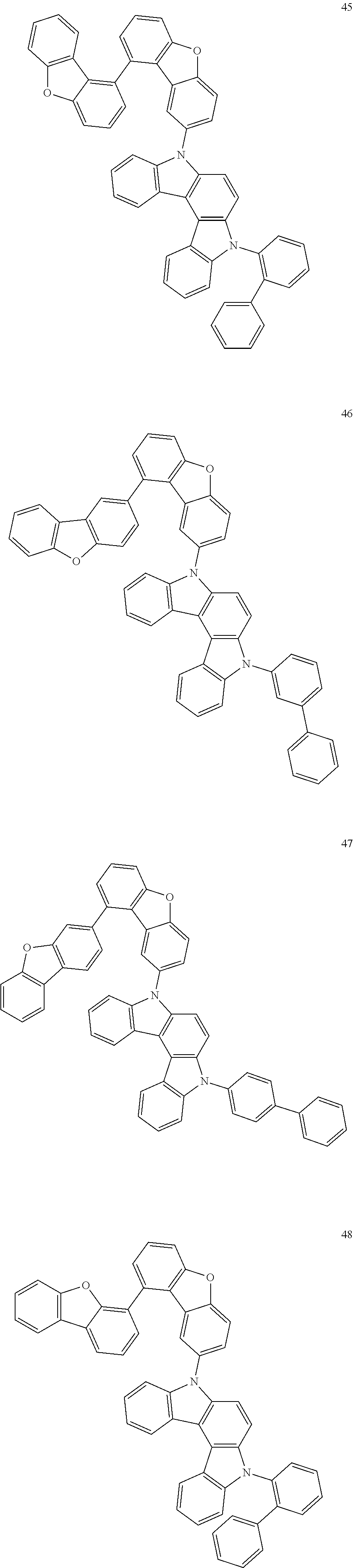

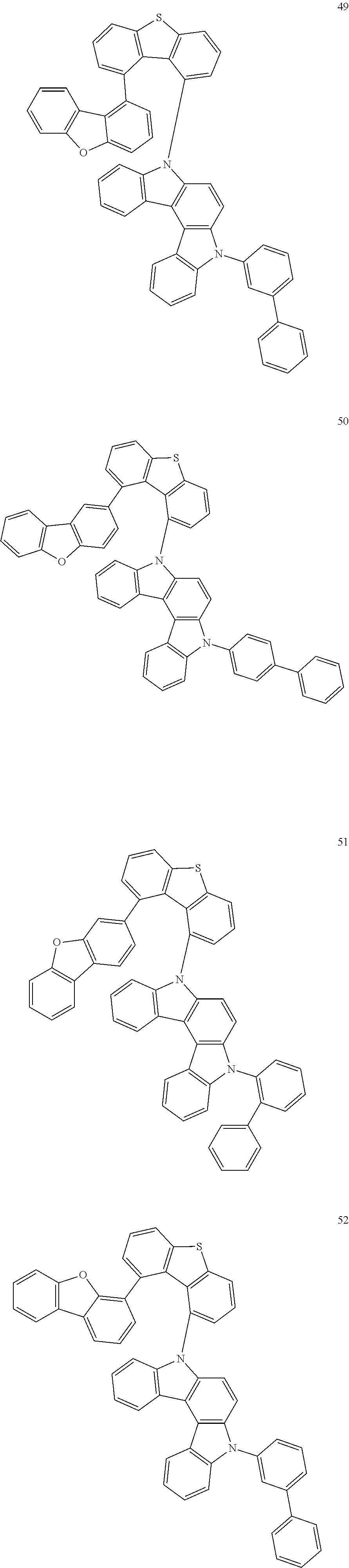 [SMILES: c1ccc(-c2cccc(-n3c4ccccc4c4c5c6ccccc6n(-c6cccc7sc8cccc(-c9cccc%10oc%11ccccc%11c9%10)c8c67)c5ccc43)c2)cc1 ; c1ccc(-c2ccc(-n3c4ccccc4c4c5c6ccccc6n(-c6cccc7sc8cccc(-c9ccc%10oc%11ccccc%11c%10c9)c8c67)c5ccc43)cc2)cc1 ; c1ccc(-c2ccccc2-n2c3ccccc3c3c4c5ccccc5n(-c5cccc6sc7cccc(-c8ccc9c(c8)oc8ccccc89)c7c56)c4ccc32)cc1 ; c1ccc(-c2cccc(-n3c4ccccc4c4c5c6ccccc6n(-c6cccc7sc8cccc(-c9cccc%10c9oc9ccccc9%10)c8c67)c5ccc43)c2)cc1]

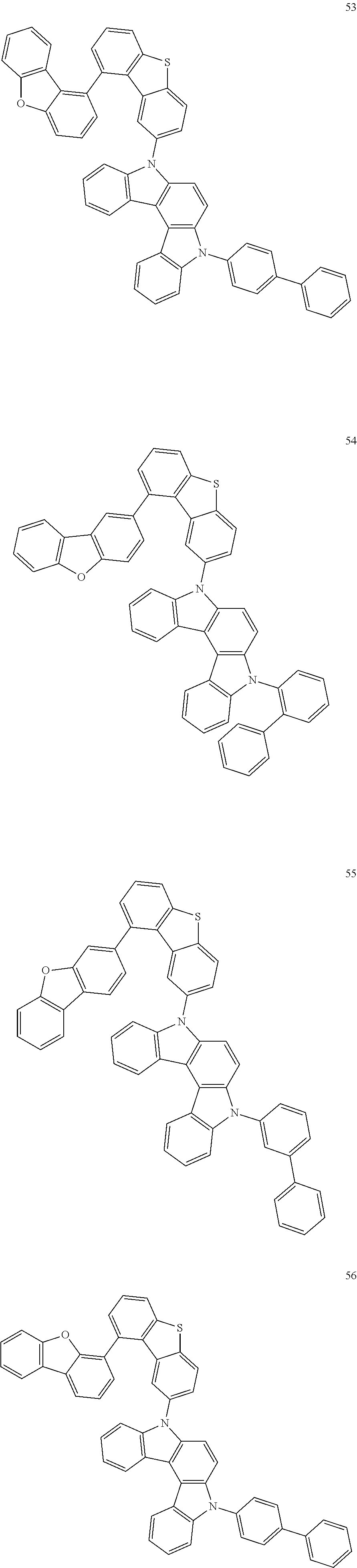

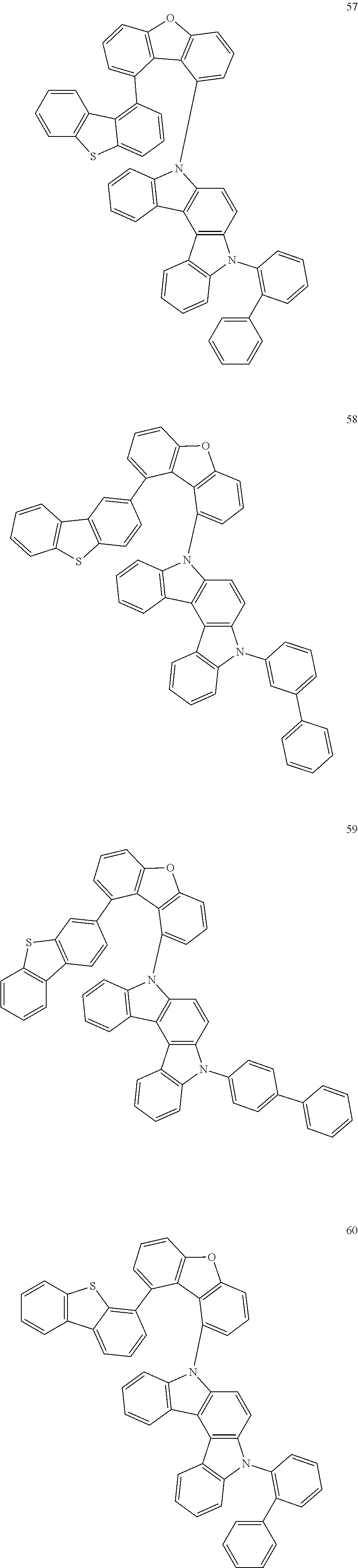 [SMILES: c1ccc(-c2ccccc2-n2c3ccccc3c3c4c5ccccc5n(-c5cccc6oc7cccc(-c8cccc9sc%10ccccc%10c89)c7c56)c4ccc32)cc1 ; c1ccc(-c2cccc(-n3c4ccccc4c4c5c6ccccc6n(-c6cccc7oc8cccc(-c9ccc%10sc%11ccccc%11c%10c9)c8c67)c5ccc43)c2)cc1 ; c1ccc(-c2ccc(-n3c4ccccc4c4c5c6ccccc6n(-c6cccc7oc8cccc(-c9ccc%10c(c9)sc9ccccc9%10)c8c67)c5ccc43)cc2)cc1 ; c1ccc(-c2ccccc2-n2c3ccccc3c3c4c5ccccc5n(-c5cccc6oc7cccc(-c8cccc9c8sc8ccccc89)c7c56)c4ccc32)cc1]

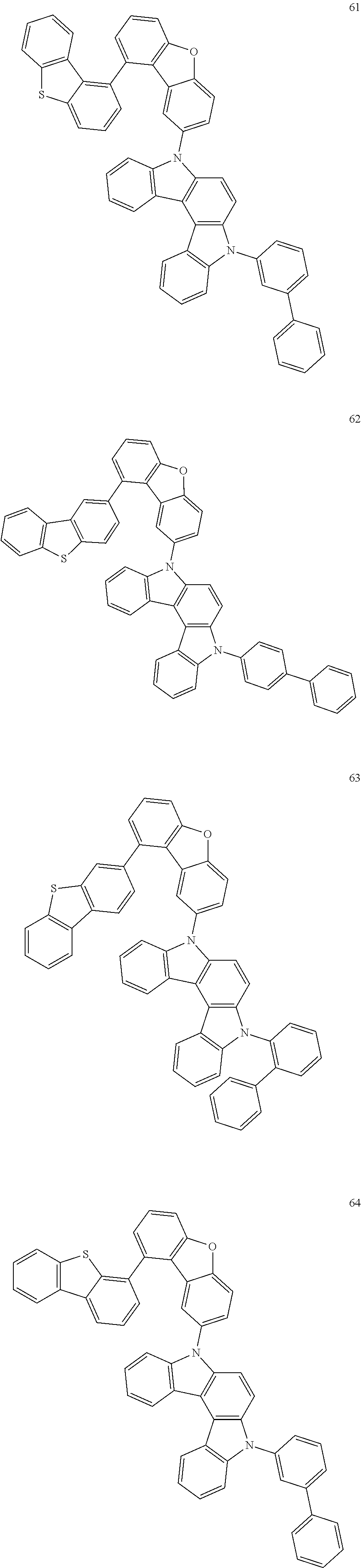 [SMILES: c1ccc(-c2cccc(-n3c4ccccc4c4c5c6ccccc6n(-c6ccc7oc8cccc(-c9cccc%10sc%11ccccc%11c9%10)c8c7c6)c5ccc43)c2)cc1 ; c1ccc(-c2ccc(-n3c4ccccc4c4c5c6ccccc6n(-c6ccc7oc8cccc(-c9ccc%10sc%11ccccc%11c%10c9)c8c7c6)c5ccc43)cc2)cc1 ; c1ccc(-c2ccccc2-n2c3ccccc3c3c4c5ccccc5n(-c5ccc6oc7cccc(-c8ccc9c(c8)sc8ccccc89)c7c6c5)c4ccc32)cc1 ; c1ccc(-c2cccc(-n3c4ccccc4c4c5c6ccccc6n(-c6ccc7oc8cccc(-c9cccc%10c9sc9ccccc9%10)c8c7c6)c5ccc43)c2)cc1]

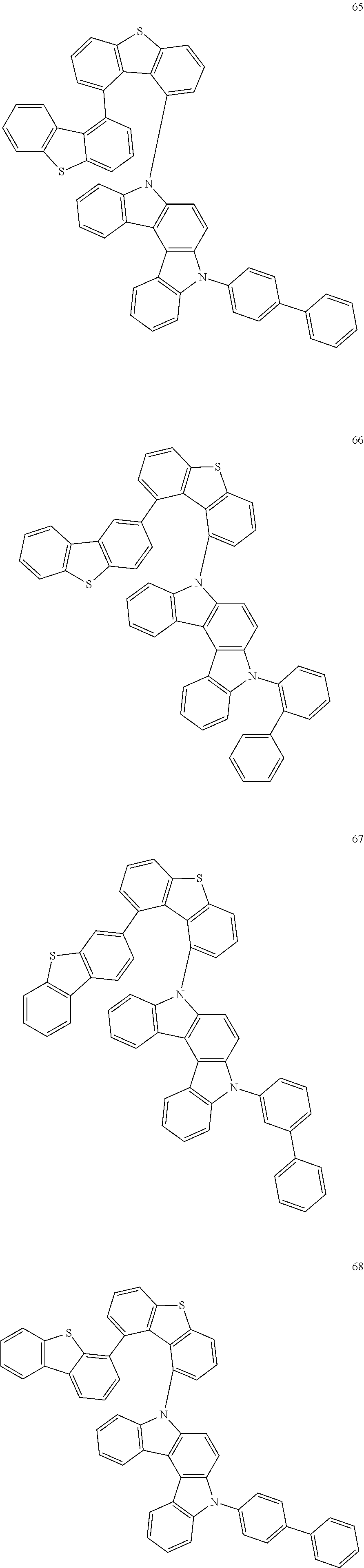

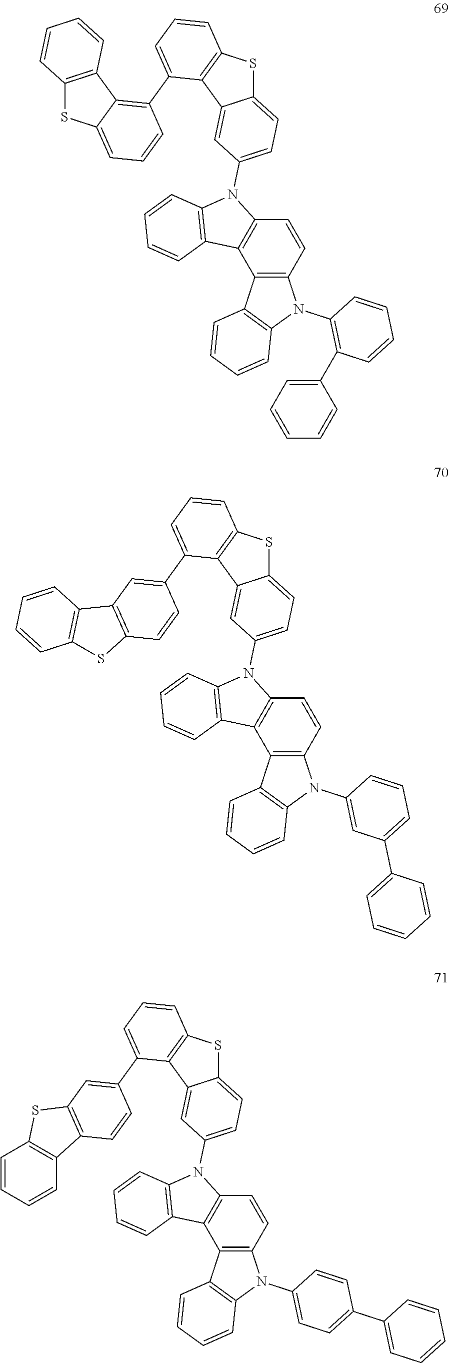 [SMILES: c1ccc(-c2ccccc2-n2c3ccccc3c3c4c5ccccc5n(-c5ccc6sc7cccc(-c8cccc9sc%10ccccc%10c89)c7c6c5)c4ccc32)cc1 ; c1ccc(-c2cccc(-n3c4ccccc4c4c5c6ccccc6n(-c6ccc7sc8cccc(-c9ccc%10sc%11ccccc%11c%10c9)c8c7c6)c5ccc43)c2)cc1 ; c1ccc(-c2ccc(-n3c4ccccc4c4c5c6ccccc6n(-c6ccc7sc8cccc(-c9ccc%10c(c9)sc9ccccc9%10)c8c7c6)c5ccc43)cc2)cc1]

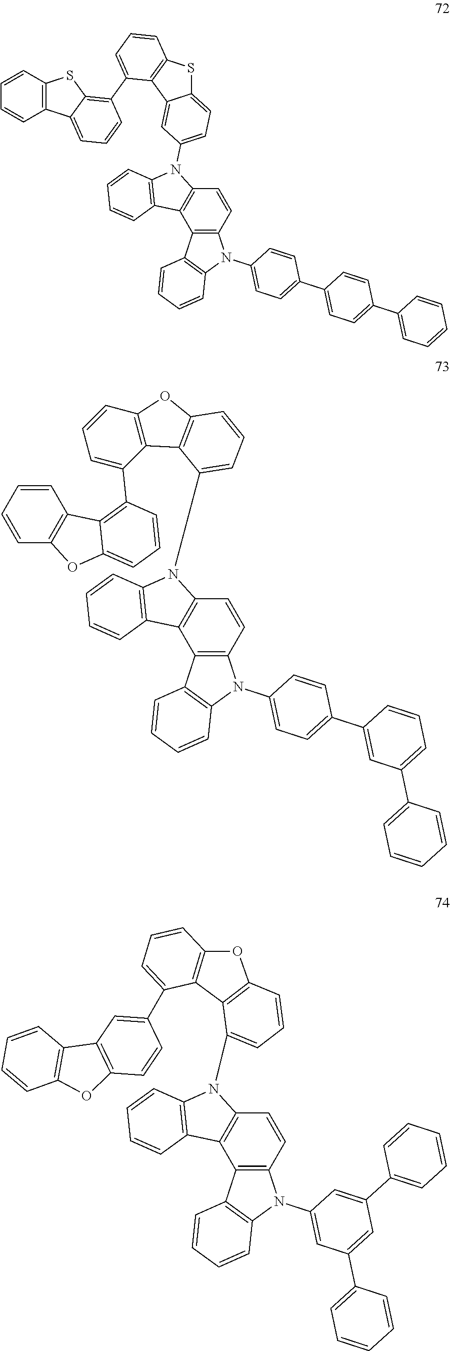 [SMILES: c1ccc(-c2ccc(-c3ccc(-n4c5ccccc5c5c6c7ccccc7n(-c7ccc8sc9cccc(-c%10cccc%11c%10sc%10ccccc%10%11)c9c8c7)c6ccc54)cc3)cc2)cc1 ; c1ccc(-c2cccc(-c3ccc(-n4c5ccccc5c5c6c7ccccc7n(-c7cccc8oc9cccc(-c%10cccc%11oc%12ccccc%12c%10%11)c9c78)c6ccc54)cc3)c2)cc1 ; c1ccc(-c2cc(-c3ccccc3)cc(-n3c4ccccc4c4c5c6ccccc6n(-c6cccc7oc8cccc(-c9ccc%10oc%11ccccc%11c%10c9)c8c67)c5ccc43)c2)cc1]

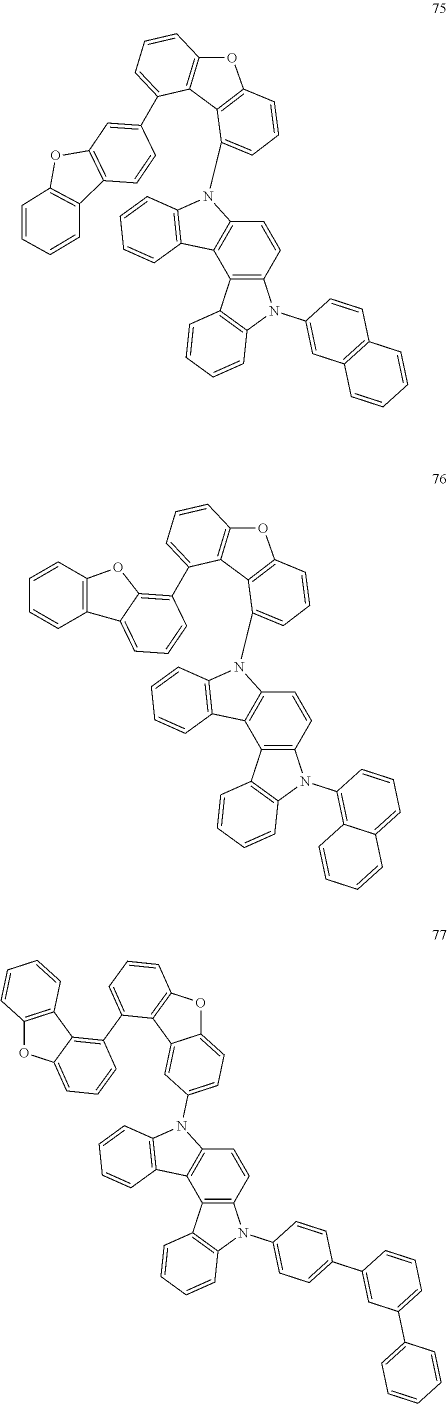 [SMILES: c1ccc2cc(-n3c4ccccc4c4c5c6ccccc6n(-c6cccc7oc8cccc(-c9ccc%10c(c9)oc9ccccc9%10)c8c67)c5ccc43)ccc2c1 ; c1ccc2c(-n3c4ccccc4c4c5c6ccccc6n(-c6cccc7oc8cccc(-c9cccc%10c9oc9ccccc9%10)c8c67)c5ccc43)cccc2c1 ; c1ccc(-c2cccc(-c3ccc(-n4c5ccccc5c5c6c7ccccc7n(-c7ccc8oc9cccc(-c%10cccc%11oc%12ccccc%12c%10%11)c9c8c7)c6ccc54)cc3)c2)cc1]

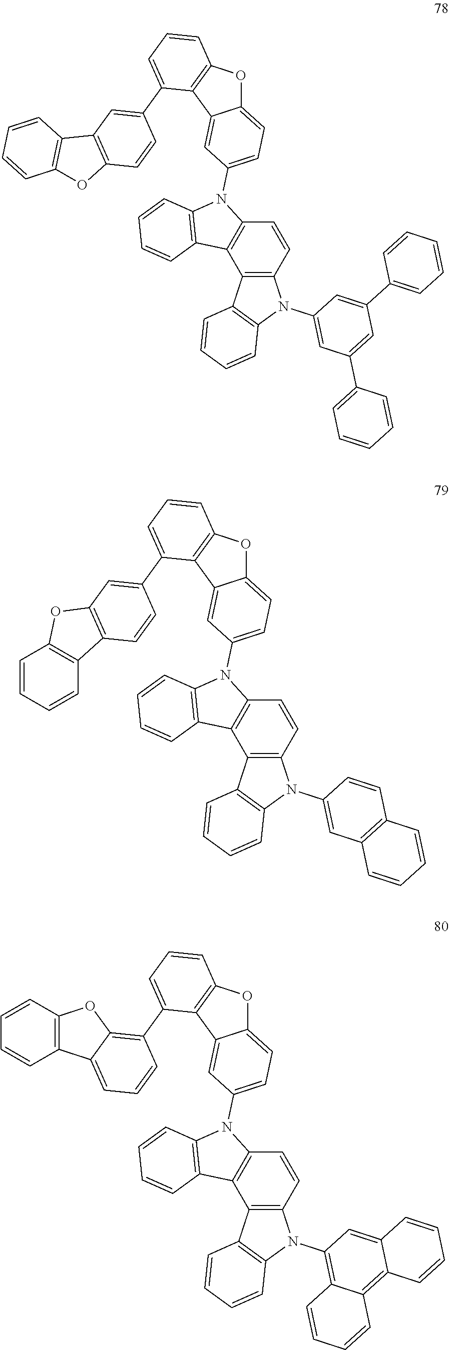 [SMILES: c1ccc(-c2cc(-c3ccccc3)cc(-n3c4ccccc4c4c5c6ccccc6n(-c6ccc7oc8cccc(-c9ccc%10oc%11ccccc%11c%10c9)c8c7c6)c5ccc43)c2)cc1 ; c1ccc2cc(-n3c4ccccc4c4c5c6ccccc6n(-c6ccc7oc8cccc(-c9ccc%10c(c9)oc9ccccc9%10)c8c7c6)c5ccc43)ccc2c1 ; c1ccc2c(c1)cc(-n1c3ccccc3c3c4c5ccccc5n(-c5ccc6oc7cccc(-c8cccc9c8oc8ccccc89)c7c6c5)c4ccc31)c1ccccc12]

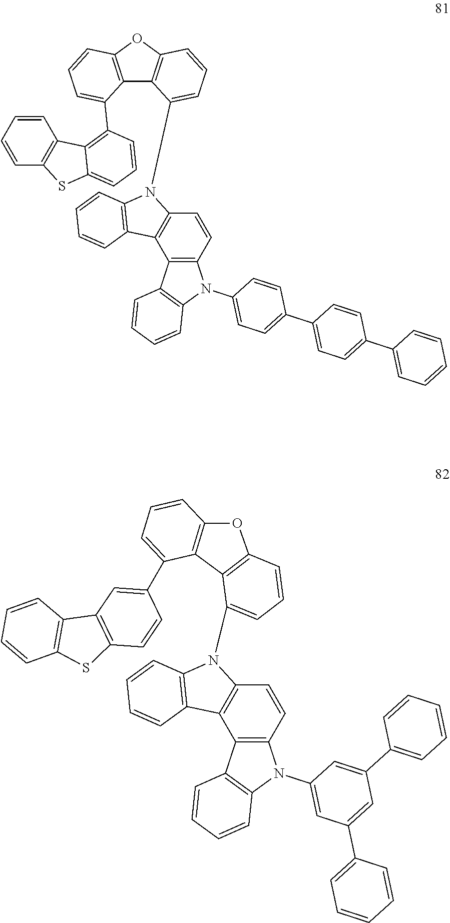 [SMILES: c1ccc(-c2ccc(-c3ccc(-n4c5ccccc5c5c6c7ccccc7n(-c7cccc8oc9cccc(-c%10cccc%11sc%12ccccc%12c%10%11)c9c78)c6ccc54)cc3)cc2)cc1 ; c1ccc(-c2cc(-c3ccccc3)cc(-n3c4ccccc4c4c5c6ccccc6n(-c6cccc7oc8cccc(-c9ccc%10sc%11ccccc%11c%10c9)c8c67)c5ccc43)c2)cc1]

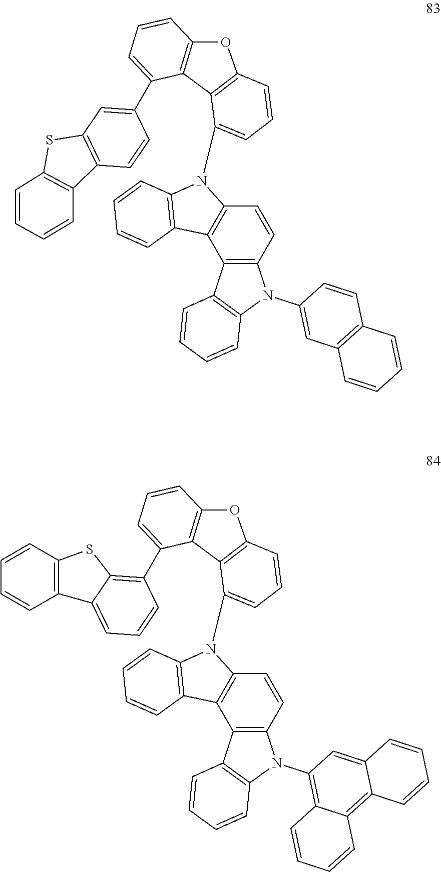 [SMILES: c1ccc2cc(-n3c4ccccc4c4c5c6ccccc6n(-c6cccc7oc8cccc(-c9ccc%10c(c9)sc9ccccc9%10)c8c67)c5ccc43)ccc2c1 ; c1ccc2c(c1)cc(-n1c3ccccc3c3c4c5ccccc5n(-c5cccc6oc7cccc(-c8cccc9c8sc8ccccc89)c7c56)c4ccc31)c1ccccc12]

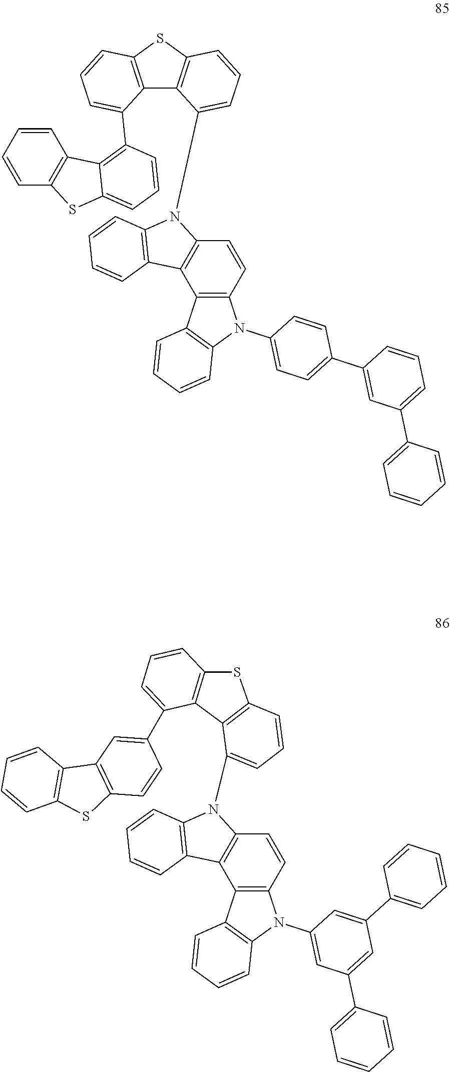 [SMILES: c1ccc(-c2cccc(-c3ccc(-n4c5ccccc5c5c6c7ccccc7n(-c7cccc8sc9cccc(-c%10cccc%11sc%12ccccc%12c%10%11)c9c78)c6ccc54)cc3)c2)cc1 ; c1ccc(-c2cc(-c3ccccc3)cc(-n3c4ccccc4c4c5c6ccccc6n(-c6cccc7sc8cccc(-c9ccc%10sc%11ccccc%11c%10c9)c8c67)c5ccc43)c2)cc1]

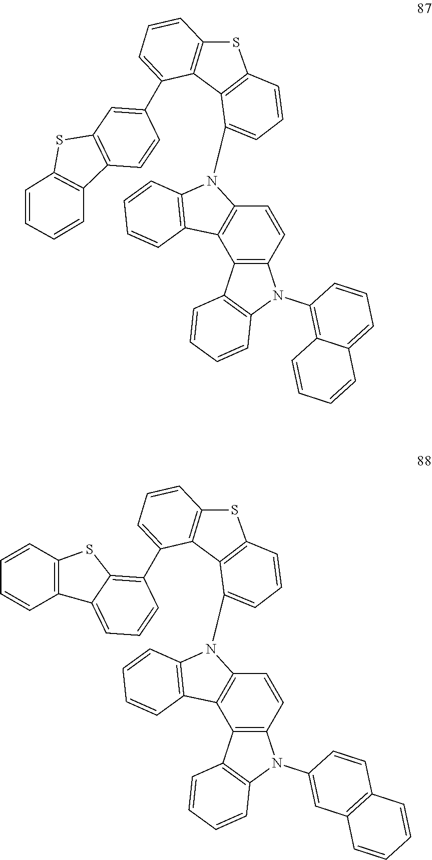 [SMILES: c1ccc2c(-n3c4ccccc4c4c5c6ccccc6n(-c6cccc7sc8cccc(-c9ccc%10c(c9)sc9ccccc9%10)c8c67)c5ccc43)cccc2c1 ; c1ccc2cc(-n3c4ccccc4c4c5c6ccccc6n(-c6cccc7sc8cccc(-c9cccc%10c9sc9ccccc9%10)c8c67)c5ccc43)ccc2c1]

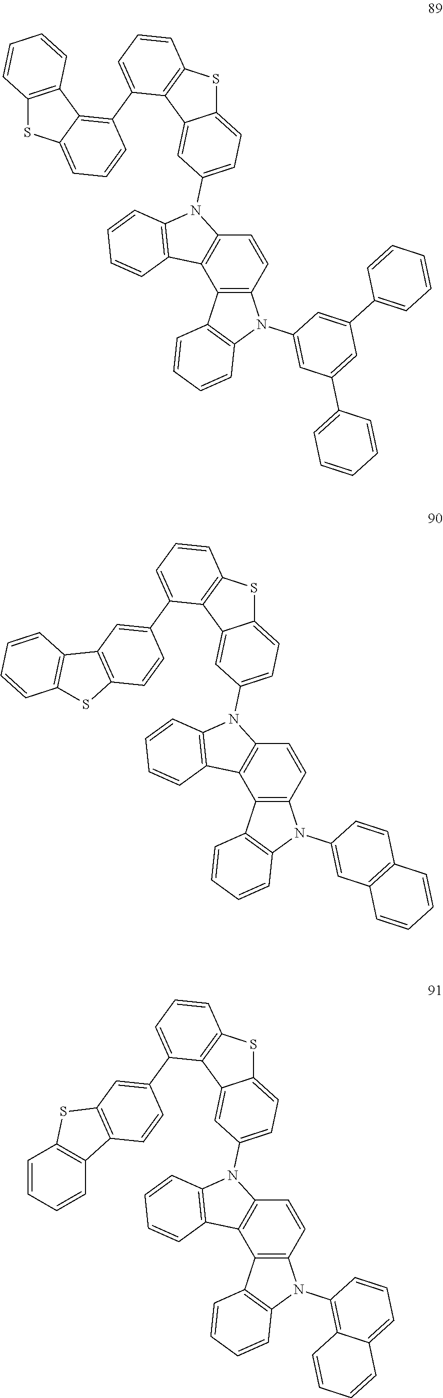 [SMILES: c1ccc(-c2cc(-c3ccccc3)cc(-n3c4ccccc4c4c5c6ccccc6n(-c6ccc7sc8cccc(-c9cccc%10sc%11ccccc%11c9%10)c8c7c6)c5ccc43)c2)cc1 ; c1ccc2cc(-n3c4ccccc4c4c5c6ccccc6n(-c6ccc7sc8cccc(-c9ccc%10sc%11ccccc%11c%10c9)c8c7c6)c5ccc43)ccc2c1 ; c1ccc2c(-n3c4ccccc4c4c5c6ccccc6n(-c6ccc7sc8cccc(-c9ccc%10c(c9)sc9ccccc9%10)c8c7c6)c5ccc43)cccc2c1]

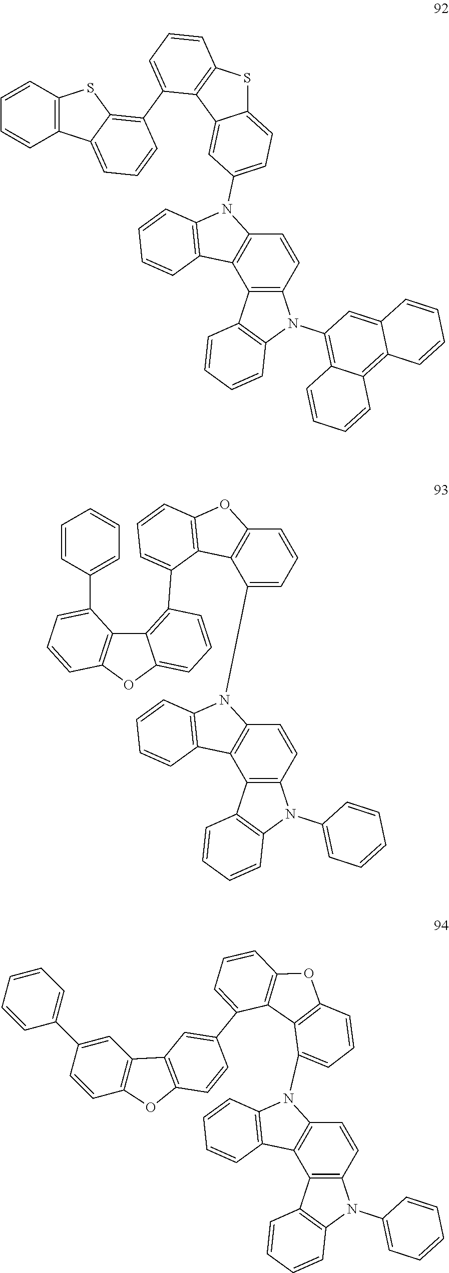 [SMILES: c1ccc2c(c1)cc(-n1c3ccccc3c3c4c5ccccc5n(-c5ccc6sc7cccc(-c8cccc9c8sc8ccccc89)c7c6c5)c4ccc31)c1ccccc12 ; c1ccc(-c2cccc3oc4cccc(-c5cccc6oc7cccc(-n8c9ccccc9c9c%10c%11ccccc%11n(-c%11ccccc%11)c%10ccc98)c7c56)c4c23)cc1 ; c1ccc(-c2ccc3oc4ccc(-c5cccc6oc7cccc(-n8c9ccccc9c9c%10c%11ccccc%11n(-c%11ccccc%11)c%10ccc98)c7c56)cc4c3c2)cc1]

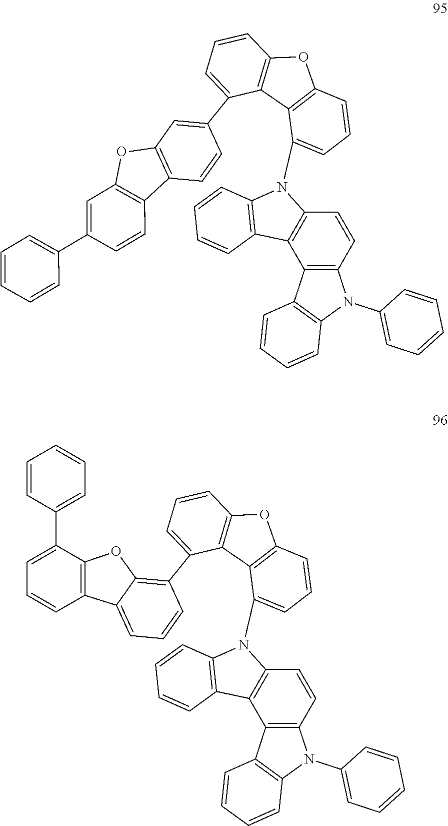 [SMILES: c1ccc(-c2ccc3c(c2)oc2cc(-c4cccc5oc6cccc(-n7c8ccccc8c8c9c%10ccccc%10n(-c%10ccccc%10)c9ccc87)c6c45)ccc23)cc1 ; c1ccc(-c2cccc3c2oc2c(-c4cccc5oc6cccc(-n7c8ccccc8c8c9c%10ccccc%10n(-c%10ccccc%10)c9ccc87)c6c45)cccc23)cc1]

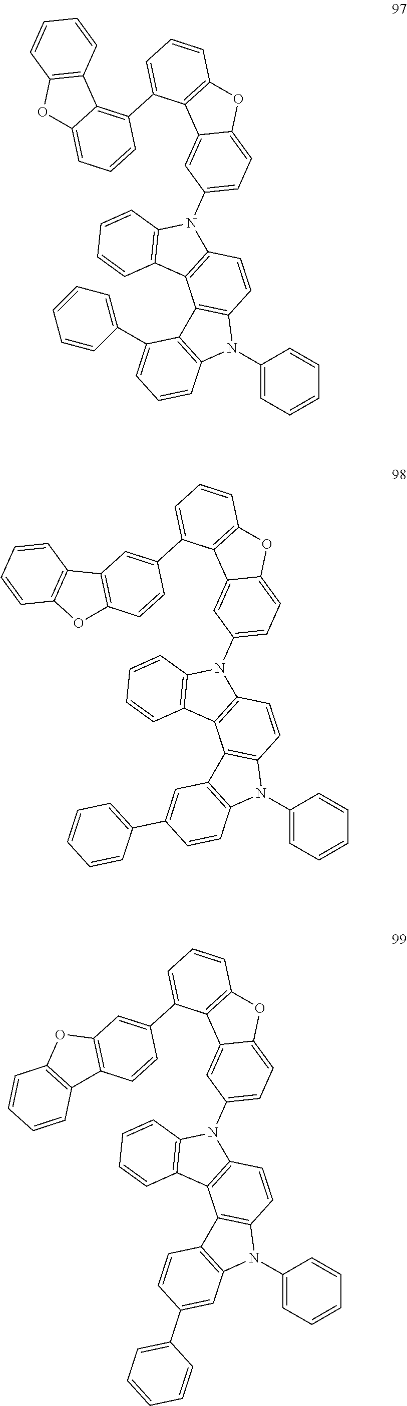 [SMILES: c1ccc(-c2cccc3c2c2c4c5ccccc5n(-c5ccc6oc7cccc(-c8cccc9oc%10ccccc%10c89)c7c6c5)c4ccc2n3-c2ccccc2)cc1 ; c1ccc(-c2ccc3c(c2)c2c4c5ccccc5n(-c5ccc6oc7cccc(-c8ccc9oc%10ccccc%10c9c8)c7c6c5)c4ccc2n3-c2ccccc2)cc1 ; c1ccc(-c2ccc3c4c5c6ccccc6n(-c6ccc7oc8cccc(-c9ccc%10c(c9)oc9ccccc9%10)c8c7c6)c5ccc4n(-c4ccccc4)c3c2)cc1]

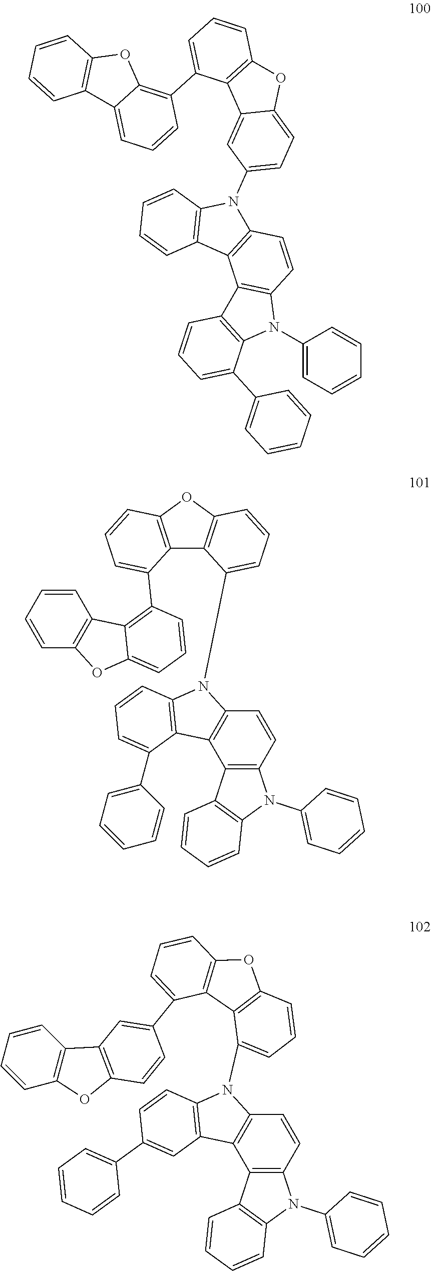 [SMILES: c1ccc(-c2cccc3c4c5c6ccccc6n(-c6ccc7oc8cccc(-c9cccc%10c9oc9ccccc9%10)c8c7c6)c5ccc4n(-c4ccccc4)c23)cc1 ; c1ccc(-c2cccc3c2c2c4c5ccccc5n(-c5ccccc5)c4ccc2n3-c2cccc3oc4cccc(-c5cccc6oc7ccccc7c56)c4c23)cc1 ; c1ccc(-c2ccc3c(c2)c2c4c5ccccc5n(-c5ccccc5)c4ccc2n3-c2cccc3oc4cccc(-c5ccc6oc7ccccc7c6c5)c4c23)cc1]

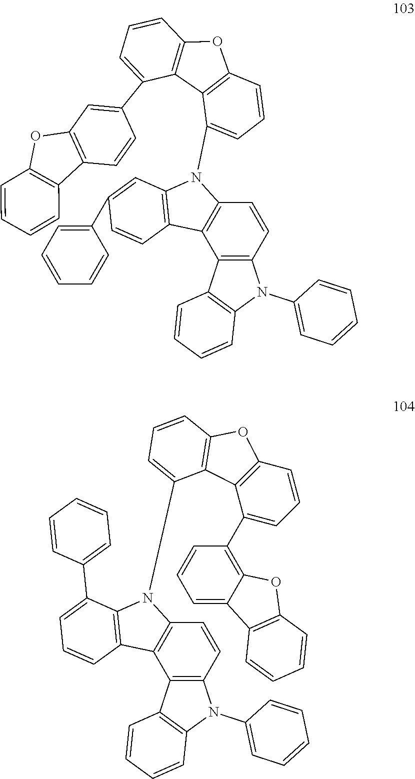 [SMILES: c1ccc(-c2ccc3c4c5c6ccccc6n(-c6ccccc6)c5ccc4n(-c4cccc5oc6cccc(-c7ccc8c(c7)oc7ccccc78)c6c45)c3c2)cc1 ; c1ccc(-c2cccc3c4c5c6ccccc6n(-c6ccccc6)c5ccc4n(-c4cccc5oc6cccc(-c7cccc8c7oc7ccccc78)c6c45)c23)cc1]

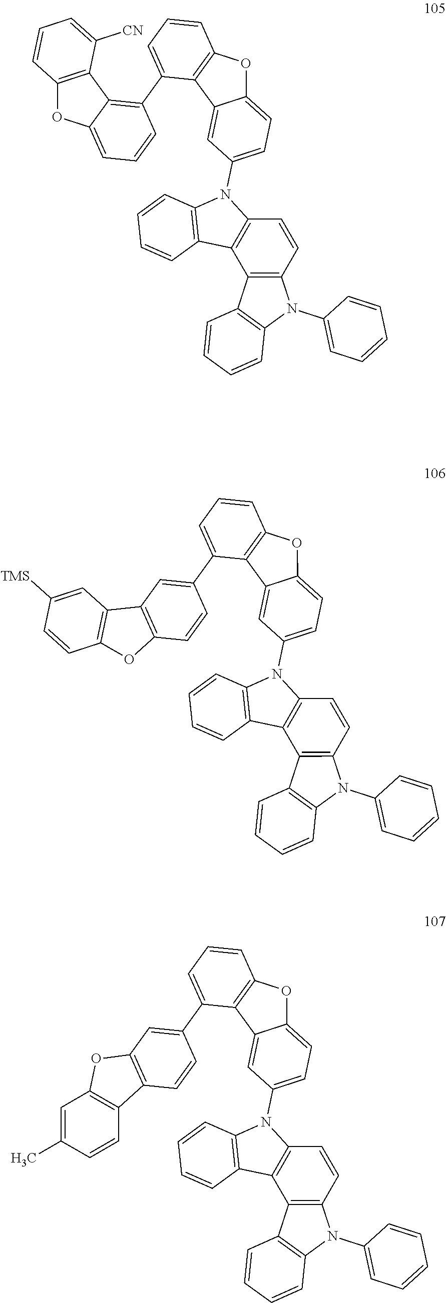 [SMILES: N#Cc1cccc2oc3cccc(-c4cccc5oc6ccc(-n7c8ccccc8c8c9c%10ccccc%10n(-c%10ccccc%10)c9ccc87)cc6c45)c3c12 ; C[Si](C)(C)c1ccc2oc3ccc(-c4cccc5oc6ccc(-n7c8ccccc8c8c9c%10ccccc%10n(-c%10ccccc%10)c9ccc87)cc6c45)cc3c2c1 ; Cc1ccc2c(c1)oc1cc(-c3cccc4oc5ccc(-n6c7ccccc7c7c8c9ccccc9n(-c9ccccc9)c8ccc76)cc5c34)ccc12]

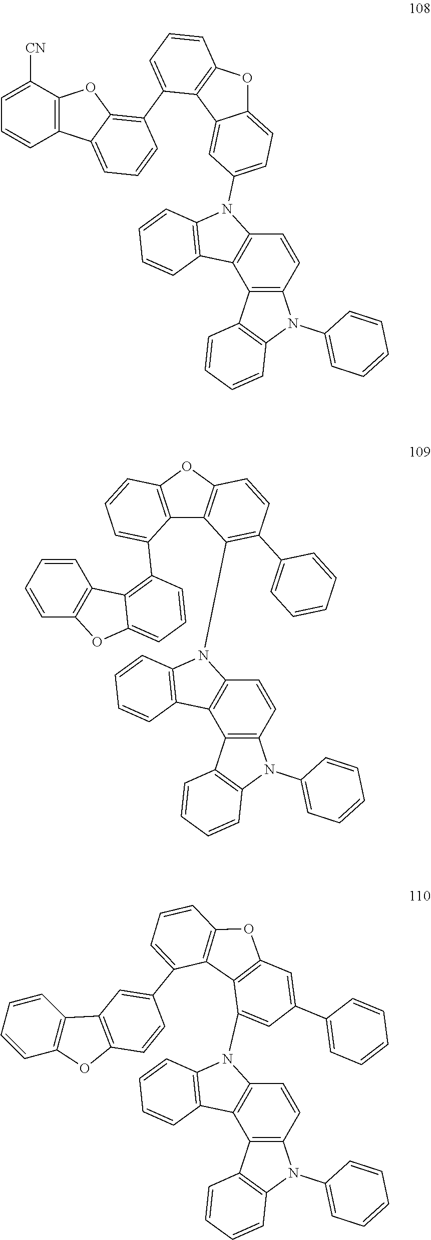 [SMILES: N#Cc1cccc2c1oc1c(-c3cccc4oc5ccc(-n6c7ccccc7c7c8c9ccccc9n(-c9ccccc9)c8ccc76)cc5c34)cccc12 ; c1ccc(-c2ccc3oc4cccc(-c5cccc6oc7ccccc7c56)c4c3c2-n2c3ccccc3c3c4c5ccccc5n(-c5ccccc5)c4ccc32)cc1 ; c1ccc(-c2cc(-n3c4ccccc4c4c5c6ccccc6n(-c6ccccc6)c5ccc43)c3c(c2)oc2cccc(-c4ccc5oc6ccccc6c5c4)c23)cc1]

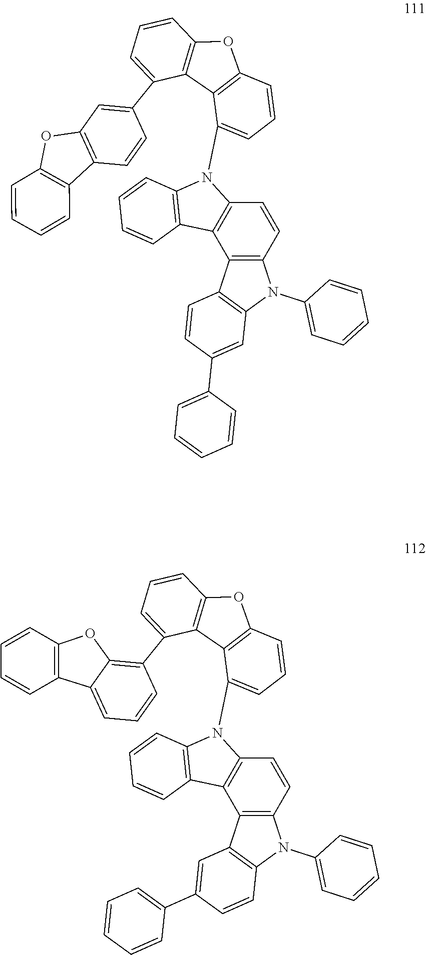 [SMILES: c1ccc(-c2ccc3c4c5c6ccccc6n(-c6cccc7oc8cccc(-c9ccc%10c(c9)oc9ccccc9%10)c8c67)c5ccc4n(-c4ccccc4)c3c2)cc1 ; c1ccc(-c2ccc3c(c2)c2c4c5ccccc5n(-c5cccc6oc7cccc(-c8cccc9c8oc8ccccc89)c7c56)c4ccc2n3-c2ccccc2)cc1]

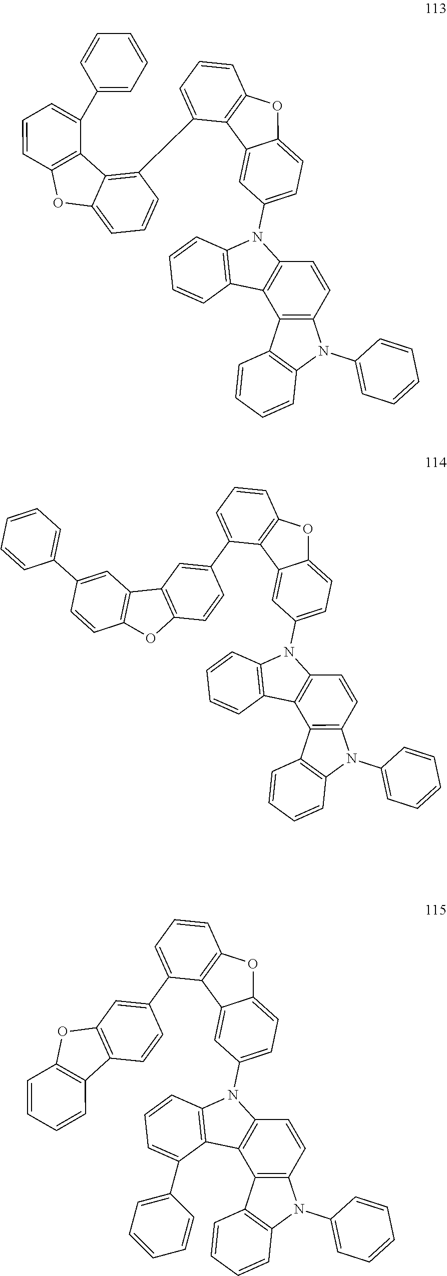 [SMILES: c1ccc(-c2cccc3oc4cccc(-c5cccc6oc7ccc(-n8c9ccccc9c9c%10c%11ccccc%11n(-c%11ccccc%11)c%10ccc98)cc7c56)c4c23)cc1 ; c1ccc(-c2ccc3oc4ccc(-c5cccc6oc7ccc(-n8c9ccccc9c9c%10c%11ccccc%11n(-c%11ccccc%11)c%10ccc98)cc7c56)cc4c3c2)cc1 ; c1ccc(-c2cccc3c2c2c4c5ccccc5n(-c5ccccc5)c4ccc2n3-c2ccc3oc4cccc(-c5ccc6c(c5)oc5ccccc56)c4c3c2)cc1]

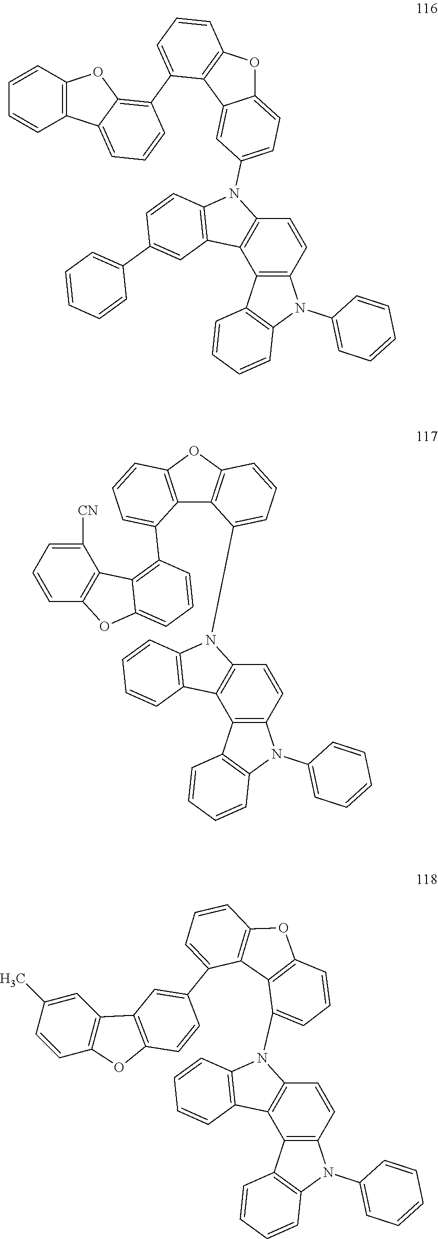 [SMILES: c1ccc(-c2ccc3c(c2)c2c4c5ccccc5n(-c5ccccc5)c4ccc2n3-c2ccc3oc4cccc(-c5cccc6c5oc5ccccc56)c4c3c2)cc1 ; N#Cc1cccc2oc3cccc(-c4cccc5oc6cccc(-n7c8ccccc8c8c9c%10ccccc%10n(-c%10ccccc%10)c9ccc87)c6c45)c3c12 ; Cc1ccc2oc3ccc(-c4cccc5oc6cccc(-n7c8ccccc8c8c9c%10ccccc%10n(-c%10ccccc%10)c9ccc87)c6c45)cc3c2c1]

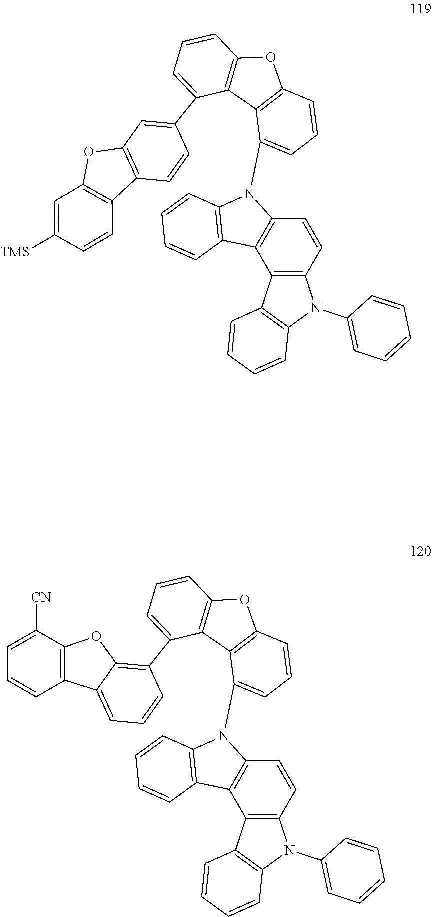 [SMILES: C[Si](C)(C)c1ccc2c(c1)oc1cc(-c3cccc4oc5cccc(-n6c7ccccc7c7c8c9ccccc9n(-c9ccccc9)c8ccc76)c5c34)ccc12 ; N#Cc1cccc2c1oc1c(-c3cccc4oc5cccc(-n6c7ccccc7c7c8c9ccccc9n(-c9ccccc9)c8ccc76)c5c34)cccc12]

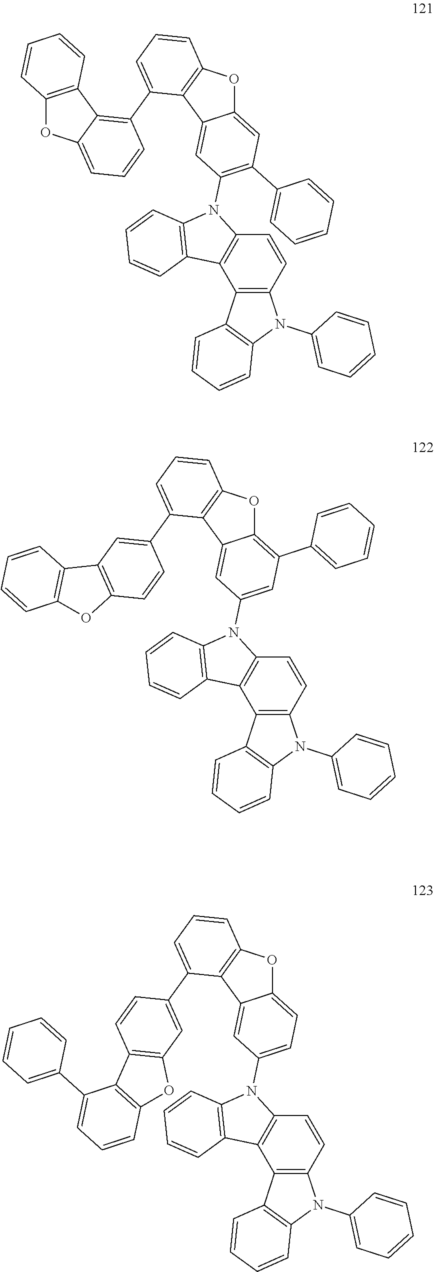 [SMILES: c1ccc(-c2cc3oc4cccc(-c5cccc6oc7ccccc7c56)c4c3cc2-n2c3ccccc3c3c4c5ccccc5n(-c5ccccc5)c4ccc32)cc1 ; c1ccc(-c2cc(-n3c4ccccc4c4c5c6ccccc6n(-c6ccccc6)c5ccc43)cc3c2oc2cccc(-c4ccc5oc6ccccc6c5c4)c23)cc1 ; c1ccc(-c2cccc3oc4cc(-c5cccc6oc7ccc(-n8c9ccccc9c9c%10c%11ccccc%11n(-c%11ccccc%11)c%10ccc98)cc7c56)ccc4c23)cc1]

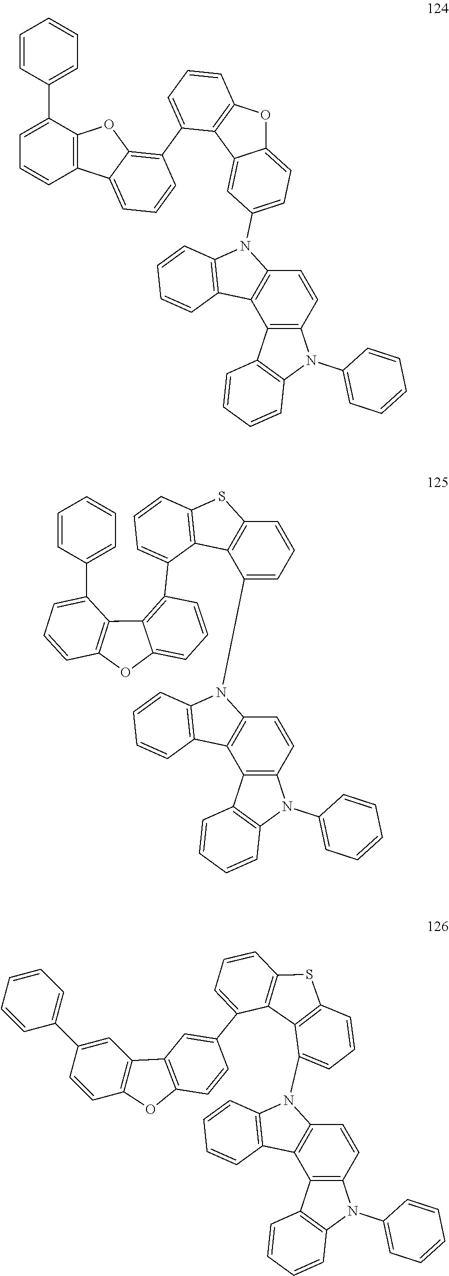 [SMILES: c1ccc(-c2cccc3c2oc2c(-c4cccc5oc6ccc(-n7c8ccccc8c8c9c%10ccccc%10n(-c%10ccccc%10)c9ccc87)cc6c45)cccc23)cc1 ; c1ccc(-c2cccc3oc4cccc(-c5cccc6sc7cccc(-n8c9ccccc9c9c%10c%11ccccc%11n(-c%11ccccc%11)c%10ccc98)c7c56)c4c23)cc1 ; c1ccc(-c2ccc3oc4ccc(-c5cccc6sc7cccc(-n8c9ccccc9c9c%10c%11ccccc%11n(-c%11ccccc%11)c%10ccc98)c7c56)cc4c3c2)cc1]

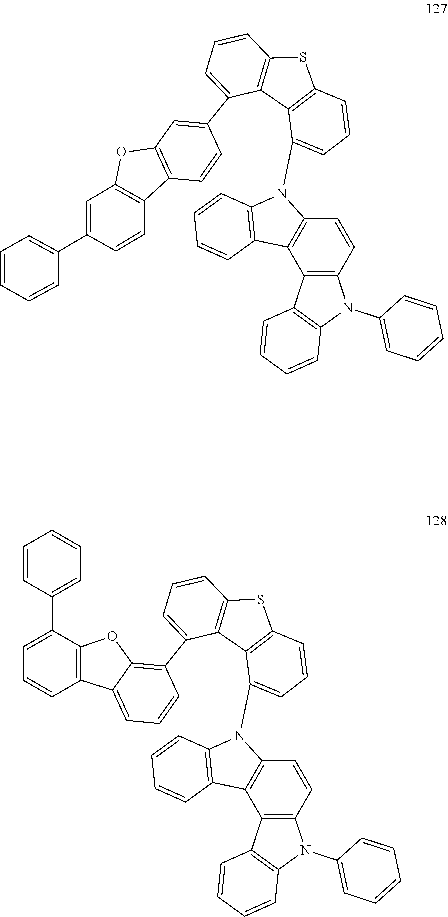 [SMILES: c1ccc(-c2ccc3c(c2)oc2cc(-c4cccc5sc6cccc(-n7c8ccccc8c8c9c%10ccccc%10n(-c%10ccccc%10)c9ccc87)c6c45)ccc23)cc1 ; c1ccc(-c2cccc3c2oc2c(-c4cccc5sc6cccc(-n7c8ccccc8c8c9c%10ccccc%10n(-c%10ccccc%10)c9ccc87)c6c45)cccc23)cc1]

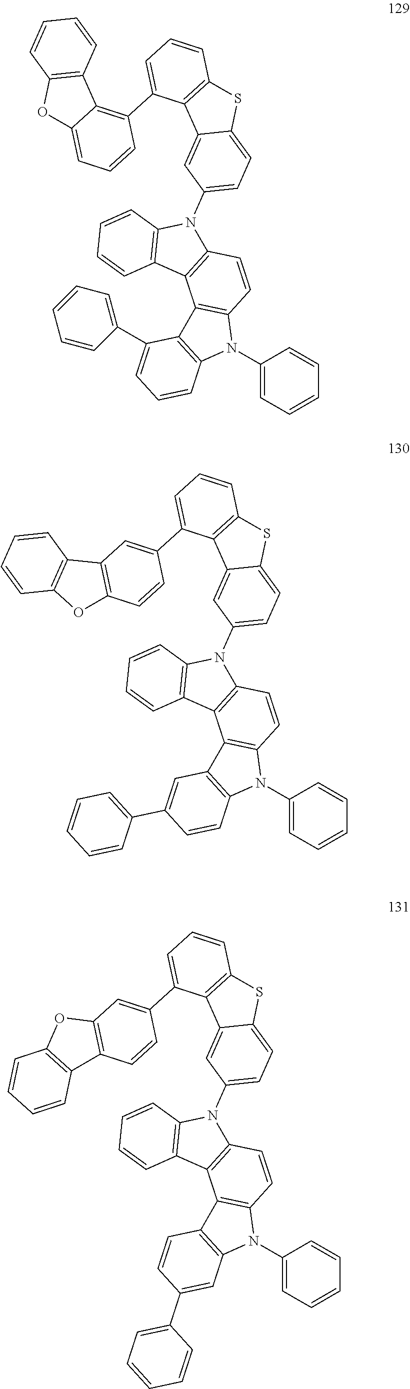 [SMILES: c1ccc(-c2cccc3c2c2c4c5ccccc5n(-c5ccc6sc7cccc(-c8cccc9oc%10ccccc%10c89)c7c6c5)c4ccc2n3-c2ccccc2)cc1 ; c1ccc(-c2ccc3c(c2)c2c4c5ccccc5n(-c5ccc6sc7cccc(-c8ccc9oc%10ccccc%10c9c8)c7c6c5)c4ccc2n3-c2ccccc2)cc1 ; c1ccc(-c2ccc3c4c5c6ccccc6n(-c6ccc7sc8cccc(-c9ccc%10c(c9)oc9ccccc9%10)c8c7c6)c5ccc4n(-c4ccccc4)c3c2)cc1]

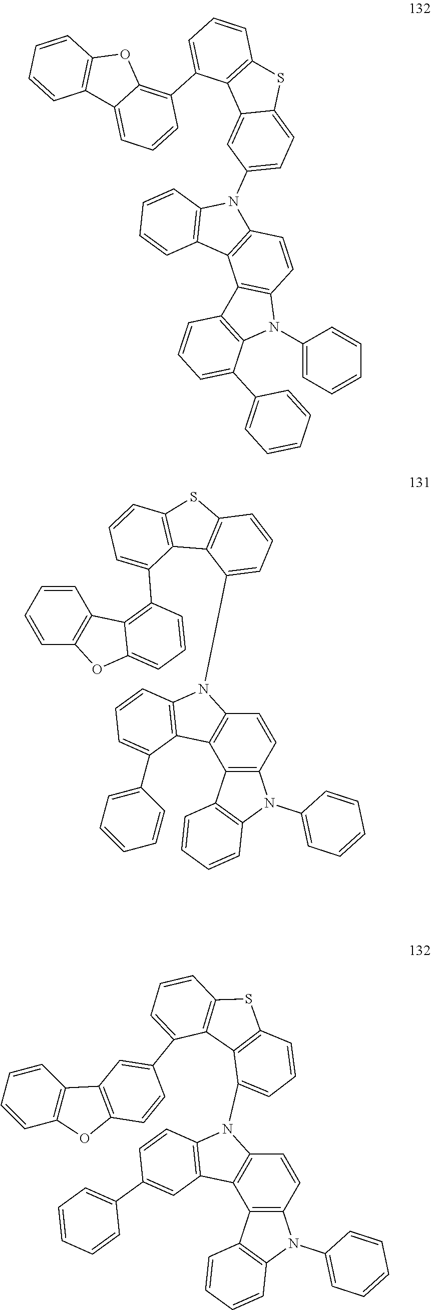 [SMILES: c1ccc(-c2cccc3c4c5c6ccccc6n(-c6ccc7sc8cccc(-c9cccc%10c9oc9ccccc9%10)c8c7c6)c5ccc4n(-c4ccccc4)c23)cc1 ; c1ccc(-c2cccc3c2c2c4c5ccccc5n(-c5ccccc5)c4ccc2n3-c2cccc3sc4cccc(-c5cccc6oc7ccccc7c56)c4c23)cc1 ; c1ccc(-c2ccc3c(c2)c2c4c5ccccc5n(-c5ccccc5)c4ccc2n3-c2cccc3sc4cccc(-c5ccc6oc7ccccc7c6c5)c4c23)cc1]

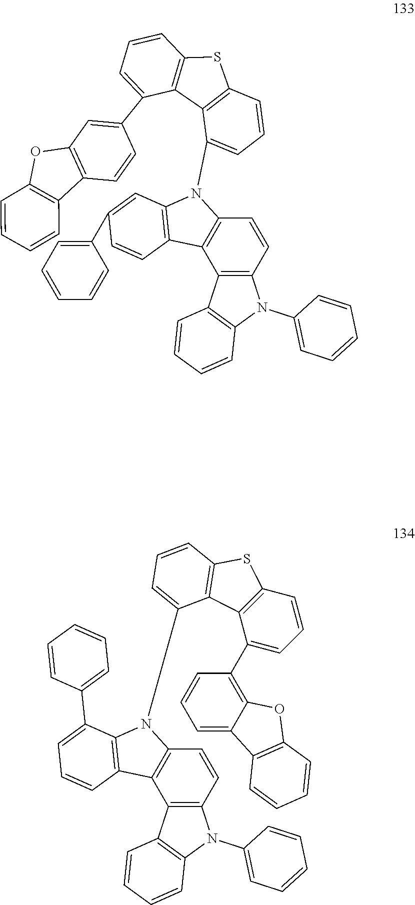 [SMILES: c1ccc(-c2ccc3c4c5c6ccccc6n(-c6ccccc6)c5ccc4n(-c4cccc5sc6cccc(-c7ccc8c(c7)oc7ccccc78)c6c45)c3c2)cc1 ; c1ccc(-c2cccc3c4c5c6ccccc6n(-c6ccccc6)c5ccc4n(-c4cccc5sc6cccc(-c7cccc8c7oc7ccccc78)c6c45)c23)cc1]

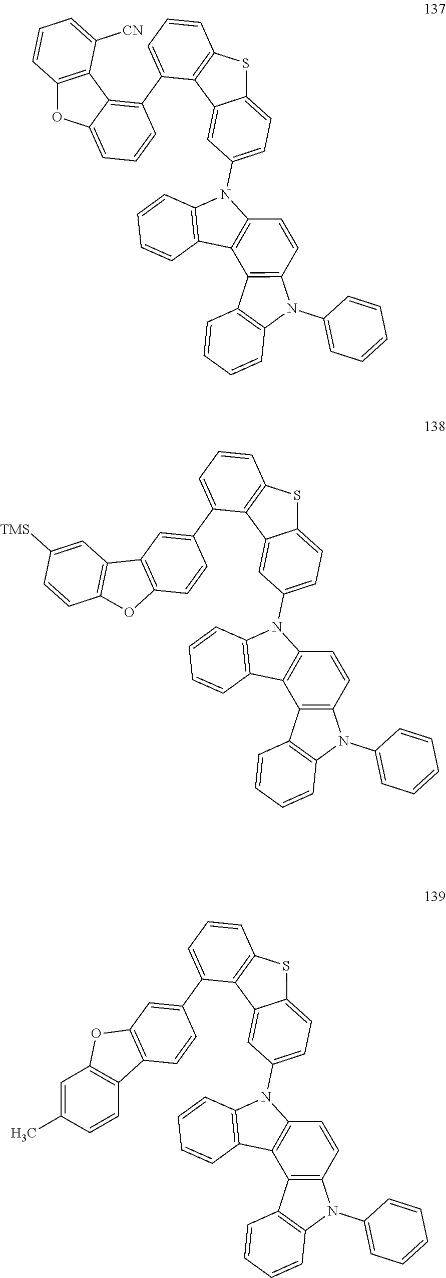 [SMILES: N#Cc1cccc2oc3cccc(-c4cccc5sc6ccc(-n7c8ccccc8c8c9c%10ccccc%10n(-c%10ccccc%10)c9ccc87)cc6c45)c3c12 ; C[Si](C)(C)c1ccc2oc3ccc(-c4cccc5sc6ccc(-n7c8ccccc8c8c9c%10ccccc%10n(-c%10ccccc%10)c9ccc87)cc6c45)cc3c2c1 ; Cc1ccc2c(c1)oc1cc(-c3cccc4sc5ccc(-n6c7ccccc7c7c8c9ccccc9n(-c9ccccc9)c8ccc76)cc5c34)ccc12]

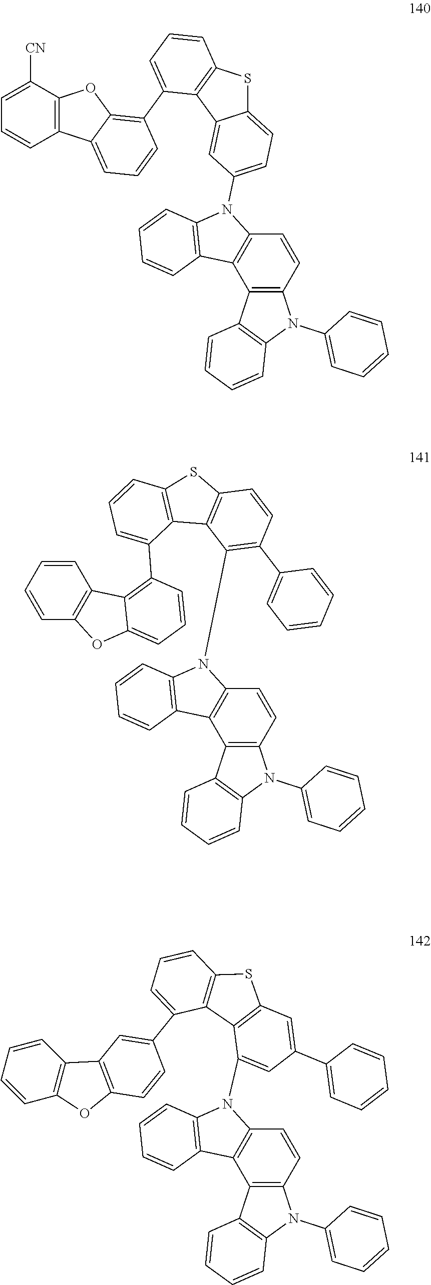 [SMILES: N#Cc1cccc2c1oc1c(-c3cccc4sc5ccc(-n6c7ccccc7c7c8c9ccccc9n(-c9ccccc9)c8ccc76)cc5c34)cccc12 ; c1ccc(-c2ccc3sc4cccc(-c5cccc6oc7ccccc7c56)c4c3c2-n2c3ccccc3c3c4c5ccccc5n(-c5ccccc5)c4ccc32)cc1 ; c1ccc(-c2cc(-n3c4ccccc4c4c5c6ccccc6n(-c6ccccc6)c5ccc43)c3c(c2)sc2cccc(-c4ccc5oc6ccccc6c5c4)c23)cc1]

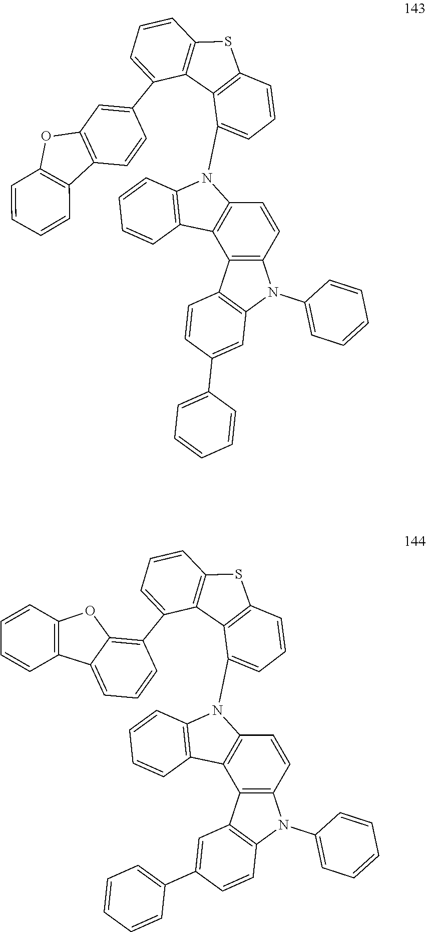 [SMILES: c1ccc(-c2ccc3c4c5c6ccccc6n(-c6cccc7sc8cccc(-c9ccc%10c(c9)oc9ccccc9%10)c8c67)c5ccc4n(-c4ccccc4)c3c2)cc1 ; c1ccc(-c2ccc3c(c2)c2c4c5ccccc5n(-c5cccc6sc7cccc(-c8cccc9c8oc8ccccc89)c7c56)c4ccc2n3-c2ccccc2)cc1]

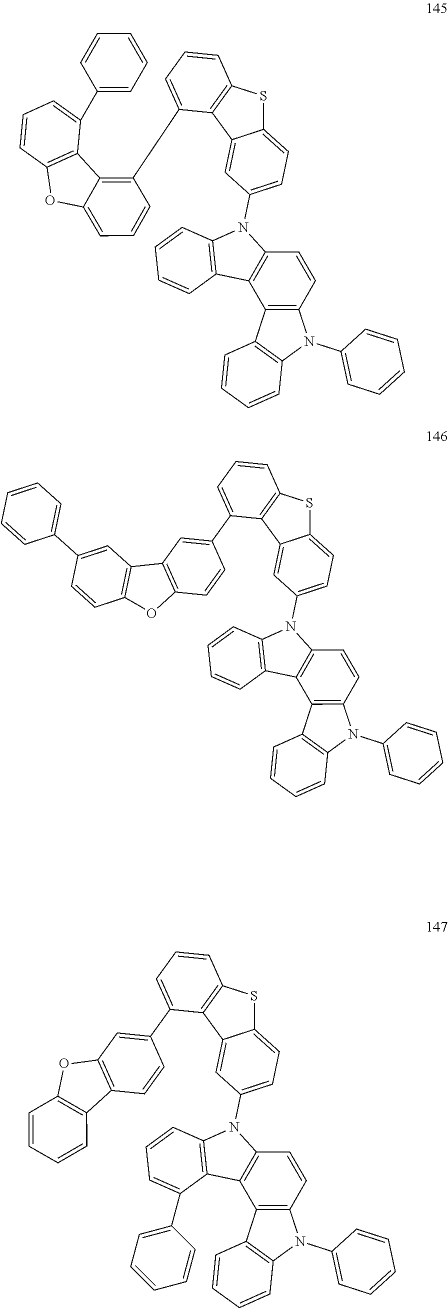 [SMILES: c1ccc(-c2cccc3oc4cccc(-c5cccc6sc7ccc(-n8c9ccccc9c9c%10c%11ccccc%11n(-c%11ccccc%11)c%10ccc98)cc7c56)c4c23)cc1 ; c1ccc(-c2ccc3oc4ccc(-c5cccc6sc7ccc(-n8c9ccccc9c9c%10c%11ccccc%11n(-c%11ccccc%11)c%10ccc98)cc7c56)cc4c3c2)cc1 ; c1ccc(-c2cccc3c2c2c4c5ccccc5n(-c5ccccc5)c4ccc2n3-c2ccc3sc4cccc(-c5ccc6c(c5)oc5ccccc56)c4c3c2)cc1]

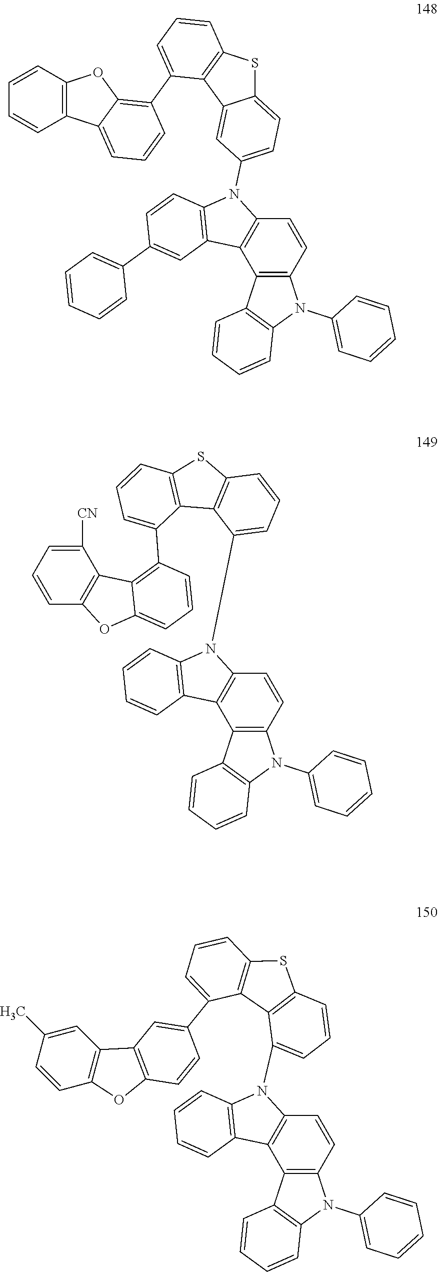 [SMILES: c1ccc(-c2ccc3c(c2)c2c4c5ccccc5n(-c5ccccc5)c4ccc2n3-c2ccc3sc4cccc(-c5cccc6c5oc5ccccc56)c4c3c2)cc1 ; N#Cc1cccc2oc3cccc(-c4cccc5sc6cccc(-n7c8ccccc8c8c9c%10ccccc%10n(-c%10ccccc%10)c9ccc87)c6c45)c3c12 ; Cc1ccc2oc3ccc(-c4cccc5sc6cccc(-n7c8ccccc8c8c9c%10ccccc%10n(-c%10ccccc%10)c9ccc87)c6c45)cc3c2c1]

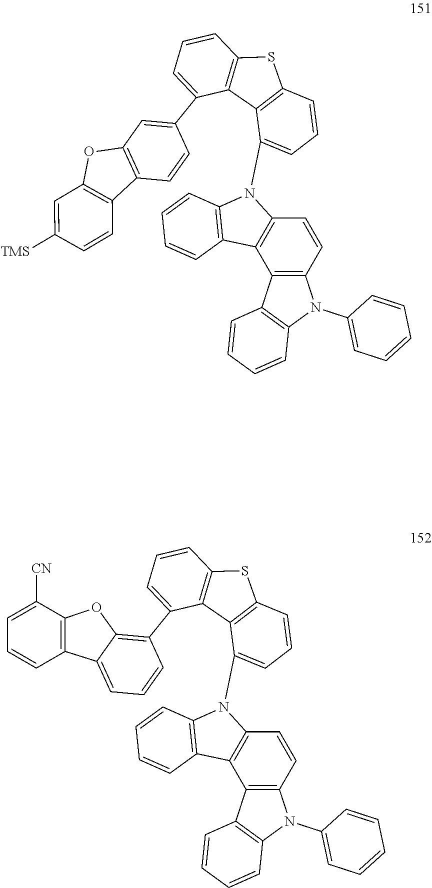 [SMILES: C[Si](C)(C)c1ccc2c(c1)oc1cc(-c3cccc4sc5cccc(-n6c7ccccc7c7c8c9ccccc9n(-c9ccccc9)c8ccc76)c5c34)ccc12 ; N#Cc1cccc2c1oc1c(-c3cccc4sc5cccc(-n6c7ccccc7c7c8c9ccccc9n(-c9ccccc9)c8ccc76)c5c34)cccc12]

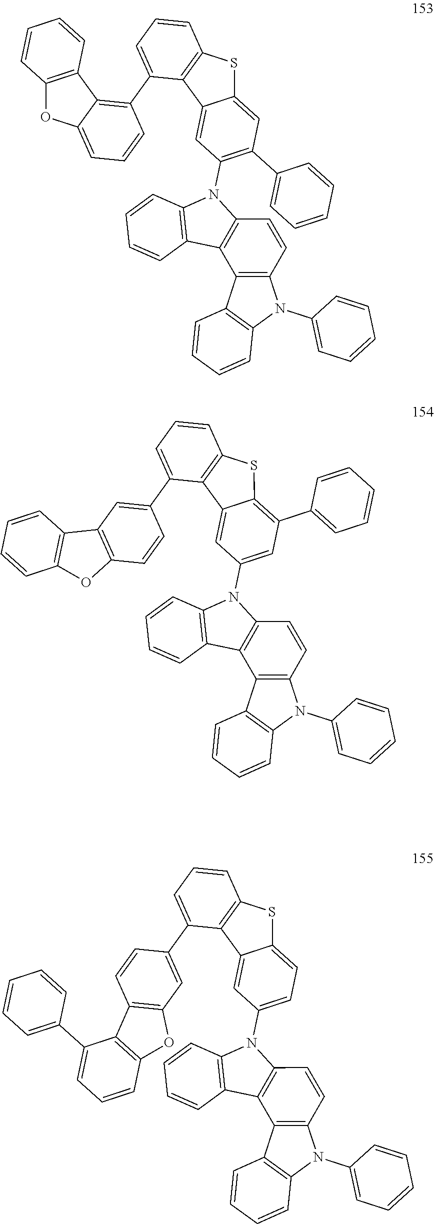 [SMILES: c1ccc(-c2cc3sc4cccc(-c5cccc6oc7ccccc7c56)c4c3cc2-n2c3ccccc3c3c4c5ccccc5n(-c5ccccc5)c4ccc32)cc1 ; c1ccc(-c2cc(-n3c4ccccc4c4c5c6ccccc6n(-c6ccccc6)c5ccc43)cc3c2sc2cccc(-c4ccc5oc6ccccc6c5c4)c23)cc1 ; c1ccc(-c2cccc3oc4cc(-c5cccc6sc7ccc(-n8c9ccccc9c9c%10c%11ccccc%11n(-c%11ccccc%11)c%10ccc98)cc7c56)ccc4c23)cc1]

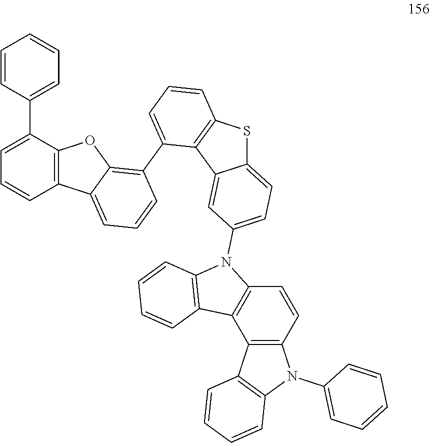 [SMILES: c1ccc(-c2cccc3c2oc2c(-c4cccc5sc6ccc(-n7c8ccccc8c8c9c%10ccccc%10n(-c%10ccccc%10)c9ccc87)cc6c45)cccc23)cc1]

In Group 1, TMS is —Si(CH₃)₃.

In addition, the form in which deuterium is substituted in the compounds listed in Group I may include, e.g., a compound of Group 1-1.

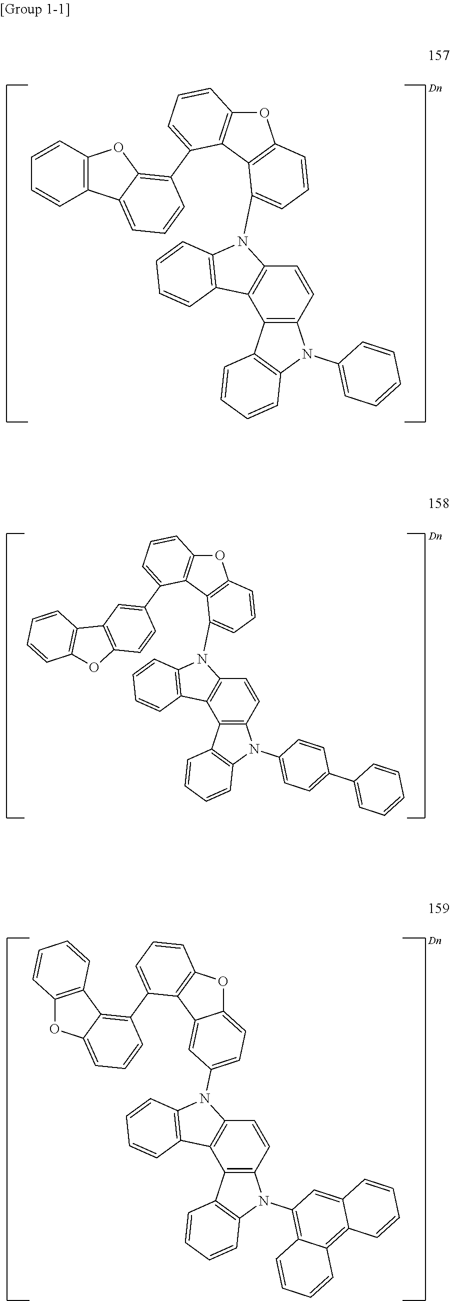

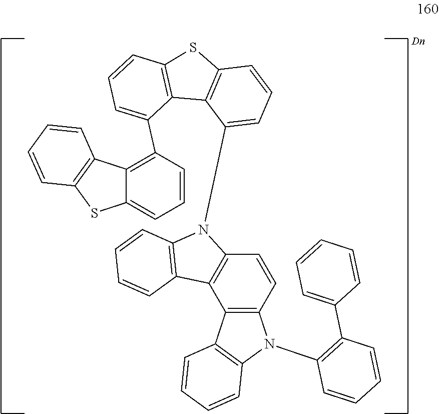

(Dn refers to the number of deuterium substitutions and indicates a structure substituted with one or more deuterium atoms).

More specific examples of the compounds listed in Group 1-1 may include, e.g., a compound of Group 1-2.

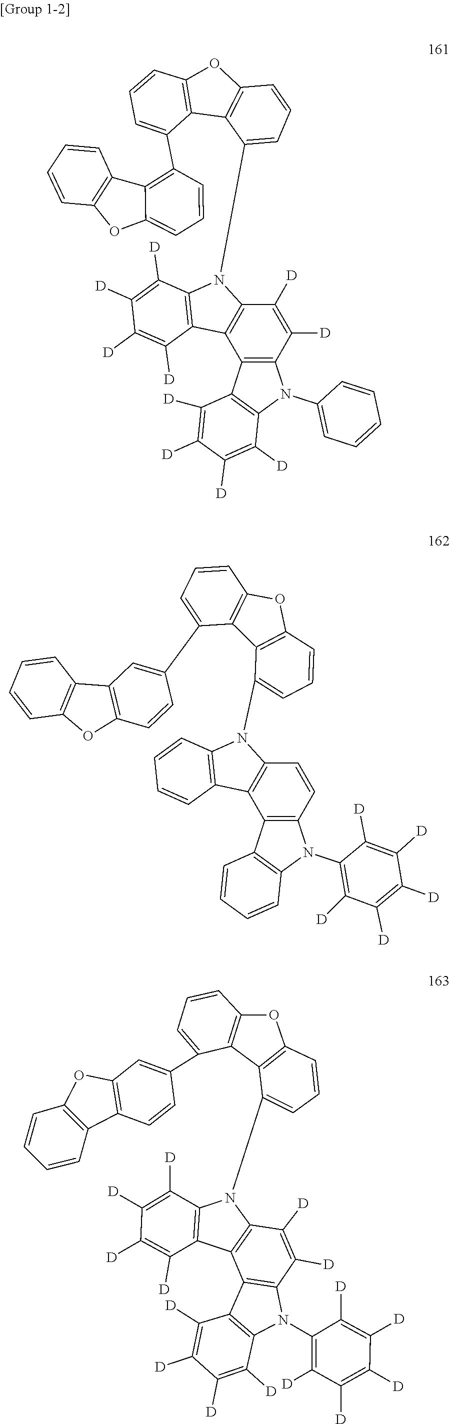 [SMILES: [2H]c1c([2H])c([2H])c2c(c1[2H])c1c3c4c([2H])c([2H])c([2H])c([2H])c4n(-c4cccc5oc6cccc(-c7cccc8oc9ccccc9c78)c6c45)c3c([2H])c([2H])c1n2-c1ccccc1 ; [2H]c1c([2H])c([2H])c(-n2c3ccccc3c3c4c5ccccc5n(-c5cccc6oc7cccc(-c8ccc9oc%10ccccc%10c9c8)c7c56)c4ccc32)c([2H])c1[2H] ; [2H]c1c([2H])c([2H])c(-n2c3c([2H])c([2H])c([2H])c([2H])c3c3c4c5c([2H])c([2H])c([2H])c([2H])c5n(-c5cccc6oc7cccc(-c8ccc9c(c8)oc8ccccc89)c7c56)c4c([2H])c([2H])c32)c([2H])c1[2H]]

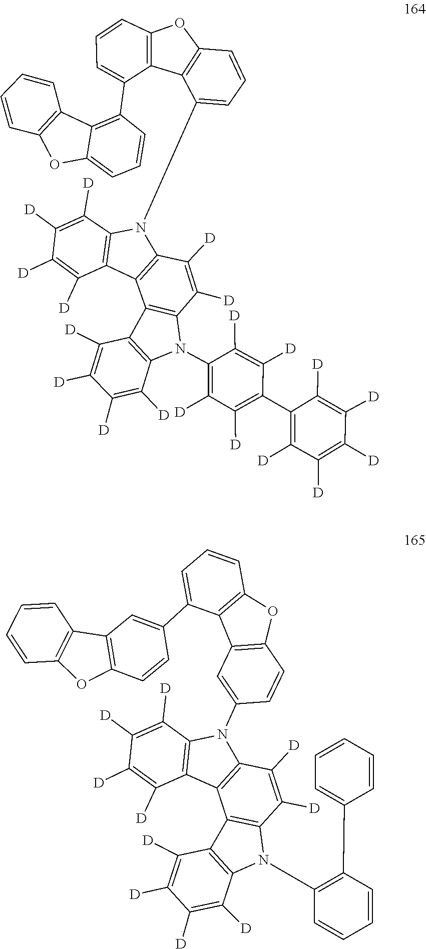 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6cccc7oc8cccc(-c9cccc%10oc%11ccccc%11c9%10)c8c67)c5c([2H])c([2H])c43)c([2H])c2[2H])c([2H])c1[2H] ; [2H]c1c([2H])c([2H])c2c(c1[2H])c1c3c4c([2H])c([2H])c([2H])c([2H])c4n(-c4ccccc4-c4ccccc4)c3c([2H])c([2H])c1n2-c1ccc2oc3cccc(-c4ccc5oc6ccccc6c5c4)c3c2c1]

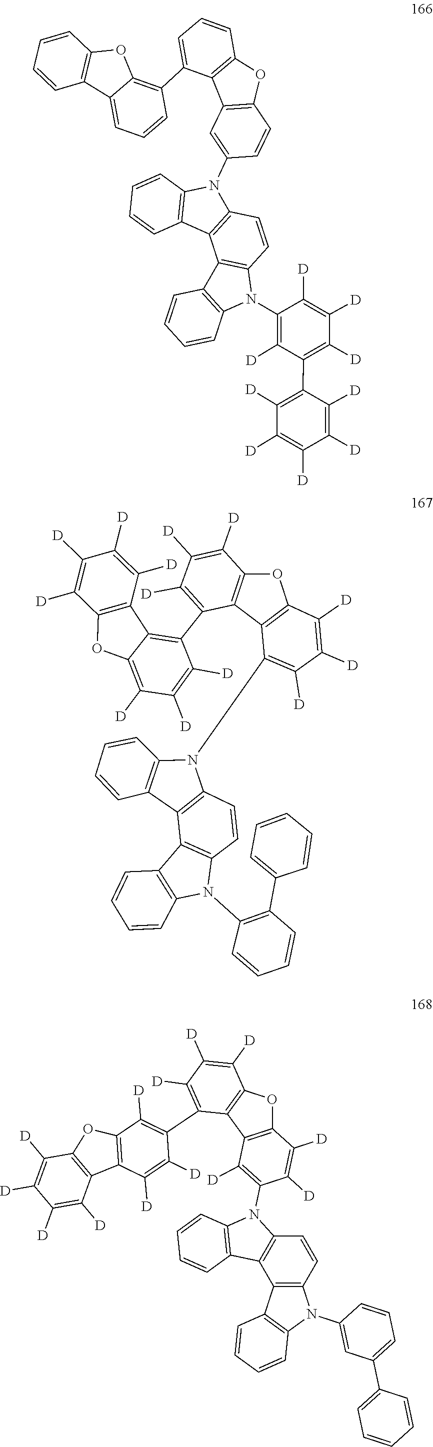

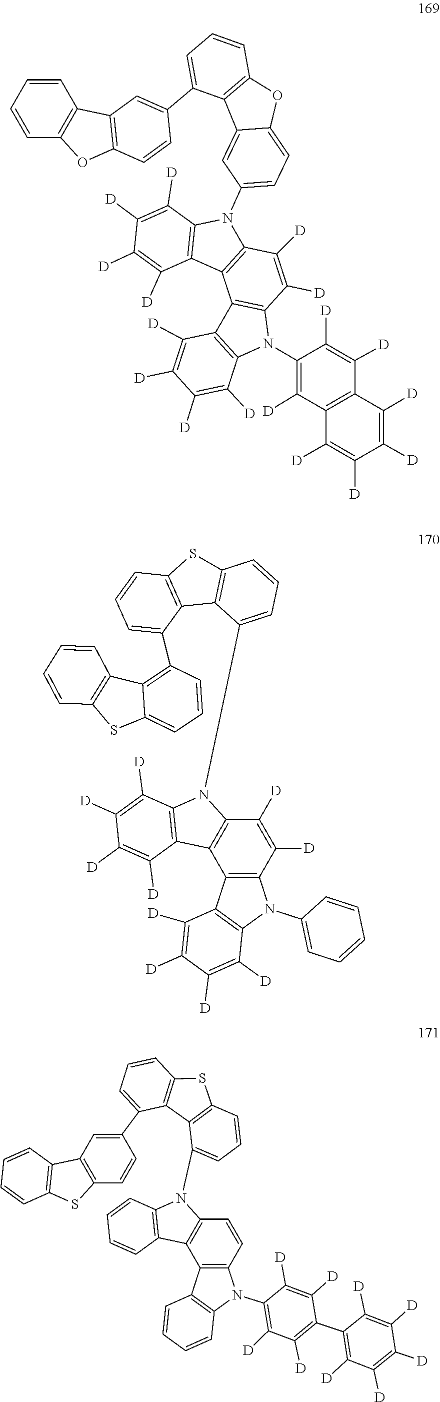 [SMILES: [2H]c1c([2H])c([2H])c2c([2H])c(-n3c4c([2H])c([2H])c([2H])c([2H])c4c4c5c6c([2H])c([2H])c([2H])c([2H])c6n(-c6ccc7oc8cccc(-c9ccc%10oc%11ccccc%11c%10c9)c8c7c6)c5c([2H])c([2H])c43)c([2H])c([2H])c2c1[2H] ; [2H]c1c([2H])c([2H])c2c(c1[2H])c1c3c4c([2H])c([2H])c([2H])c([2H])c4n(-c4cccc5sc6cccc(-c7cccc8sc9ccccc9c78)c6c45)c3c([2H])c([2H])c1n2-c1ccccc1 ; [2H]c1c([2H])c([2H])c(-c2c([2H])c([2H])c(-n3c4ccccc4c4c5c6ccccc6n(-c6cccc7sc8cccc(-c9ccc%10sc%11ccccc%11c%10c9)c8c67)c5ccc43)c([2H])c2[2H])c([2H])c1[2H]]

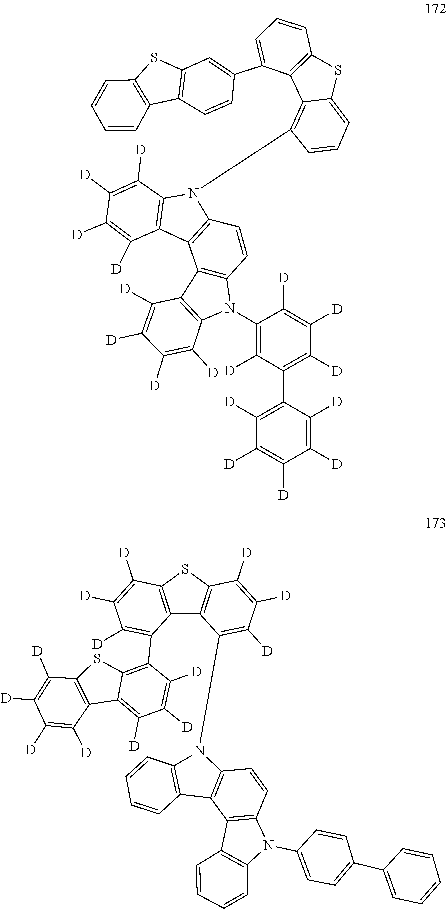 [SMILES: [2H]c1c([2H])c([2H])c(-c2c([2H])c([2H])c([2H])c(-n3c4ccc5c(c6c([2H])c([2H])c([2H])c([2H])c6n5-c5cccc6sc7cccc(-c8ccc9c(c8)sc8ccccc89)c7c56)c4c4c([2H])c([2H])c([2H])c([2H])c43)c2[2H])c([2H])c1[2H] ; [2H]c1c([2H])c([2H])c2c(sc3c(-c4c([2H])c([2H])c([2H])c5sc6c([2H])c([2H])c([2H])c(-n7c8ccccc8c8c9c%10ccccc%10n(-c%10ccc(-c%11ccccc%11)cc%10)c9ccc87)c6c45)c([2H])c([2H])c([2H])c32)c1[2H]]

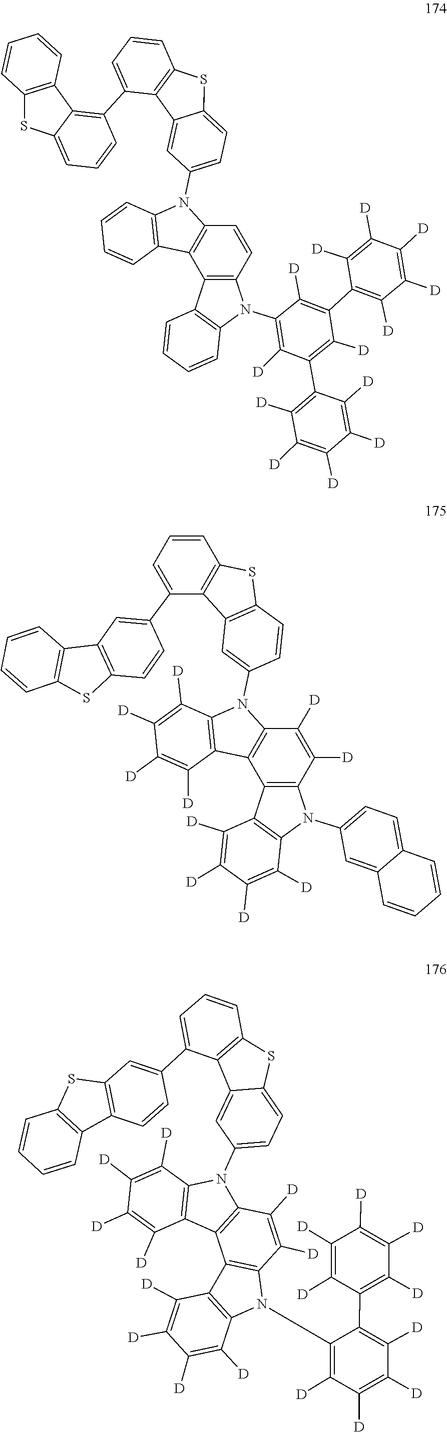

A composition for an organic optoelectronic device according to some example embodiments includes a first compound, and a second compound, wherein the first compound may be the aforementioned compound for an organic optoelectronic device and the second compound may be represented by Chemical Formula 3.

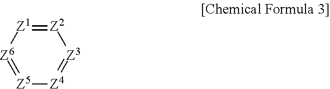

In Chemical Formula 3, Z¹ to Z⁶ may each independently be or include, e.g., N or C-L^{a}-R^{a}.

In an implementation, at least two of Z¹ to Z⁶ may be N.

Each L^{a} may independently be or include, e.g., a single bond, a substituted or

unsubstituted C6 to C20 arylene group, a substituted or unsubstituted C2 to C20 heterocyclic group, or a combination thereof.

Each R^{a} may independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

Each R^{a} may be separately present or adjacent groups thereof may be linked to each other to form a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring.

Because the second compound may help effectively expand the LUMO energy band by including a nitrogen-containing hexagonal moiety, it may be included together with the aforementioned first compound to help increase the balance between holes and electrons, thereby helping significantly improve the life-span characteristics of a device to which it is applied.

In an implementation, two of Z¹ to Z⁶ may be nitrogen (N) and the rest may be C-L^{a}-R^{a}.

In an implementation, Z¹ and Z³ may be nitrogen, Z² may be N or C-L^{a}-R^{a}, Z⁴ may be N or C-L^{a}-R^{a}, Z⁵ may be N or C-L^{a}-R^{a}, and Z⁶ may be N or C-L^{a}-R^{a}.

In an implementation, three of Z¹ to Z⁶ may be nitrogen (N) and the rest may be C-L^{a}-R^{a}.

In an implementation, Z¹, Z³, and Z³ may be nitrogen, Z² may be N or C-L^{a}-R^{a}, Z⁴ may be N or C-L^{a}-R^{a}, and Z⁶ may be N or C-L^{a}-R^{a}.

In an implementation, depending on the specific substituent of R^{a}, the second compound may be, e.g., represented by one of Chemical Formula 3A to Chemical Formula 3C.

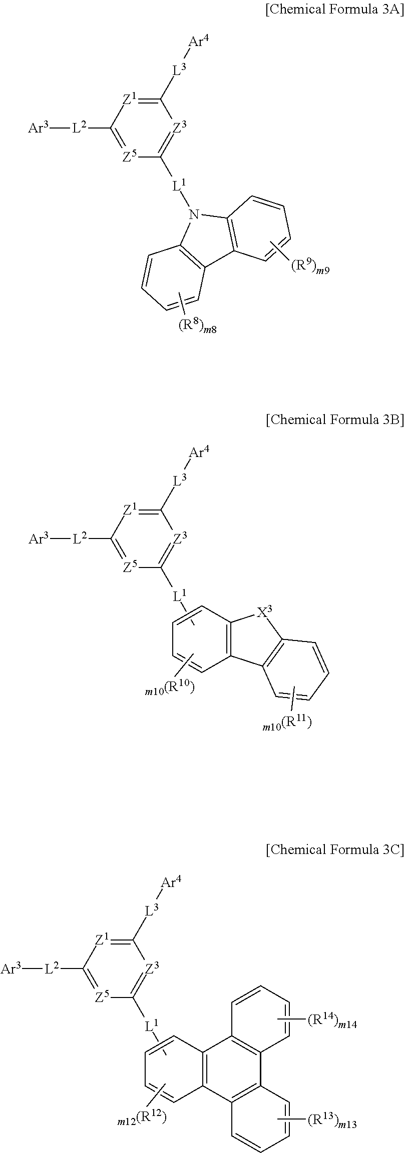

In Chemical Formulas 3A to 3C, Z¹, Z³, and Z⁵ may each independently be, e.g., N or C-L^{a}-R^{a}.

In an implementation, at least two of Z¹, Z³, and Z⁵ may be N.

X³ may be, e.g., O, S, or NR^{b}.

L^{a} and L¹ to L³ may each independently be, e.g., a single bond, a substituted or unsubstituted C6 to C20 arylene group, a substituted or unsubstituted C2 to C20 heterocyclic group, or a combination thereof.

R^{a}, R^{b}, and R⁸ to R¹⁴ may each independently be, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

R⁸ and R⁹ may each be separately present or adjacent groups thereof may be linked to each other to form a substituted or unsubstituted aromatic monocyclic or polycyclic ring.

Ar³ and Ar⁴ may each independently be, e.g., a substituted or unsubstituted C6 to C30 aryl group, a substituted or unsubstituted C2 to C30 heterocyclic group, or a combination thereof.

R^{a}, Ar³, and Ar⁴ may each be separately present or adjacent groups of R^{a}, Ar³, and Ar⁴ may be linked to each other to form a substituted or unsubstituted aromatic monocyclic or polycyclic ring or a substituted or unsubstituted heteroaromatic monocyclic or polycyclic ring,

m8, m9, m11, m13, and m14 may each independently be, e.g., an integer of 1 to 4.

m10 and m12 may each independently be, e.g., an integer of 1 to 3.

In an implementation, m8 may be 2, 3, or 4, and each R⁸ may be the same or different from each other.

In an implementation, m9 may be 2, 3, or 4, and each R⁹ may be the same or different from each other.

In an implementation, m10 may be 2 or 3, and each R¹⁰ may be the same or different from each other.

In an implementation, m11 may be 2, 3, or 4, and each R¹¹ may be the same or different from each other.

In an implementation, m12 may be 2 or 3, and each R¹² may be the same or different from each other.

In an implementation, m13 may be 2, 3, or 4, and each R¹³ may be the same or different from each other.

In an implementation, m14 may be 2, 3, or 4, and each R¹⁴ may be the same or different from each other.

As used herein, the indication that adjacent groups may be linked to each other to form a substituted or unsubstituted aromatic or heteroaromatic monocyclic or polycyclic ring means that any two adjacent substituents may be linked to each other to form a ring. In an implementation, in Chemical Formula 3A, adjacent groups of R⁸ or adjacent groups of R⁹ may be linked to each other to form a substituted or unsubstituted aromatic monocyclic ring. Here, the aromatic monocyclic ring formed may be, e.g., a substituted or unsubstituted phenyl group.

In an implementation, Chemical Formula 3A may be, e.g., represented by one of Chemical Formula 3A-I to Chemical Formula 3A-XIII.

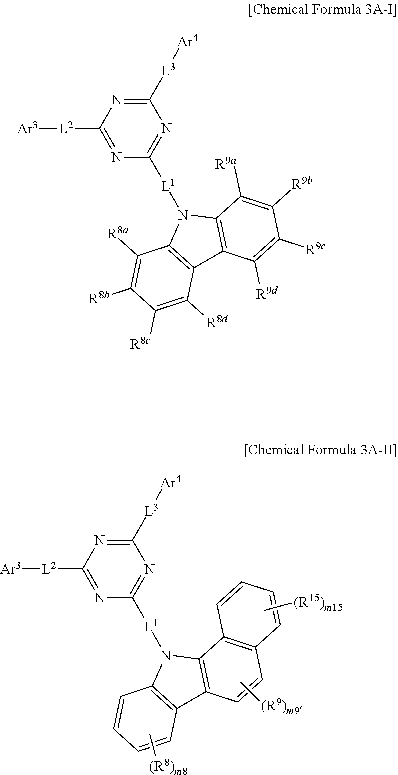

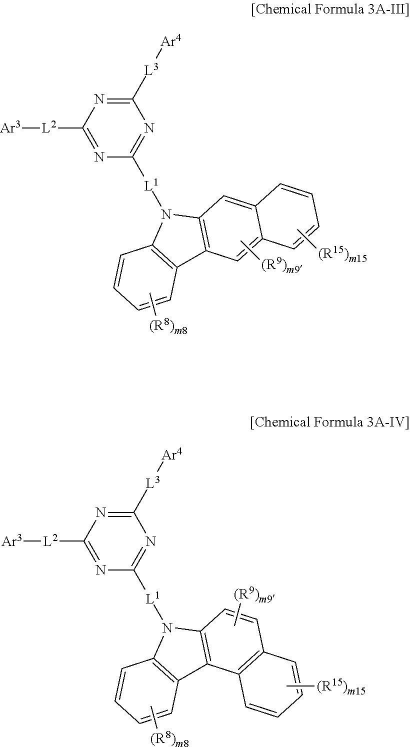

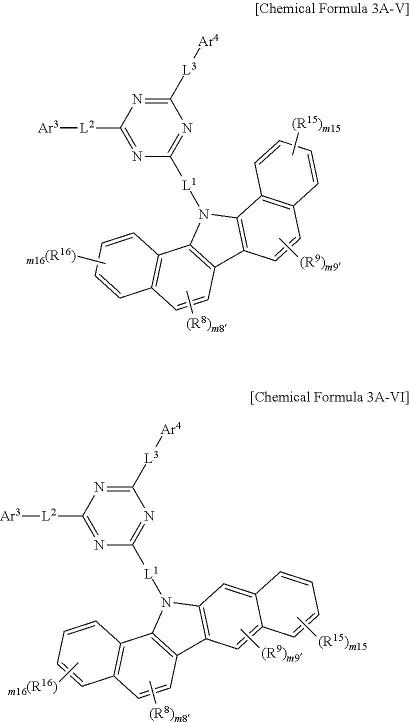

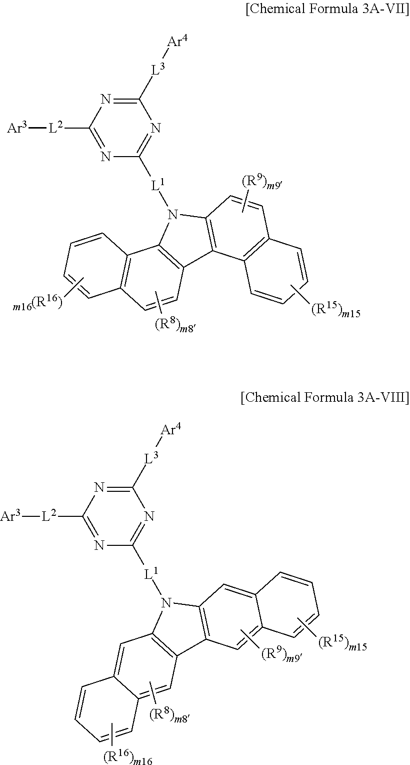

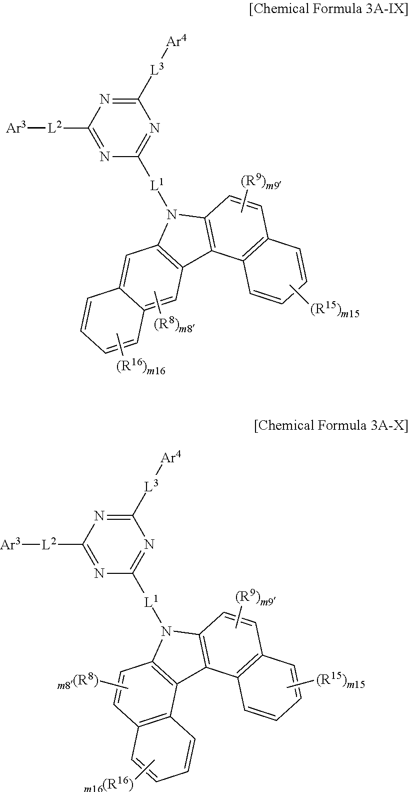

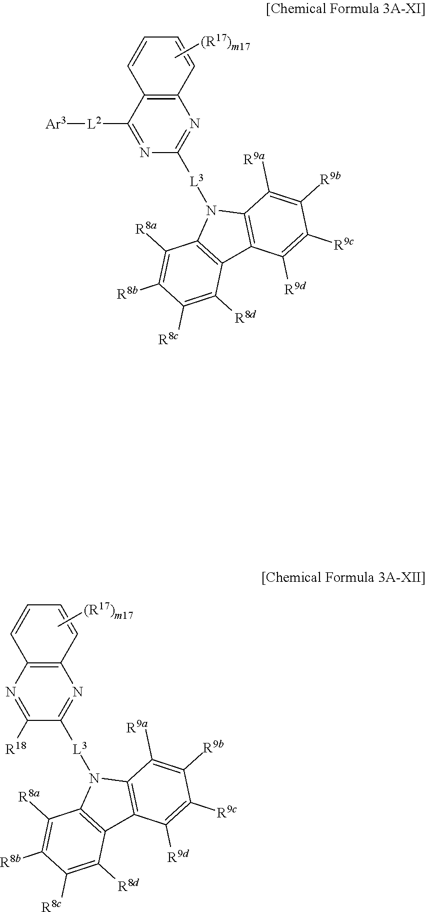

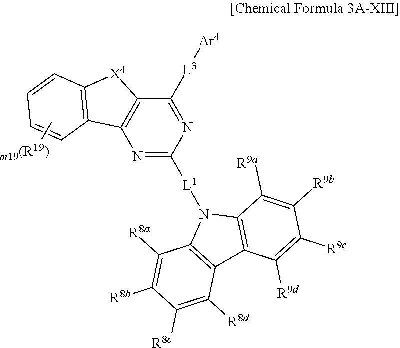

In Chemical Formula 3A-I to Chemical Formula 3A-XIII, m8 may be defined the same as described above.

X² may be, e.g., O or S.

L¹ to L³ may each independently be, e.g., a single bond, a substituted or unsubstituted phenylene group, a substituted or unsubstituted biphenylene group, a substituted or unsubstituted naphthylene group, a substituted or unsubstituted dibenzofuranylene group, or a substituted or unsubstituted dibenzothiophenylene group.

Ar³ and Ar⁴ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted carbazolyl group, a substituted or unsubstituted dibenzofuranyl group, or a substituted or unsubstituted dibenzothiophenyl group.

R⁸, R⁹, R¹⁵ to R¹⁹, R^{8a}, R^{8b}, R^{8c}, R^{8d}, R^{9a}, R^{9b}, R^{9c}, and R^{9d} may each independently be, e.g., hydrogen, deuterium, a substituted or unsubstituted phenyl group, a substituted or unsubstituted dibenzofuranyl group, or a substituted or unsubstituted dibenzothiophenyl group.

m8′ and m9′ may each independently be, e.g., an integer of 1 or 2.

m15, m16, m17, and m19 may each independently be, e.g., an integer of 1 to 4.

In an implementation, Chemical Formula 3B may be, e.g., represented by one of Chemical Formula 3B-I to Chemical Formula 3B-IV.

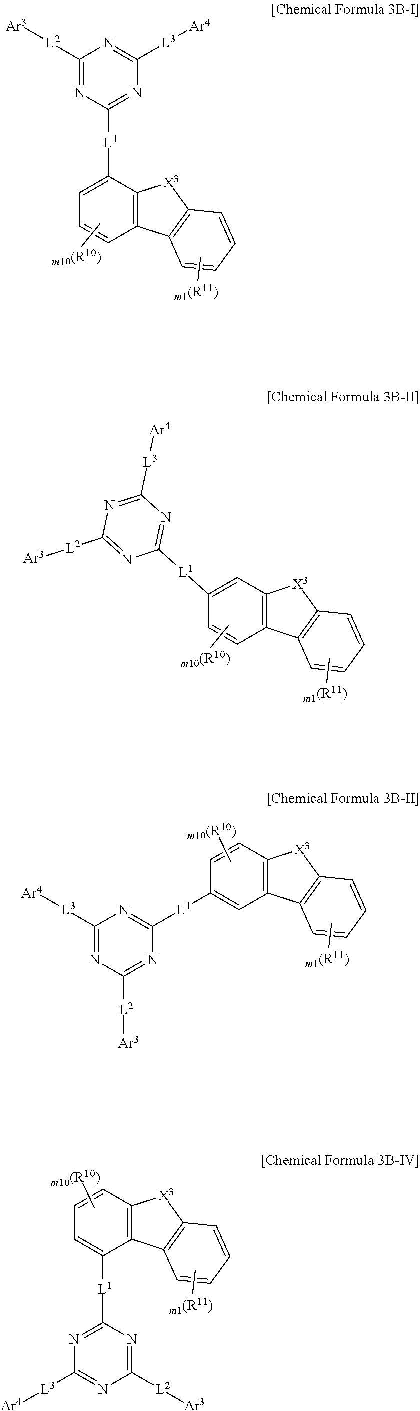

In Chemical Formula 3B-I to Chemical Formula 3B-IV, the definition of each substituent may be the same as those of Chemical Formula 3B.

In an implementation, Chemical Formula 3C may be, e.g., represented by Chemical Formula 3C-I or Chemical Formula 3C-II.

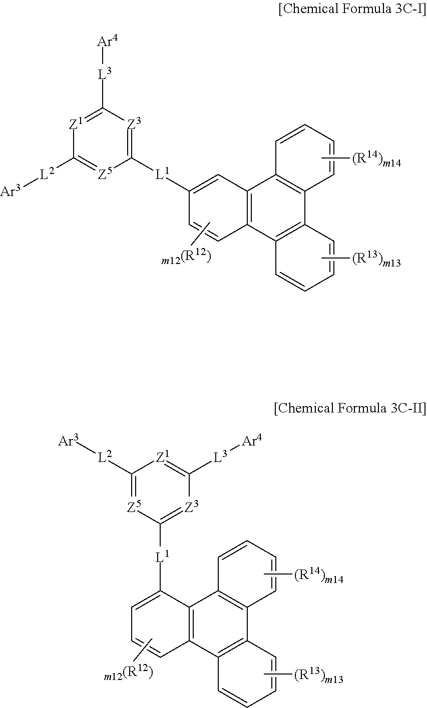

In Chemical Formula 3C-I and Chemical Formula 3C-II, each substituent may be defined the same as those of Chemical Formula 3C.

In an implementation, Chemical Formula 3 may be represented by Chemical Formula 3A-I.

In an implementation, in Chemical Formula 3A-I, L¹ to L³ may each independently be, e.g., a single bond or a substituted or unsubstituted C6 to C12 aryl group, Ar³ and Ar⁴ may each independently be, e.g., a substituted or unsubstituted phenyl group, a substituted or unsubstituted biphenyl group, a substituted or unsubstituted terphenyl group, or a substituted or unsubstituted naphthyl group, and R^{8a}, R^{8b}, R^{8c}, R^{8d}, R^{9a}, R^{9b}, R^{9c}, and R^{9d} may each independently be, e.g., hydrogen, deuterium, a cyano group, a substituted or unsubstituted C1 to C10 alkyl group, or a substituted or unsubstituted C6 to C12 aryl group.

The second compound may be, e.g., a compound of Group 2.

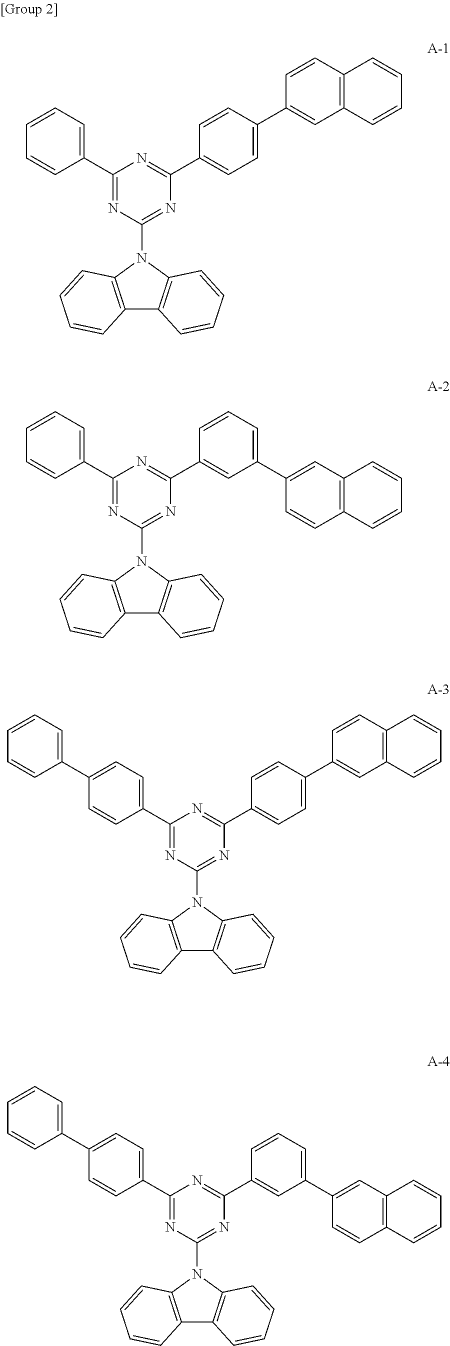 [SMILES: c1ccc(-c2nc(-c3ccc(-c4ccc5ccccc5c4)cc3)nc(-n3c4ccccc4c4ccccc43)n2)cc1 ; c1ccc(-c2nc(-c3cccc(-c4ccc5ccccc5c4)c3)nc(-n3c4ccccc4c4ccccc43)n2)cc1 ; c1ccc(-c2ccc(-c3nc(-c4ccc(-c5ccc6ccccc6c5)cc4)nc(-n4c5ccccc5c5ccccc54)n3)cc2)cc1 ; c1ccc(-c2ccc(-c3nc(-c4cccc(-c5ccc6ccccc6c5)c4)nc(-n4c5ccccc5c5ccccc54)n3)cc2)cc1]

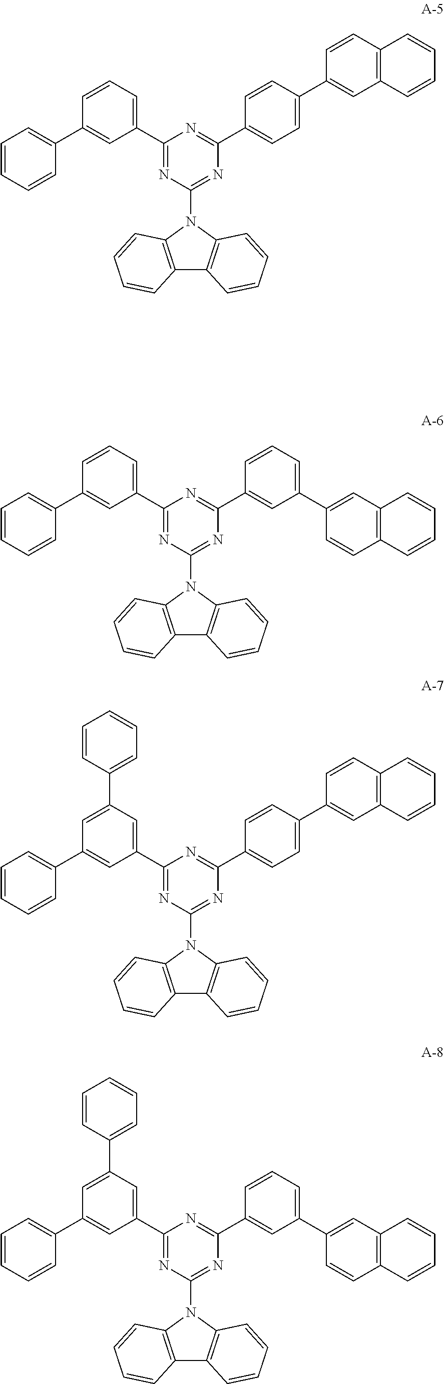 [SMILES: c1ccc(-c2cccc(-c3nc(-c4ccc(-c5ccc6ccccc6c5)cc4)nc(-n4c5ccccc5c5ccccc54)n3)c2)cc1 ; c1ccc(-c2cccc(-c3nc(-c4cccc(-c5ccc6ccccc6c5)c4)nc(-n4c5ccccc5c5ccccc54)n3)c2)cc1 ; c1ccc(-c2cc(-c3ccccc3)cc(-c3nc(-c4ccc(-c5ccc6ccccc6c5)cc4)nc(-n4c5ccccc5c5ccccc54)n3)c2)cc1 ; c1ccc(-c2cc(-c3ccccc3)cc(-c3nc(-c4cccc(-c5ccc6ccccc6c5)c4)nc(-n4c5ccccc5c5ccccc54)n3)c2)cc1]

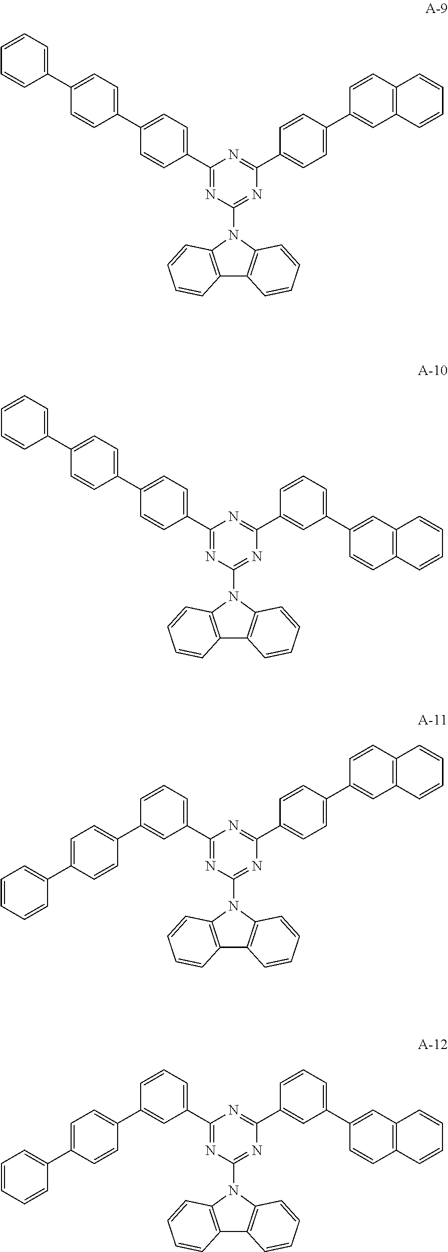 [SMILES: c1ccc(-c2ccc(-c3ccc(-c4nc(-c5ccc(-c6ccc7ccccc7c6)cc5)nc(-n5c6ccccc6c6ccccc65)n4)cc3)cc2)cc1 ; c1ccc(-c2ccc(-c3ccc(-c4nc(-c5cccc(-c6ccc7ccccc7c6)c5)nc(-n5c6ccccc6c6ccccc65)n4)cc3)cc2)cc1 ; c1ccc(-c2ccc(-c3cccc(-c4nc(-c5ccc(-c6ccc7ccccc7c6)cc5)nc(-n5c6ccccc6c6ccccc65)n4)c3)cc2)cc1 ; c1ccc(-c2ccc(-c3cccc(-c4nc(-c5cccc(-c6ccc7ccccc7c6)c5)nc(-n5c6ccccc6c6ccccc65)n4)c3)cc2)cc1]

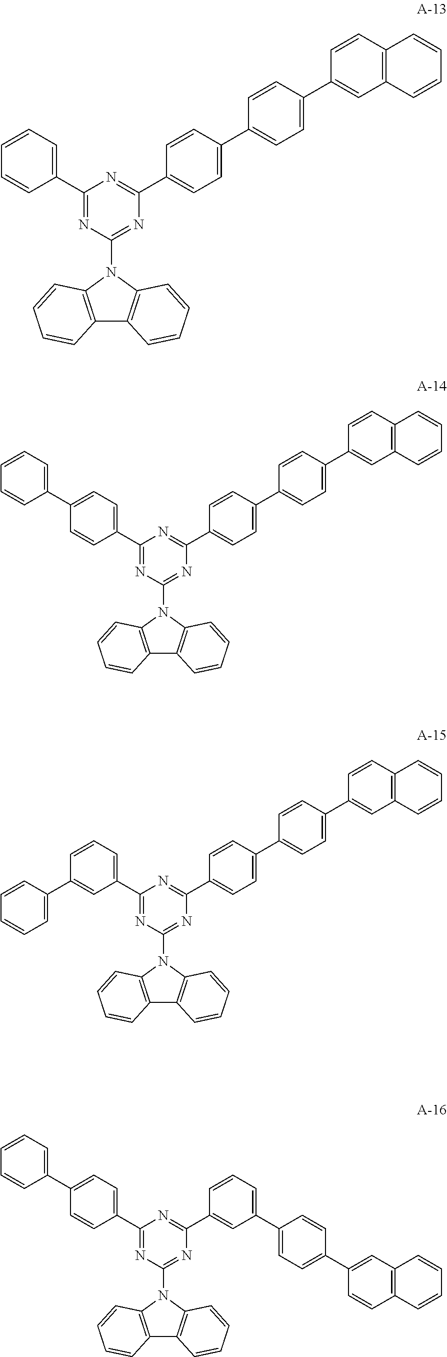 [SMILES: c1ccc(-c2nc(-c3ccc(-c4ccc(-c5ccc6ccccc6c5)cc4)cc3)nc(-n3c4ccccc4c4ccccc43)n2)cc1 ; c1ccc(-c2ccc(-c3nc(-c4ccc(-c5ccc(-c6ccc7ccccc7c6)cc5)cc4)nc(-n4c5ccccc5c5ccccc54)n3)cc2)cc1 ; c1ccc(-c2cccc(-c3nc(-c4ccc(-c5ccc(-c6ccc7ccccc7c6)cc5)cc4)nc(-n4c5ccccc5c5ccccc54)n3)c2)cc1 ; c1ccc(-c2ccc(-c3nc(-c4cccc(-c5ccc(-c6ccc7ccccc7c6)cc5)c4)nc(-n4c5ccccc5c5ccccc54)n3)cc2)cc1]

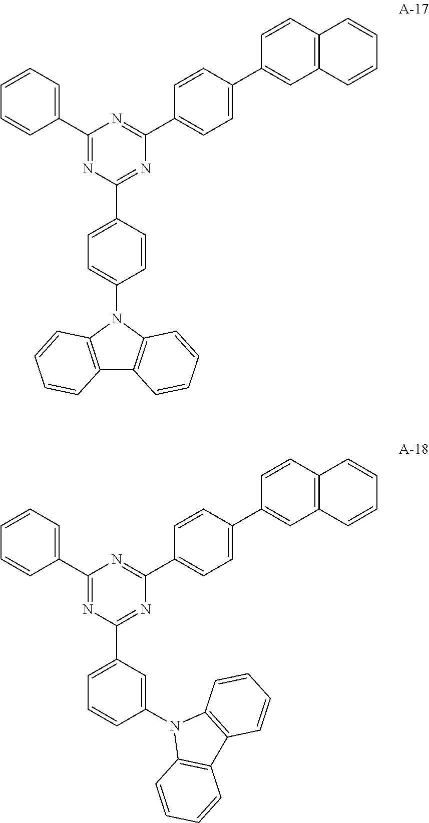 [SMILES: c1ccc(-c2nc(-c3ccc(-c4ccc5ccccc5c4)cc3)nc(-c3ccc(-n4c5ccccc5c5ccccc54)cc3)n2)cc1 ; c1ccc(-c2nc(-c3ccc(-c4ccc5ccccc5c4)cc3)nc(-c3cccc(-n4c5ccccc5c5ccccc54)c3)n2)cc1]

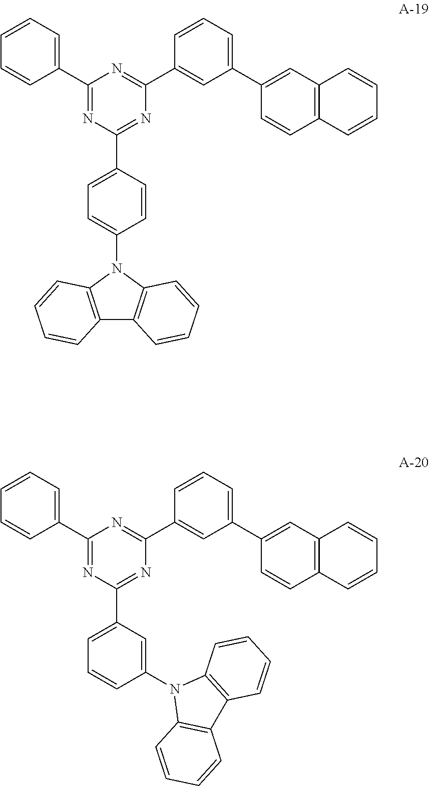 [SMILES: c1ccc(-c2nc(-c3ccc(-n4c5ccccc5c5ccccc54)cc3)nc(-c3cccc(-c4ccc5ccccc5c4)c3)n2)cc1 ; c1ccc(-c2nc(-c3cccc(-c4ccc5ccccc5c4)c3)nc(-c3cccc(-n4c5ccccc5c5ccccc54)c3)n2)cc1]

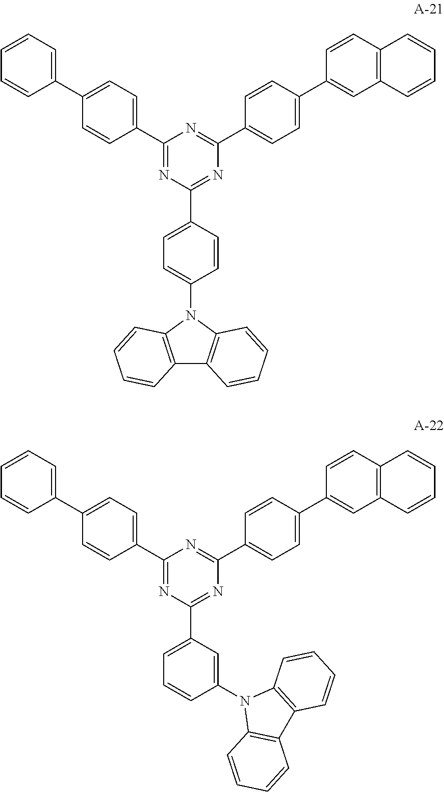 [SMILES: c1ccc(-c2ccc(-c3nc(-c4ccc(-c5ccc6ccccc6c5)cc4)nc(-c4ccc(-n5c6ccccc6c6ccccc65)cc4)n3)cc2)cc1 ; c1ccc(-c2ccc(-c3nc(-c4ccc(-c5ccc6ccccc6c5)cc4)nc(-c4cccc(-n5c6ccccc6c6ccccc65)c4)n3)cc2)cc1]

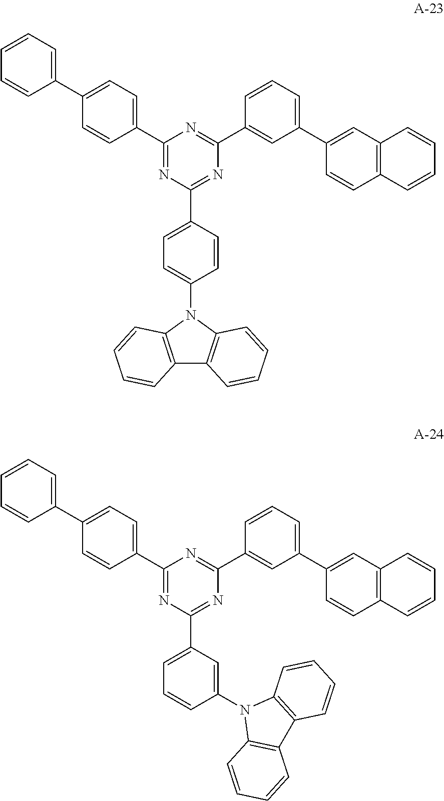 [SMILES: c1ccc(-c2ccc(-c3nc(-c4ccc(-n5c6ccccc6c6ccccc65)cc4)nc(-c4cccc(-c5ccc6ccccc6c5)c4)n3)cc2)cc1 ; c1ccc(-c2ccc(-c3nc(-c4cccc(-c5ccc6ccccc6c5)c4)nc(-c4cccc(-n5c6ccccc6c6ccccc65)c4)n3)cc2)cc1]

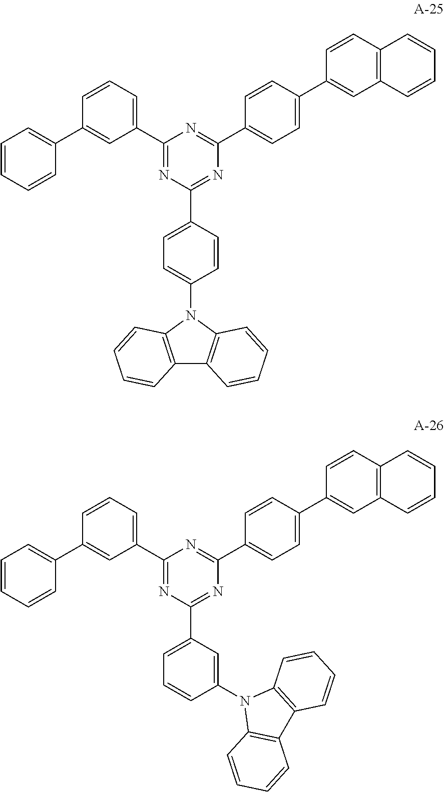 [SMILES: c1ccc(-c2cccc(-c3nc(-c4ccc(-c5ccc6ccccc6c5)cc4)nc(-c4ccc(-n5c6ccccc6c6ccccc65)cc4)n3)c2)cc1 ; c1ccc(-c2cccc(-c3nc(-c4ccc(-c5ccc6ccccc6c5)cc4)nc(-c4cccc(-n5c6ccccc6c6ccccc65)c4)n3)c2)cc1]

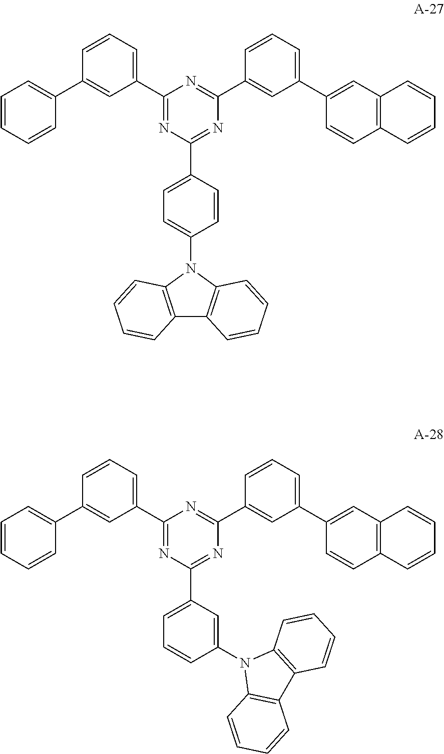 [SMILES: c1ccc(-c2cccc(-c3nc(-c4ccc(-n5c6ccccc6c6ccccc65)cc4)nc(-c4cccc(-c5ccc6ccccc6c5)c4)n3)c2)cc1 ; c1ccc(-c2cccc(-c3nc(-c4cccc(-c5ccc6ccccc6c5)c4)nc(-c4cccc(-n5c6ccccc6c6ccccc65)c4)n3)c2)cc1]

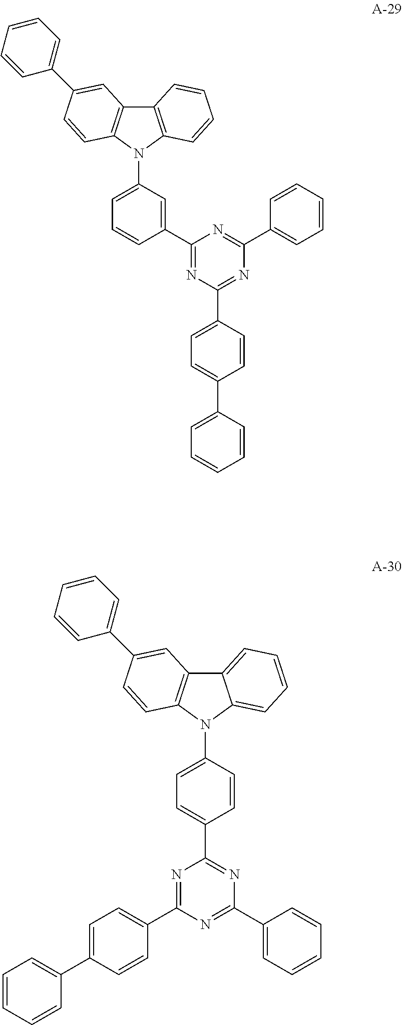 [SMILES: c1ccc(-c2ccc(-c3nc(-c4ccccc4)nc(-c4cccc(-n5c6ccccc6c6cc(-c7ccccc7)ccc65)c4)n3)cc2)cc1 ; c1ccc(-c2ccc(-c3nc(-c4ccccc4)nc(-c4ccc(-n5c6ccccc6c6cc(-c7ccccc7)ccc65)cc4)n3)cc2)cc1]

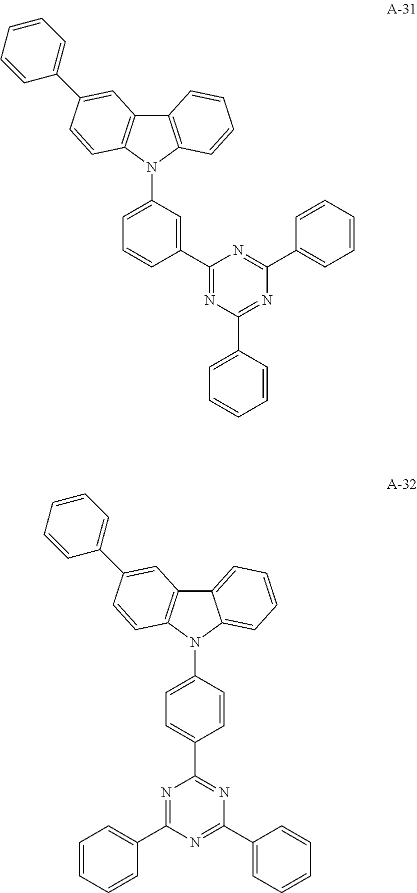 [SMILES: c1ccc(-c2ccc3c(c2)c2ccccc2n3-c2cccc(-c3nc(-c4ccccc4)nc(-c4ccccc4)n3)c2)cc1 ; c1ccc(-c2ccc3c(c2)c2ccccc2n3-c2ccc(-c3nc(-c4ccccc4)nc(-c4ccccc4)n3)cc2)cc1]

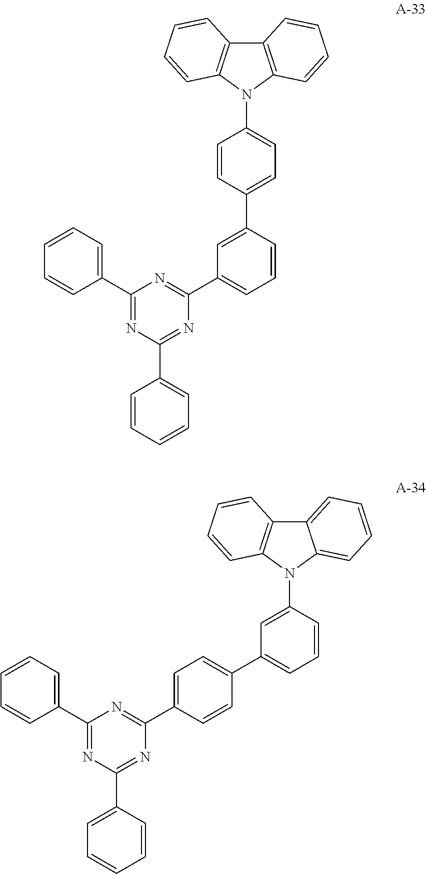 [SMILES: c1ccc(-c2nc(-c3ccccc3)nc(-c3cccc(-c4ccc(-n5c6ccccc6c6ccccc65)cc4)c3)n2)cc1 ; c1ccc(-c2nc(-c3ccccc3)nc(-c3ccc(-c4cccc(-n5c6ccccc6c6ccccc65)c4)cc3)n2)cc1]

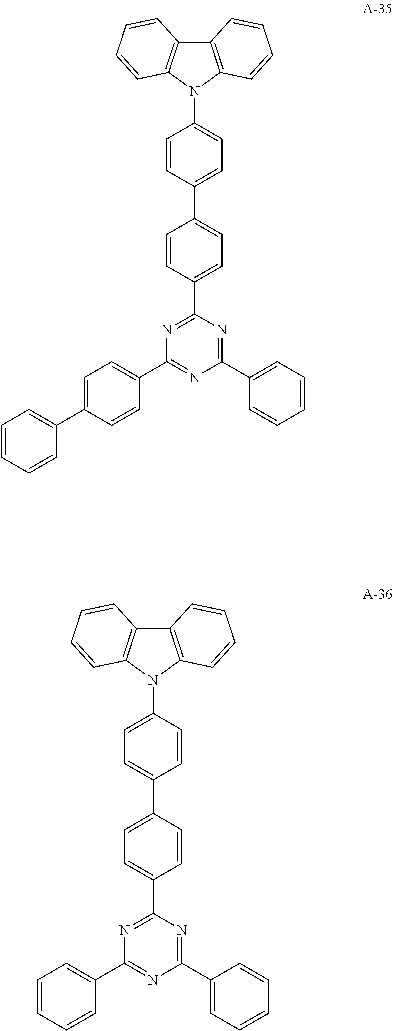 [SMILES: c1ccc(-c2ccc(-c3nc(-c4ccccc4)nc(-c4ccc(-c5ccc(-n6c7ccccc7c7ccccc76)cc5)cc4)n3)cc2)cc1 ; c1ccc(-c2nc(-c3ccccc3)nc(-c3ccc(-c4ccc(-n5c6ccccc6c6ccccc65)cc4)cc3)n2)cc1]

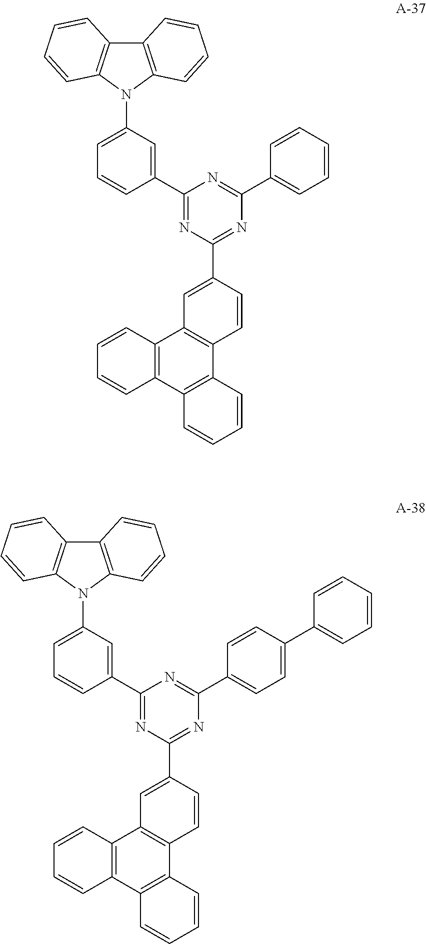 [SMILES: c1ccc(-c2nc(-c3cccc(-n4c5ccccc5c5ccccc54)c3)nc(-c3ccc4c5ccccc5c5ccccc5c4c3)n2)cc1 ; c1ccc(-c2ccc(-c3nc(-c4cccc(-n5c6ccccc6c6ccccc65)c4)nc(-c4ccc5c6ccccc6c6ccccc6c5c4)n3)cc2)cc1]

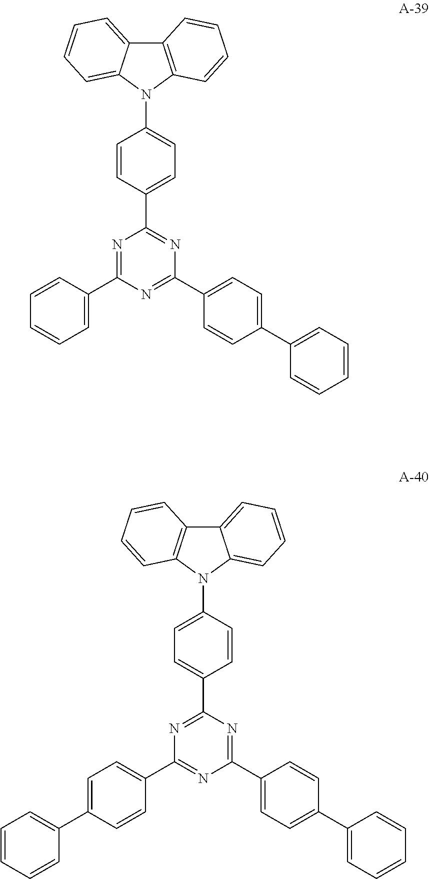 [SMILES: c1ccc(-c2ccc(-c3nc(-c4ccccc4)nc(-c4ccc(-n5c6ccccc6c6ccccc65)cc4)n3)cc2)cc1 ; c1ccc(-c2ccc(-c3nc(-c4ccc(-c5ccccc5)cc4)nc(-c4ccc(-n5c6ccccc6c6ccccc65)cc4)n3)cc2)cc1]

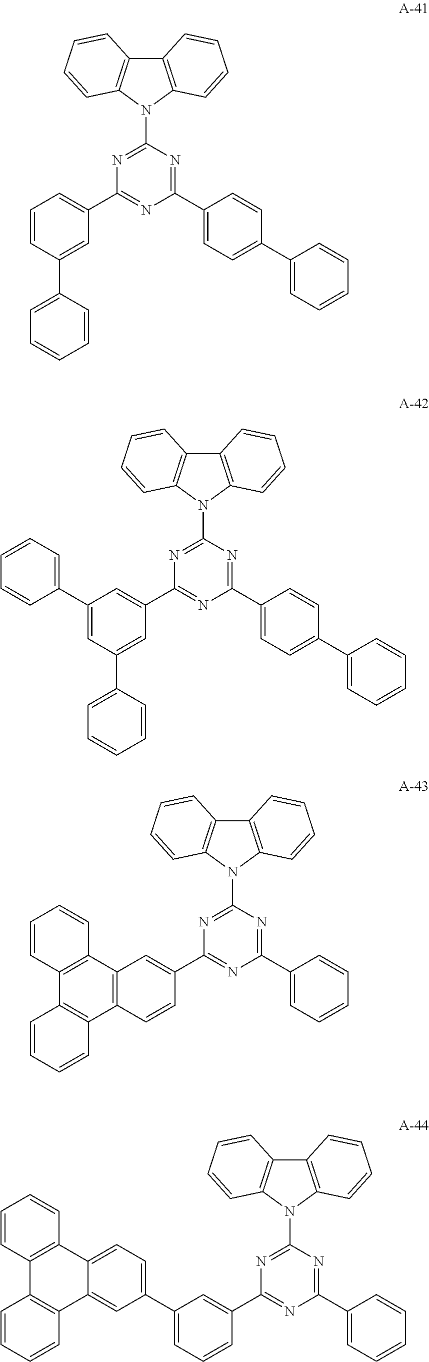 [SMILES: c1ccc(-c2ccc(-c3nc(-c4cccc(-c5ccccc5)c4)nc(-n4c5ccccc5c5ccccc54)n3)cc2)cc1 ; c1ccc(-c2ccc(-c3nc(-c4cc(-c5ccccc5)cc(-c5ccccc5)c4)nc(-n4c5ccccc5c5ccccc54)n3)cc2)cc1 ; c1ccc(-c2nc(-c3ccc4c5ccccc5c5ccccc5c4c3)nc(-n3c4ccccc4c4ccccc43)n2)cc1 ; c1ccc(-c2nc(-c3cccc(-c4ccc5c6ccccc6c6ccccc6c5c4)c3)nc(-n3c4ccccc4c4ccccc43)n2)cc1]

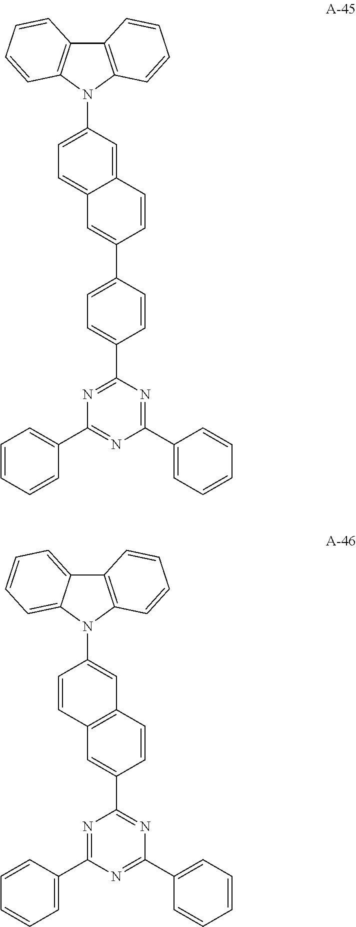 [SMILES: c1ccc(-c2nc(-c3ccccc3)nc(-c3ccc(-c4ccc5cc(-n6c7ccccc7c7ccccc76)ccc5c4)cc3)n2)cc1 ; c1ccc(-c2nc(-c3ccccc3)nc(-c3ccc4cc(-n5c6ccccc6c6ccccc65)ccc4c3)n2)cc1]

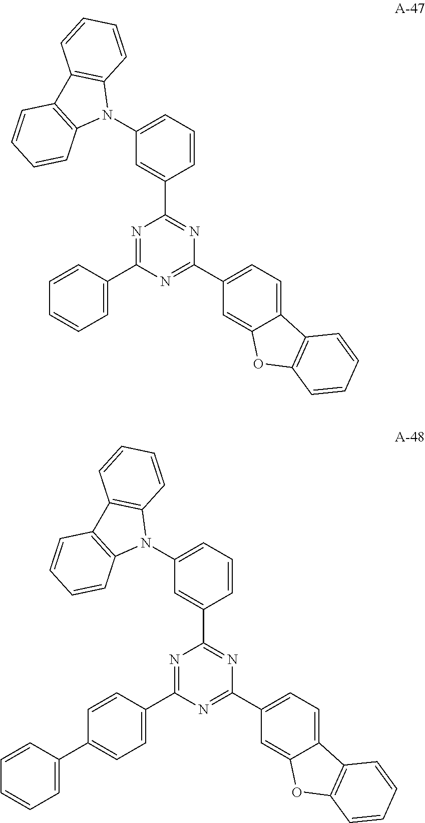 [SMILES: c1ccc(-c2nc(-c3cccc(-n4c5ccccc5c5ccccc54)c3)nc(-c3ccc4c(c3)oc3ccccc34)n2)cc1 ; c1ccc(-c2ccc(-c3nc(-c4cccc(-n5c6ccccc6c6ccccc65)c4)nc(-c4ccc5c(c4)oc4ccccc45)n3)cc2)cc1]

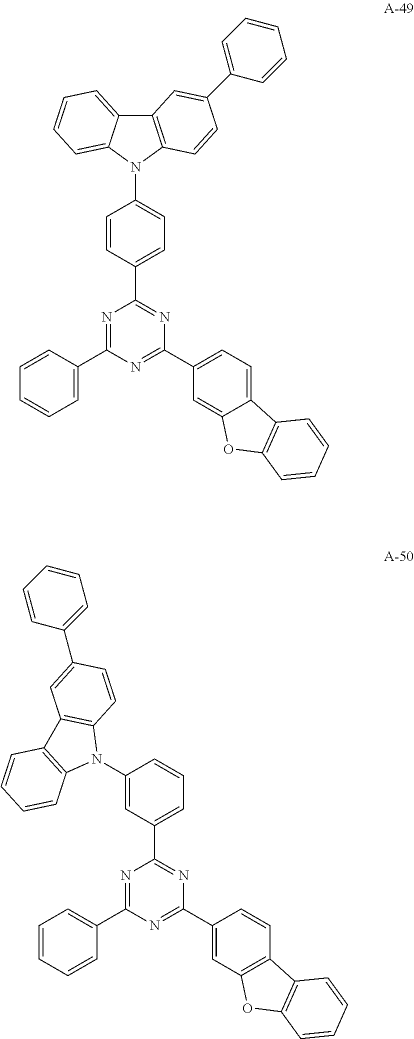 [SMILES: c1ccc(-c2ccc3c(c2)c2ccccc2n3-c2ccc(-c3nc(-c4ccccc4)nc(-c4ccc5c(c4)oc4ccccc45)n3)cc2)cc1 ; c1ccc(-c2ccc3c(c2)c2ccccc2n3-c2cccc(-c3nc(-c4ccccc4)nc(-c4ccc5c(c4)oc4ccccc45)n3)c2)cc1]

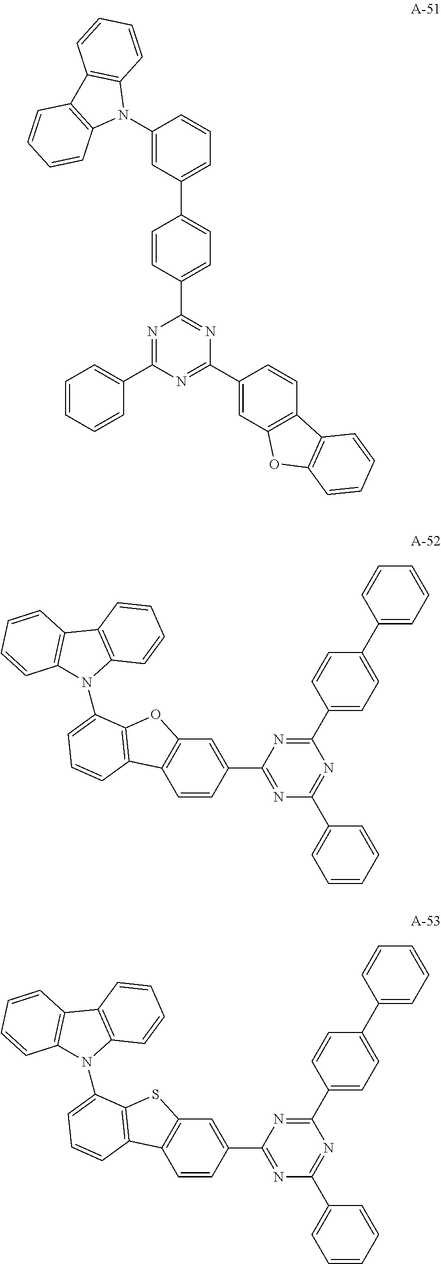 [SMILES: c1ccc(-c2nc(-c3ccc(-c4cccc(-n5c6ccccc6c6ccccc65)c4)cc3)nc(-c3ccc4c(c3)oc3ccccc34)n2)cc1 ; c1ccc(-c2ccc(-c3nc(-c4ccccc4)nc(-c4ccc5c(c4)oc4c(-n6c7ccccc7c7ccccc76)cccc45)n3)cc2)cc1 ; c1ccc(-c2ccc(-c3nc(-c4ccccc4)nc(-c4ccc5c(c4)sc4c(-n6c7ccccc7c7ccccc76)cccc45)n3)cc2)cc1]

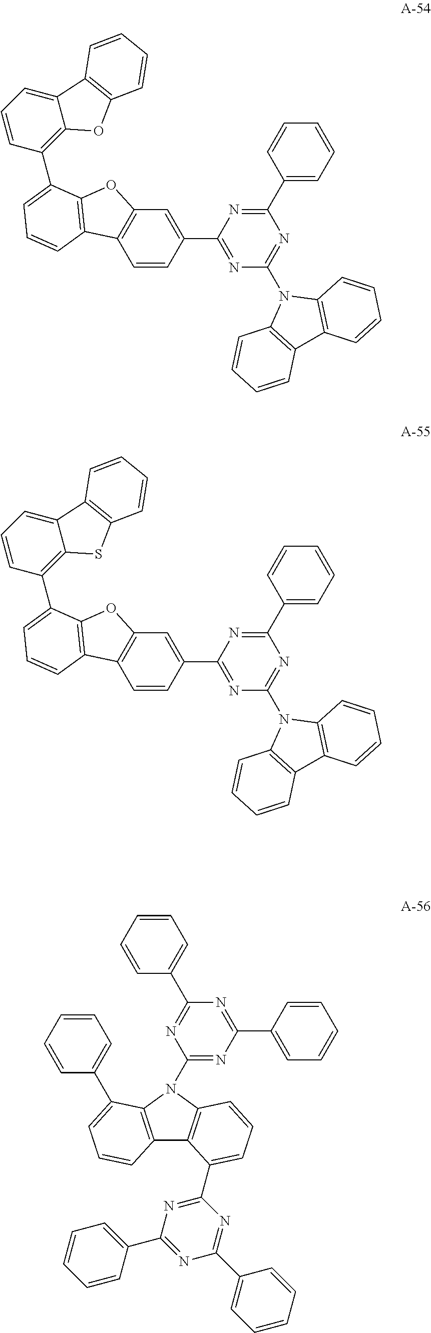 [SMILES: c1ccc(-c2nc(-c3ccc4c(c3)oc3c(-c5cccc6c5oc5ccccc56)cccc34)nc(-n3c4ccccc4c4ccccc43)n2)cc1 ; c1ccc(-c2nc(-c3ccc4c(c3)oc3c(-c5cccc6c5sc5ccccc56)cccc34)nc(-n3c4ccccc4c4ccccc43)n2)cc1 ; c1ccc(-c2nc(-c3ccccc3)nc(-c3cccc4c3c3cccc(-c5ccccc5)c3n4-c3nc(-c4ccccc4)nc(-c4ccccc4)n3)n2)cc1]

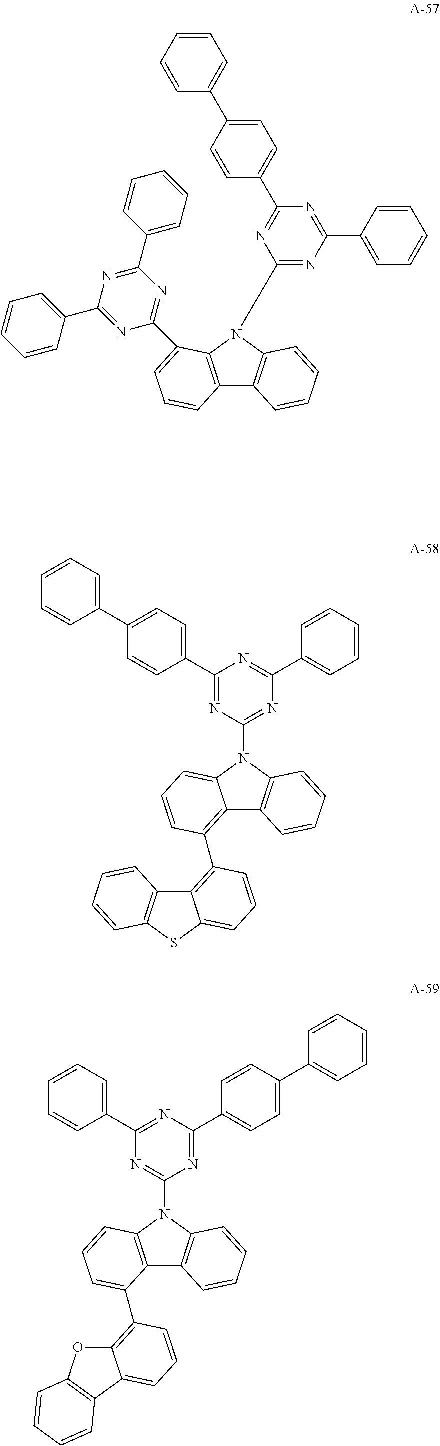 [SMILES: c1ccc(-c2ccc(-c3nc(-c4ccccc4)nc(-n4c5ccccc5c5cccc(-c6nc(-c7ccccc7)nc(-c7ccccc7)n6)c54)n3)cc2)cc1 ; c1ccc(-c2ccc(-c3nc(-c4ccccc4)nc(-n4c5ccccc5c5c(-c6cccc7sc8ccccc8c67)cccc54)n3)cc2)cc1 ; c1ccc(-c2ccc(-c3nc(-c4ccccc4)nc(-n4c5ccccc5c5c(-c6cccc7c6oc6ccccc67)cccc54)n3)cc2)cc1]

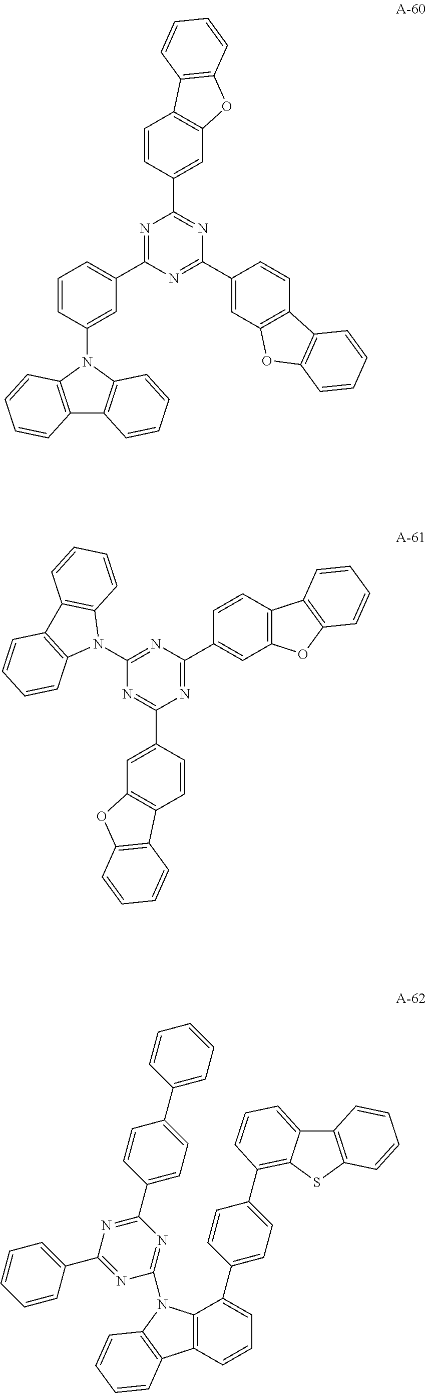 [SMILES: c1cc(-c2nc(-c3ccc4c(c3)oc3ccccc34)nc(-c3ccc4c(c3)oc3ccccc34)n2)cc(-n2c3ccccc3c3ccccc32)c1 ; c1ccc2c(c1)oc1cc(-c3nc(-c4ccc5c(c4)oc4ccccc45)nc(-n4c5ccccc5c5ccccc54)n3)ccc12 ; c1ccc(-c2ccc(-c3nc(-c4ccccc4)nc(-n4c5ccccc5c5cccc(-c6ccc(-c7cccc8c7sc7ccccc78)cc6)c54)n3)cc2)cc1]

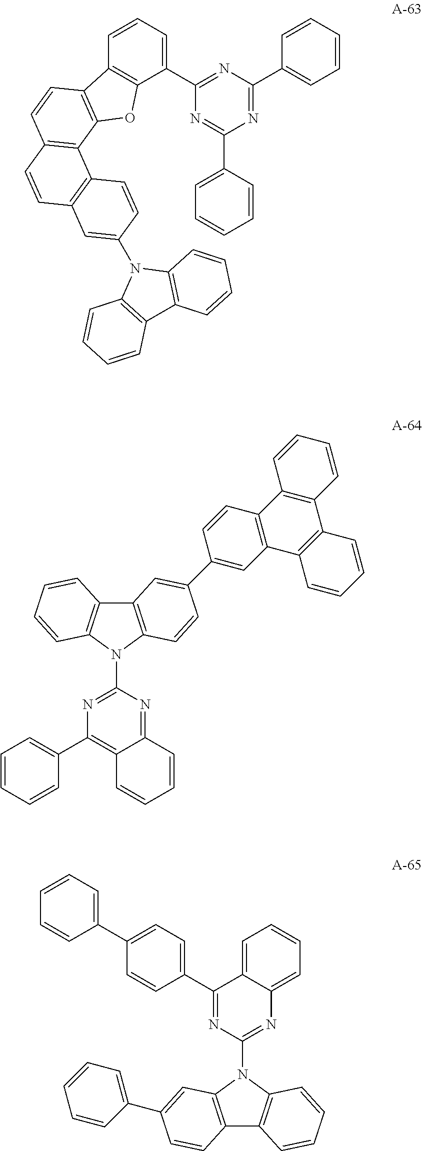 [SMILES: c1ccc(-c2nc(-c3ccccc3)nc(-c3cccc4c3oc3c4ccc4ccc5cc(-n6c7ccccc7c7ccccc76)ccc5c43)n2)cc1 ; c1ccc(-c2nc(-n3c4ccccc4c4cc(-c5ccc6c7ccccc7c7ccccc7c6c5)ccc43)nc3ccccc23)cc1 ; c1ccc(-c2ccc(-c3nc(-n4c5ccccc5c5ccc(-c6ccccc6)cc54)nc4ccccc34)cc2)cc1]

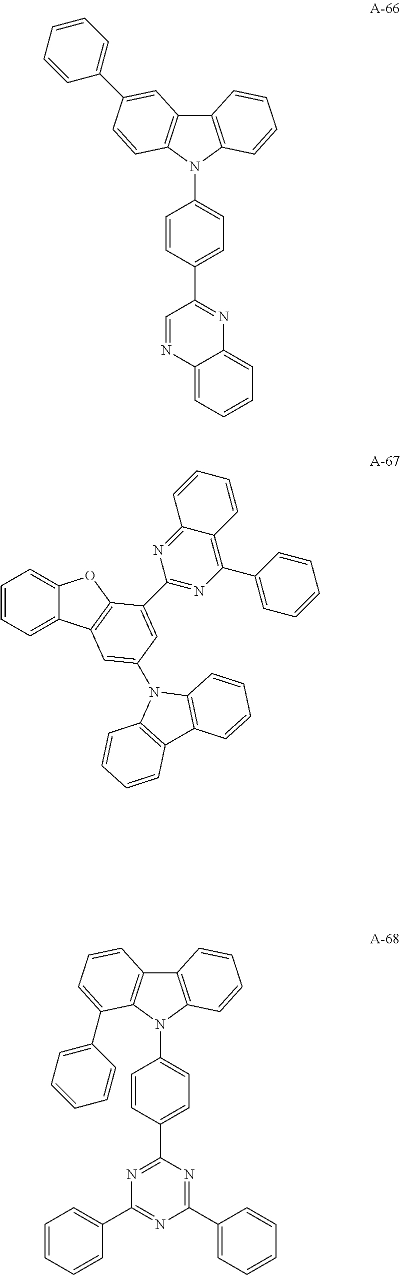 [SMILES: c1ccc(-c2ccc3c(c2)c2ccccc2n3-c2ccc(-c3cnc4ccccc4n3)cc2)cc1 ; c1ccc(-c2nc(-c3cc(-n4c5ccccc5c5ccccc54)cc4c3oc3ccccc34)nc3ccccc23)cc1 ; c1ccc(-c2nc(-c3ccccc3)nc(-c3ccc(-n4c5ccccc5c5cccc(-c6ccccc6)c54)cc3)n2)cc1]

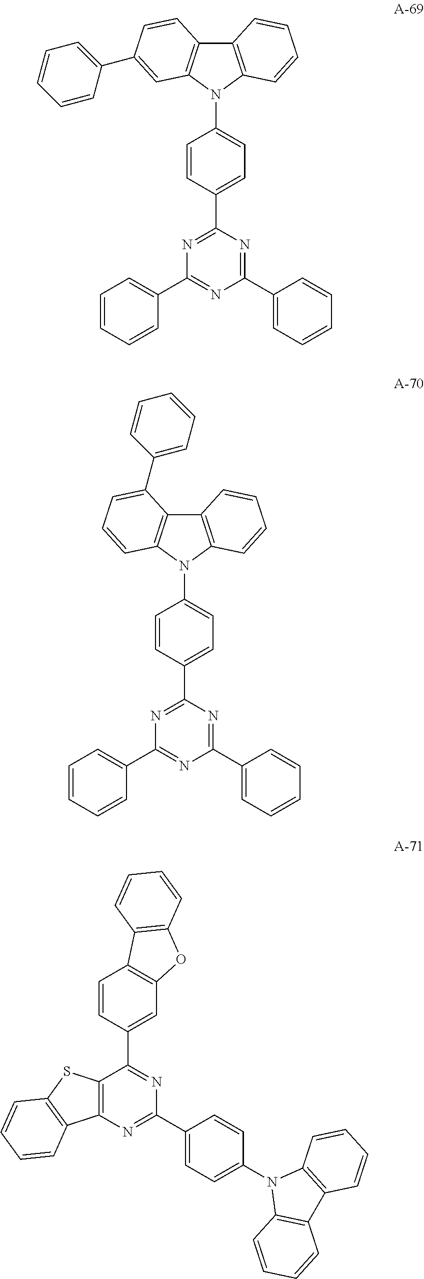 [SMILES: c1ccc(-c2ccc3c4ccccc4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5ccccc5c5c(-c6ccccc6)cccc54)cc3)n2)cc1 ; c1ccc2c(c1)oc1cc(-c3nc(-c4ccc(-n5c6ccccc6c6ccccc65)cc4)nc4c3sc3ccccc34)ccc12]

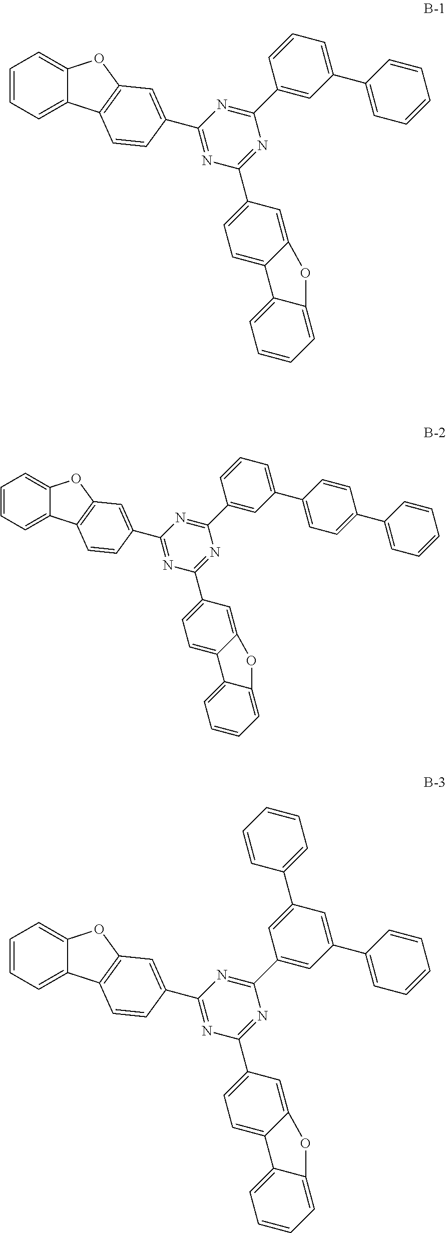 [SMILES: c1ccc(-c2cccc(-c3nc(-c4ccc5c(c4)oc4ccccc45)nc(-c4ccc5c(c4)oc4ccccc45)n3)c2)cc1 ; c1ccc(-c2ccc(-c3cccc(-c4nc(-c5ccc6c(c5)oc5ccccc56)nc(-c5ccc6c(c5)oc5ccccc56)n4)c3)cc2)cc1 ; c1ccc(-c2cc(-c3ccccc3)cc(-c3nc(-c4ccc5c(c4)oc4ccccc45)nc(-c4ccc5c(c4)oc4ccccc45)n3)c2)cc1]

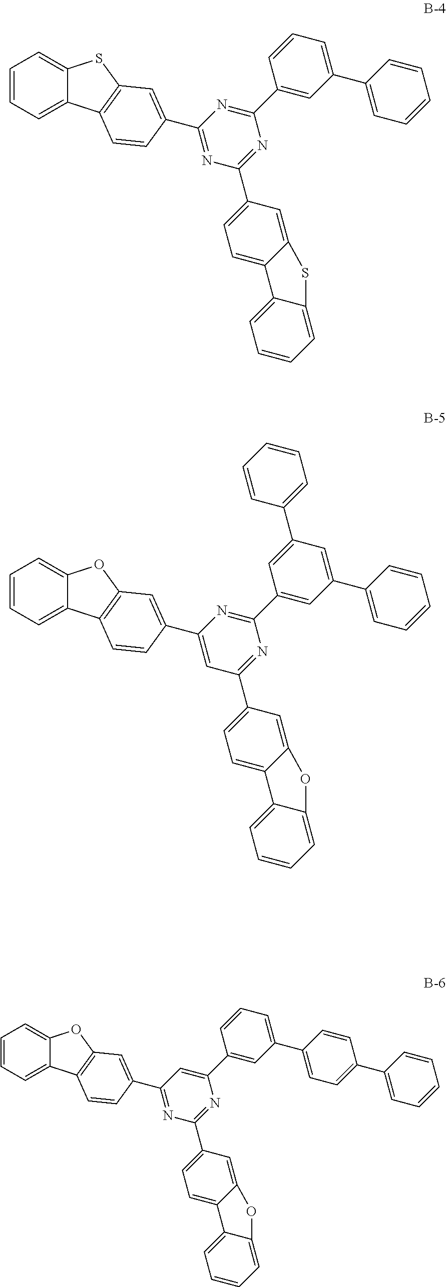 [SMILES: c1ccc(-c2cccc(-c3nc(-c4ccc5c(c4)sc4ccccc45)nc(-c4ccc5c(c4)sc4ccccc45)n3)c2)cc1 ; c1ccc(-c2cc(-c3ccccc3)cc(-c3nc(-c4ccc5c(c4)oc4ccccc45)cc(-c4ccc5c(c4)oc4ccccc45)n3)c2)cc1 ; c1ccc(-c2ccc(-c3cccc(-c4cc(-c5ccc6c(c5)oc5ccccc56)nc(-c5ccc6c(c5)oc5ccccc56)n4)c3)cc2)cc1]

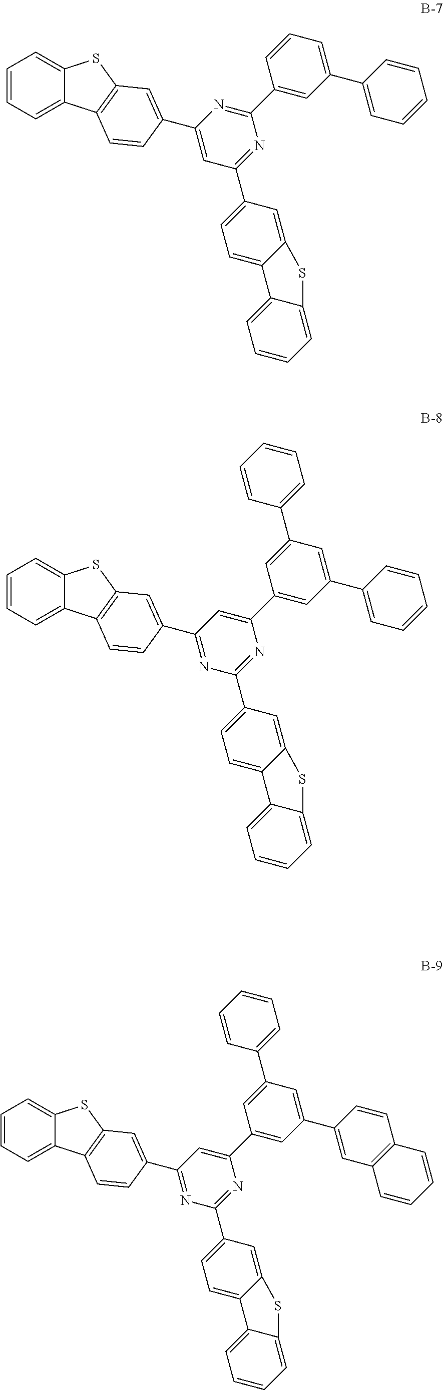 [SMILES: c1ccc(-c2cccc(-c3nc(-c4ccc5c(c4)sc4ccccc45)cc(-c4ccc5c(c4)sc4ccccc45)n3)c2)cc1 ; c1ccc(-c2cc(-c3ccccc3)cc(-c3cc(-c4ccc5c(c4)sc4ccccc45)nc(-c4ccc5c(c4)sc4ccccc45)n3)c2)cc1 ; c1ccc(-c2cc(-c3ccc4ccccc4c3)cc(-c3cc(-c4ccc5c(c4)sc4ccccc45)nc(-c4ccc5c(c4)sc4ccccc45)n3)c2)cc1]

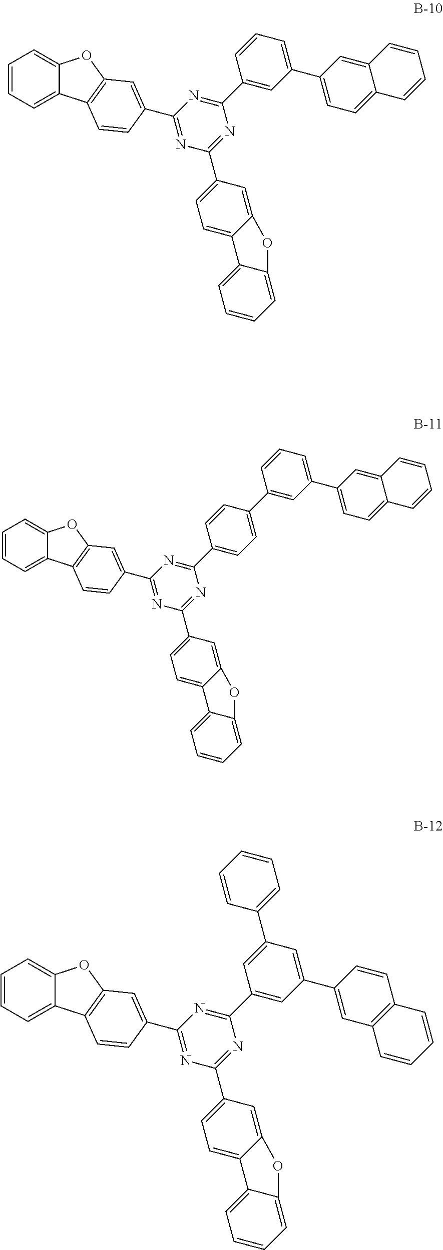 [SMILES: c1cc(-c2ccc3ccccc3c2)cc(-c2nc(-c3ccc4c(c3)oc3ccccc34)nc(-c3ccc4c(c3)oc3ccccc34)n2)c1 ; c1cc(-c2ccc(-c3nc(-c4ccc5c(c4)oc4ccccc45)nc(-c4ccc5c(c4)oc4ccccc45)n3)cc2)cc(-c2ccc3ccccc3c2)c1 ; c1ccc(-c2cc(-c3ccc4ccccc4c3)cc(-c3nc(-c4ccc5c(c4)oc4ccccc45)nc(-c4ccc5c(c4)oc4ccccc45)n3)c2)cc1]

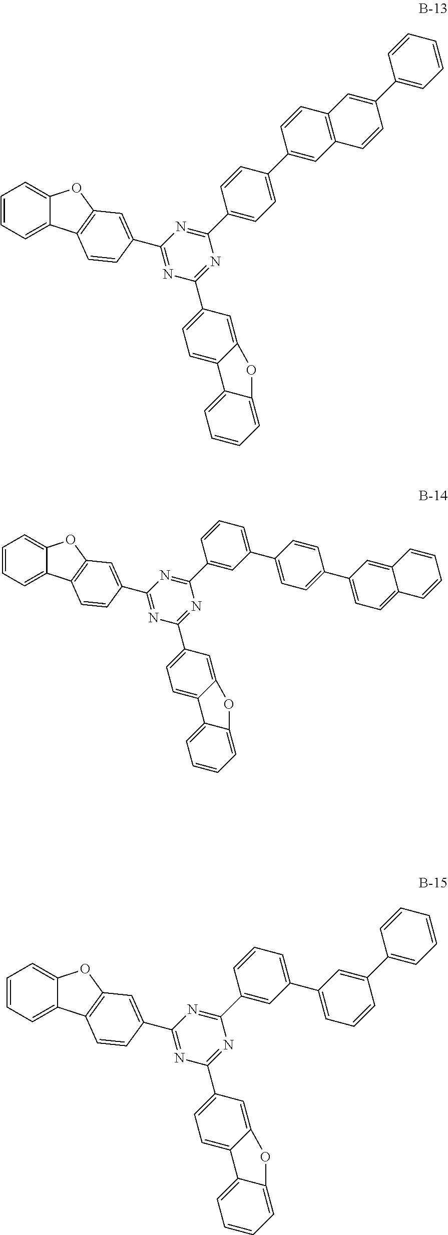 [SMILES: c1ccc(-c2ccc3cc(-c4ccc(-c5nc(-c6ccc7c(c6)oc6ccccc67)nc(-c6ccc7c(c6)oc6ccccc67)n5)cc4)ccc3c2)cc1 ; c1cc(-c2ccc(-c3ccc4ccccc4c3)cc2)cc(-c2nc(-c3ccc4c(c3)oc3ccccc34)nc(-c3ccc4c(c3)oc3ccccc34)n2)c1 ; c1ccc(-c2cccc(-c3cccc(-c4nc(-c5ccc6c(c5)oc5ccccc56)nc(-c5ccc6c(c5)oc5ccccc56)n4)c3)c2)cc1]

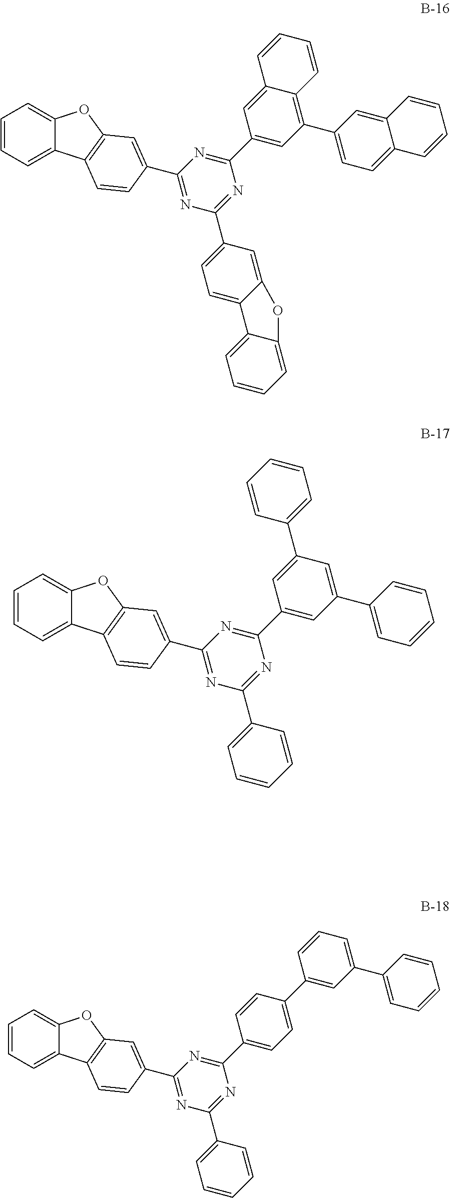 [SMILES: c1ccc2cc(-c3cc(-c4nc(-c5ccc6c(c5)oc5ccccc56)nc(-c5ccc6c(c5)oc5ccccc56)n4)cc4ccccc34)ccc2c1 ; c1ccc(-c2cc(-c3ccccc3)cc(-c3nc(-c4ccccc4)nc(-c4ccc5c(c4)oc4ccccc45)n3)c2)cc1 ; c1ccc(-c2cccc(-c3ccc(-c4nc(-c5ccccc5)nc(-c5ccc6c(c5)oc5ccccc56)n4)cc3)c2)cc1]

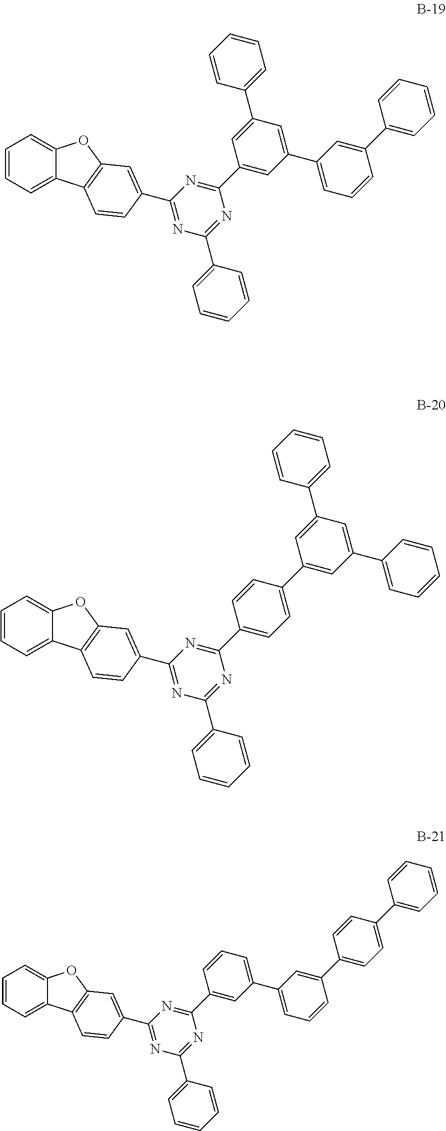 [SMILES: c1ccc(-c2cccc(-c3cc(-c4ccccc4)cc(-c4nc(-c5ccccc5)nc(-c5ccc6c(c5)oc5ccccc56)n4)c3)c2)cc1 ; c1ccc(-c2cc(-c3ccccc3)cc(-c3ccc(-c4nc(-c5ccccc5)nc(-c5ccc6c(c5)oc5ccccc56)n4)cc3)c2)cc1 ; c1ccc(-c2ccc(-c3cccc(-c4cccc(-c5nc(-c6ccccc6)nc(-c6ccc7c(c6)oc6ccccc67)n5)c4)c3)cc2)cc1]

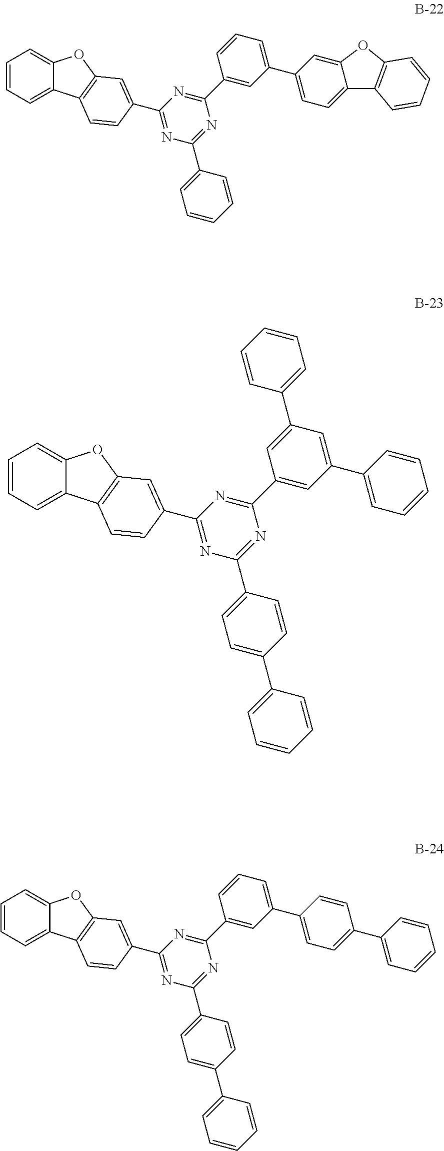 [SMILES: c1ccc(-c2nc(-c3cccc(-c4ccc5c(c4)oc4ccccc45)c3)nc(-c3ccc4c(c3)oc3ccccc34)n2)cc1 ; c1ccc(-c2ccc(-c3nc(-c4cc(-c5ccccc5)cc(-c5ccccc5)c4)nc(-c4ccc5c(c4)oc4ccccc45)n3)cc2)cc1 ; c1ccc(-c2ccc(-c3cccc(-c4nc(-c5ccc(-c6ccccc6)cc5)nc(-c5ccc6c(c5)oc5ccccc56)n4)c3)cc2)cc1]

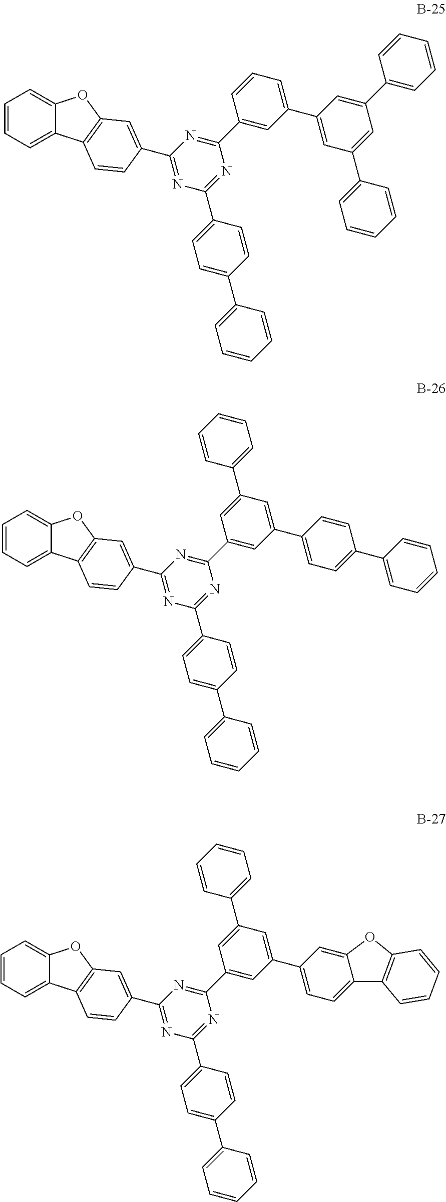 [SMILES: c1ccc(-c2ccc(-c3nc(-c4cccc(-c5cc(-c6ccccc6)cc(-c6ccccc6)c5)c4)nc(-c4ccc5c(c4)oc4ccccc45)n3)cc2)cc1 ; c1ccc(-c2ccc(-c3cc(-c4ccccc4)cc(-c4nc(-c5ccc(-c6ccccc6)cc5)nc(-c5ccc6c(c5)oc5ccccc56)n4)c3)cc2)cc1 ; c1ccc(-c2ccc(-c3nc(-c4cc(-c5ccccc5)cc(-c5ccc6c(c5)oc5ccccc56)c4)nc(-c4ccc5c(c4)oc4ccccc45)n3)cc2)cc1]

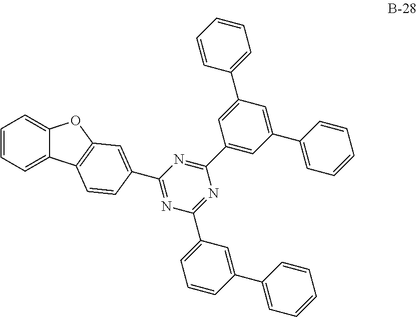 [SMILES: c1ccc(-c2cccc(-c3nc(-c4cc(-c5ccccc5)cc(-c5ccccc5)c4)nc(-c4ccc5c(c4)oc4ccccc45)n3)c2)cc1]

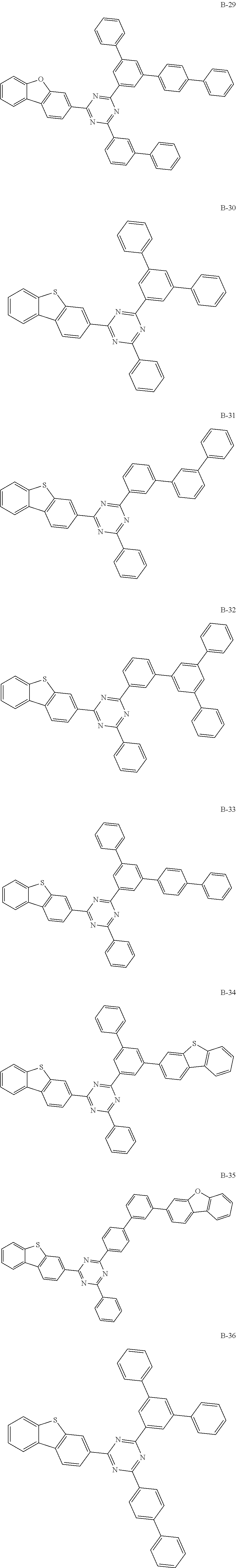

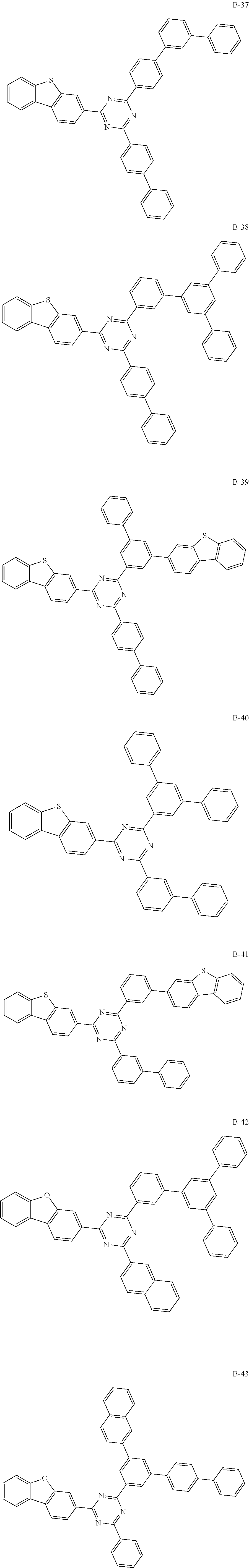

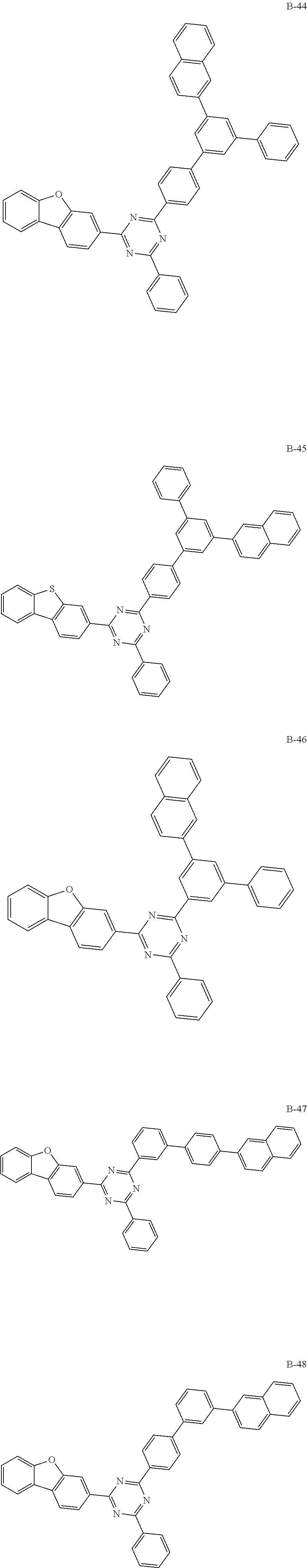 [SMILES: c1ccc(-c2cc(-c3ccc(-c4nc(-c5ccccc5)nc(-c5ccc6c(c5)oc5ccccc56)n4)cc3)cc(-c3ccc4ccccc4c3)c2)cc1 ; c1ccc(-c2cc(-c3ccc(-c4nc(-c5ccccc5)nc(-c5ccc6c(c5)sc5ccccc56)n4)cc3)cc(-c3ccc4ccccc4c3)c2)cc1 ; c1ccc(-c2cc(-c3ccc4ccccc4c3)cc(-c3nc(-c4ccccc4)nc(-c4ccc5c(c4)oc4ccccc45)n3)c2)cc1 ; c1ccc(-c2nc(-c3cccc(-c4ccc(-c5ccc6ccccc6c5)cc4)c3)nc(-c3ccc4c(c3)oc3ccccc34)n2)cc1 ; c1ccc(-c2nc(-c3ccc(-c4cccc(-c5ccc6ccccc6c5)c4)cc3)nc(-c3ccc4c(c3)oc3ccccc34)n2)cc1]

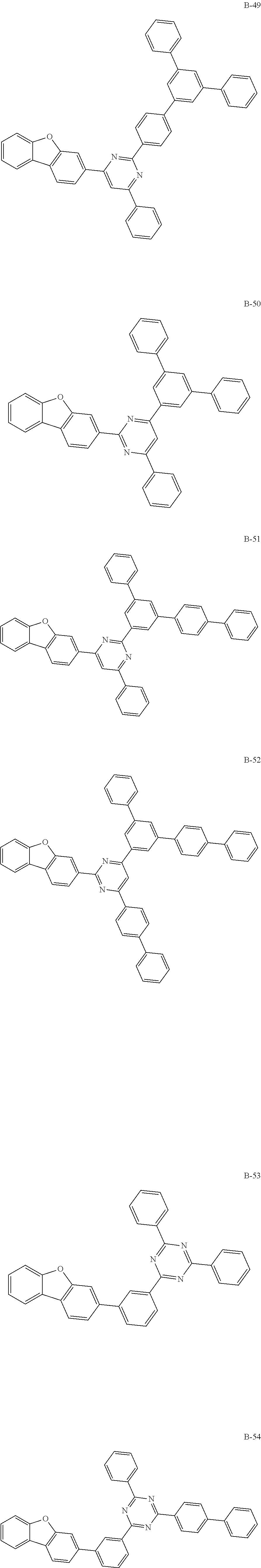

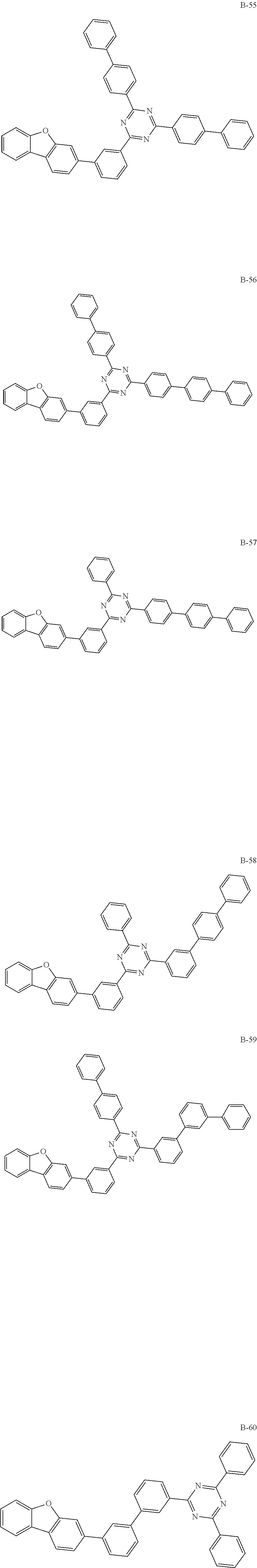

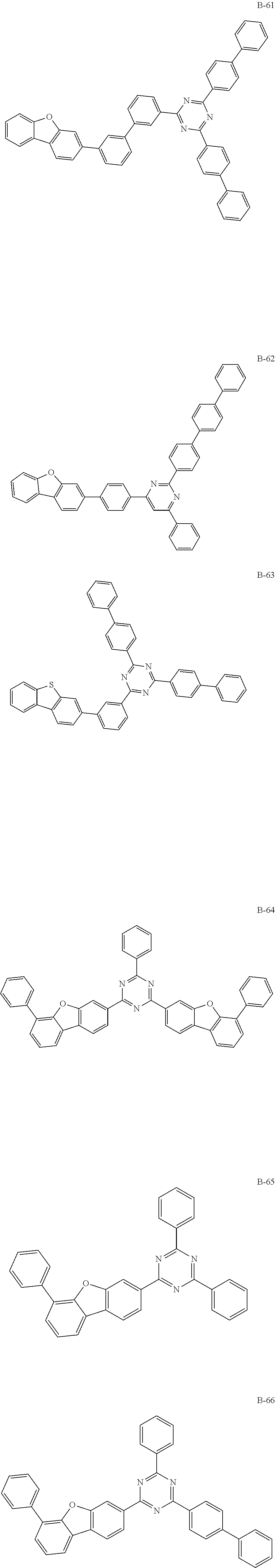

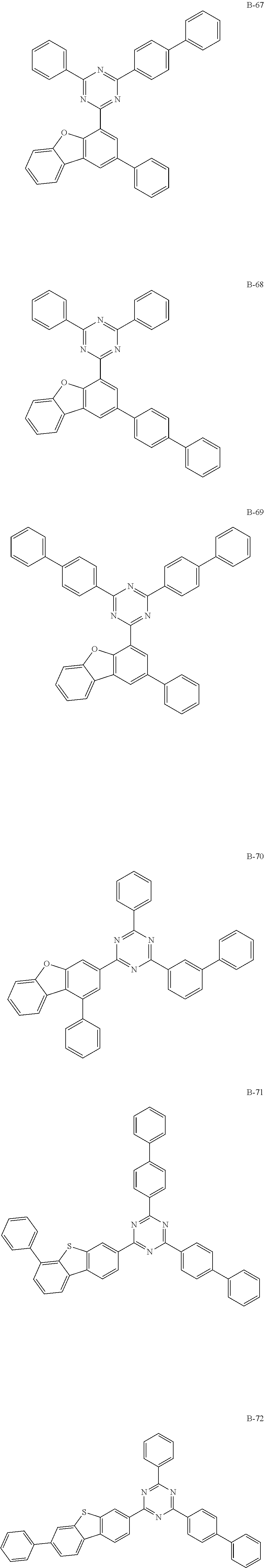 [SMILES: c1ccc(-c2ccc(-c3nc(-c4ccccc4)nc(-c4cc(-c5ccccc5)cc5c4oc4ccccc45)n3)cc2)cc1 ; c1ccc(-c2ccc(-c3cc(-c4nc(-c5ccccc5)nc(-c5ccccc5)n4)c4oc5ccccc5c4c3)cc2)cc1 ; c1ccc(-c2ccc(-c3nc(-c4ccc(-c5ccccc5)cc4)nc(-c4cc(-c5ccccc5)cc5c4oc4ccccc45)n3)cc2)cc1 ; c1ccc(-c2cccc(-c3nc(-c4ccccc4)nc(-c4cc(-c5ccccc5)c5c(c4)oc4ccccc45)n3)c2)cc1 ; c1ccc(-c2ccc(-c3nc(-c4ccc(-c5ccccc5)cc4)nc(-c4ccc5c(c4)sc4c(-c6ccccc6)cccc45)n3)cc2)cc1 ; c1ccc(-c2ccc(-c3nc(-c4ccccc4)nc(-c4ccc5c(c4)sc4cc(-c6ccccc6)ccc45)n3)cc2)cc1]

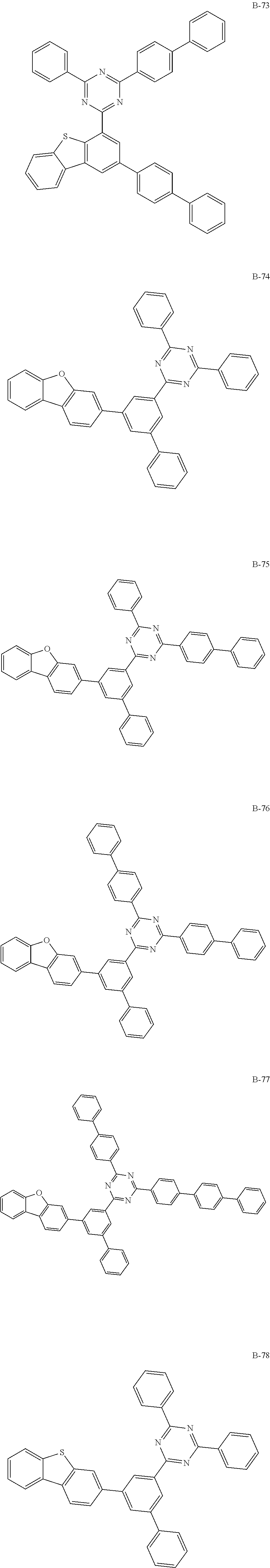

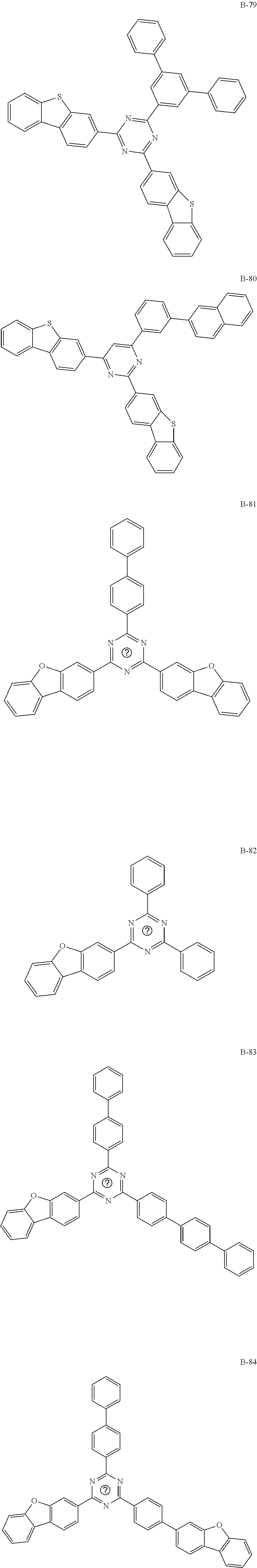 [SMILES: c1ccc(-c2cc(-c3ccccc3)cc(-c3nc(-c4ccc5c(c4)sc4ccccc45)nc(-c4ccc5c(c4)sc4ccccc45)n3)c2)cc1 ; c1cc(-c2ccc3ccccc3c2)cc(-c2cc(-c3ccc4c(c3)sc3ccccc34)nc(-c3ccc4c(c3)sc3ccccc34)n2)c1 ; c1ccc(-c2ccc(-c3nc(-c4ccc5c(c4)oc4ccccc45)nc(-c4ccc5c(c4)oc4ccccc45)n3)cc2)cc1 ; c1ccc(-c2nc(-c3ccccc3)nc(-c3ccc4c(c3)oc3ccccc34)n2)cc1 ; c1ccc(-c2ccc(-c3ccc(-c4nc(-c5ccc(-c6ccccc6)cc5)nc(-c5ccc6c(c5)oc5ccccc56)n4)cc3)cc2)cc1 ; c1ccc(-c2ccc(-c3nc(-c4ccc(-c5ccc6c(c5)oc5ccccc56)cc4)nc(-c4ccc5c(c4)oc4ccccc45)n3)cc2)cc1]

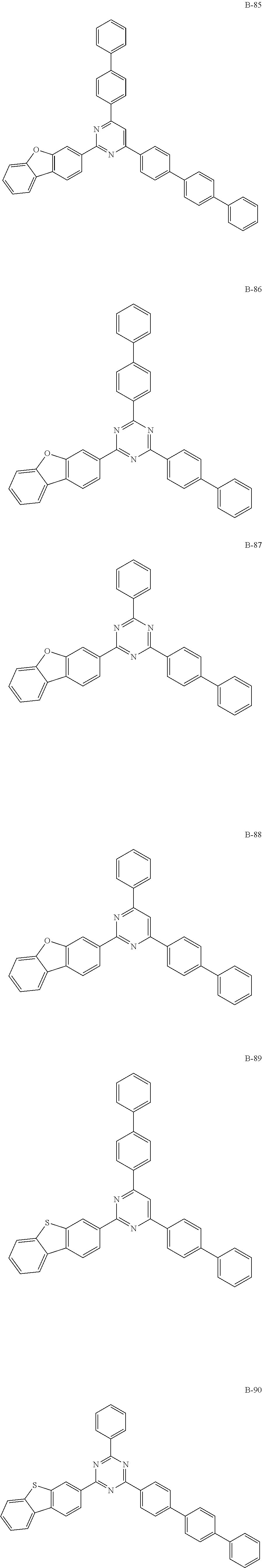 [SMILES: c1ccc(-c2ccc(-c3ccc(-c4cc(-c5ccc(-c6ccccc6)cc5)nc(-c5ccc6c(c5)oc5ccccc56)n4)cc3)cc2)cc1 ; c1ccc(-c2ccc(-c3nc(-c4ccc(-c5ccccc5)cc4)nc(-c4ccc5c(c4)oc4ccccc45)n3)cc2)cc1 ; c1ccc(-c2ccc(-c3nc(-c4ccccc4)nc(-c4ccc5c(c4)oc4ccccc45)n3)cc2)cc1 ; c1ccc(-c2ccc(-c3cc(-c4ccccc4)nc(-c4ccc5c(c4)oc4ccccc45)n3)cc2)cc1 ; c1ccc(-c2ccc(-c3cc(-c4ccc(-c5ccccc5)cc4)nc(-c4ccc5c(c4)sc4ccccc45)n3)cc2)cc1 ; c1ccc(-c2ccc(-c3ccc(-c4nc(-c5ccccc5)nc(-c5ccc6c(c5)sc5ccccc56)n4)cc3)cc2)cc1]

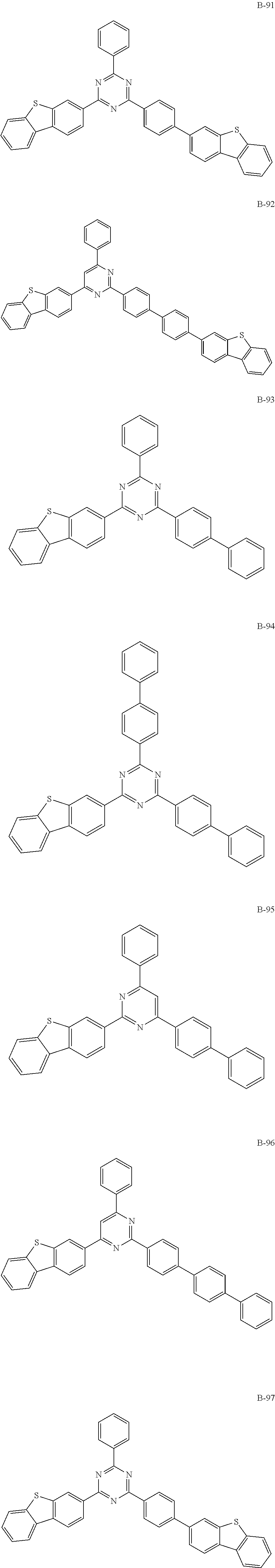

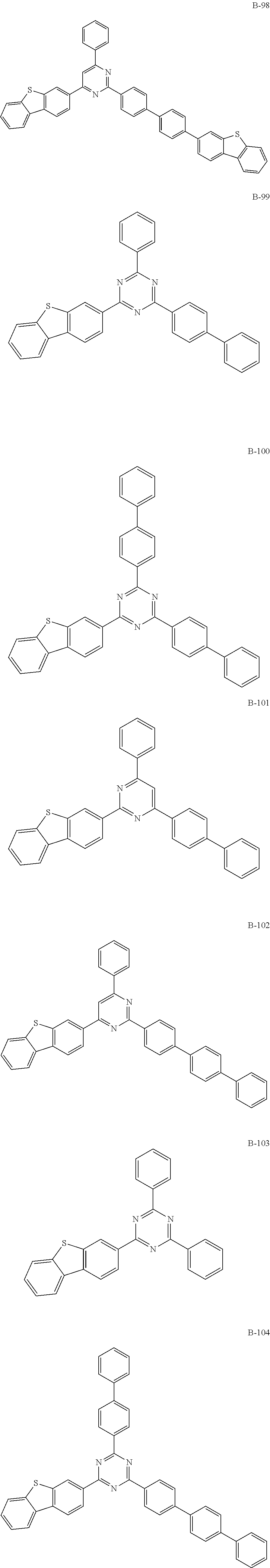

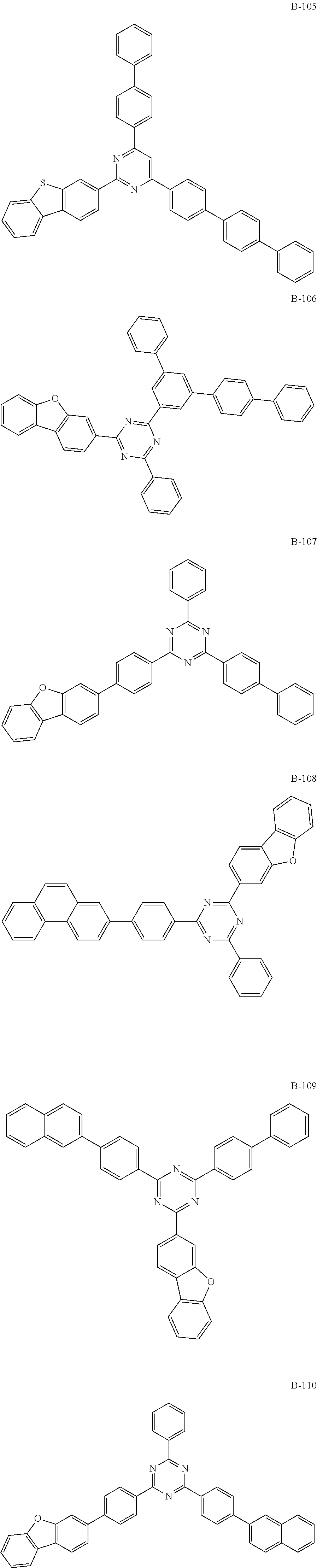

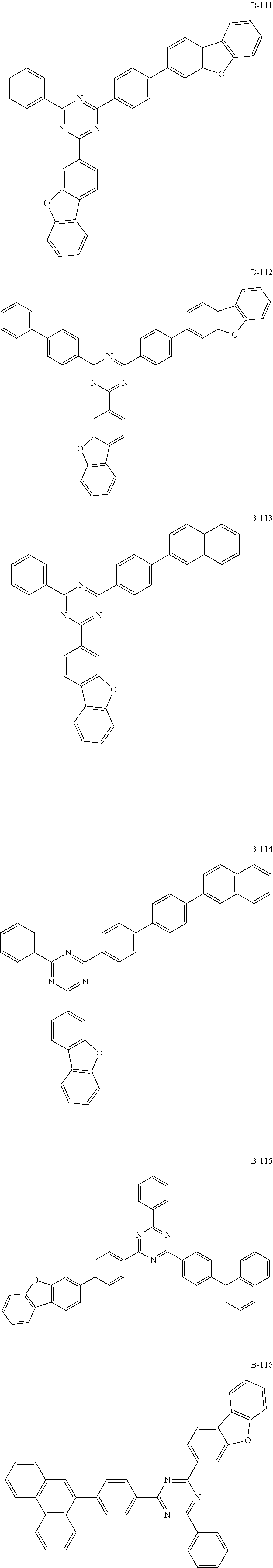 [SMILES: c1ccc(-c2nc(-c3ccc(-c4ccc5c(c4)oc4ccccc45)cc3)nc(-c3ccc4c(c3)oc3ccccc34)n2)cc1 ; c1ccc(-c2ccc(-c3nc(-c4ccc(-c5ccc6c(c5)oc5ccccc56)cc4)nc(-c4ccc5c(c4)oc4ccccc45)n3)cc2)cc1 ; c1ccc(-c2nc(-c3ccc(-c4ccc5ccccc5c4)cc3)nc(-c3ccc4c(c3)oc3ccccc34)n2)cc1 ; c1ccc(-c2nc(-c3ccc(-c4ccc(-c5ccc6ccccc6c5)cc4)cc3)nc(-c3ccc4c(c3)oc3ccccc34)n2)cc1 ; c1ccc(-c2nc(-c3ccc(-c4ccc5c(c4)oc4ccccc45)cc3)nc(-c3ccc(-c4cccc5ccccc45)cc3)n2)cc1 ; c1ccc(-c2nc(-c3ccc(-c4cc5ccccc5c5ccccc45)cc3)nc(-c3ccc4c(c3)oc3ccccc34)n2)cc1]

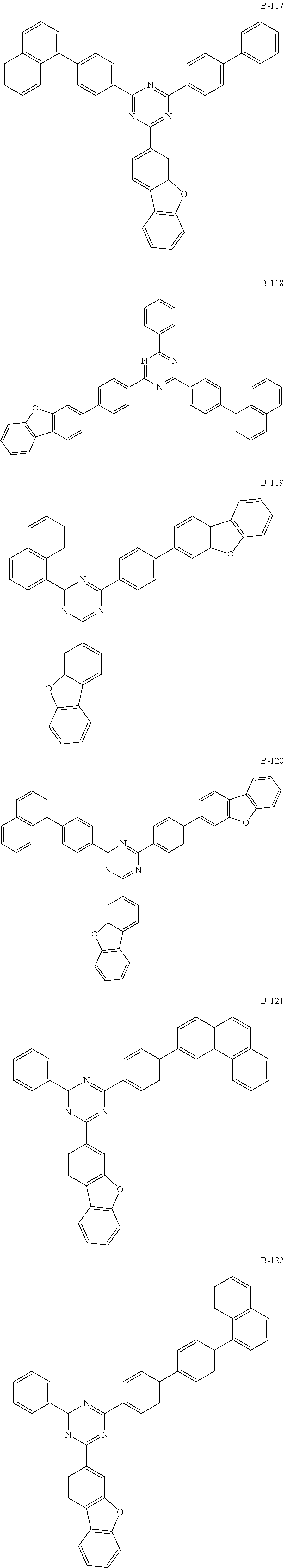

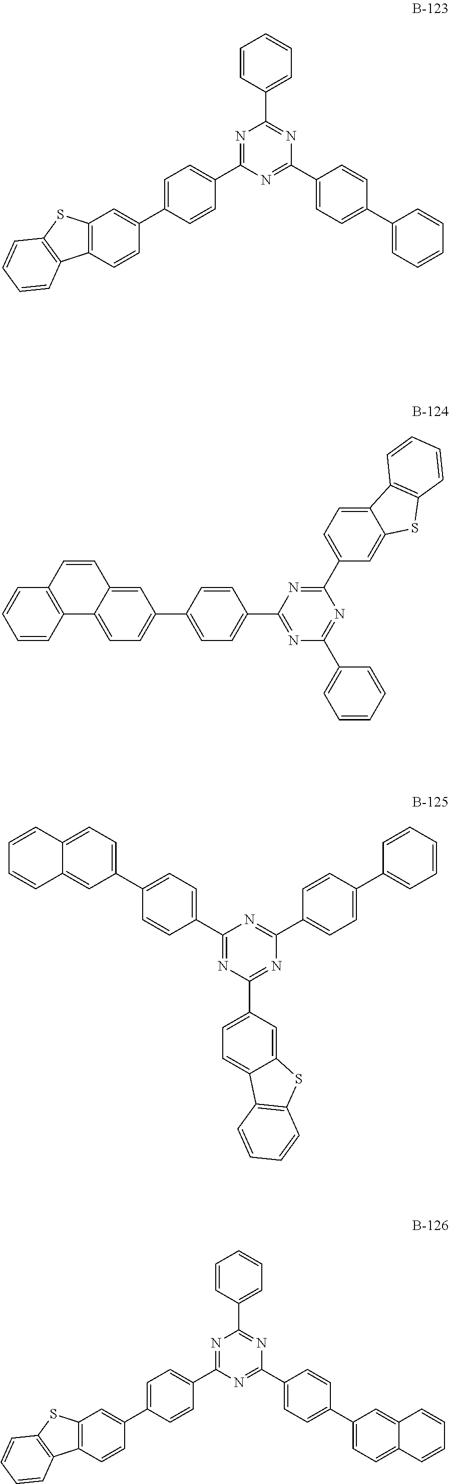 [SMILES: c1ccc(-c2ccc(-c3nc(-c4ccccc4)nc(-c4ccc(-c5ccc6c(c5)sc5ccccc56)cc4)n3)cc2)cc1 ; c1ccc(-c2nc(-c3ccc(-c4ccc5c(ccc6ccccc65)c4)cc3)nc(-c3ccc4c(c3)sc3ccccc34)n2)cc1 ; c1ccc(-c2ccc(-c3nc(-c4ccc(-c5ccc6ccccc6c5)cc4)nc(-c4ccc5c(c4)sc4ccccc45)n3)cc2)cc1 ; c1ccc(-c2nc(-c3ccc(-c4ccc5ccccc5c4)cc3)nc(-c3ccc(-c4ccc5c(c4)sc4ccccc45)cc3)n2)cc1]

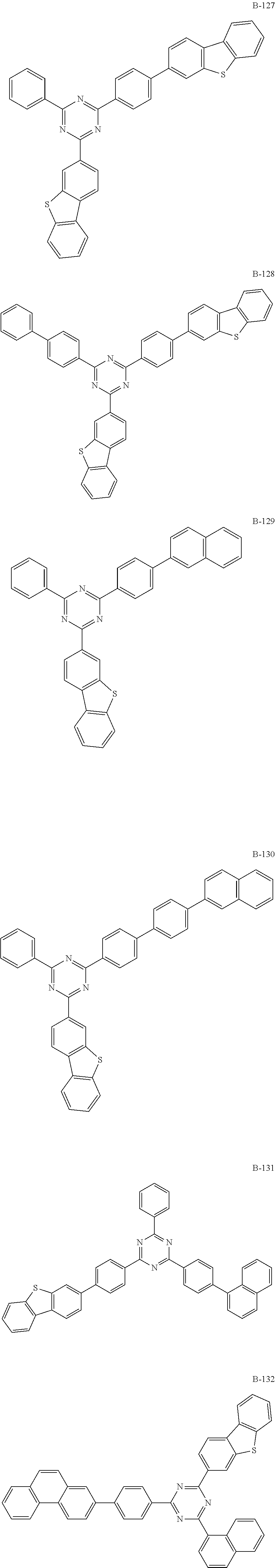

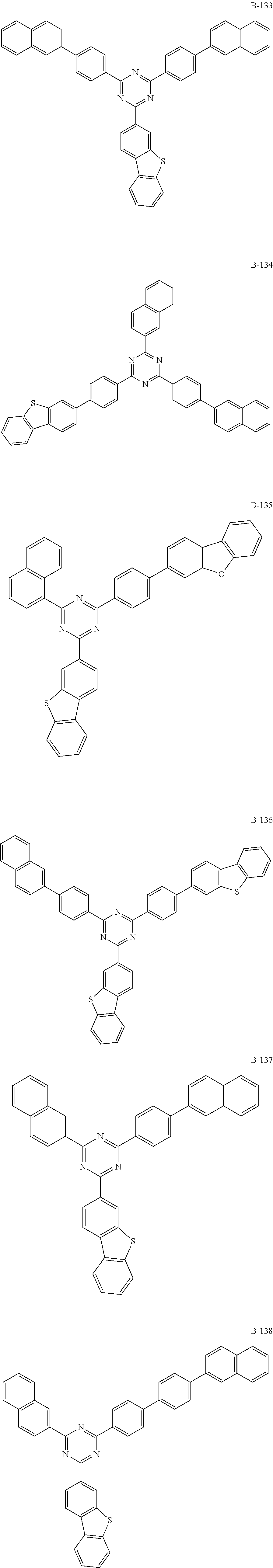

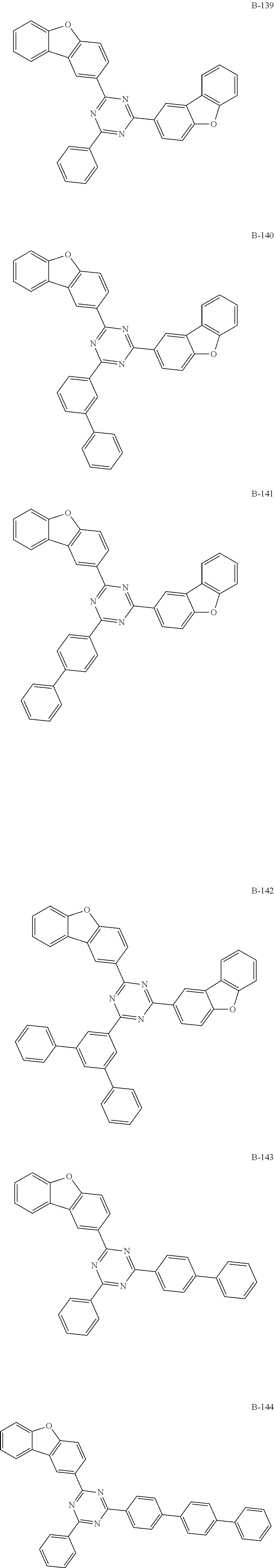 [SMILES: c1ccc(-c2nc(-c3ccc4oc5ccccc5c4c3)nc(-c3ccc4oc5ccccc5c4c3)n2)cc1 ; c1ccc(-c2cccc(-c3nc(-c4ccc5oc6ccccc6c5c4)nc(-c4ccc5oc6ccccc6c5c4)n3)c2)cc1 ; c1ccc(-c2ccc(-c3nc(-c4ccc5oc6ccccc6c5c4)nc(-c4ccc5oc6ccccc6c5c4)n3)cc2)cc1 ; c1ccc(-c2cc(-c3ccccc3)cc(-c3nc(-c4ccc5oc6ccccc6c5c4)nc(-c4ccc5oc6ccccc6c5c4)n3)c2)cc1 ; c1ccc(-c2ccc(-c3nc(-c4ccccc4)nc(-c4ccc5oc6ccccc6c5c4)n3)cc2)cc1 ; c1ccc(-c2ccc(-c3ccc(-c4nc(-c5ccccc5)nc(-c5ccc6oc7ccccc7c6c5)n4)cc3)cc2)cc1]

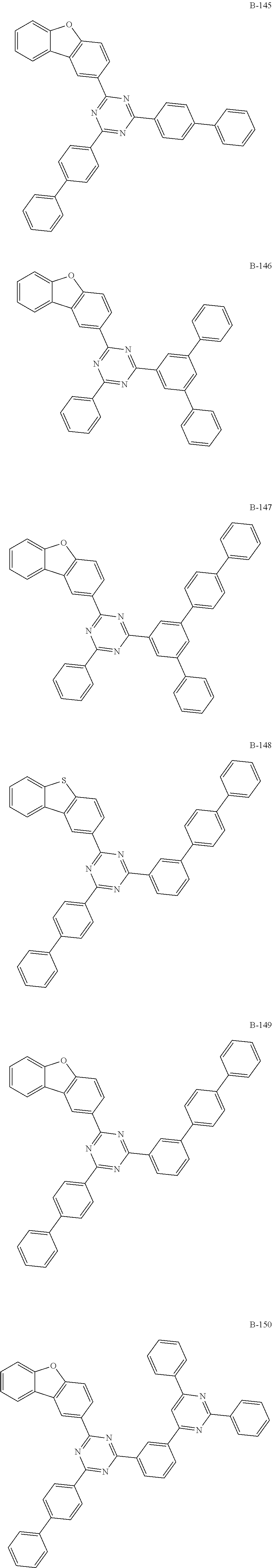

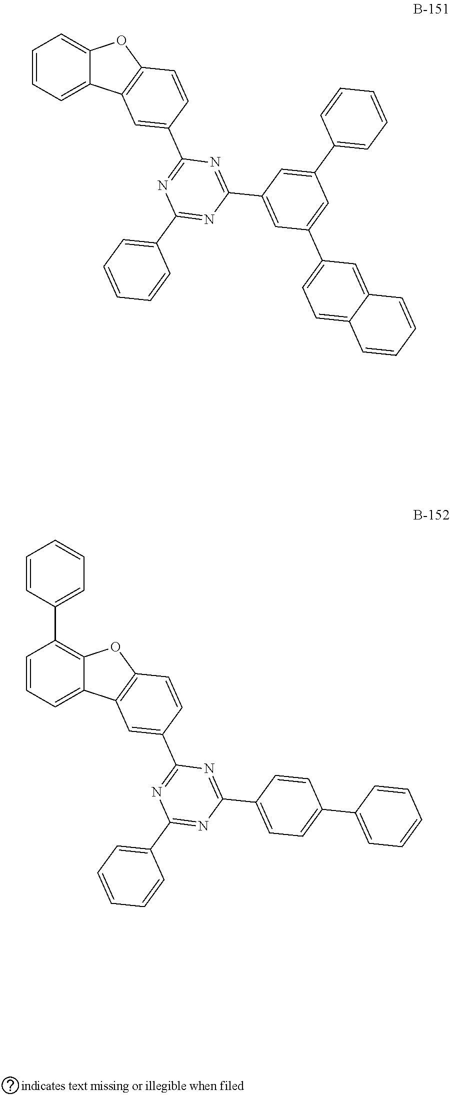 [SMILES: c1ccc(-c2cc(-c3ccc4ccccc4c3)cc(-c3nc(-c4ccccc4)nc(-c4ccc5oc6ccccc6c5c4)n3)c2)cc1 ; c1ccc(-c2ccc(-c3nc(-c4ccccc4)nc(-c4ccc5oc6c(-c7ccccc7)cccc6c5c4)n3)cc2)cc1]

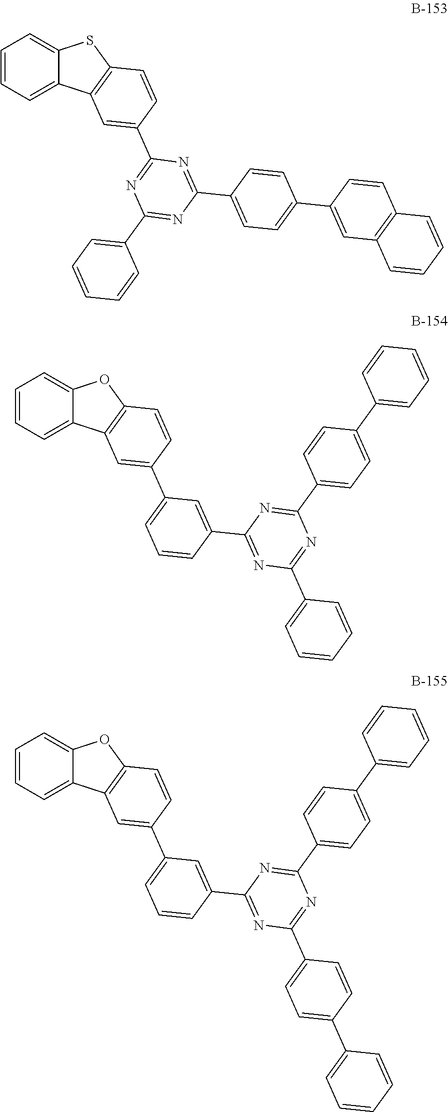 [SMILES: c1ccc(-c2nc(-c3ccc(-c4ccc5ccccc5c4)cc3)nc(-c3ccc4sc5ccccc5c4c3)n2)cc1 ; c1ccc(-c2ccc(-c3nc(-c4ccccc4)nc(-c4cccc(-c5ccc6oc7ccccc7c6c5)c4)n3)cc2)cc1 ; c1ccc(-c2ccc(-c3nc(-c4ccc(-c5ccccc5)cc4)nc(-c4cccc(-c5ccc6oc7ccccc7c6c5)c4)n3)cc2)cc1]

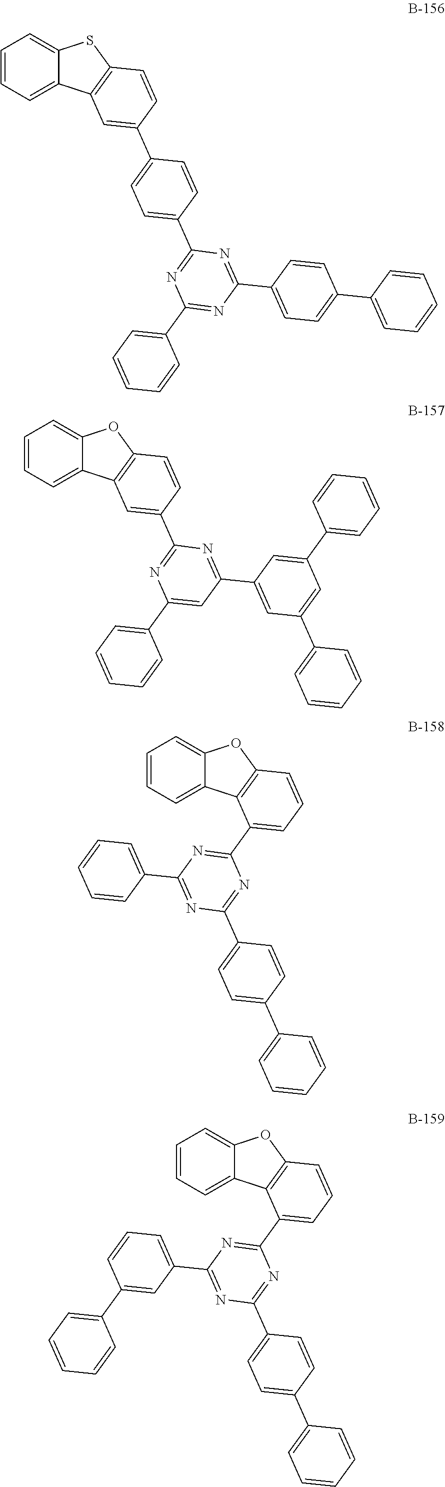

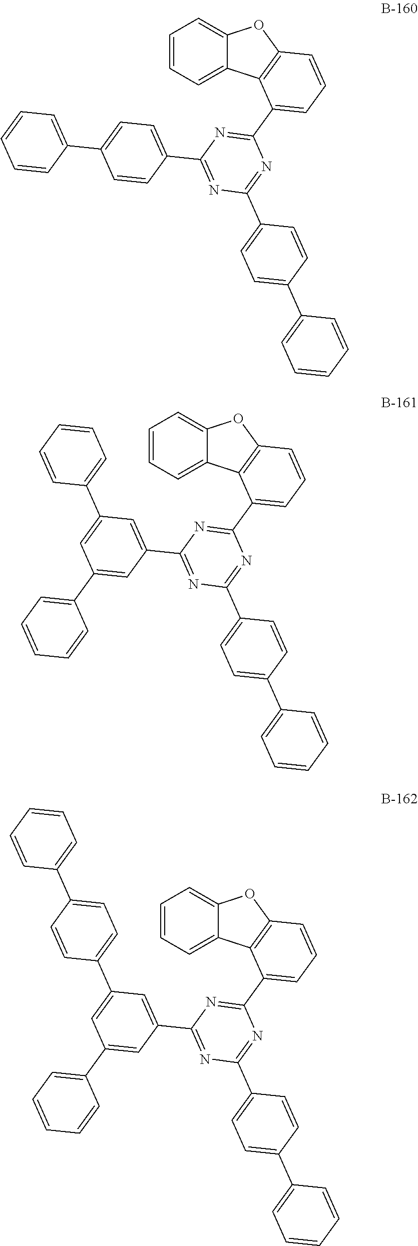 [SMILES: c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cc(-c5ccccc5)cc(-c5ccccc5)c4)nc(-c4cccc5oc6ccccc6c45)n3)cc2)cc1 ; c1ccc(-c2ccc(-c3cc(-c4ccccc4)cc(-c4nc(-c5ccc(-c6ccccc6)cc5)nc(-c5cccc6oc7ccccc7c56)n4)c3)cc2)cc1]

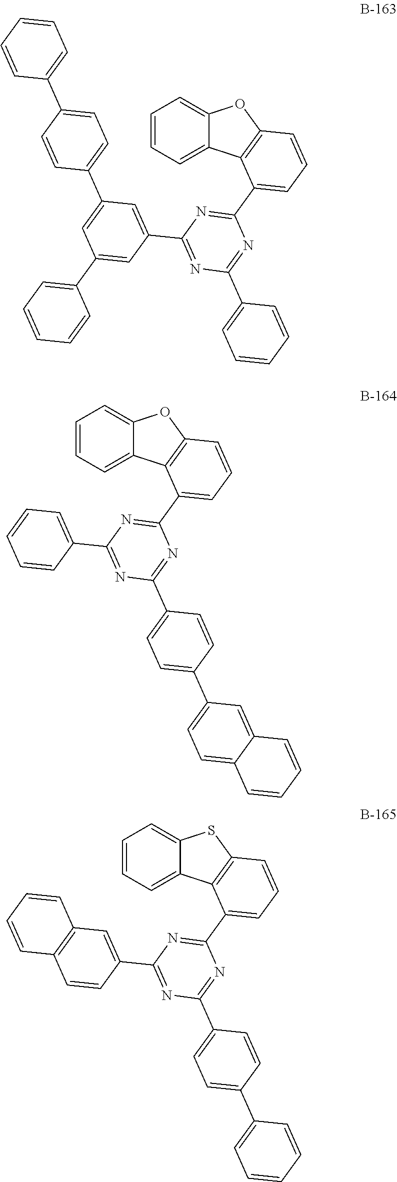 [SMILES: c1ccc(-c2ccc(-c3cc(-c4ccccc4)cc(-c4nc(-c5ccccc5)nc(-c5cccc6oc7ccccc7c56)n4)c3)cc2)cc1 ; c1ccc(-c2nc(-c3ccc(-c4ccc5ccccc5c4)cc3)nc(-c3cccc4oc5ccccc5c34)n2)cc1 ; c1ccc(-c2ccc(-c3nc(-c4ccc5ccccc5c4)nc(-c4cccc5sc6ccccc6c45)n3)cc2)cc1]

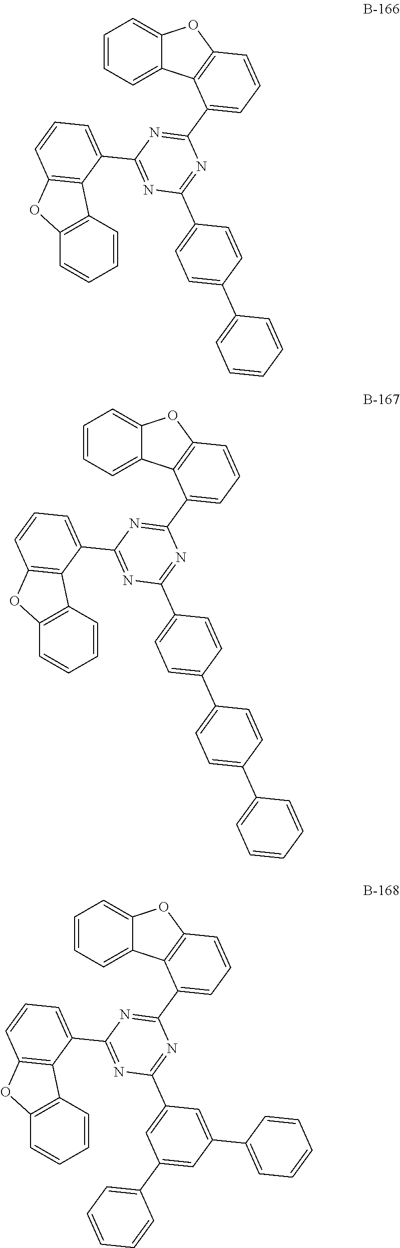 [SMILES: c1ccc(-c2ccc(-c3nc(-c4cccc5oc6ccccc6c45)nc(-c4cccc5oc6ccccc6c45)n3)cc2)cc1 ; c1ccc(-c2ccc(-c3ccc(-c4nc(-c5cccc6oc7ccccc7c56)nc(-c5cccc6oc7ccccc7c56)n4)cc3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1]

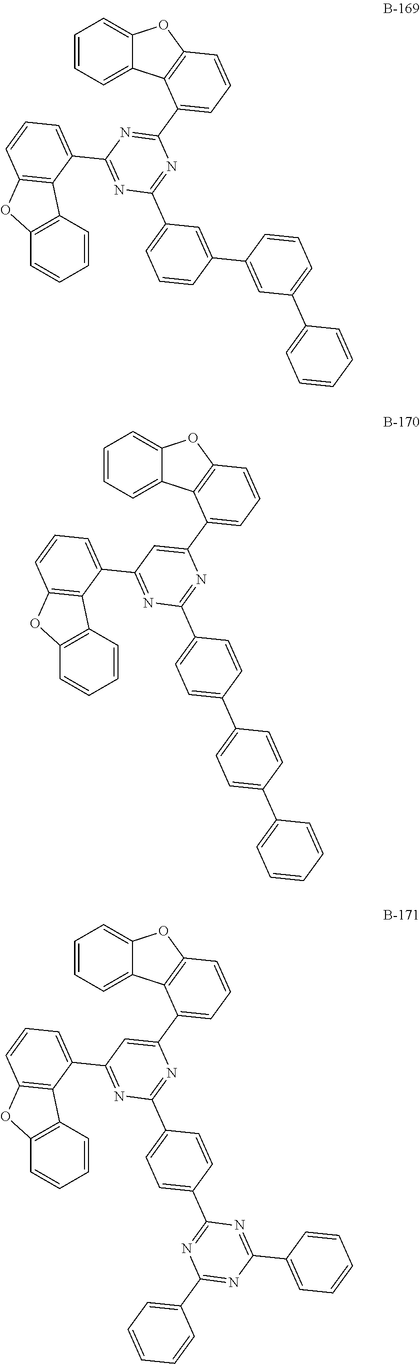 [SMILES: c1ccc(-c2cccc(-c3cccc(-c4nc(-c5cccc6oc7ccccc7c56)nc(-c5cccc6oc7ccccc7c56)n4)c3)c2)cc1 ; c1ccc(-c2ccc(-c3ccc(-c4nc(-c5cccc6oc7ccccc7c56)cc(-c5cccc6oc7ccccc7c56)n4)cc3)cc2)cc1 ; c1ccc(-c2nc(-c3ccccc3)nc(-c3ccc(-c4nc(-c5cccc6oc7ccccc7c56)cc(-c5cccc6oc7ccccc7c56)n4)cc3)n2)cc1]

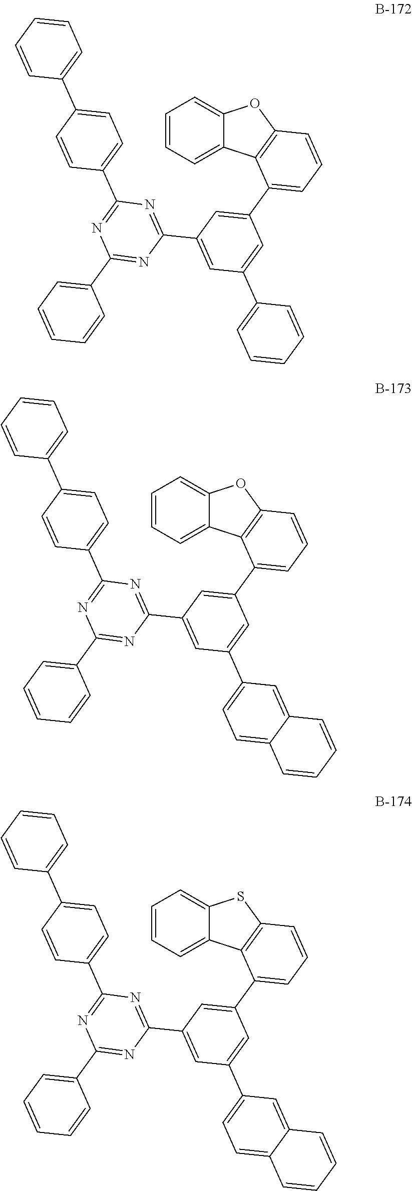 [SMILES: c1ccc(-c2ccc(-c3nc(-c4ccccc4)nc(-c4cc(-c5ccccc5)cc(-c5cccc6oc7ccccc7c56)c4)n3)cc2)cc1 ; c1ccc(-c2ccc(-c3nc(-c4ccccc4)nc(-c4cc(-c5ccc6ccccc6c5)cc(-c5cccc6oc7ccccc7c56)c4)n3)cc2)cc1 ; c1ccc(-c2ccc(-c3nc(-c4ccccc4)nc(-c4cc(-c5ccc6ccccc6c5)cc(-c5cccc6sc7ccccc7c56)c4)n3)cc2)cc1]

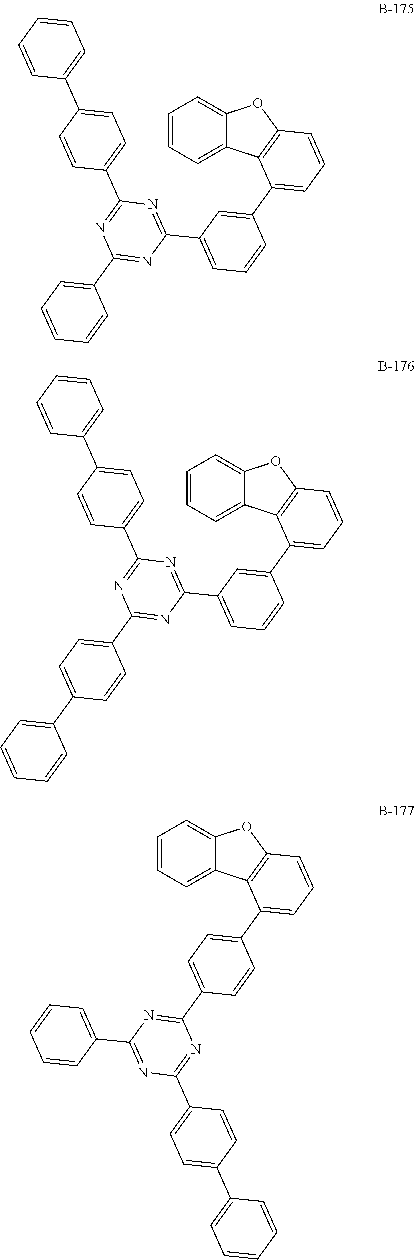

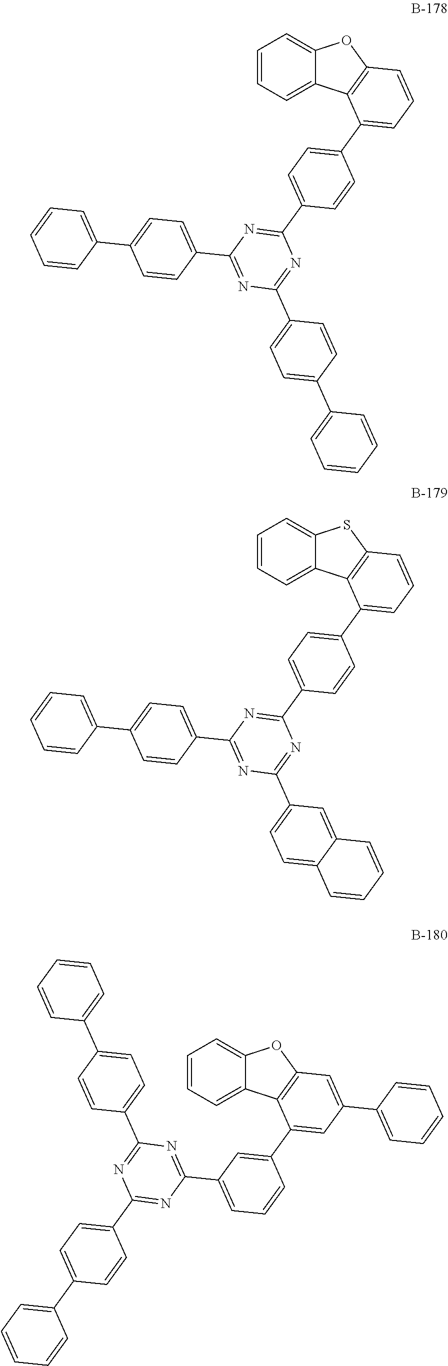 [SMILES: c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5cccc6sc7ccccc7c56)cc4)nc(-c4ccc5ccccc5c4)n3)cc2)cc1 ; c1ccc(-c2ccc(-c3nc(-c4ccc(-c5ccccc5)cc4)nc(-c4cccc(-c5cc(-c6ccccc6)cc6oc7ccccc7c56)c4)n3)cc2)cc1]

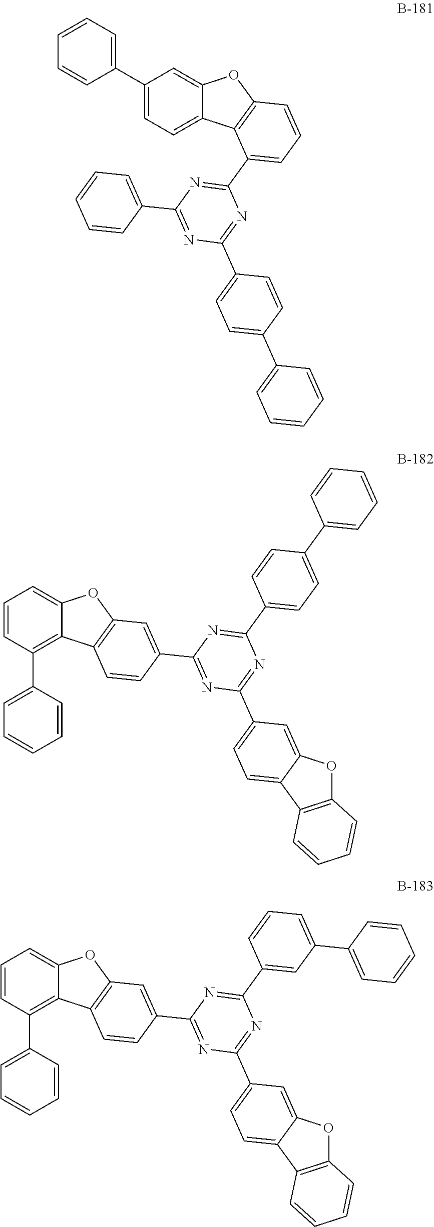 [SMILES: c1ccc(-c2ccc(-c3nc(-c4ccccc4)nc(-c4cccc5oc6cc(-c7ccccc7)ccc6c45)n3)cc2)cc1 ; c1ccc(-c2ccc(-c3nc(-c4ccc5c(c4)oc4ccccc45)nc(-c4ccc5c(c4)oc4cccc(-c6ccccc6)c45)n3)cc2)cc1 ; c1ccc(-c2cccc(-c3nc(-c4ccc5c(c4)oc4ccccc45)nc(-c4ccc5c(c4)oc4cccc(-c6ccccc6)c45)n3)c2)cc1]

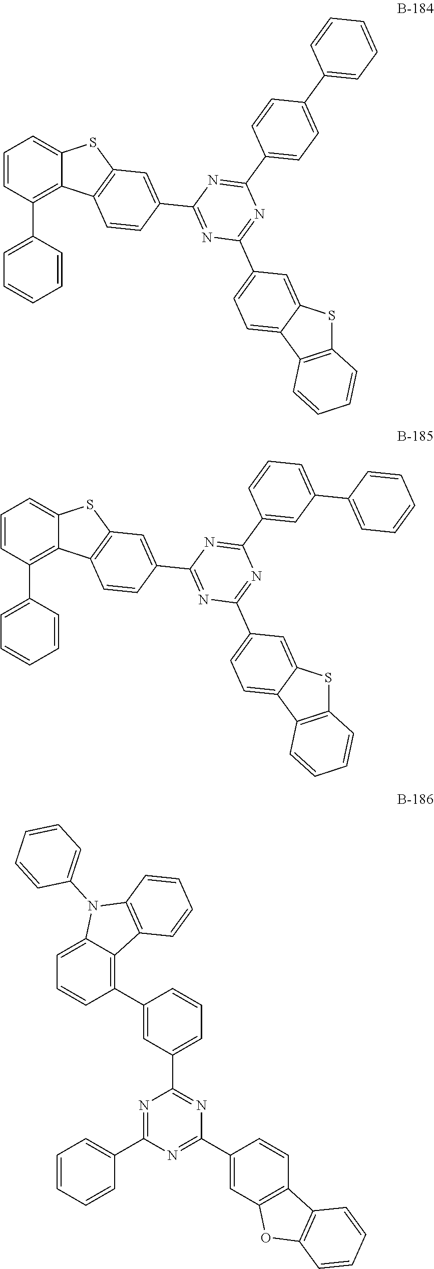 [SMILES: c1ccc(-c2ccc(-c3nc(-c4ccc5c(c4)sc4ccccc45)nc(-c4ccc5c(c4)sc4cccc(-c6ccccc6)c45)n3)cc2)cc1 ; c1ccc(-c2cccc(-c3nc(-c4ccc5c(c4)sc4ccccc45)nc(-c4ccc5c(c4)sc4cccc(-c6ccccc6)c45)n3)c2)cc1 ; c1ccc(-c2nc(-c3cccc(-c4cccc5c4c4ccccc4n5-c4ccccc4)c3)nc(-c3ccc4c(c3)oc3ccccc34)n2)cc1]

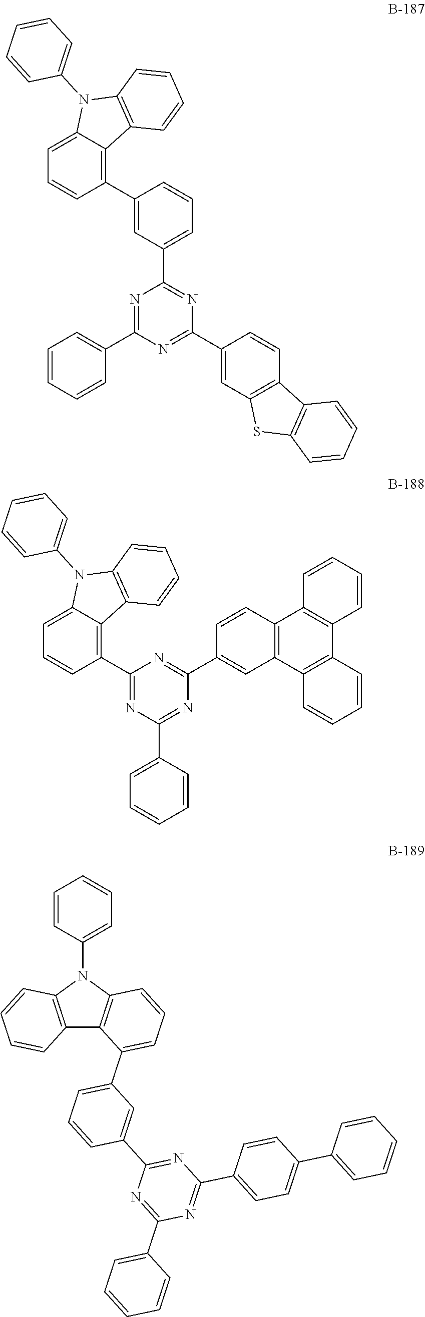 [SMILES: c1ccc(-c2nc(-c3cccc(-c4cccc5c4c4ccccc4n5-c4ccccc4)c3)nc(-c3ccc4c(c3)sc3ccccc34)n2)cc1 ; c1ccc(-c2nc(-c3ccc4c5ccccc5c5ccccc5c4c3)nc(-c3cccc4c3c3ccccc3n4-c3ccccc3)n2)cc1 ; c1ccc(-c2ccc(-c3nc(-c4ccccc4)nc(-c4cccc(-c5cccc6c5c5ccccc5n6-c5ccccc5)c4)n3)cc2)cc1]

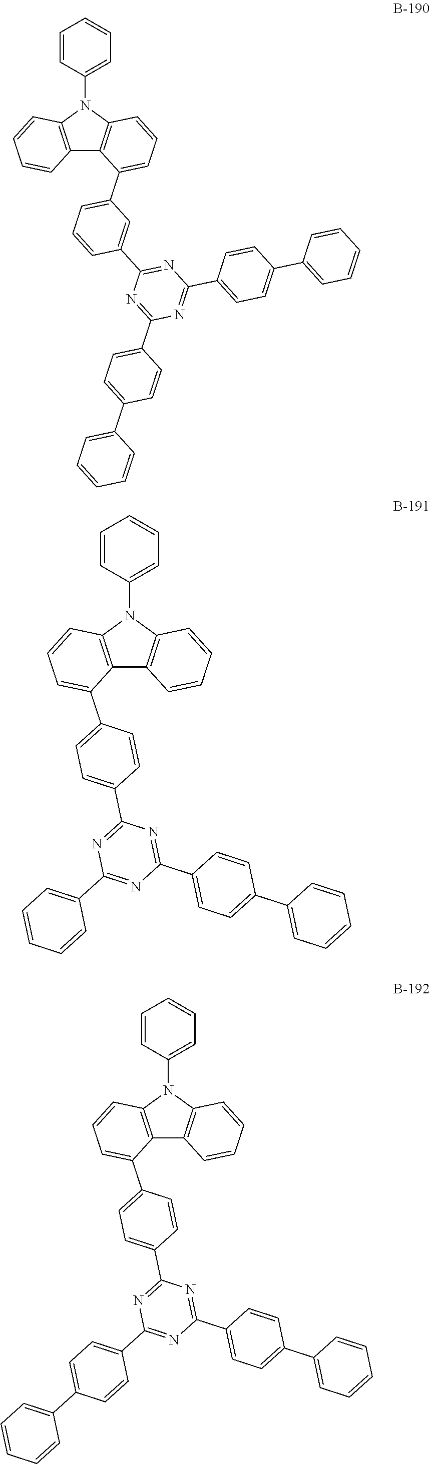

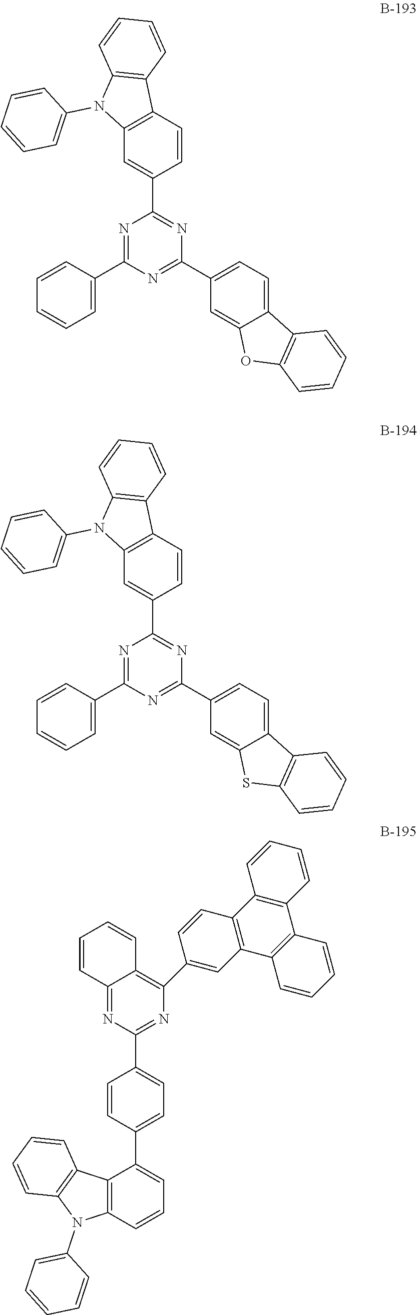

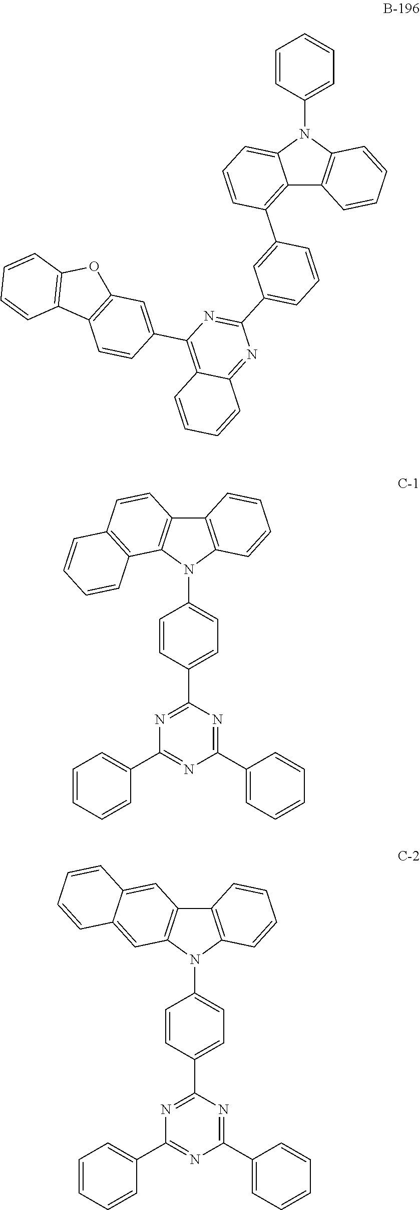

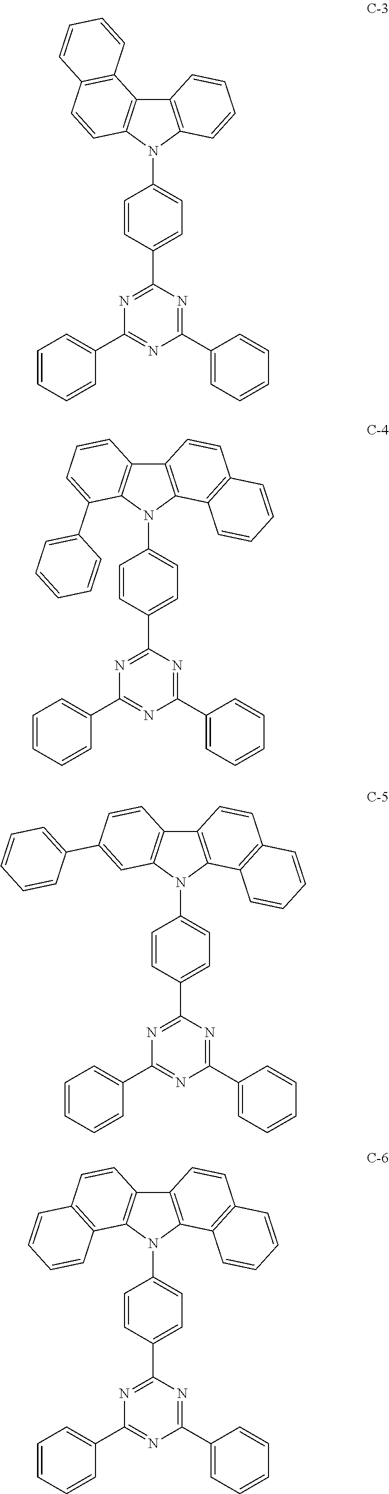

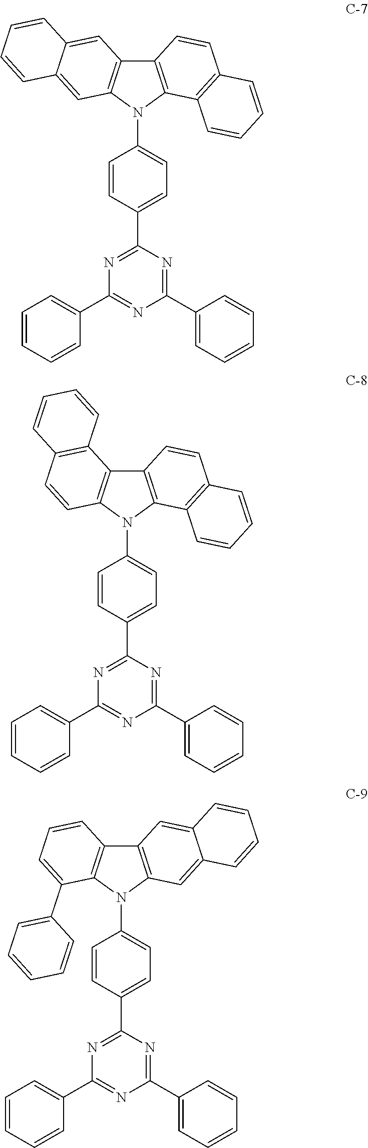

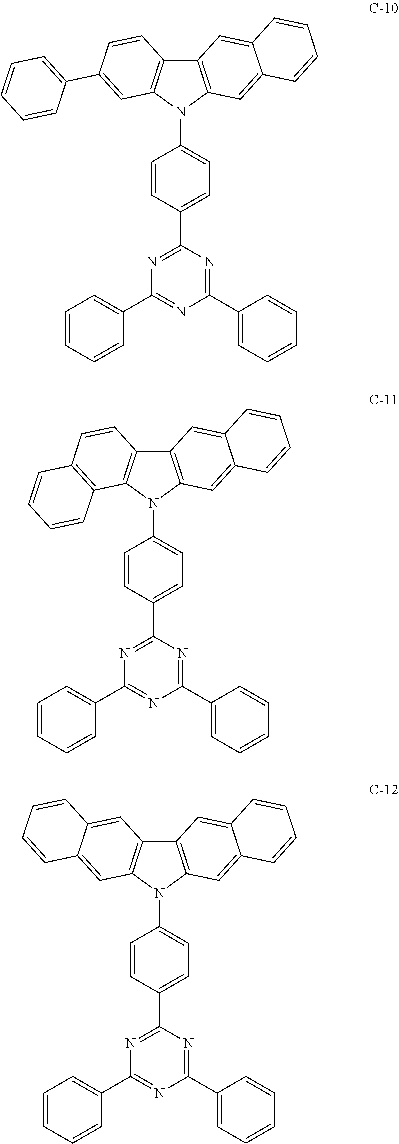

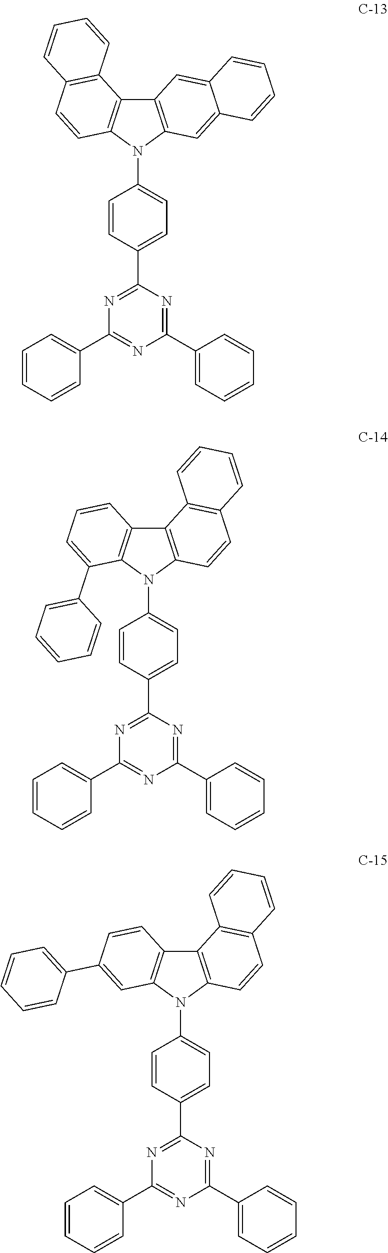

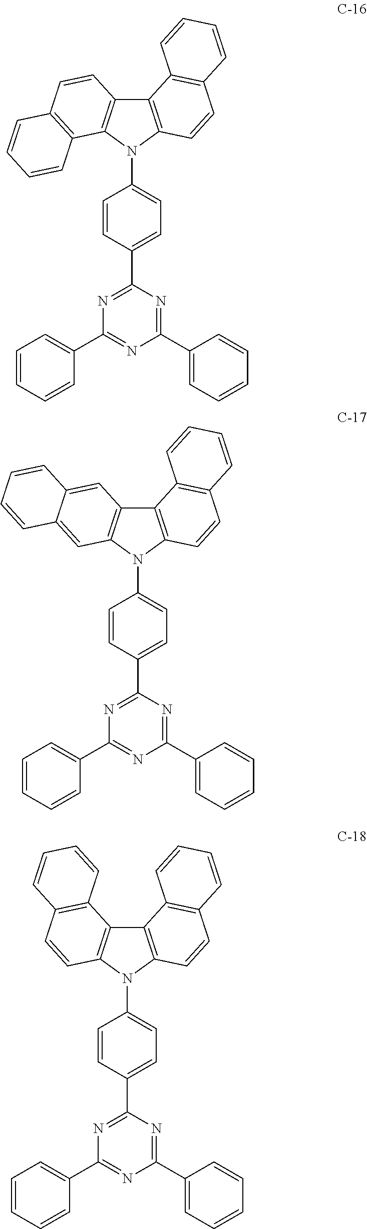

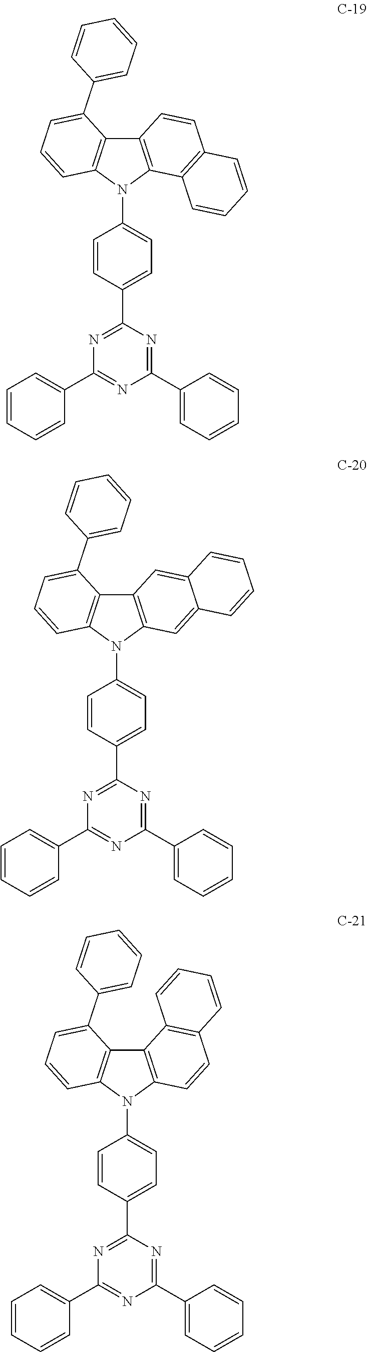 [SMILES: c1ccc(-c2nc(-c3ccccc3)nc(-c3ccc(-n4c5cccc(-c6ccccc6)c5c5ccc6ccccc6c54)cc3)n2)cc1 ; c1ccc(-c2nc(-c3ccccc3)nc(-c3ccc(-n4c5cc6ccccc6cc5c5c(-c6ccccc6)cccc54)cc3)n2)cc1 ; c1ccc(-c2nc(-c3ccccc3)nc(-c3ccc(-n4c5cccc(-c6ccccc6)c5c5c6ccccc6ccc54)cc3)n2)cc1]

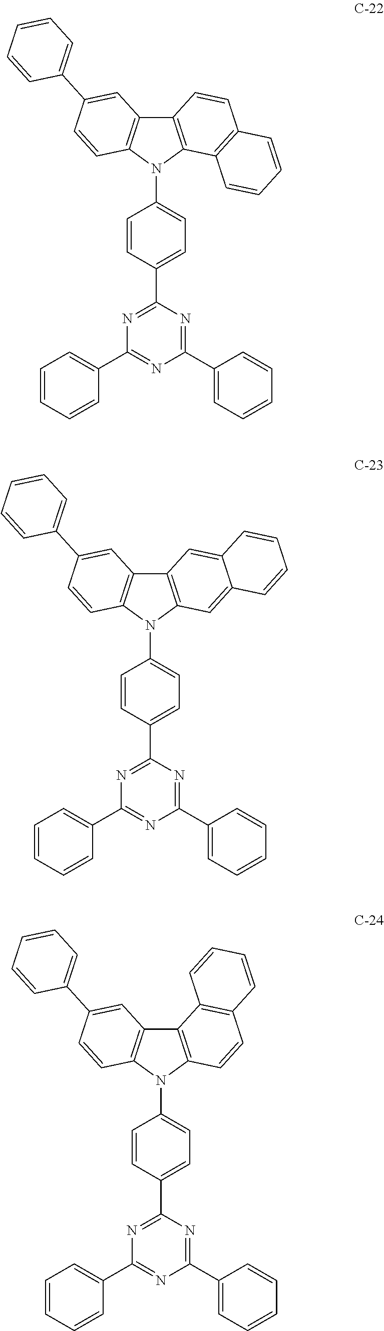 [SMILES: c1ccc(-c2ccc3c(c2)c2ccc4ccccc4c2n3-c2ccc(-c3nc(-c4ccccc4)nc(-c4ccccc4)n3)cc2)cc1 ; c1ccc(-c2ccc3c(c2)c2cc4ccccc4cc2n3-c2ccc(-c3nc(-c4ccccc4)nc(-c4ccccc4)n3)cc2)cc1 ; c1ccc(-c2ccc3c(c2)c2c4ccccc4ccc2n3-c2ccc(-c3nc(-c4ccccc4)nc(-c4ccccc4)n3)cc2)cc1]

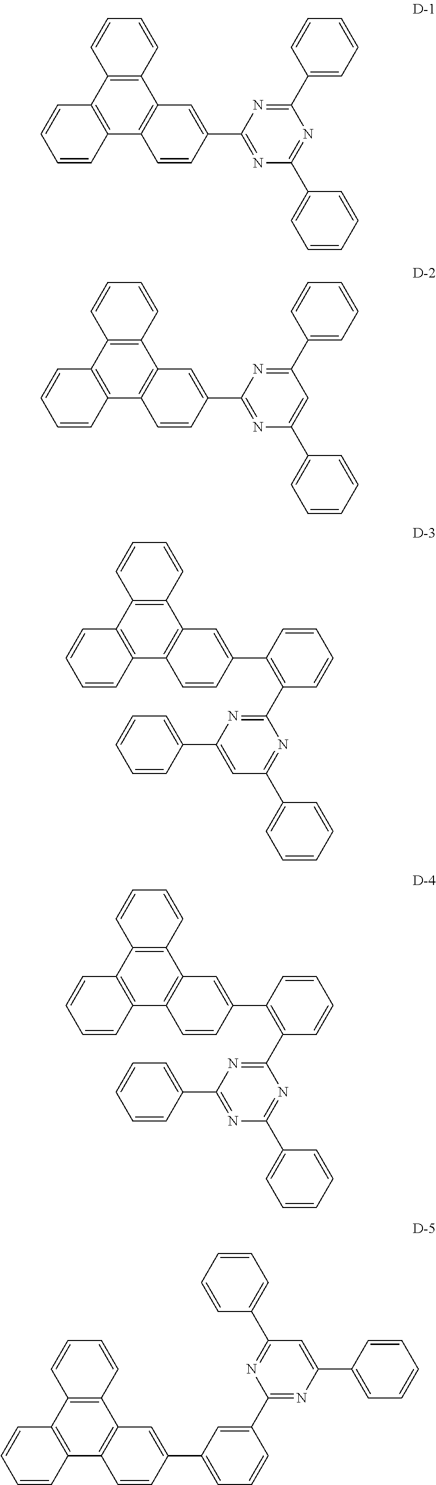

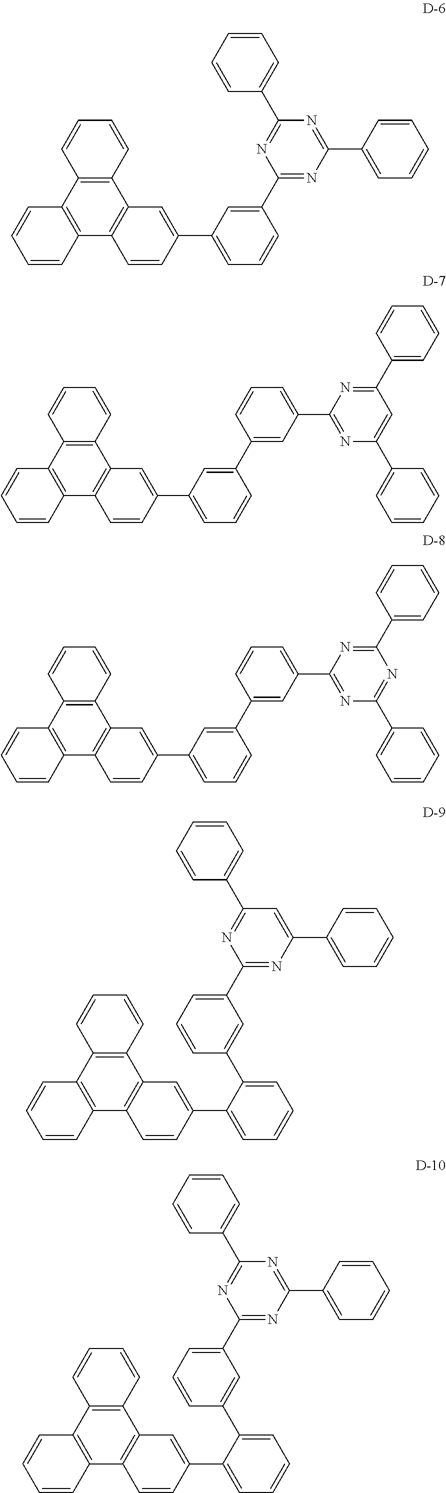

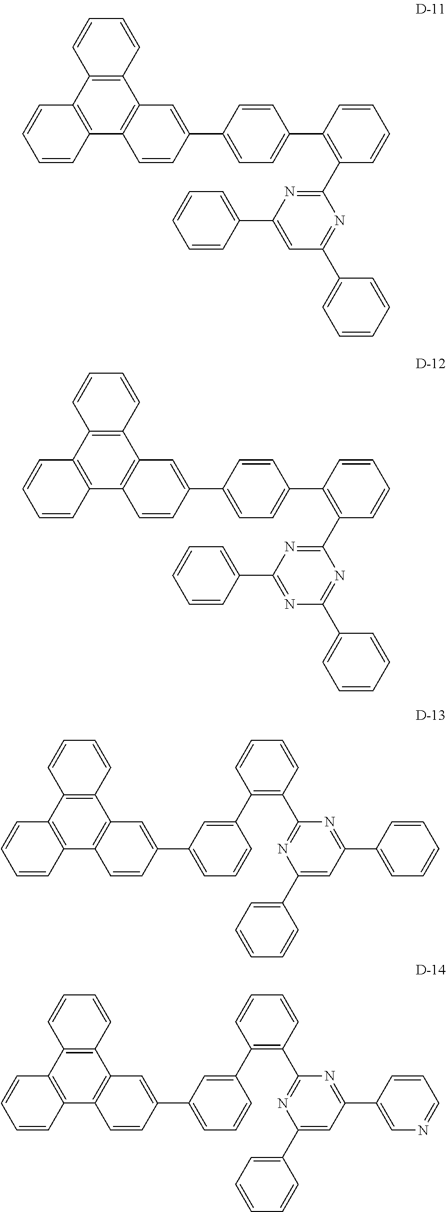

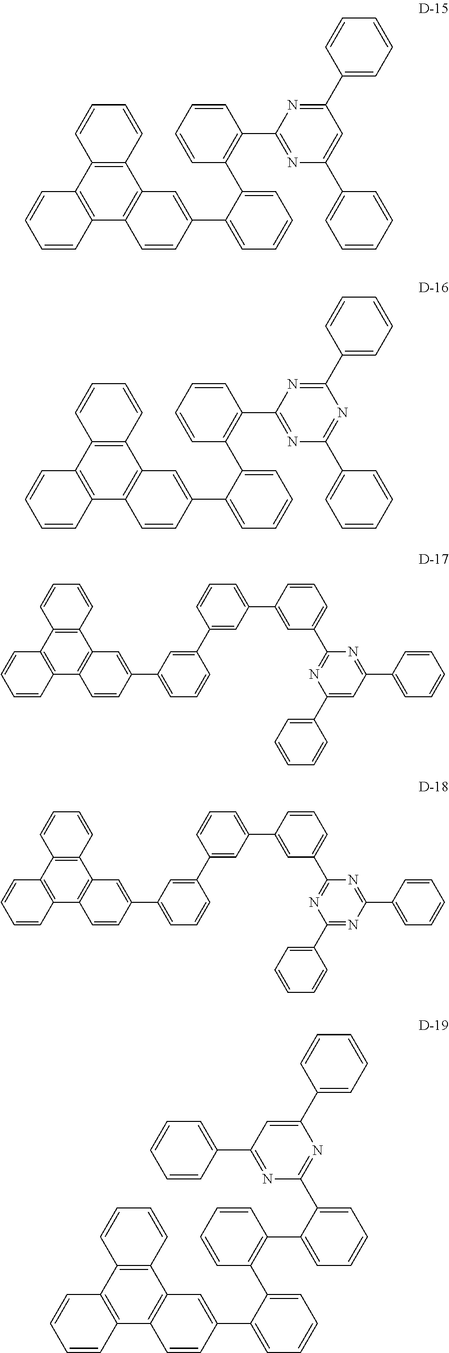

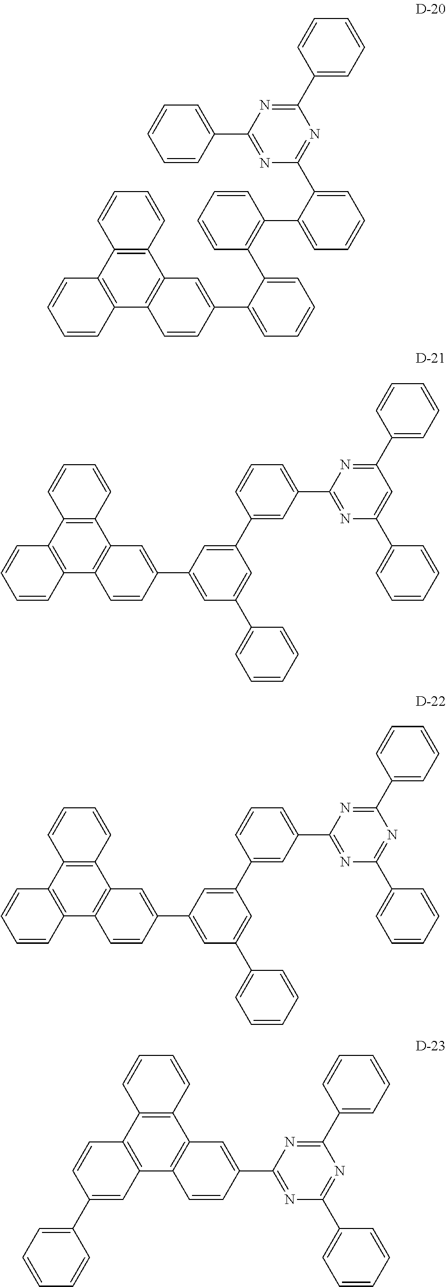 [SMILES: c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2cc(-c3cccc(-c4nc(-c5ccccc5)cc(-c5ccccc5)n4)c3)cc(-c3ccc4c5ccccc5c5ccccc5c4c3)c2)cc1 ; c1ccc(-c2cc(-c3cccc(-c4nc(-c5ccccc5)nc(-c5ccccc5)n4)c3)cc(-c3ccc4c5ccccc5c5ccccc5c4c3)c2)cc1 ; c1ccc(-c2ccc3c4ccccc4c4cc(-c5nc(-c6ccccc6)nc(-c6ccccc6)n5)ccc4c3c2)cc1]

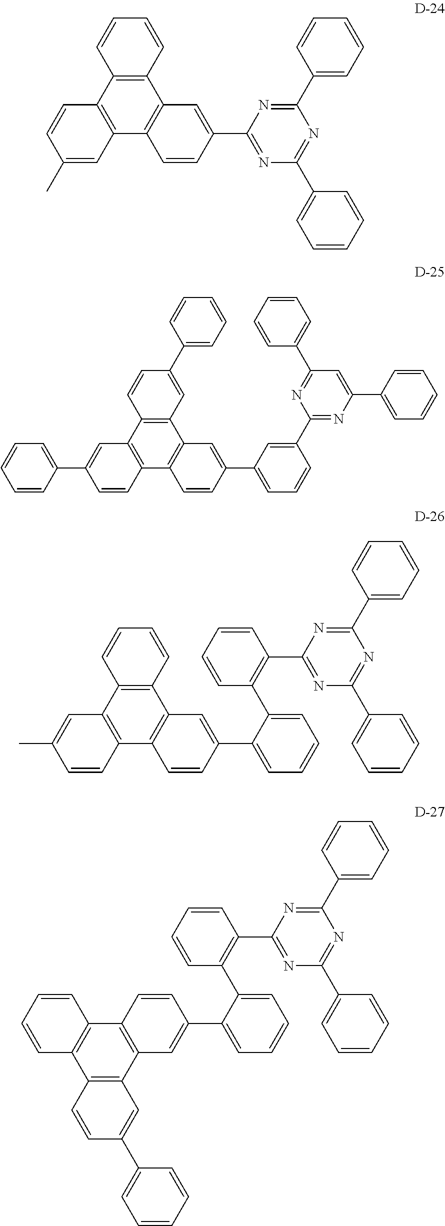 [SMILES: Cc1ccc2c3ccccc3c3cc(-c4nc(-c5ccccc5)nc(-c5ccccc5)n4)ccc3c2c1 ; c1ccc(-c2ccc3c4ccc(-c5cccc(-c6nc(-c7ccccc7)cc(-c7ccccc7)n6)c5)cc4c4cc(-c5ccccc5)ccc4c3c2)cc1 ; Cc1ccc2c3ccc(-c4ccccc4-c4ccccc4-c4nc(-c5ccccc5)nc(-c5ccccc5)n4)cc3c3ccccc3c2c1 ; c1ccc(-c2ccc3c4ccccc4c4ccc(-c5ccccc5-c5ccccc5-c5nc(-c6ccccc6)nc(-c6ccccc6)n5)cc4c3c2)cc1]

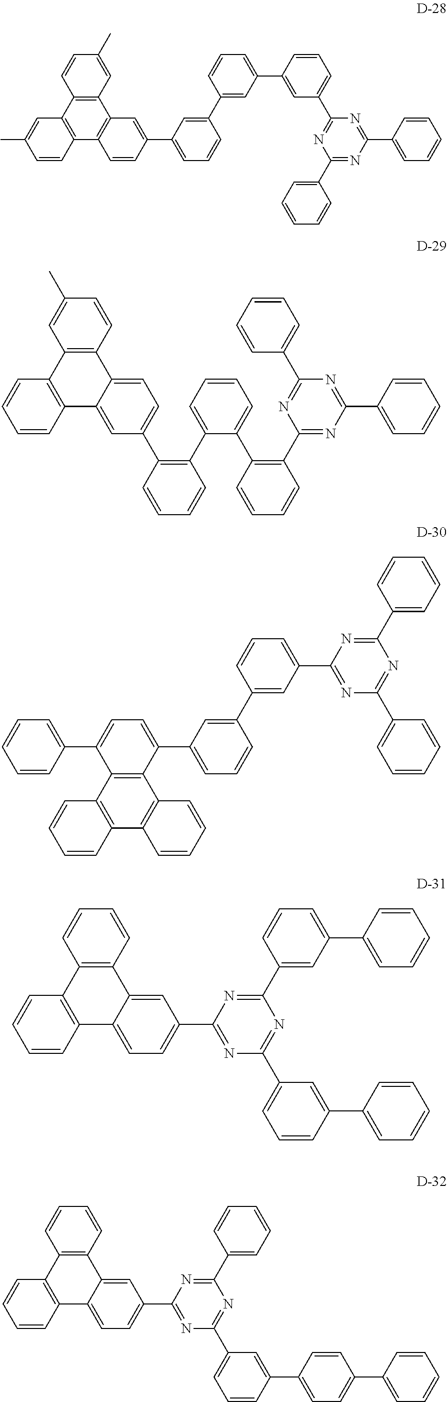 [SMILES: Cc1ccc2c3ccc(-c4cccc(-c5cccc(-c6cccc(-c7nc(-c8ccccc8)nc(-c8ccccc8)n7)c6)c5)c4)cc3c3cc(C)ccc3c2c1 ; Cc1ccc2c3ccc(-c4ccccc4-c4ccccc4-c4ccccc4-c4nc(-c5ccccc5)nc(-c5ccccc5)n4)cc3c3ccccc3c2c1 ; c1ccc(-c2nc(-c3ccccc3)nc(-c3cccc(-c4cccc(-c5ccc(-c6ccccc6)c6c7ccccc7c7ccccc7c56)c4)c3)n2)cc1 ; c1ccc(-c2cccc(-c3nc(-c4cccc(-c5ccccc5)c4)nc(-c4ccc5c6ccccc6c6ccccc6c5c4)n3)c2)cc1 ; c1ccc(-c2ccc(-c3cccc(-c4nc(-c5ccccc5)nc(-c5ccc6c7ccccc7c7ccccc7c6c5)n4)c3)cc2)cc1]

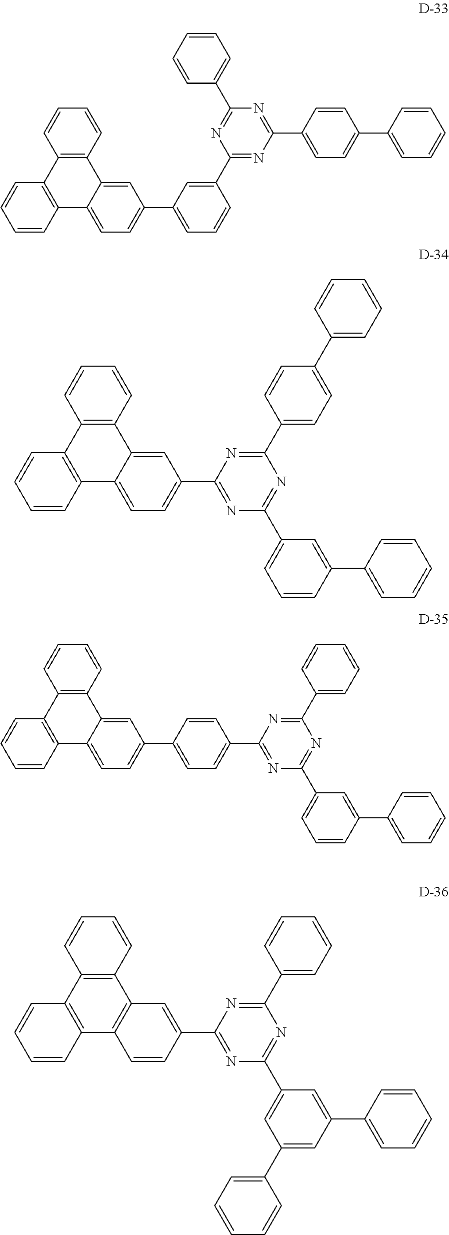 [SMILES: c1ccc(-c2ccc(-c3nc(-c4ccccc4)nc(-c4cccc(-c5ccc6c7ccccc7c7ccccc7c6c5)c4)n3)cc2)cc1 ; c1ccc(-c2ccc(-c3nc(-c4cccc(-c5ccccc5)c4)nc(-c4ccc5c6ccccc6c6ccccc6c5c4)n3)cc2)cc1 ; c1ccc(-c2cccc(-c3nc(-c4ccccc4)nc(-c4ccc(-c5ccc6c7ccccc7c7ccccc7c6c5)cc4)n3)c2)cc1 ; c1ccc(-c2cc(-c3ccccc3)cc(-c3nc(-c4ccccc4)nc(-c4ccc5c6ccccc6c6ccccc6c5c4)n3)c2)cc1]

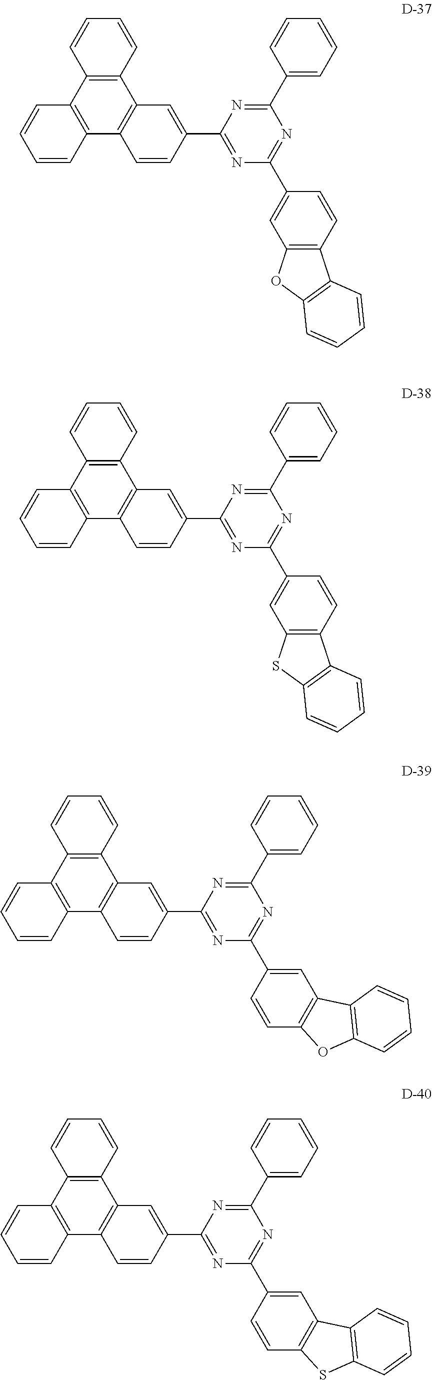 [SMILES: c1ccc(-c2nc(-c3ccc4c(c3)oc3ccccc34)nc(-c3ccc4c5ccccc5c5ccccc5c4c3)n2)cc1 ; c1ccc(-c2nc(-c3ccc4c(c3)sc3ccccc34)nc(-c3ccc4c5ccccc5c5ccccc5c4c3)n2)cc1 ; c1ccc(-c2nc(-c3ccc4oc5ccccc5c4c3)nc(-c3ccc4c5ccccc5c5ccccc5c4c3)n2)cc1 ; c1ccc(-c2nc(-c3ccc4sc5ccccc5c4c3)nc(-c3ccc4c5ccccc5c5ccccc5c4c3)n2)cc1]

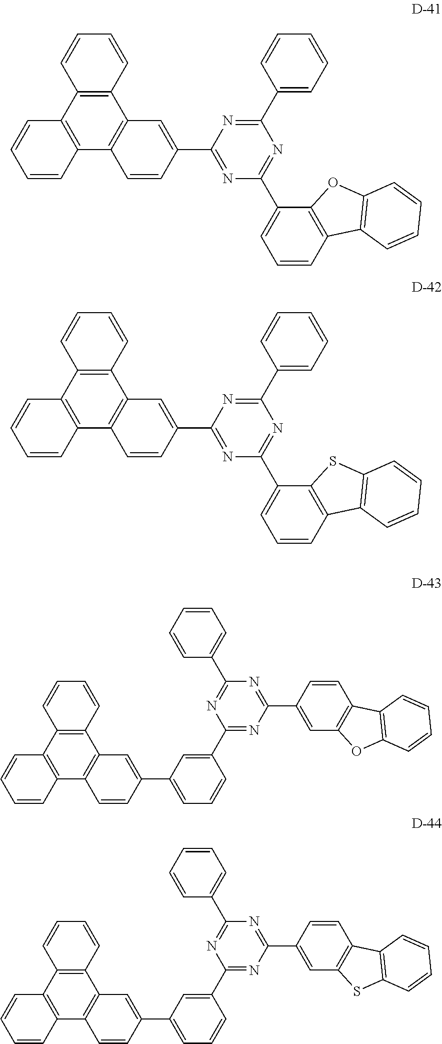 [SMILES: c1ccc(-c2nc(-c3ccc4c5ccccc5c5ccccc5c4c3)nc(-c3cccc4c3oc3ccccc34)n2)cc1 ; c1ccc(-c2nc(-c3ccc4c5ccccc5c5ccccc5c4c3)nc(-c3cccc4c3sc3ccccc34)n2)cc1 ; c1ccc(-c2nc(-c3cccc(-c4ccc5c6ccccc6c6ccccc6c5c4)c3)nc(-c3ccc4c(c3)oc3ccccc34)n2)cc1 ; c1ccc(-c2nc(-c3cccc(-c4ccc5c6ccccc6c6ccccc6c5c4)c3)nc(-c3ccc4c(c3)sc3ccccc34)n2)cc1]

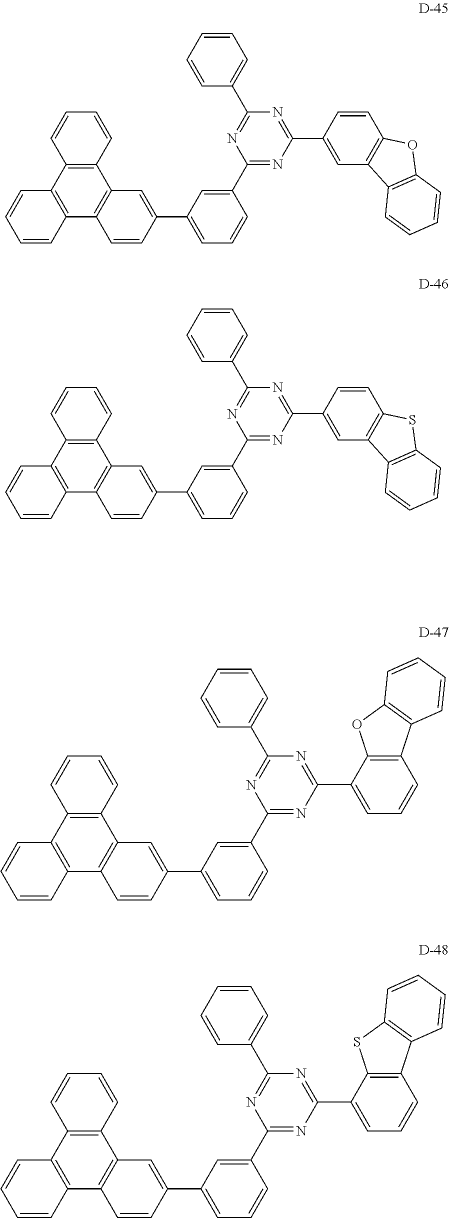 [SMILES: c1ccc(-c2nc(-c3cccc(-c4ccc5c6ccccc6c6ccccc6c5c4)c3)nc(-c3ccc4oc5ccccc5c4c3)n2)cc1 ; c1ccc(-c2nc(-c3cccc(-c4ccc5c6ccccc6c6ccccc6c5c4)c3)nc(-c3ccc4sc5ccccc5c4c3)n2)cc1 ; c1ccc(-c2nc(-c3cccc(-c4ccc5c6ccccc6c6ccccc6c5c4)c3)nc(-c3cccc4c3oc3ccccc34)n2)cc1 ; c1ccc(-c2nc(-c3cccc(-c4ccc5c6ccccc6c6ccccc6c5c4)c3)nc(-c3cccc4c3sc3ccccc34)n2)cc1]

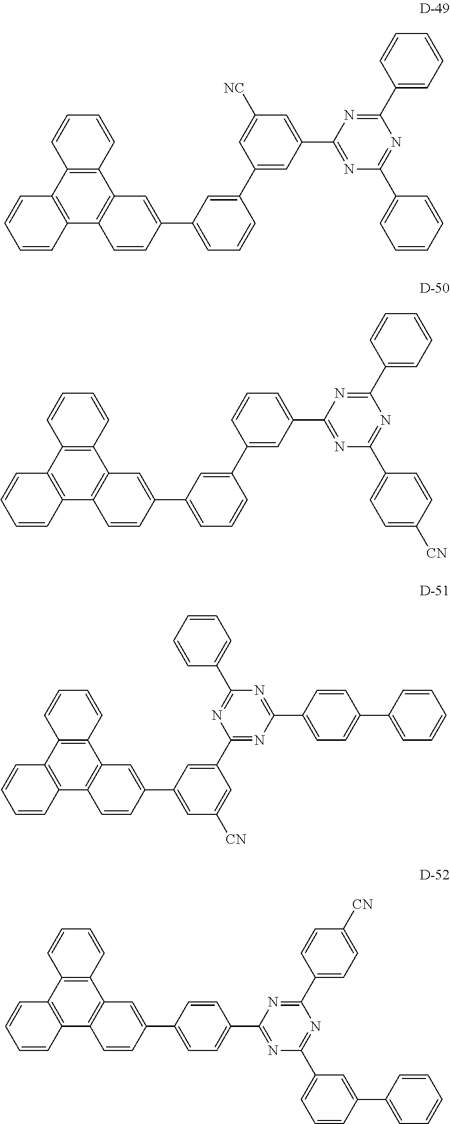 [SMILES: N#Cc1cc(-c2cccc(-c3ccc4c5ccccc5c5ccccc5c4c3)c2)cc(-c2nc(-c3ccccc3)nc(-c3ccccc3)n2)c1 ; N#Cc1ccc(-c2nc(-c3ccccc3)nc(-c3cccc(-c4cccc(-c5ccc6c7ccccc7c7ccccc7c6c5)c4)c3)n2)cc1 ; N#Cc1cc(-c2ccc3c4ccccc4c4ccccc4c3c2)cc(-c2nc(-c3ccccc3)nc(-c3ccc(-c4ccccc4)cc3)n2)c1 ; N#Cc1ccc(-c2nc(-c3ccc(-c4ccc5c6ccccc6c6ccccc6c5c4)cc3)nc(-c3cccc(-c4ccccc4)c3)n2)cc1]

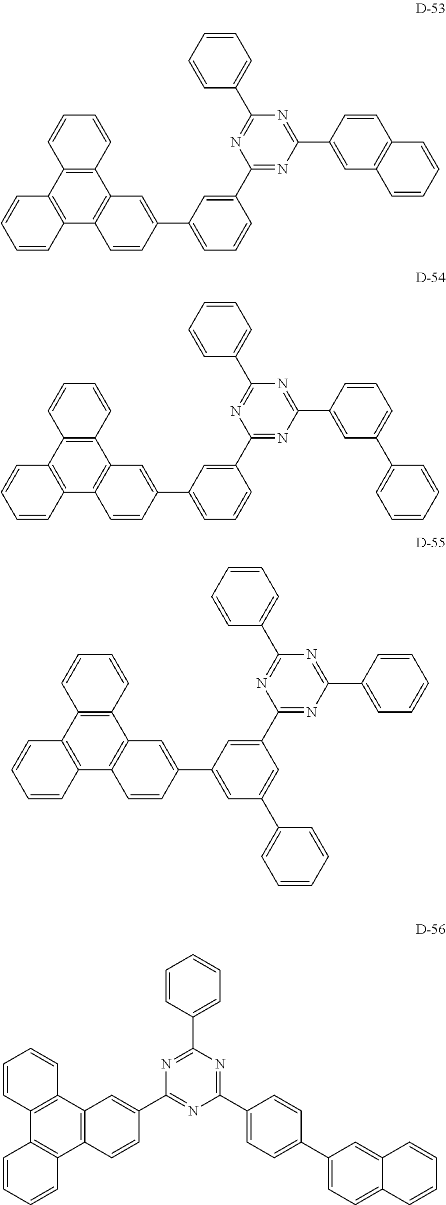 [SMILES: c1ccc(-c2nc(-c3cccc(-c4ccc5c6ccccc6c6ccccc6c5c4)c3)nc(-c3ccc4ccccc4c3)n2)cc1 ; c1ccc(-c2cccc(-c3nc(-c4ccccc4)nc(-c4cccc(-c5ccc6c7ccccc7c7ccccc7c6c5)c4)n3)c2)cc1 ; c1ccc(-c2cc(-c3ccc4c5ccccc5c5ccccc5c4c3)cc(-c3nc(-c4ccccc4)nc(-c4ccccc4)n3)c2)cc1 ; c1ccc(-c2nc(-c3ccc(-c4ccc5ccccc5c4)cc3)nc(-c3ccc4c5ccccc5c5ccccc5c4c3)n2)cc1]

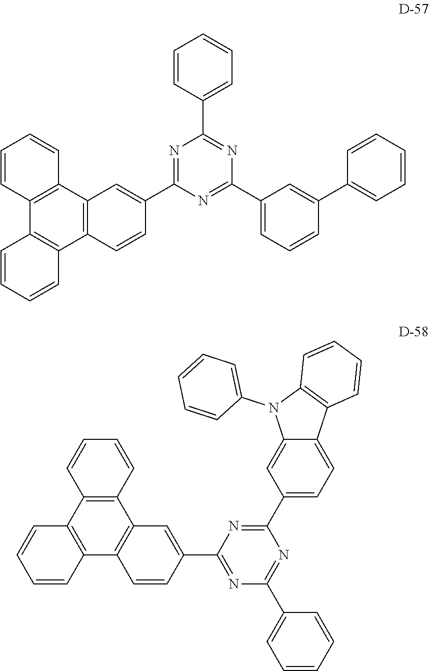

The first compound and the second compound may be included in a weight ratio of, e.g., about 1:99 to about 99:1. By being included in the above range, the hole transport ability of the first compound and the electron transport ability of the second compound can be used to achieve a proper weight ratio to implement bipolar characteristics, thereby helping improving efficiency and life-span. Within the above range, they may be included in a weight ratio of, e.g., about 90:10 to about 10:90, about 80:20 to about 20:80, e.g., about 80:20 to about 30:70, about 80:20 to about 40:60, and about 70:30 to about 40:60. In an implementation, they may be included in a weight ratio of about 60:40, about 50:50, or about 40:60.

In addition to the aforementioned first compound and second compound, one or more additional compounds may be included.

The aforementioned compound for the organic optoelectronic device or composition for the organic optoelectronic device may be a composition further including a dopant.

The dopant may be, e.g., a phosphorescent dopant, e.g., a red, green or blue phosphorescent dopant, e.g., a red or green phosphorescent dopant.

The dopant may be a material mixed with the composition for an organic optoelectronic device in a small amount to cause light emission and may be generally a material such as a metal complex that emits light by multiple excitation into a triplet or more. The dopant may be, e.g., an inorganic, organic, or organic-inorganic compound, and one or more types thereof may be used.

Examples of the dopant may be or include a phosphorescent dopant and examples of the phosphorescent dopant may be or include an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

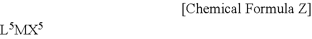

In Chemical Formula Z, M may be, e.g., a metal and L⁵ and X⁵ may be the same or different and may be, e.g., a ligand forming a complex compound with M.

The M may be, e.g., Ir, Pt, Os, Ti, Zr, Hf, Eu, Tb, Tm, Fe, Co, Ni, Ru, Rh, Pd, or a combination thereof, and L⁵ and X⁵ may be, e.g., a bidentate ligand.

An example of a bidentate may be represented by Chemical Formula Z-1.

Examples of ligands represented by L⁵ and X⁵ may be, e.g., a chemical formula of Group A.

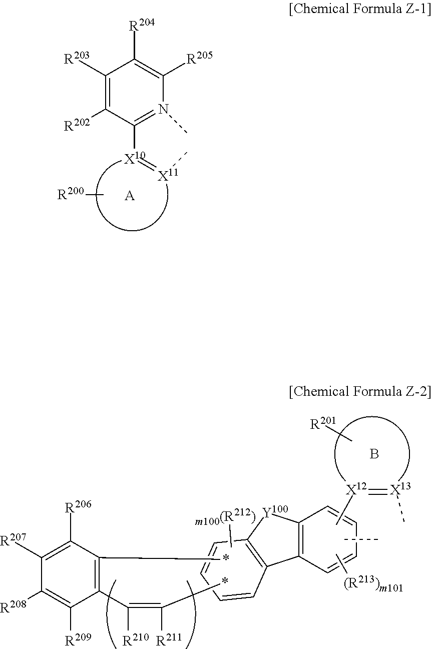

In Chemical Formula Z-1 and Chemical Formula Z-2, ring A and ring B may each independently be, e.g., a monocyclic ring or a polycyclic fused ring, wherein each ring among the monocyclic ring and polycyclic fused ring may be, e.g., a 5- or 6-membered carbocyclic or heterocyclic ring.

R²⁰⁰ and R²⁰¹ may each independently represent, e.g., one to a maximum number of monovalent substituents.

In an implementation, R²⁰⁰ and R²⁰¹ may each be two or more, and each R²⁰⁰ and R²⁰¹ may be the same or different from each other.

R²⁰² to R²¹³ may each independently be, e.g., hydrogen, deuterium, a halogen, a substituted or unsubstituted C1 to C10 alkyl group, a substituted or unsubstituted C6 to C20 aryl group, —SiR²¹⁴R²¹⁵R²¹⁶, —GeR²¹⁴R²¹⁵R²¹⁶, or a combination thereof.

R²¹⁴ to R²¹⁶ may each independently be, e.g., a substituted or unsubstituted C1 to C6 alkyl group.

X¹⁰, X¹¹, X¹², and X¹³ may each independently be, e.g., carbon or nitrogen. Y¹⁰⁰ may be O or S.

m100 may be, e.g., an integer of 1 to 3.

m101 may be, e.g., an integer of 1 to 2.

n100 may be, e.g., an integer of 0 or 1.

* is a linking point.

In an implementation, n100 is 0, and it may be formed with a monovalent substituent.

In an implementation, n100 is 1, and a fusion ring may be formed.

Examples of the ligands represented by L⁵ and X⁵ may be, e.g., a chemical formula of Group A.

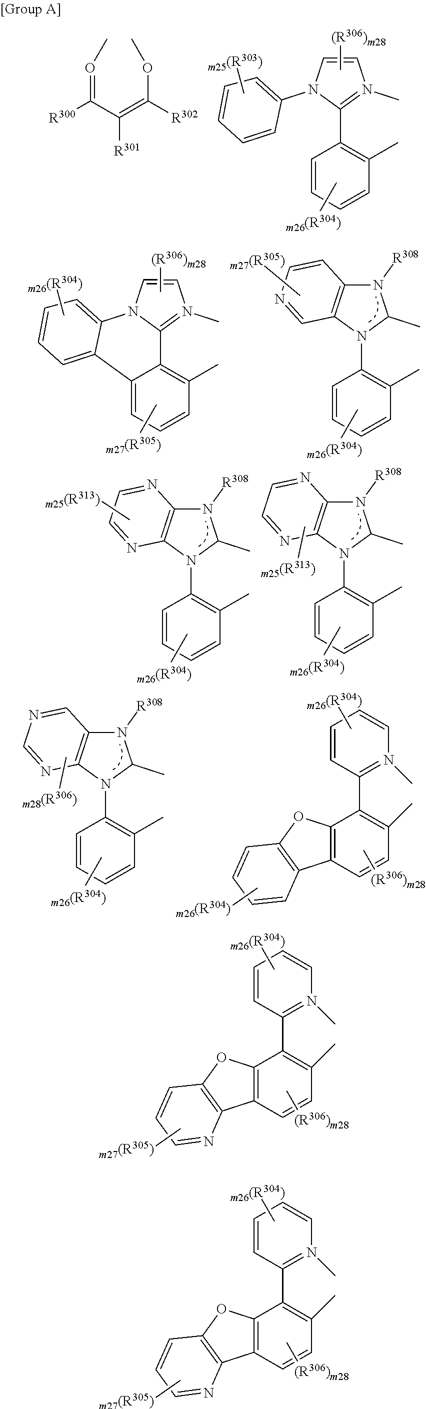

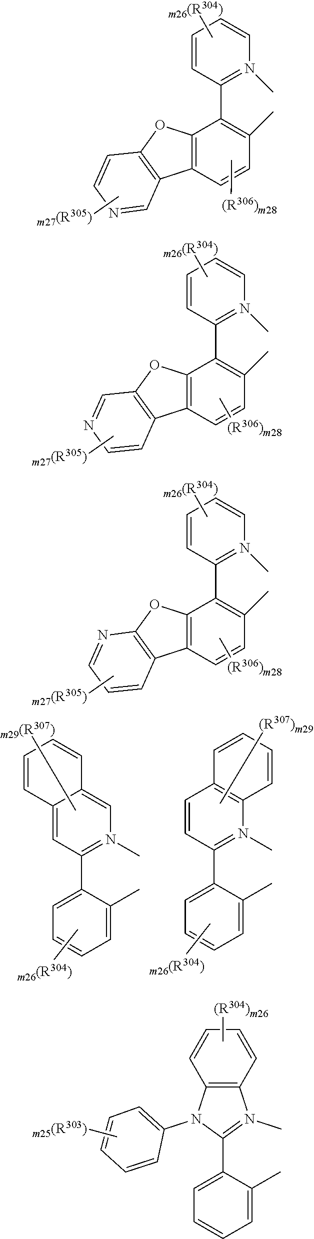

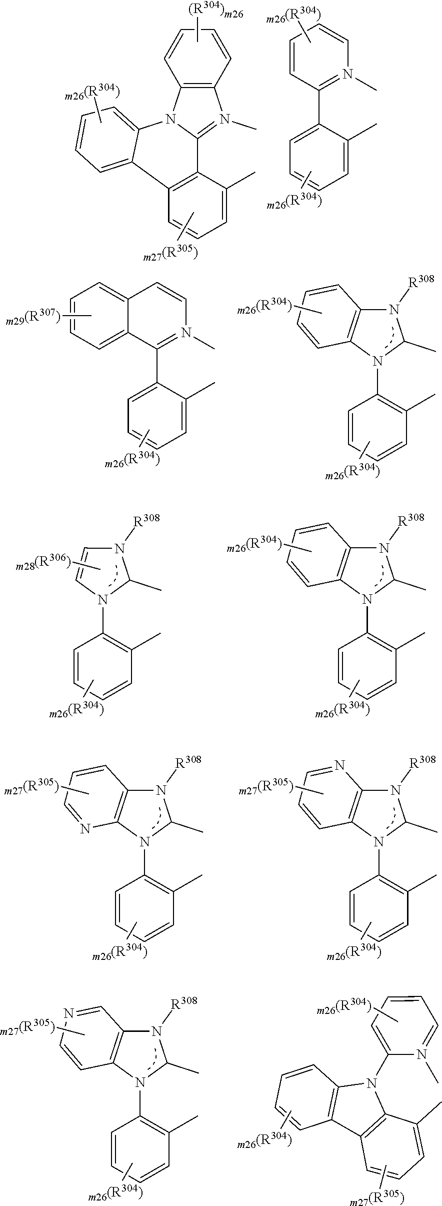

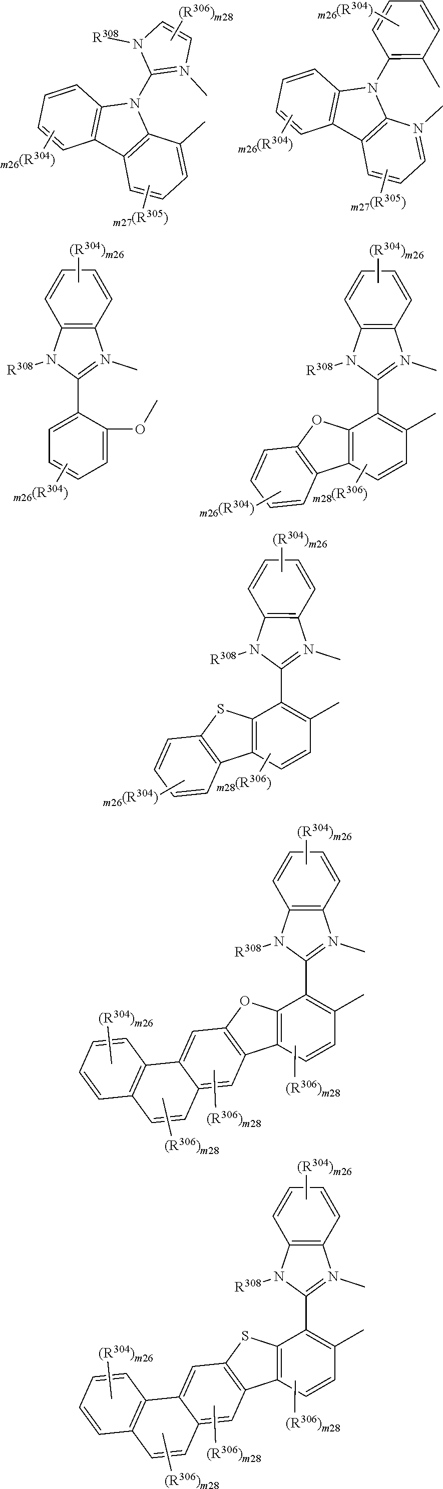

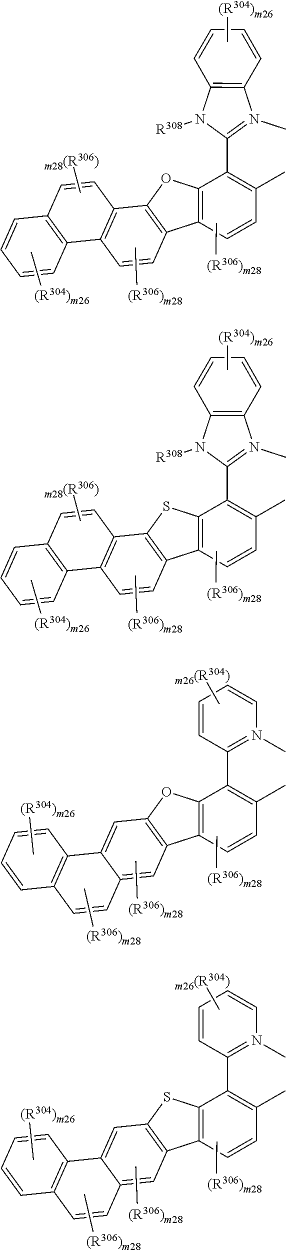

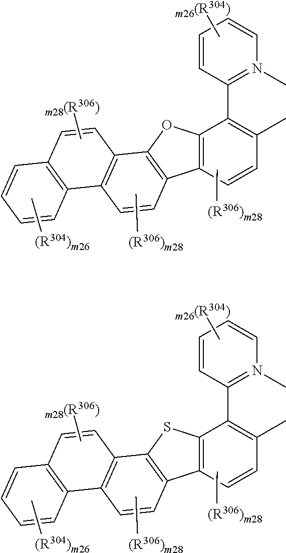

In Group A, R³⁰⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen.

R³⁰³ to R³⁰⁸ may each independently be, e.g., hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group or a substituted or unsubstituted C6 to C30 aryl group, or a triarylsilyl group having a substituted or unsubstituted C6 to C30 aryl group.

m25 may be, e.g., an integer of 1 to 5.

m26 may be, e.g., an integer of 1 to 4.

m27 may be, e.g., an integer of 1 to 3.

m28 may be, e.g., an integer of 1 or 2.

m29 may be, e.g., an integer of 1 to 6.

In an implementation, m25 to m29 may be 2 or more, and each R³⁰³ to R³⁰⁷ may be the same or different from each other.

The dopant according to some example embodiments may be an iridium complex, and may be represented, e.g., by Chemical Formula 6-1 to Chemical Formula 6-5.

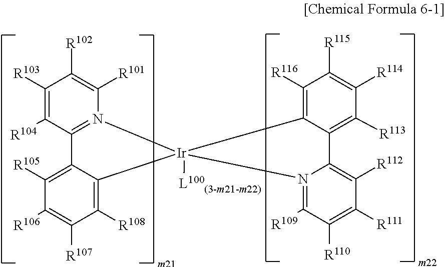

In Chemical Formula 6-1, R¹⁰¹ to R¹¹⁶ may each independently be, e.g., hydrogen, deuterium, a halogen, a substituted or unsubstituted C1 to C10 alkyl group, a substituted or unsubstituted C6 to C20 aryl group, —SiR¹³²R¹³³R¹³⁴, or —GeR¹³²R¹³³R¹³⁴.

R¹³² to R¹³⁴ may each independently be, e.g., a substituted or unsubstituted C1 to C6 alkyl group.

At least one of R¹⁰¹ to R¹¹⁶ may be, e.g., a functional group represented by Chemical Formula V-1.

L¹⁰⁰ may be, e.g., a bidentate ligand of a monovalent anion and may be, e.g., a ligand that coordinates to iridium through a lone pair of carbons or heteroatoms.

m21 and m22 may each independently be, e.g., an integer of 0 to 3 and m21+m22 may be, e.g., an integer of 1 to 3.

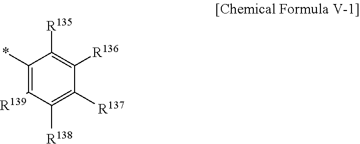

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

* refers to a portion linked to a carbon atom.

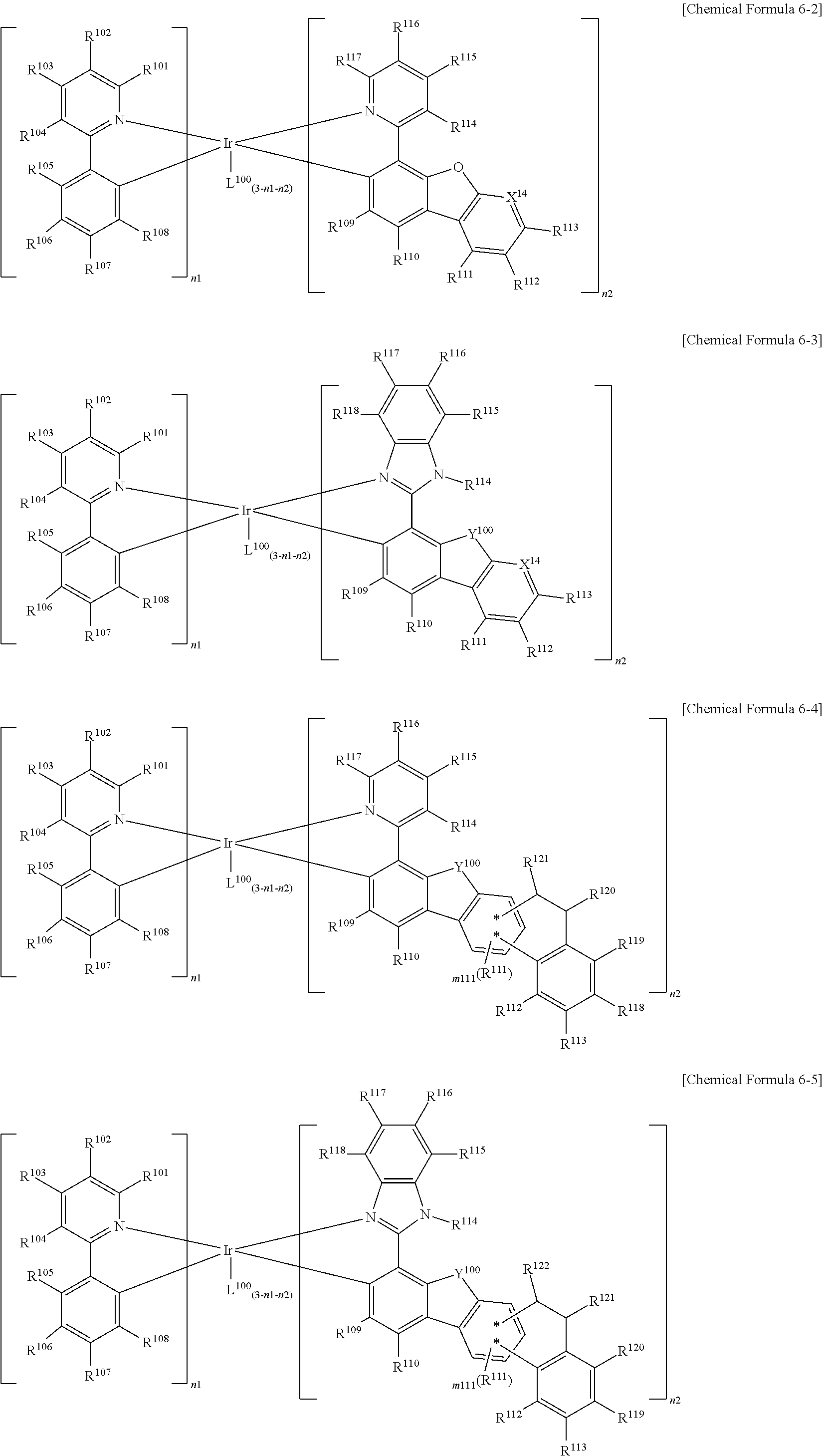

In Chemical Formula 6-2 to Chemical Formula 6-5, X¹⁴ may be, e.g., carbon or nitrogen.

Y¹⁰⁰ may be, e.g., O or S.

R¹⁰¹ to R¹²² may each independently be, e.g., hydrogen, deuterium, a halogen, a substituted or unsubstituted C1 to C10 alkyl group, a substituted or unsubstituted C6 to C20 aryl group, —SiR¹³³R¹³⁴R¹³⁵, or —GeR¹³³R¹³⁴R¹³⁵.

R¹³³ to R¹³⁵ may each independently be, e.g., a substituted or unsubstituted C1 to C6 alkyl group.

L¹⁰⁰ may be a bidentate ligand of a monovalent anion, and may be a ligand that coordinates to iridium through a lone pair of carbons or heteroatoms.

m111 may be, e.g., an integer of 1 to 2.

n1 and n2 may each independently be, e.g., an integer of 0 to 3, and n1+n2 may be, e.g., an integer of 1 to 3.

The dopant according to some example embodiments may be a platinum complex, and may be represented, e.g., by Chemical Formula Y-1.

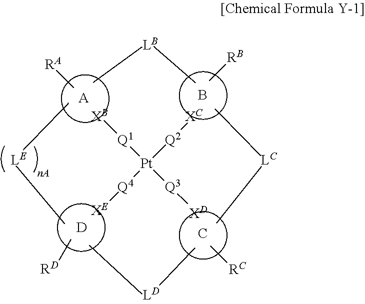

In Chemical Formula Y-1, rings A, B, C, and D may each independently be, e.g., a 5-membered or 6-membered carbocyclic or heterocyclic ring.

R^{A}, R^{B}, R^{C}, and R^{D} may each independently be, e.g., mono-, di-, tri-, or tetra-substitution, or unsubstitution.

L^{B}, L^{C}, and L^{D} may each independently be, e.g., a direct bond, BR, NR, PR, O, S, Se, C═O, S═O, SO₂, CRR′, SiRR′, GeRR′, or a combination thereof.

In an implementation, nA may be 1, and L^{E} may be a direct bond, BR, NR, PR, O, S, Se, C═O, S═O, SO₂, CRR′, SiRR′, GeRR′, or a combination thereof. In an implementation, nA may be 0, and L^{E} may not exist.

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ may be optionally linked to each other to provide a ring; X^{B}, X^{C}, X^{D}, and X^{E} may each independently be, e.g., carbon or nitrogen; and Q¹, Q², Q³, and Q⁴ may each independently be, e.g., oxygen or a direct bond.

The platinum complex may be represented, e.g., by Chemical Formula 7-1 or Chemical Formula 7-2.

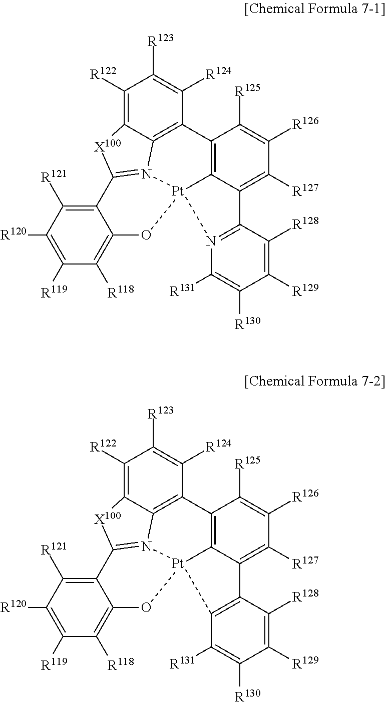

In Chemical Formula 7-1 and Chemical Formula 7-2, X¹⁰⁰ may be, e.g., O, S, or NR¹³².

R¹¹⁸ to R¹³² may each independently be, e.g., hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be, e.g., a substituted or unsubstituted C1 to C6 alkyl group.

In an implementation, at least one of R¹¹⁸ to R¹³² may be, e.g., —SiR¹³³R¹³⁴R¹³⁵ or a tert-butyl group.

R¹³³ to R¹³⁵ may each independently be, e.g., a substituted or unsubstituted C1 to C6 alkyl group.

The organic layer may further include a charge transport region in addition to the light emitting layer.

The charge transport region may be, e.g., the hole transport region 140.

The hole transport region 140 may help further increase hole injection or hole mobility between the anode 120 and the light emitting layer 130 and block electrons.

In an implementation, the hole transport region 140 may include, e.g., a hole transport layer between the anode 120 and the light emitting layer 130, and a hole transport auxiliary layer between the light emitting layer 130 and the hole transport layer, and a compound of Group B may be included in at least one of the hole transport layer and the hole transport auxiliary layer.

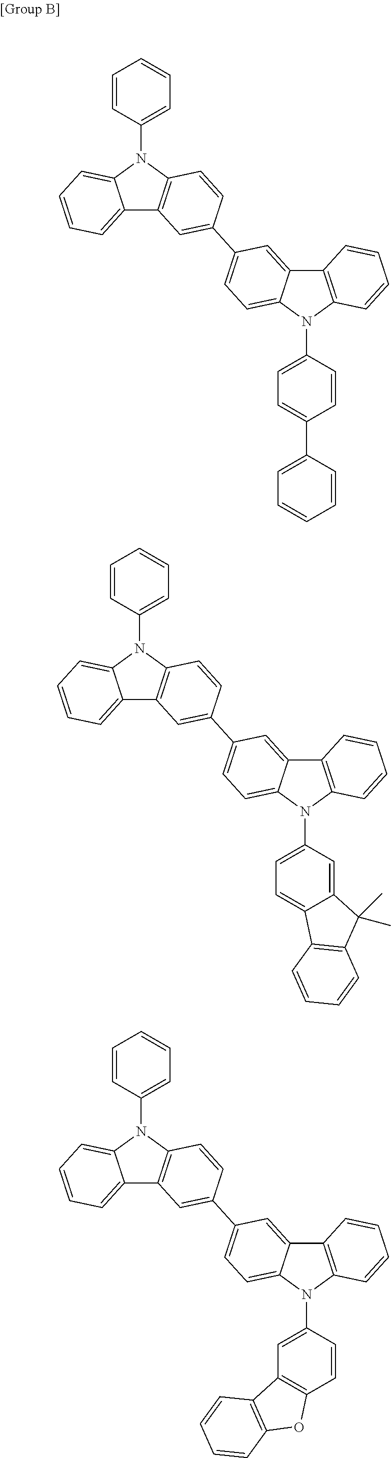 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

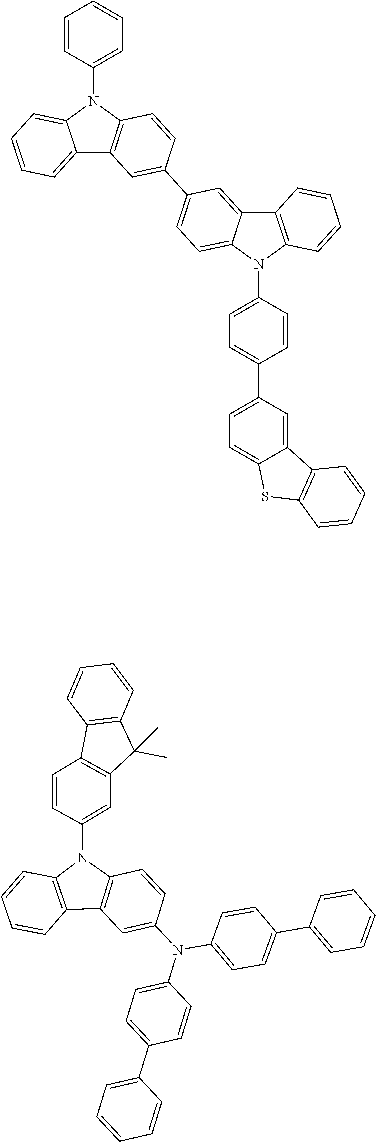 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

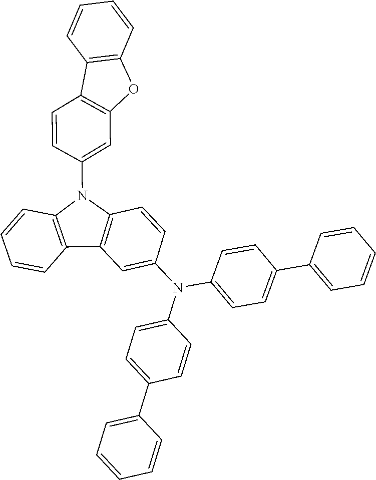 [SMILES: c1ccc(-c2ccc(N(c3ccc(-c4ccccc4)cc3)c3ccc4c(c3)c3ccccc3n4-c3ccc4c(c3)oc3ccccc34)cc2)cc1]

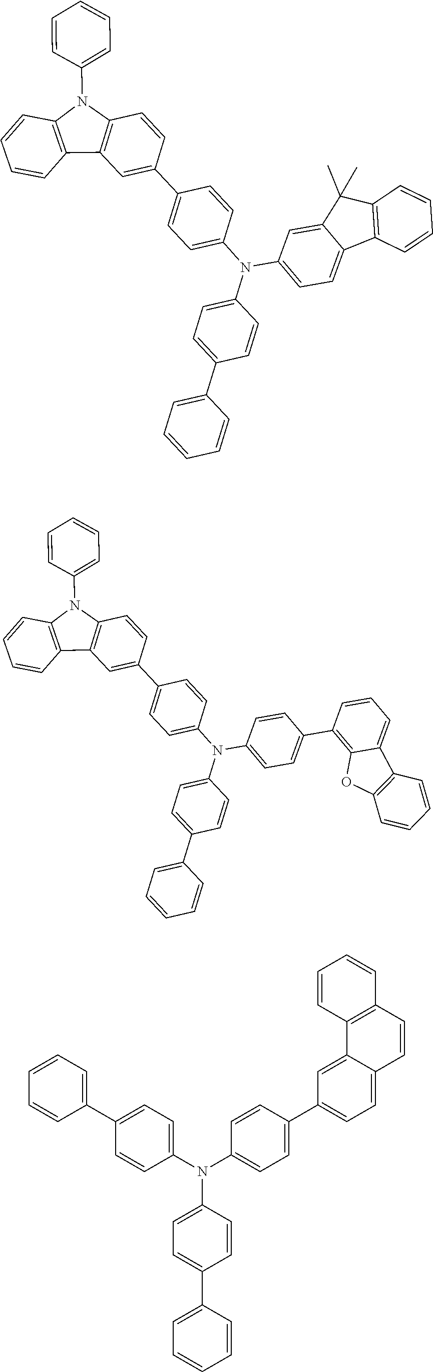 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1]

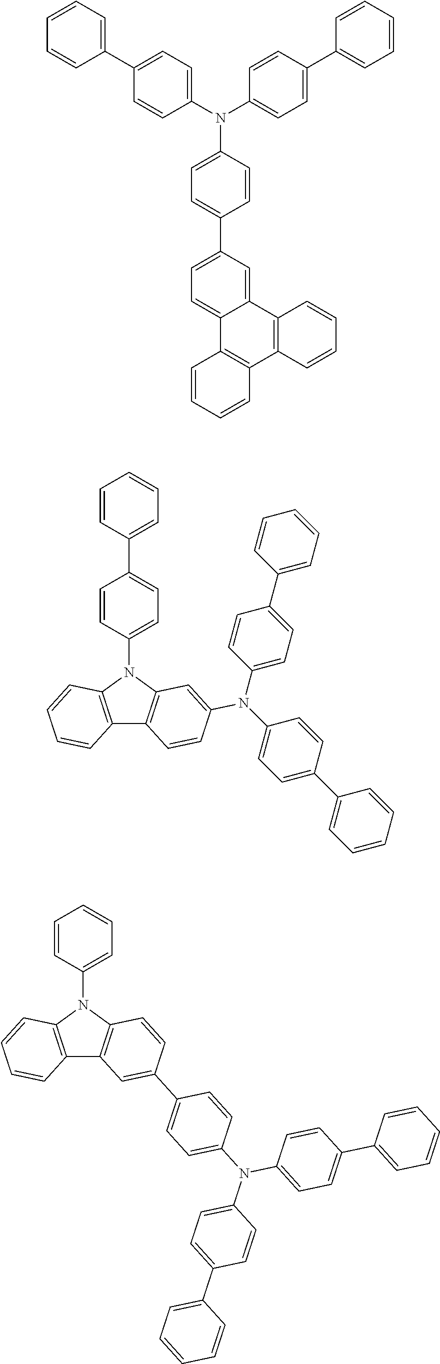 [SMILES: c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

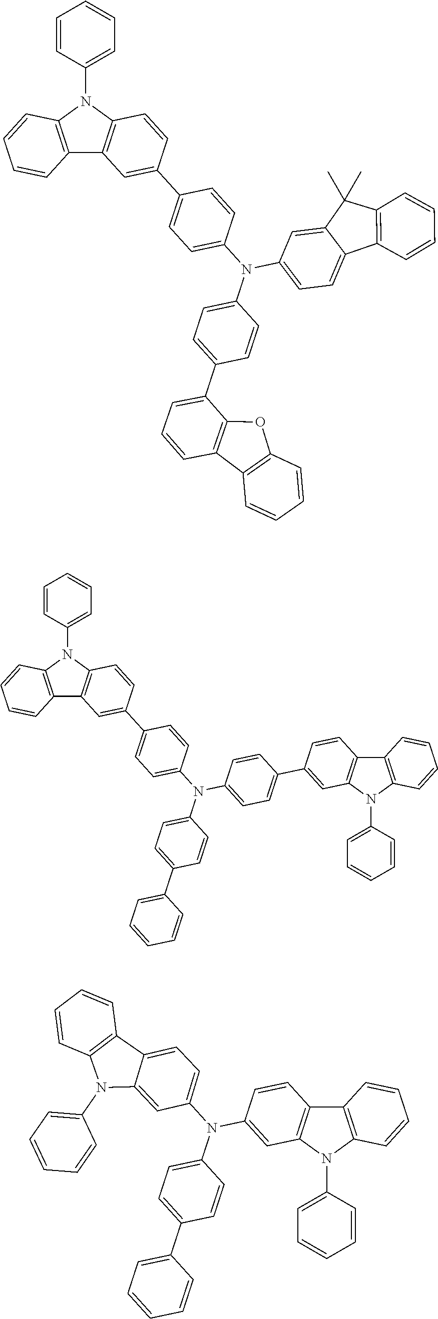 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1]

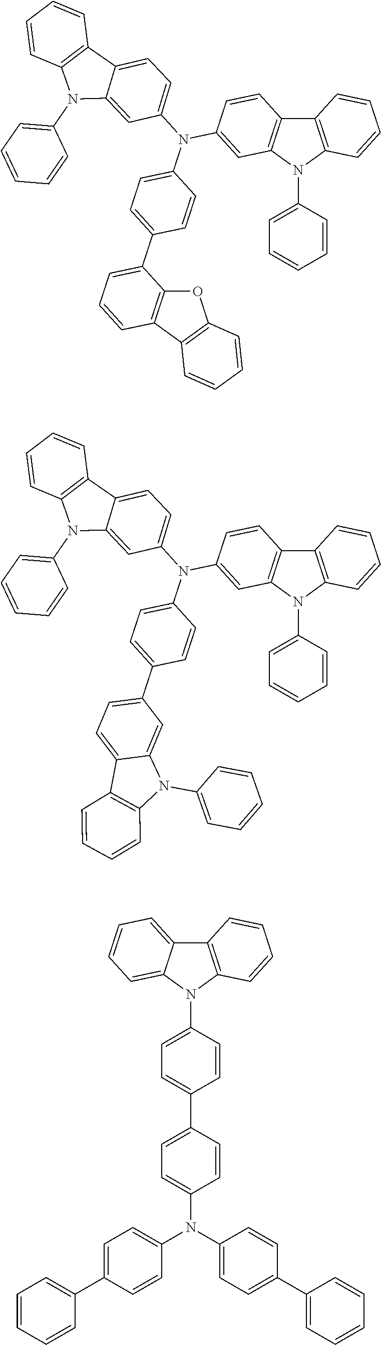 [SMILES: c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1 ; c1ccc(-c2ccc(N(c3ccc(-c4ccccc4)cc3)c3ccc(-c4ccc(-n5c6ccccc6c6ccccc65)cc4)cc3)cc2)cc1]

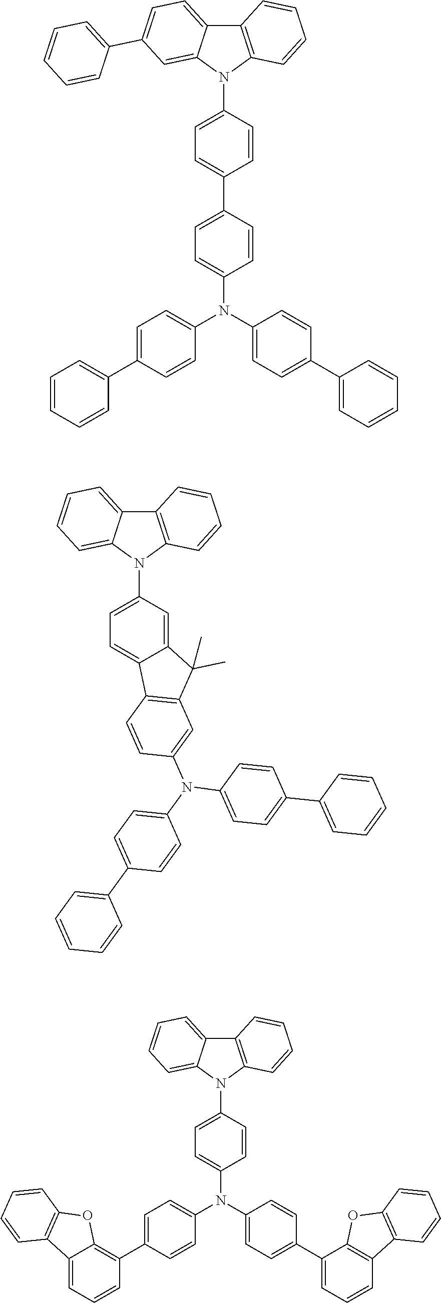 [SMILES: c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc2c(c1)oc1c(-c3ccc(N(c4ccc(-c5cccc6c5oc5ccccc56)cc4)c4ccc(-n5c6ccccc6c6ccccc65)cc4)cc3)cccc12]

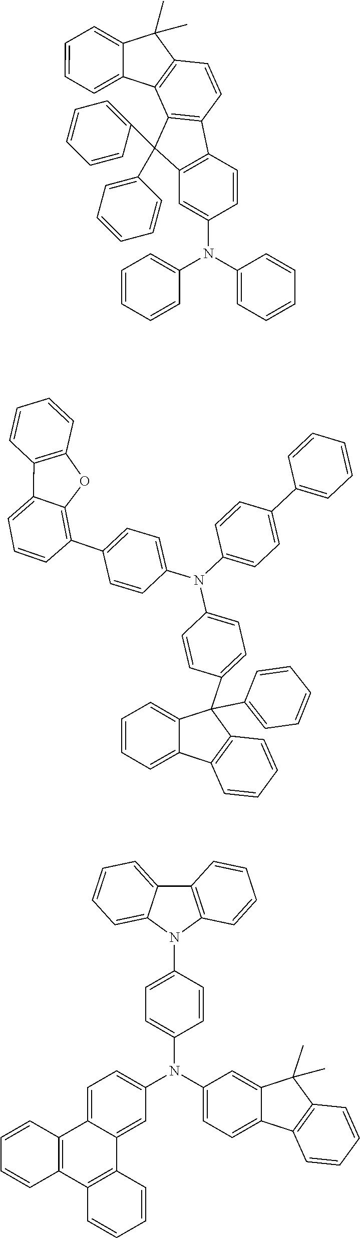 [SMILES: CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; CC1(C)c2ccccc2-c2ccc(N(c3ccc(-n4c5ccccc5c5ccccc54)cc3)c3ccc4c5ccccc5c5ccccc5c4c3)cc21]

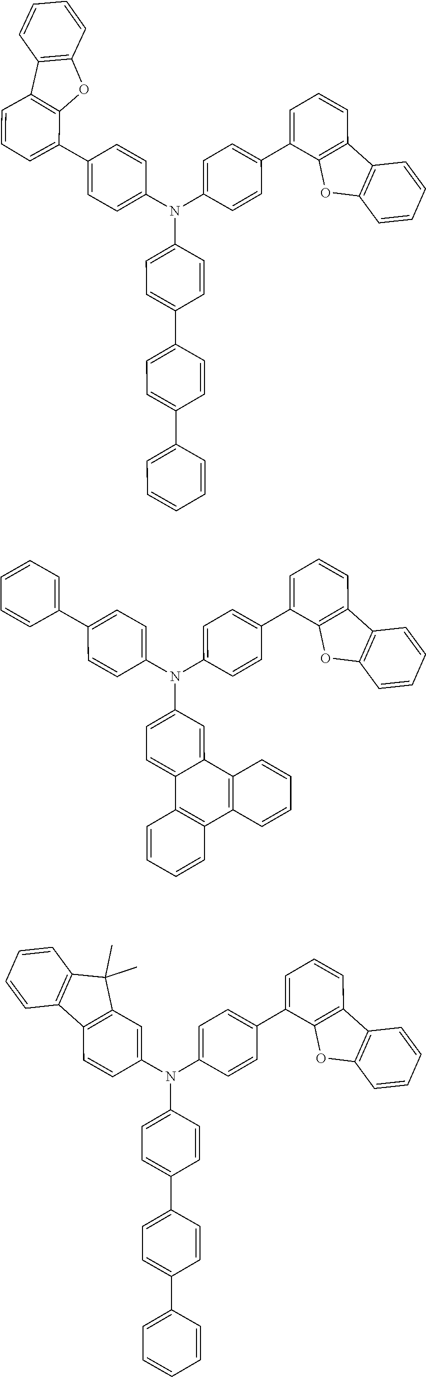 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21]

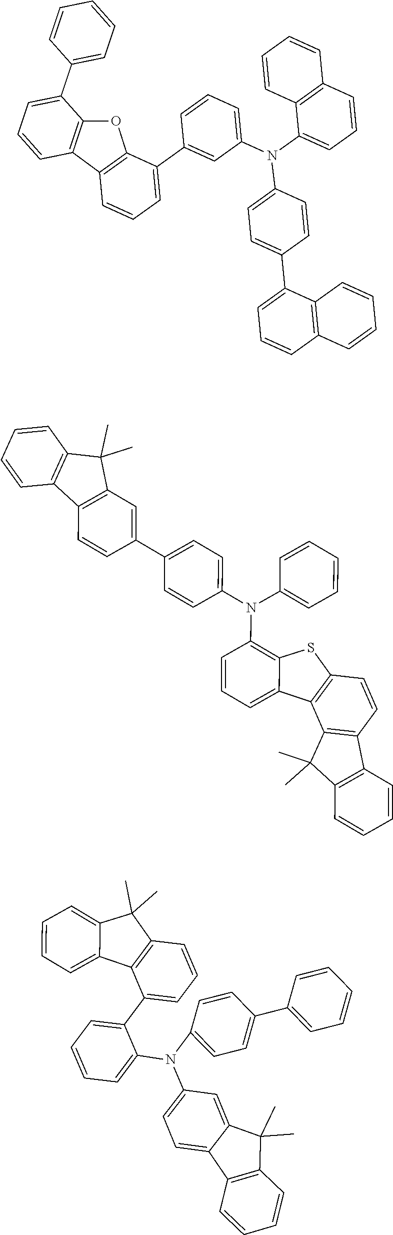 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

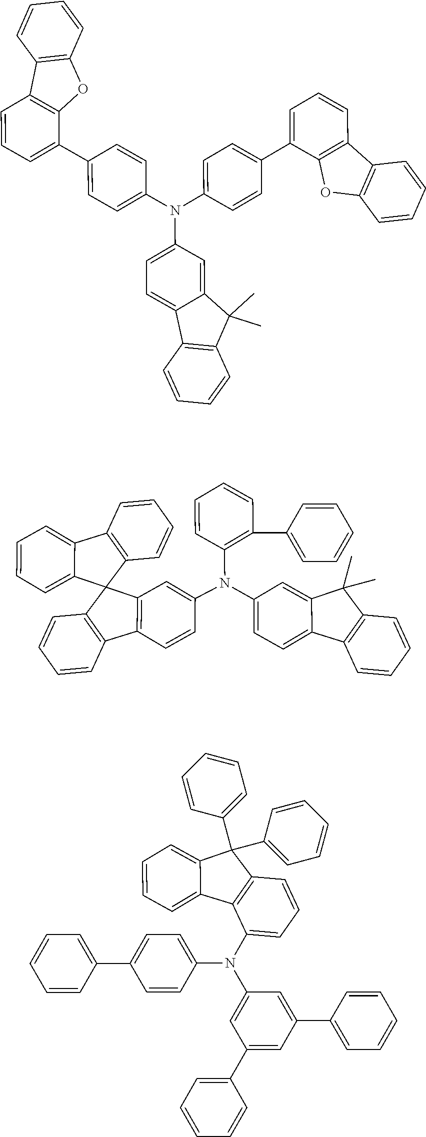 [SMILES: CC1(C)c2ccccc2-c2ccc(N(c3ccc(-c4cccc5c4oc4ccccc45)cc3)c3ccc(-c4cccc5c4oc4ccccc45)cc3)cc21 ; CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3cc(-c4ccccc4)cc(-c4ccccc4)c3)c3cccc4c3-c3ccccc3C4(c3ccccc3)c3ccccc3)cc2)cc1]

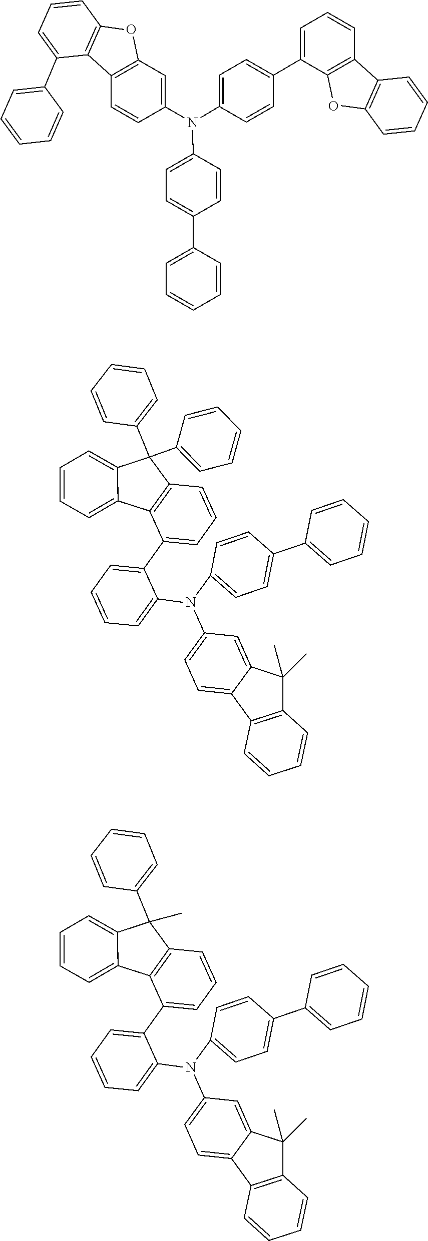 [SMILES: c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21]

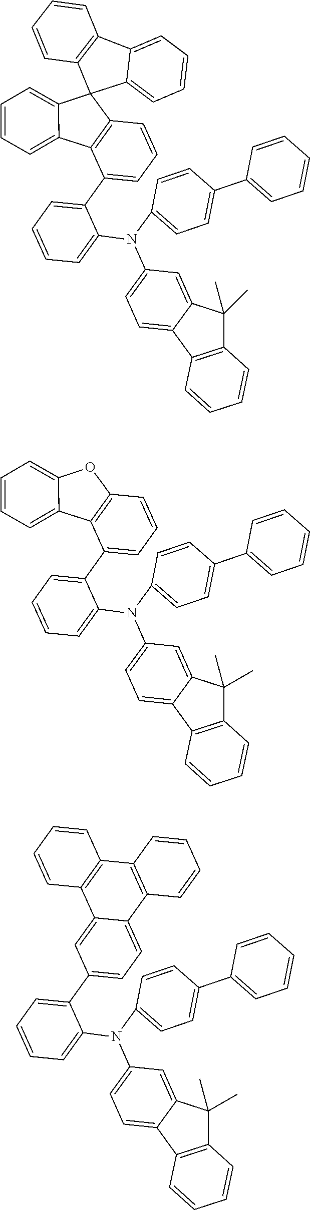 [SMILES: CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21 ; CC1(C)c2ccccc2-c2ccc(N(c3ccc(-c4ccccc4)cc3)c3ccccc3-c3ccc4c5ccccc5c5ccccc5c4c3)cc21]

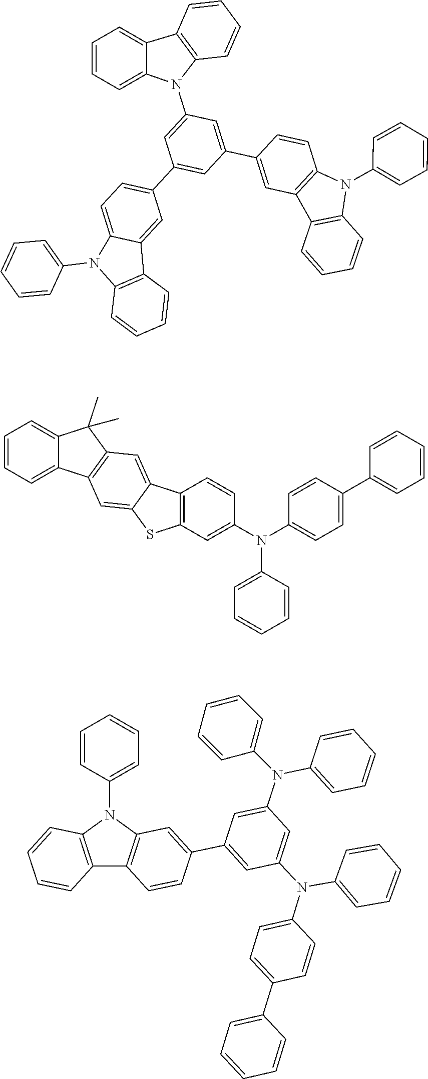 [SMILES: c1ccc(-n2c3ccccc3c3cc(-c4cc(-c5ccc6c(c5)c5ccccc5n6-c5ccccc5)cc(-n5c6ccccc6c6ccccc65)c4)ccc32)cc1 ; CC1(C)c2ccccc2-c2cc3sc4cc(N(c5ccccc5)c5ccc(-c6ccccc6)cc5)ccc4c3cc21 ; c1ccc(-c2ccc(N(c3ccccc3)c3cc(-c4ccc5c6ccccc6n(-c6ccccc6)c5c4)cc(N(c4ccccc4)c4ccccc4)c3)cc2)cc1]

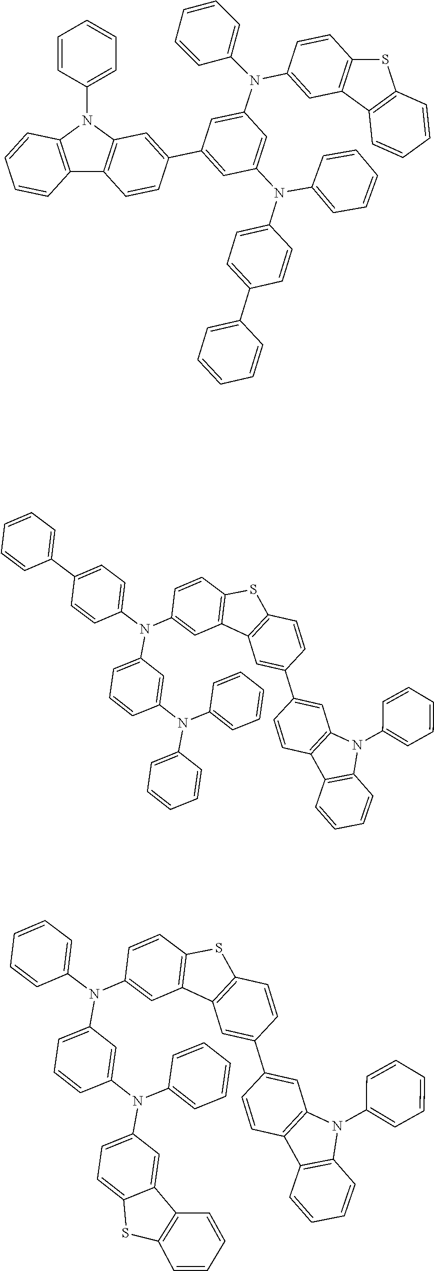 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1]

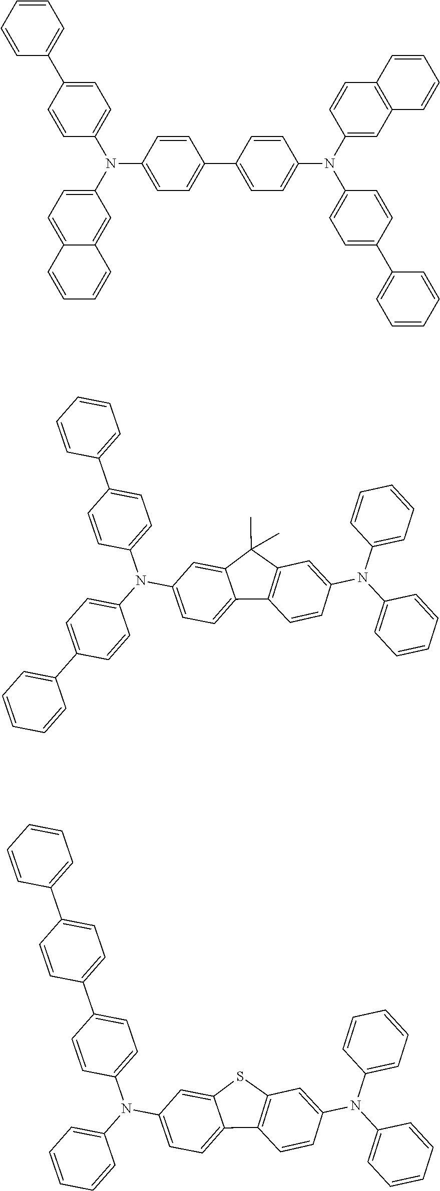 [SMILES: c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21 ; c1ccc(-c2ccc(-c3ccc(N(c4ccccc4)c4ccc5c(c4)sc4cc(N(c6ccccc6)c6ccccc6)ccc45)cc3)cc2)cc1]

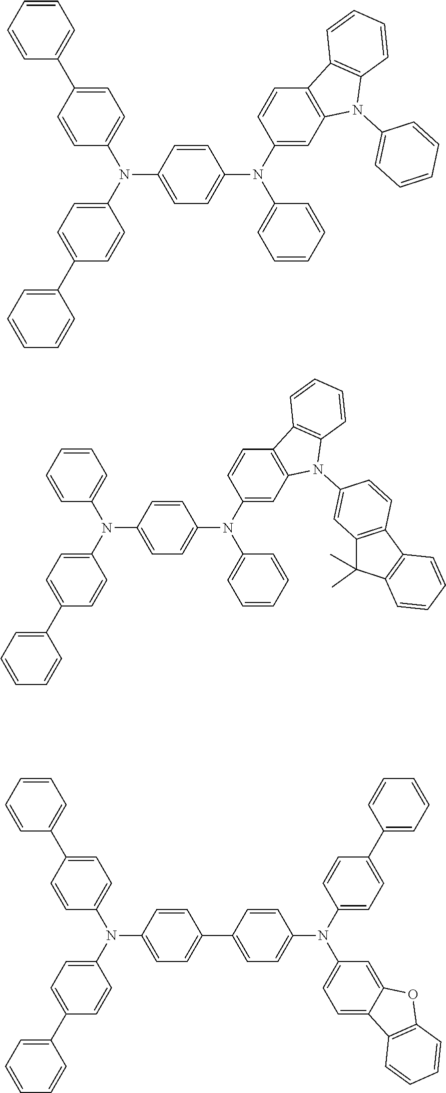 [SMILES: c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21 ; c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1]

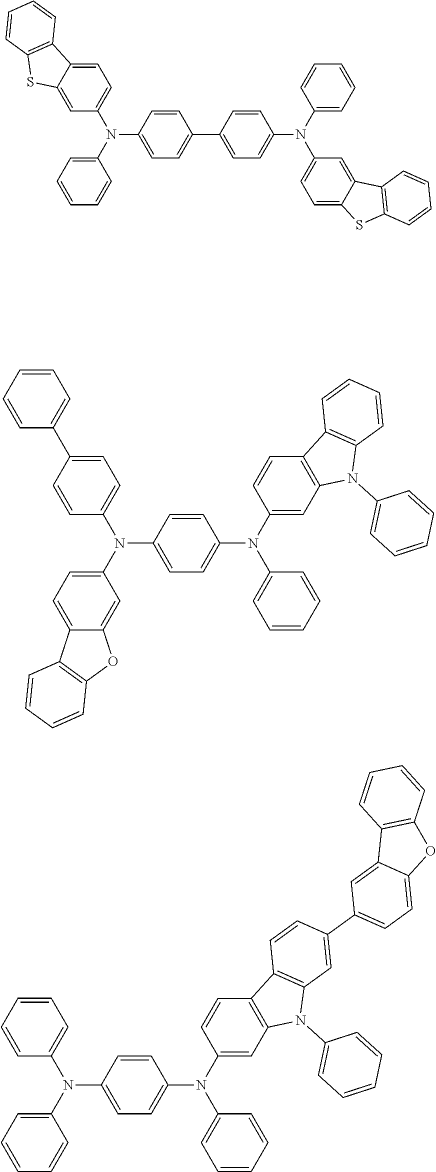 [SMILES: c1ccc(N(c2ccc(-c3ccc(N(c4ccccc4)c4ccc5sc6ccccc6c5c4)cc3)cc2)c2ccc3c(c2)sc2ccccc23)cc1 ; c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1 ; c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1]

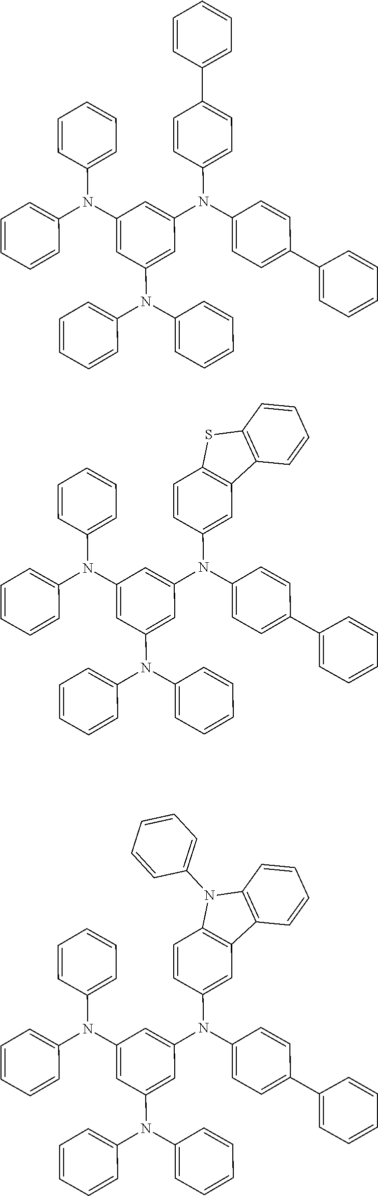 [SMILES: c1ccc(-c2ccc(N(c3ccc(-c4ccccc4)cc3)c3cc(N(c4ccccc4)c4ccccc4)cc(N(c4ccccc4)c4ccccc4)c3)cc2)cc1 ; c1ccc(-c2ccc(N(c3cc(N(c4ccccc4)c4ccccc4)cc(N(c4ccccc4)c4ccccc4)c3)c3ccc4sc5ccccc5c4c3)cc2)cc1 ; c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1]

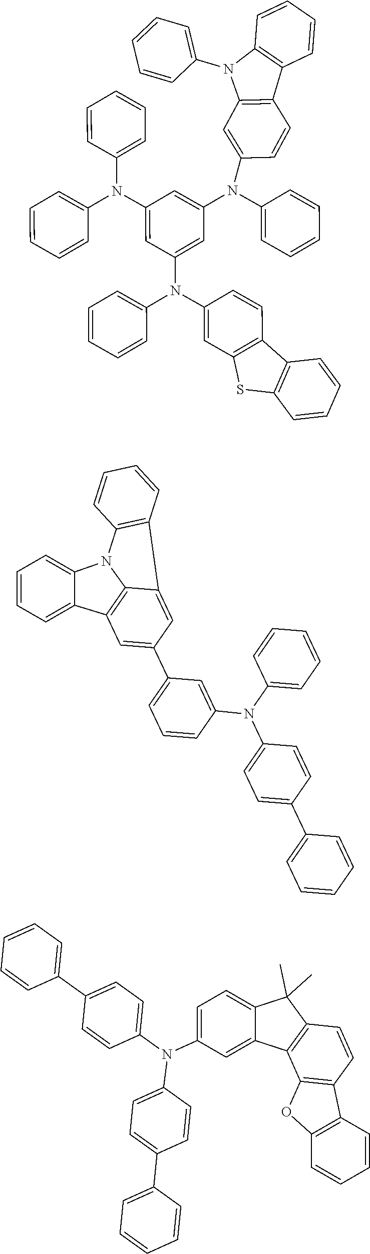 [SMILES: c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21]

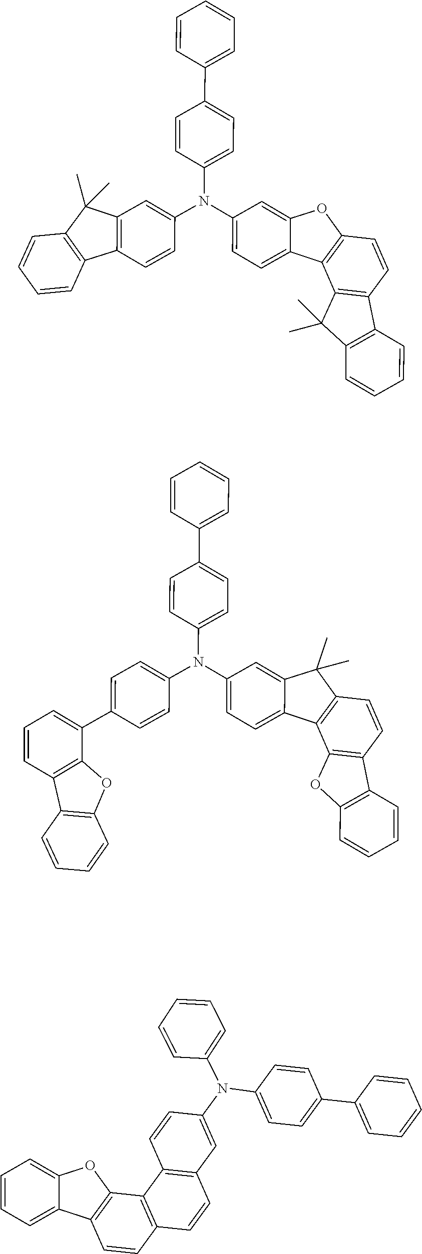

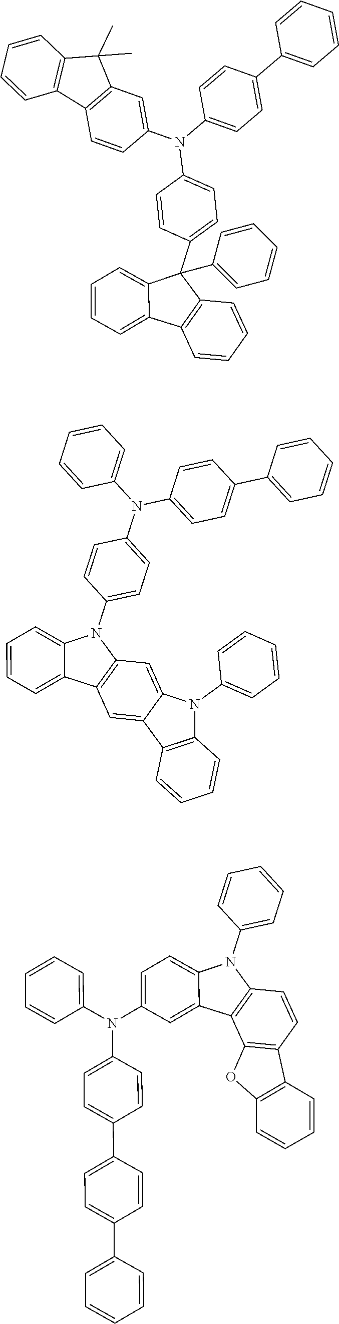 [SMILES: CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1 ; c1ccc(-c2ccc(-c3ccc(N(c4ccccc4)c4ccc5c(c4)c4c6oc7ccccc7c6ccc4n5-c4ccccc4)cc3)cc2)cc1]

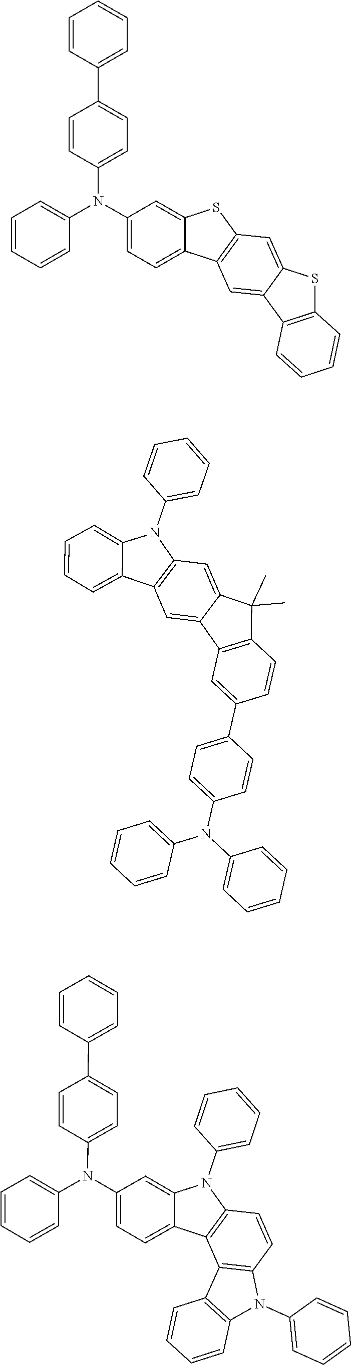 [SMILES: c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1]

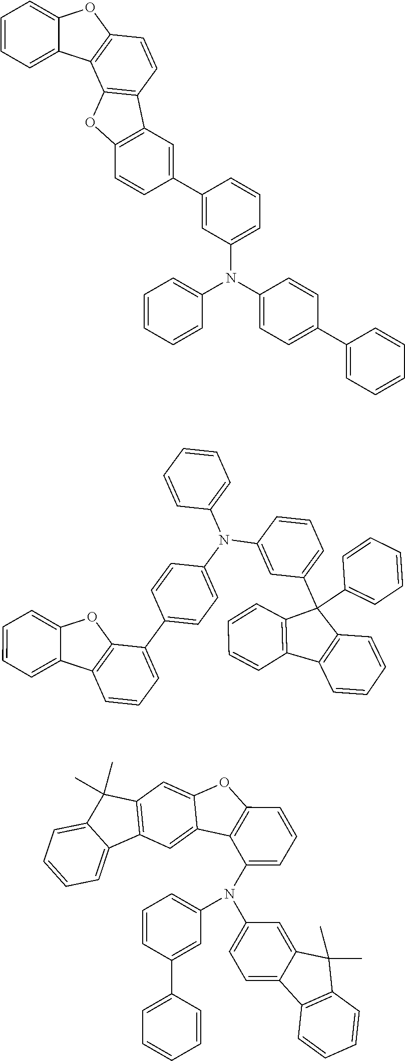 [SMILES: c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1 ; CC1(C)c2ccccc2-c2ccc(N(c3cccc(-c4ccccc4)c3)c3cccc4oc5cc6c(cc5c34)-c3ccccc3C6(C)C)cc21]

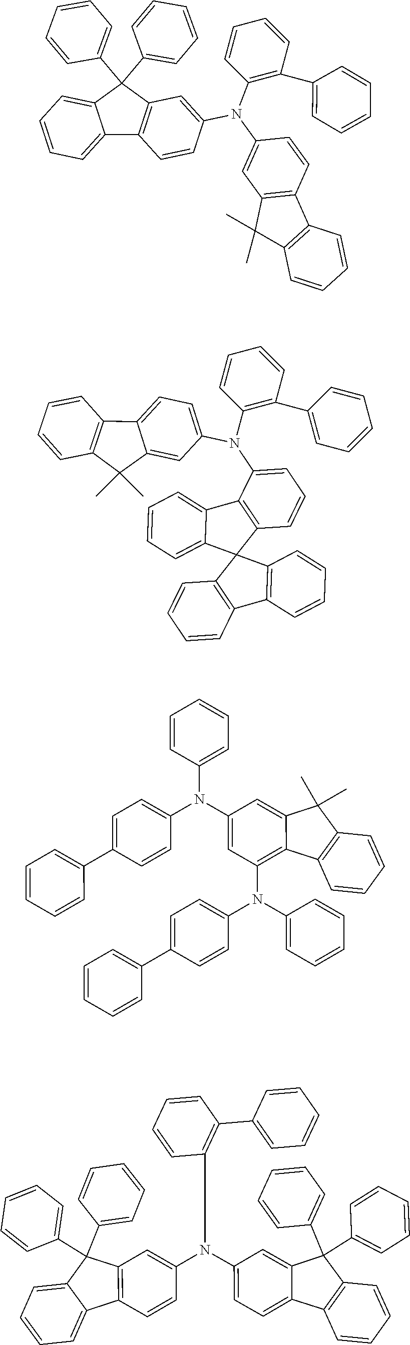 [SMILES: CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2c(N(c3ccccc3)c3ccc(-c4ccccc4)cc3)cc(N(c3ccccc3)c3ccc(-c4ccccc4)cc3)cc21 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

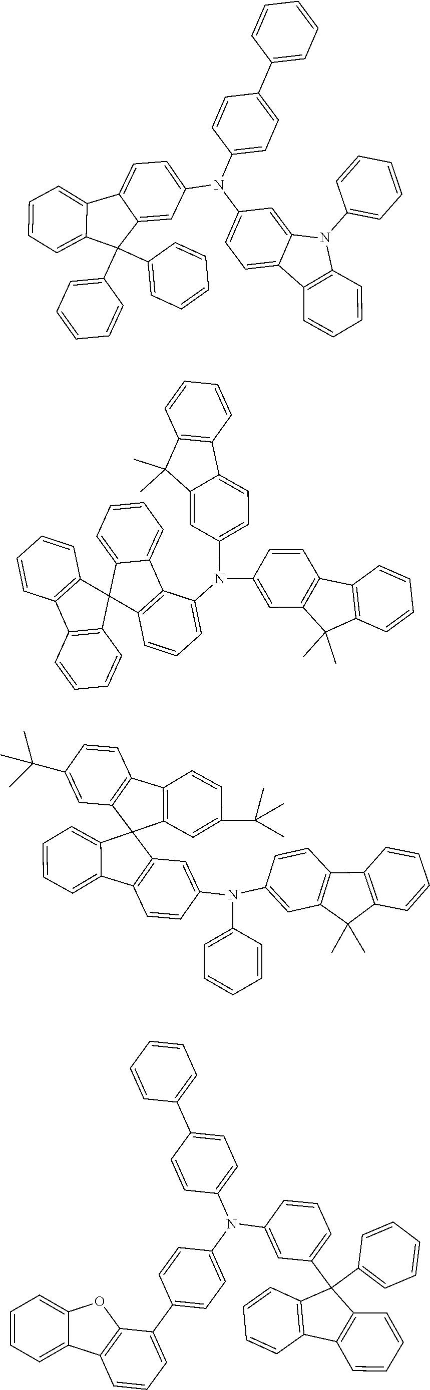 [SMILES: c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2 ; c1ccc(-c2ccc(N(c3ccc(-c4cccc5c4oc4ccccc45)cc3)c3cccc(C4(c5ccccc5)c5ccccc5-c5ccccc54)c3)cc2)cc1]

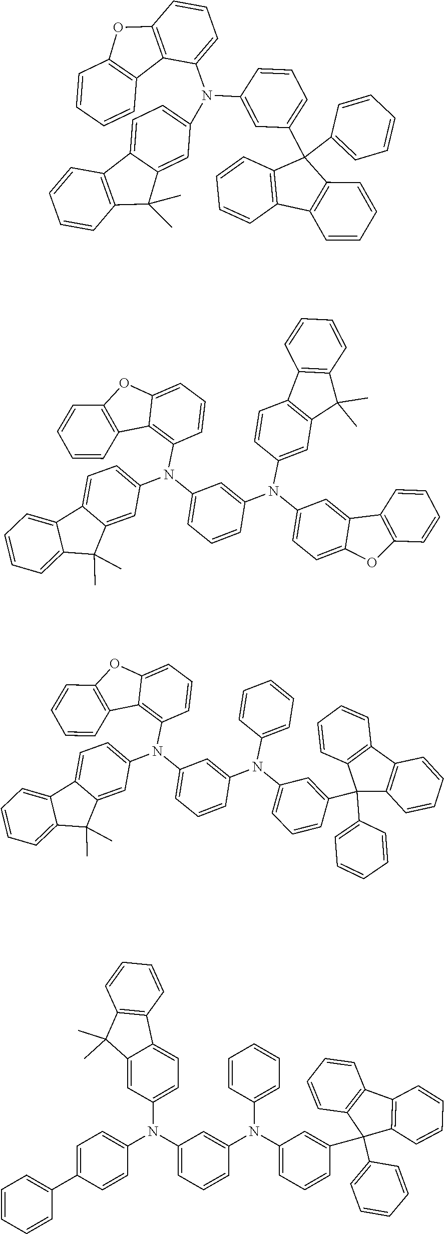 [SMILES: CC1(C)c2ccccc2-c2ccc(N(c3cccc(C4(c5ccccc5)c5ccccc5-c5ccccc54)c3)c3cccc4oc5ccccc5c34)cc21 ; CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21 ; CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21 ; CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21]

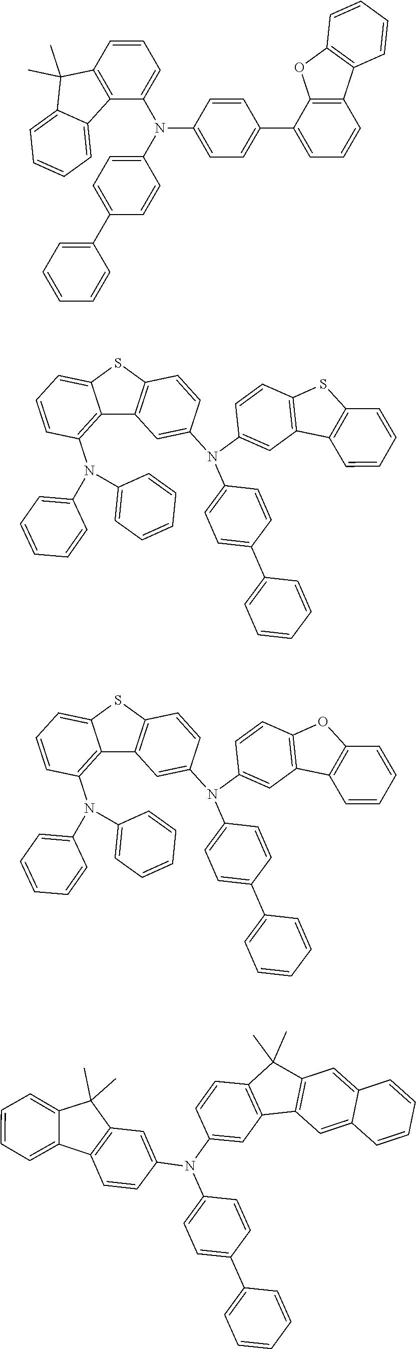 [SMILES: CC1(C)c2ccccc2-c2c(N(c3ccc(-c4ccccc4)cc3)c3ccc(-c4cccc5c4oc4ccccc45)cc3)cccc21 ; c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1 ; CC1(C)c2ccccc2-c2ccc(N(c3ccc(-c4ccccc4)cc3)c3ccc4c(c3)-c3cc5ccccc5cc3C4(C)C)cc21]

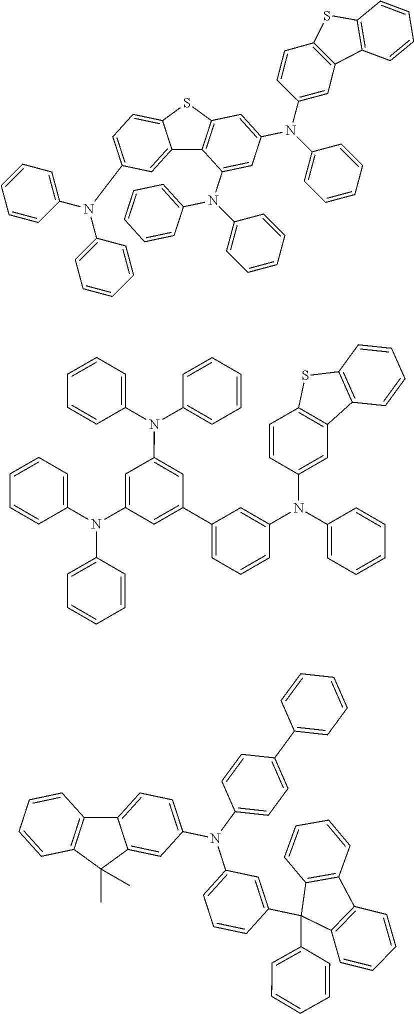 [SMILES: c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

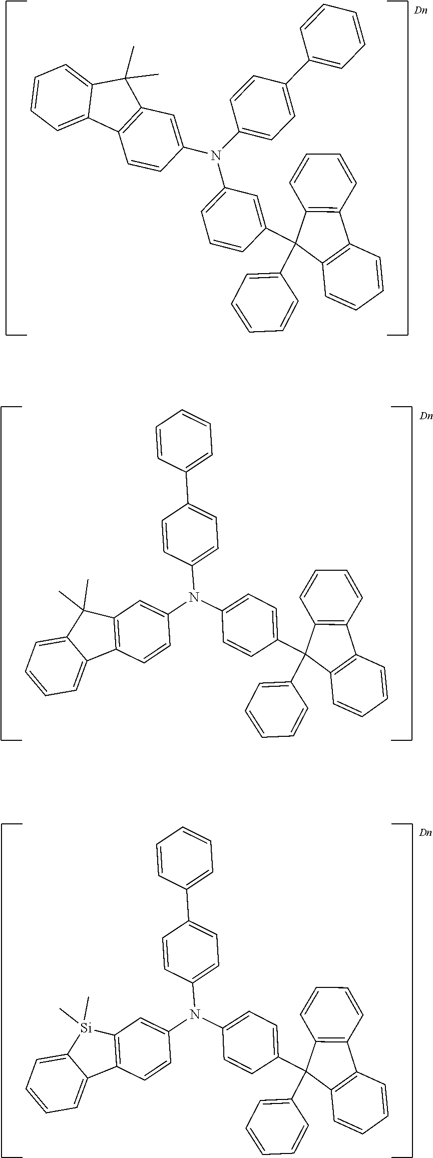

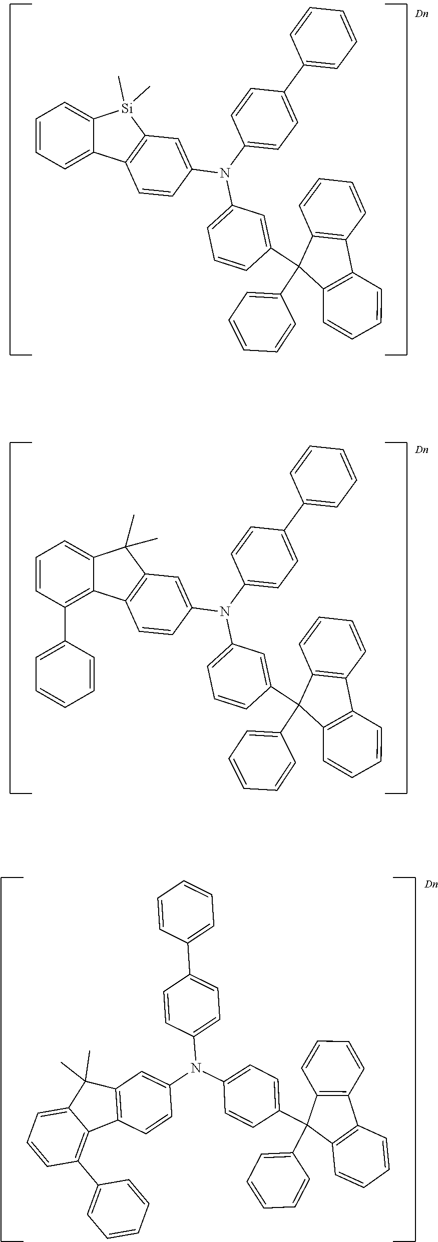

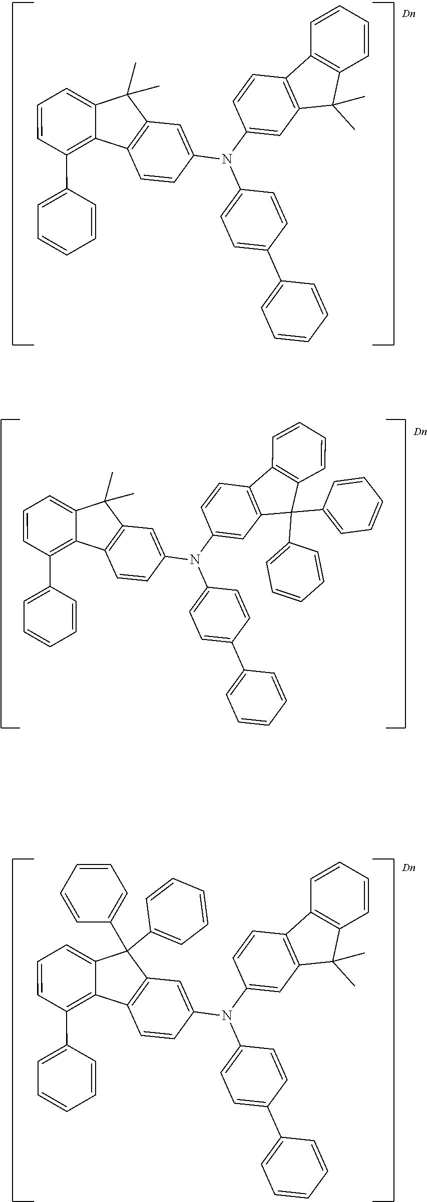

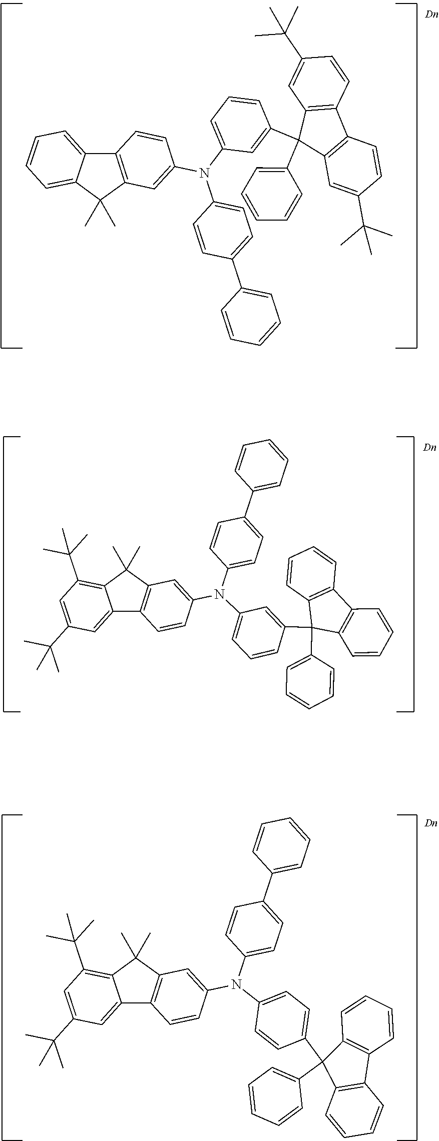

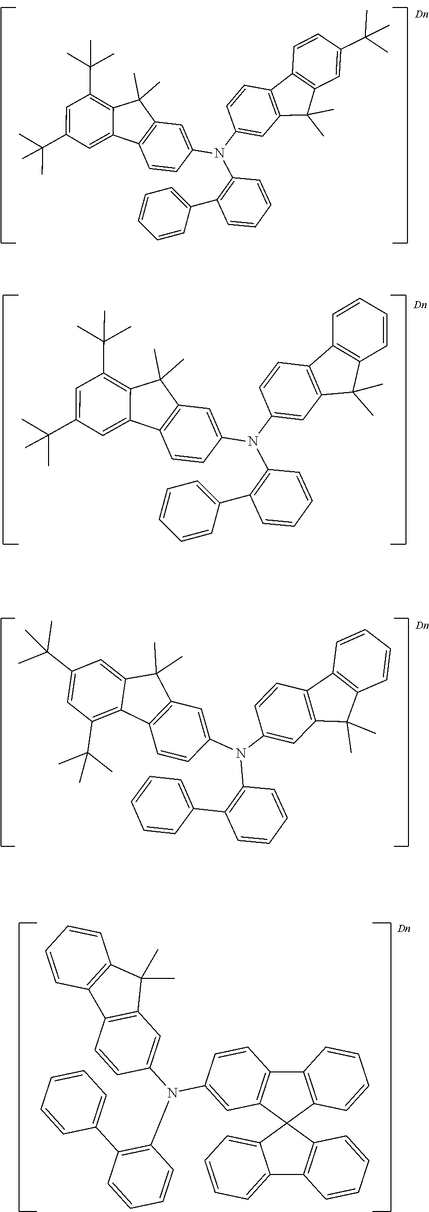

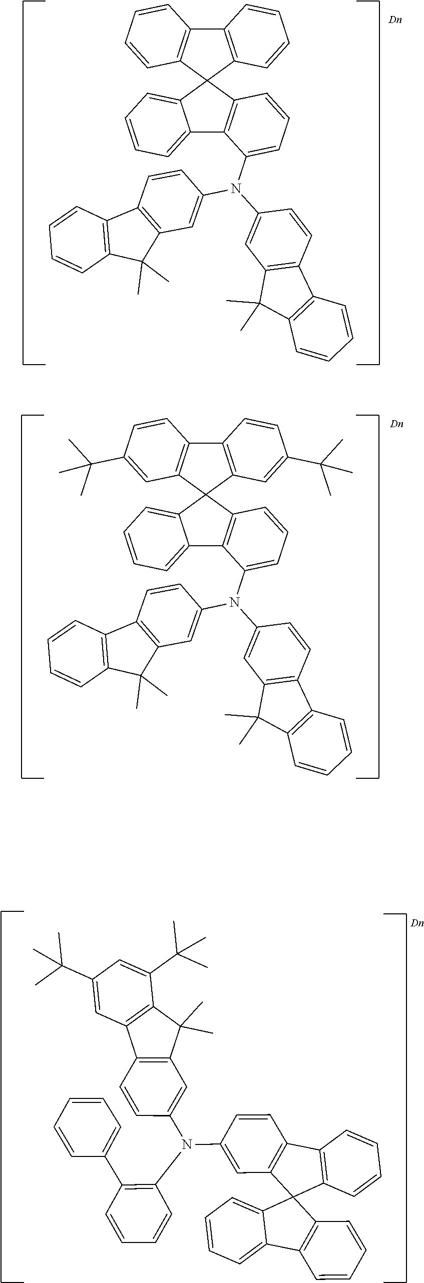

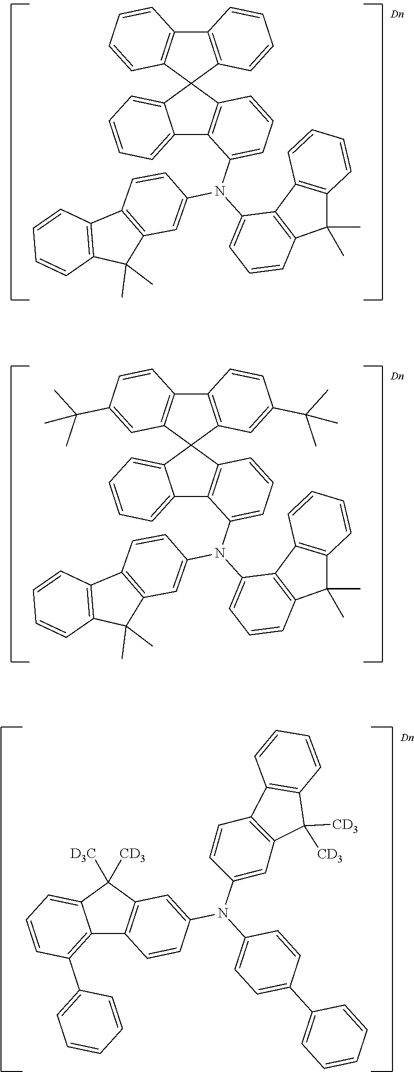

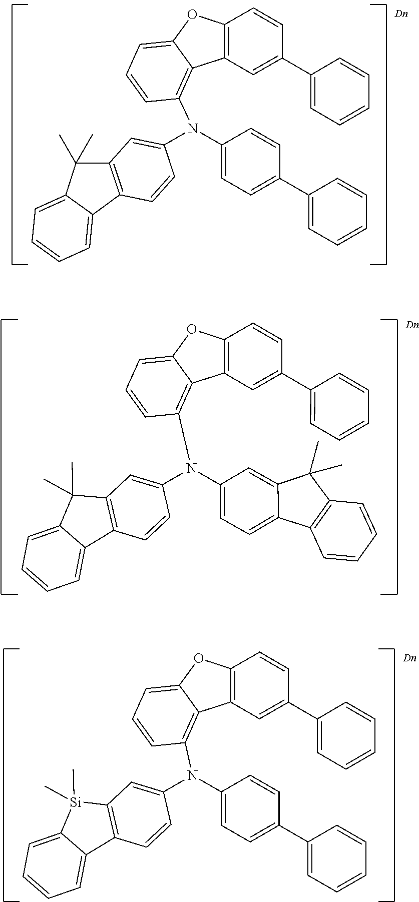

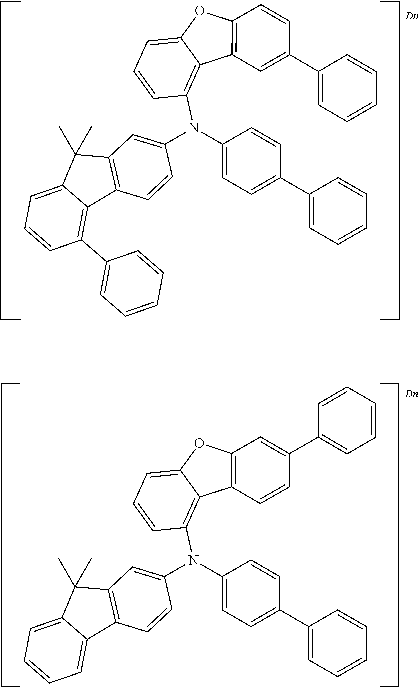

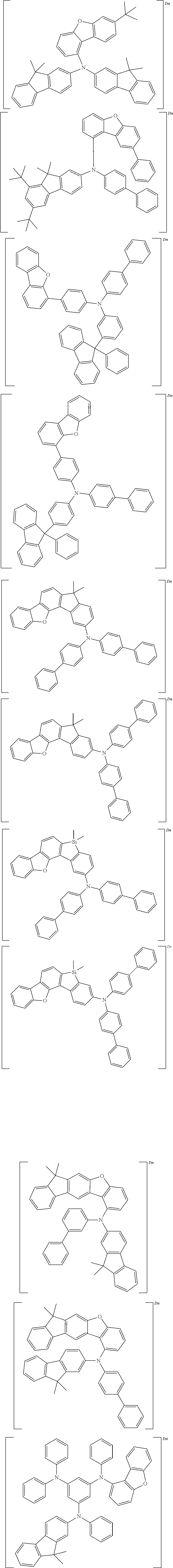

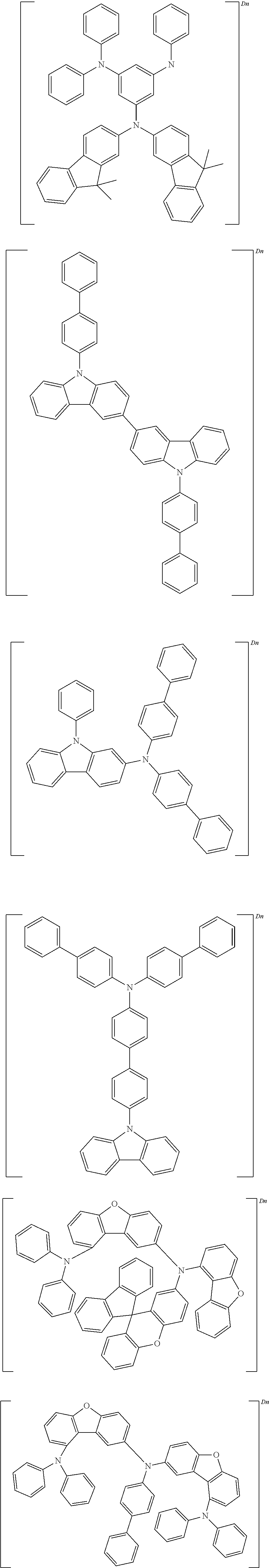

(Dn refers to the number of deuterium substitutions and indicates a structure substituted with one or more deuterium atoms)

In the hole transport region 140, in addition to the compounds described above, other suitable compounds having a similar structure may also be used.

In an implementation, the charge transport region may be, e.g., the electron transport region 150.

The electron transport region 150 may further increase electron injection or electron mobility and block holes between the cathode 110 and the light emitting layer 130.

In an implementation, the electron transport region 150 may include, e.g., an electron transport layer between the cathode 110 and the light emitting layer 130, and an electron transport auxiliary layer between the light emitting layer 130 and the electron transport layer, and a compound of Group C may be included in at least one of the electron transport layer and the electron transport auxiliary layer.

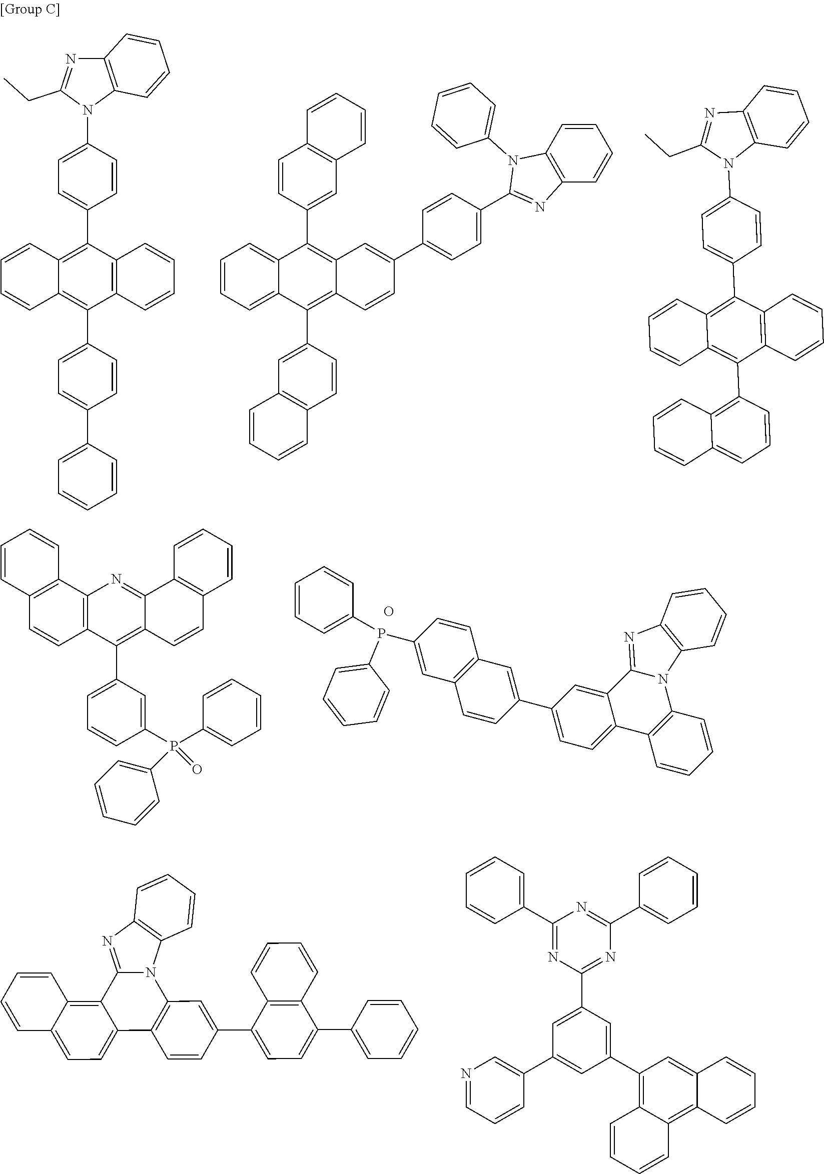

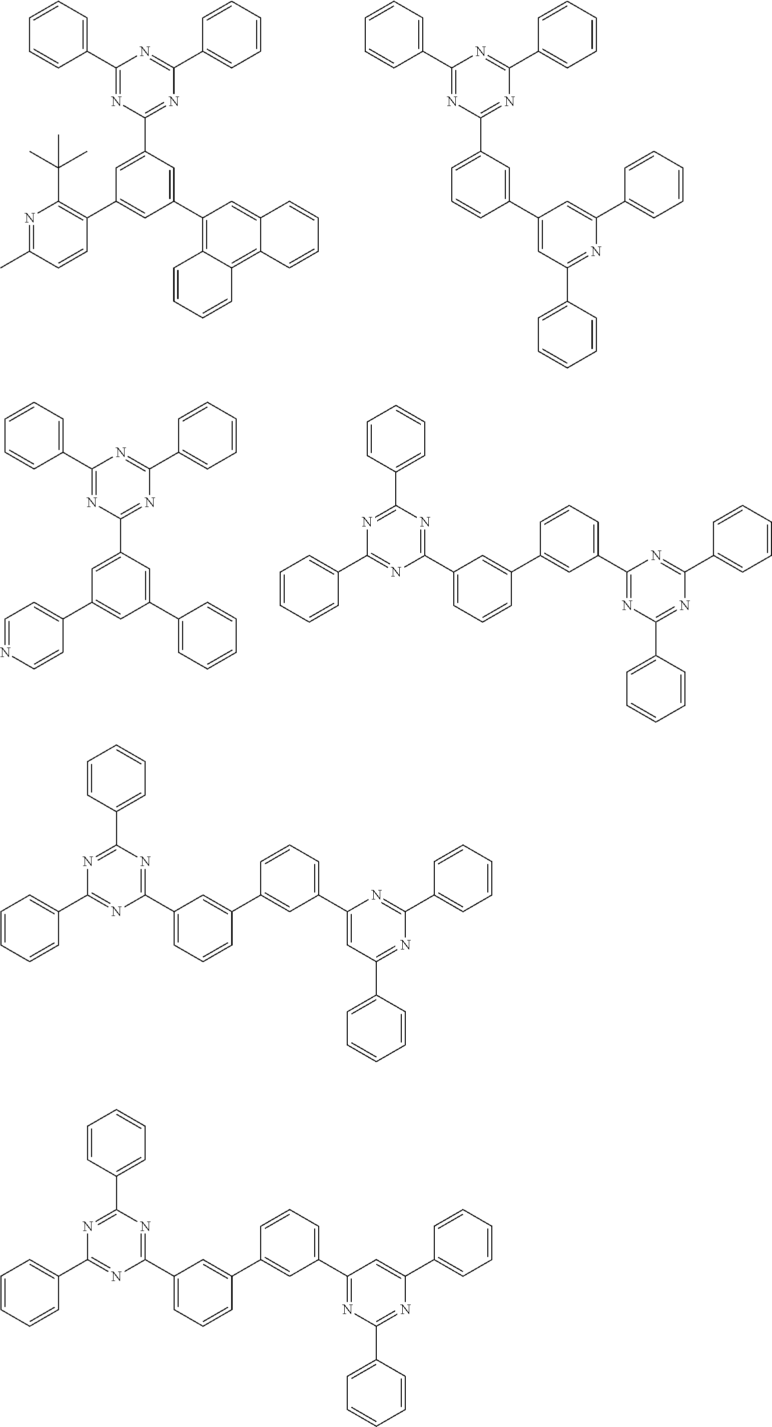

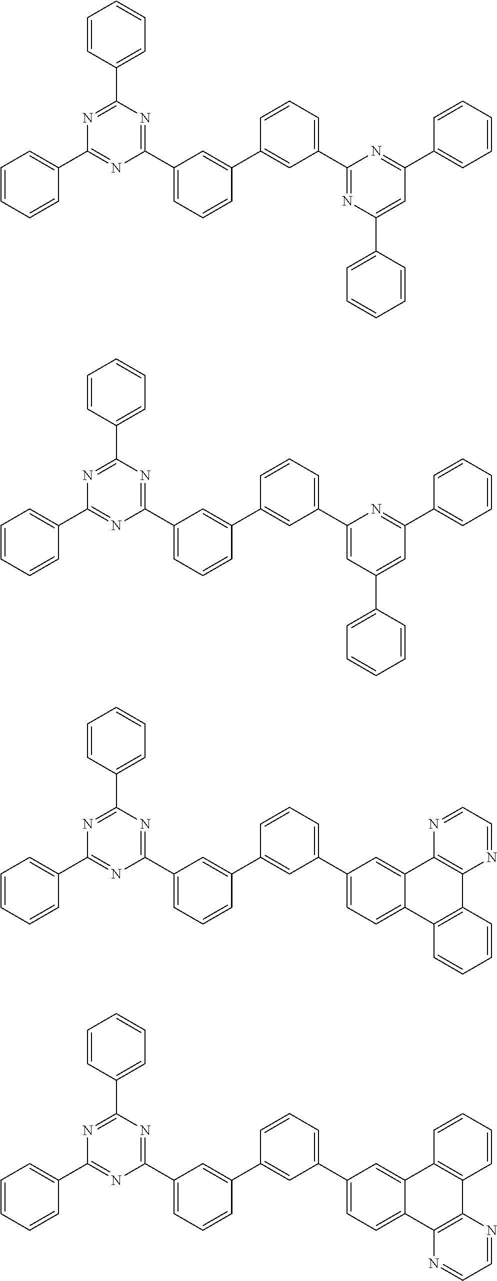 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

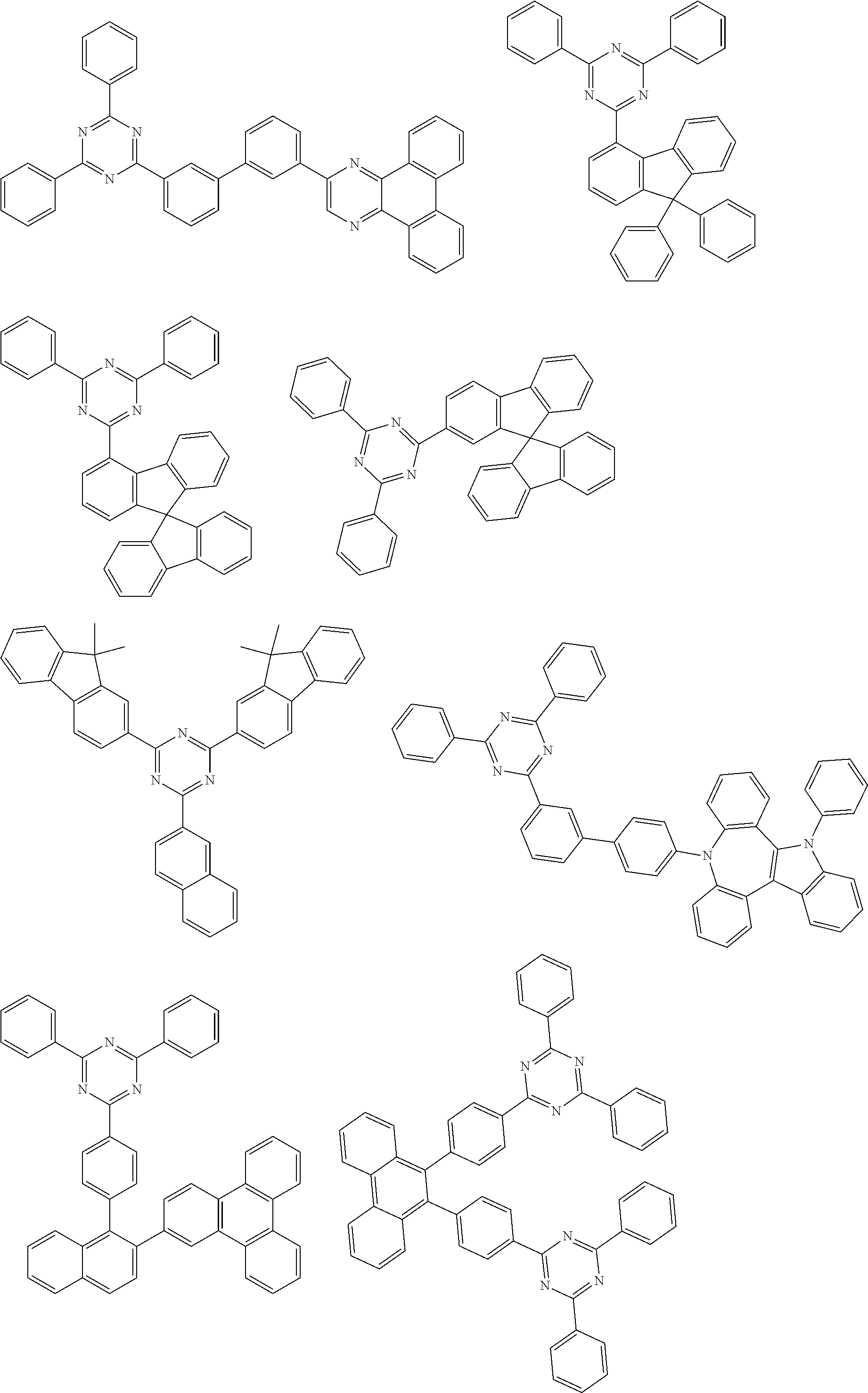

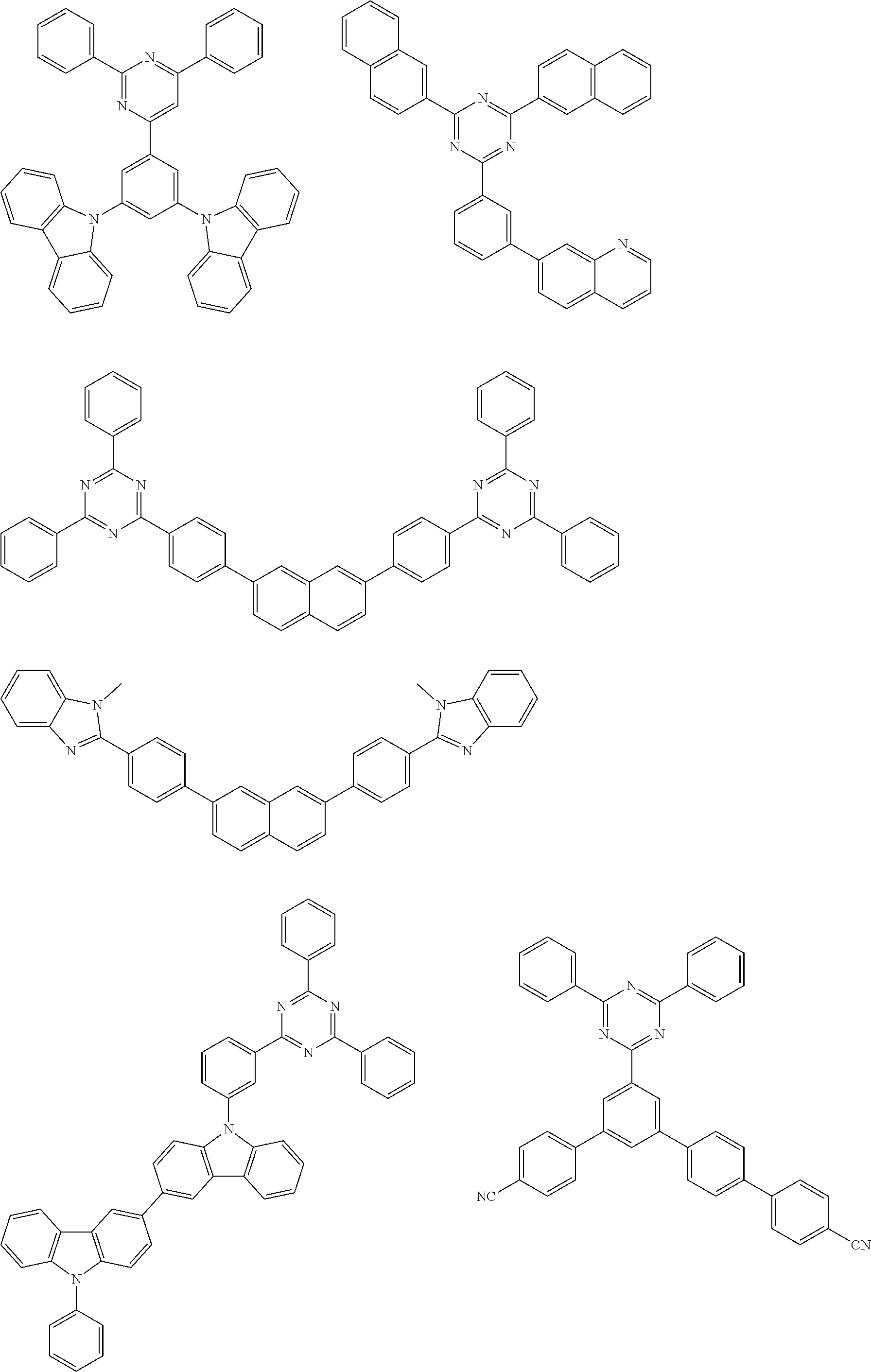

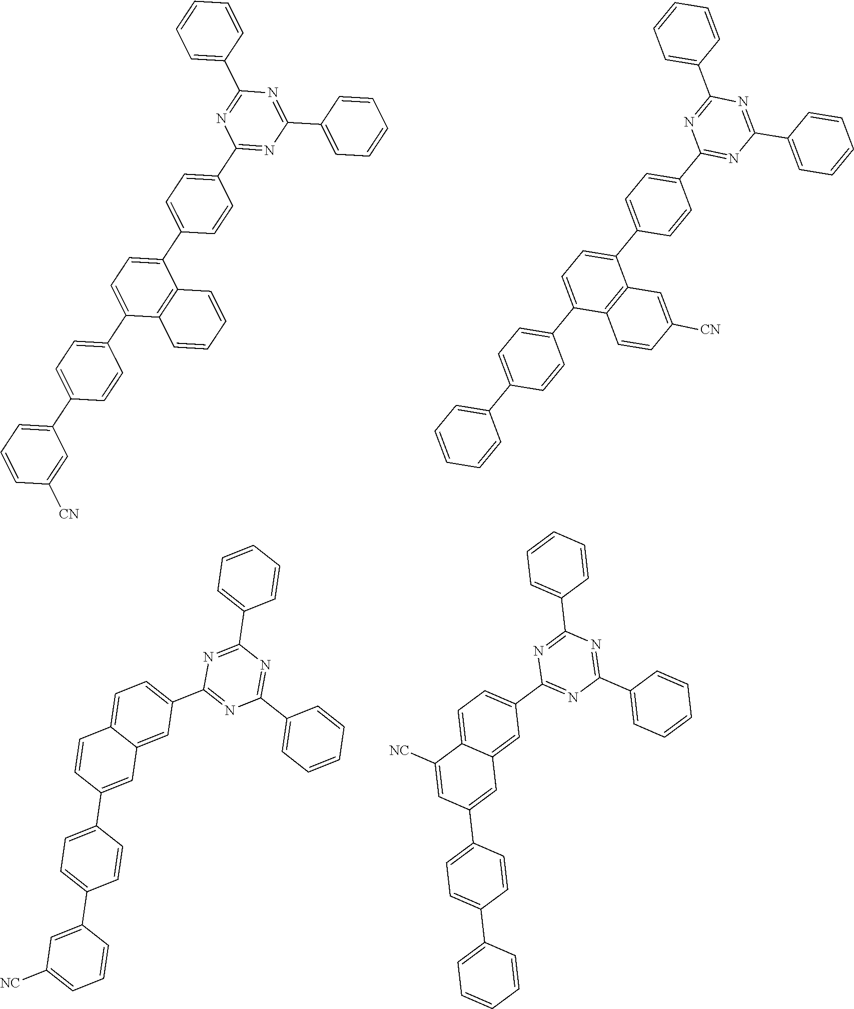 [SMILES: N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1 ; N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12]

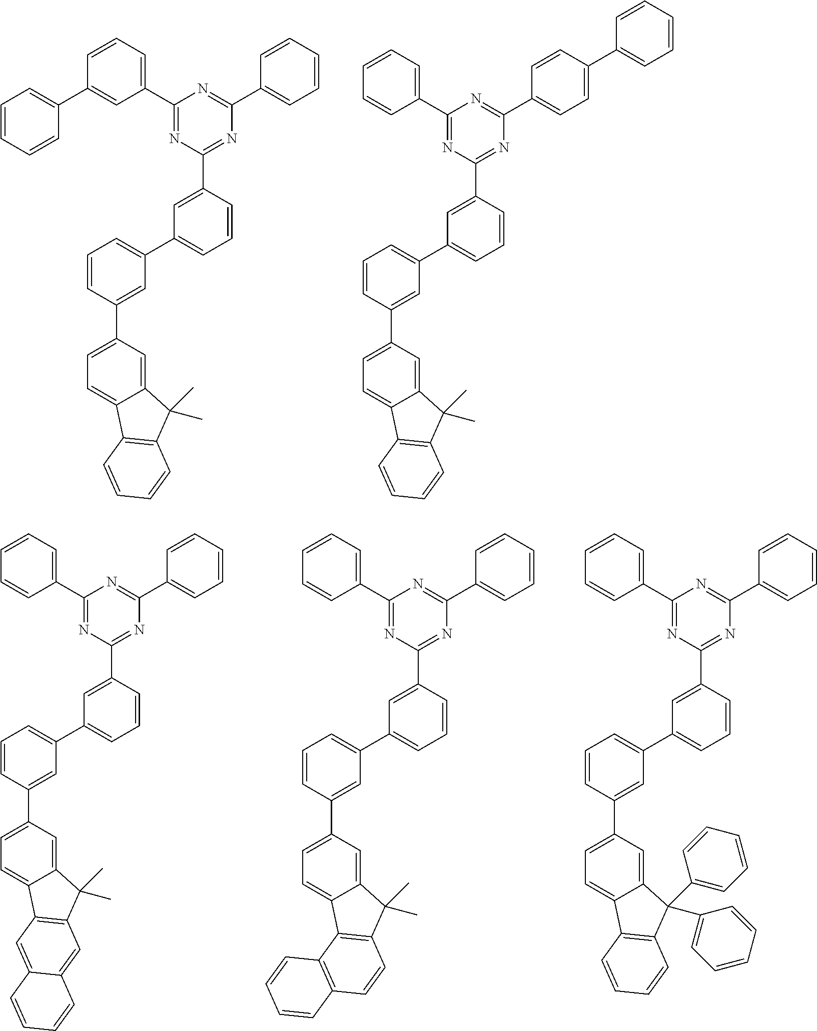

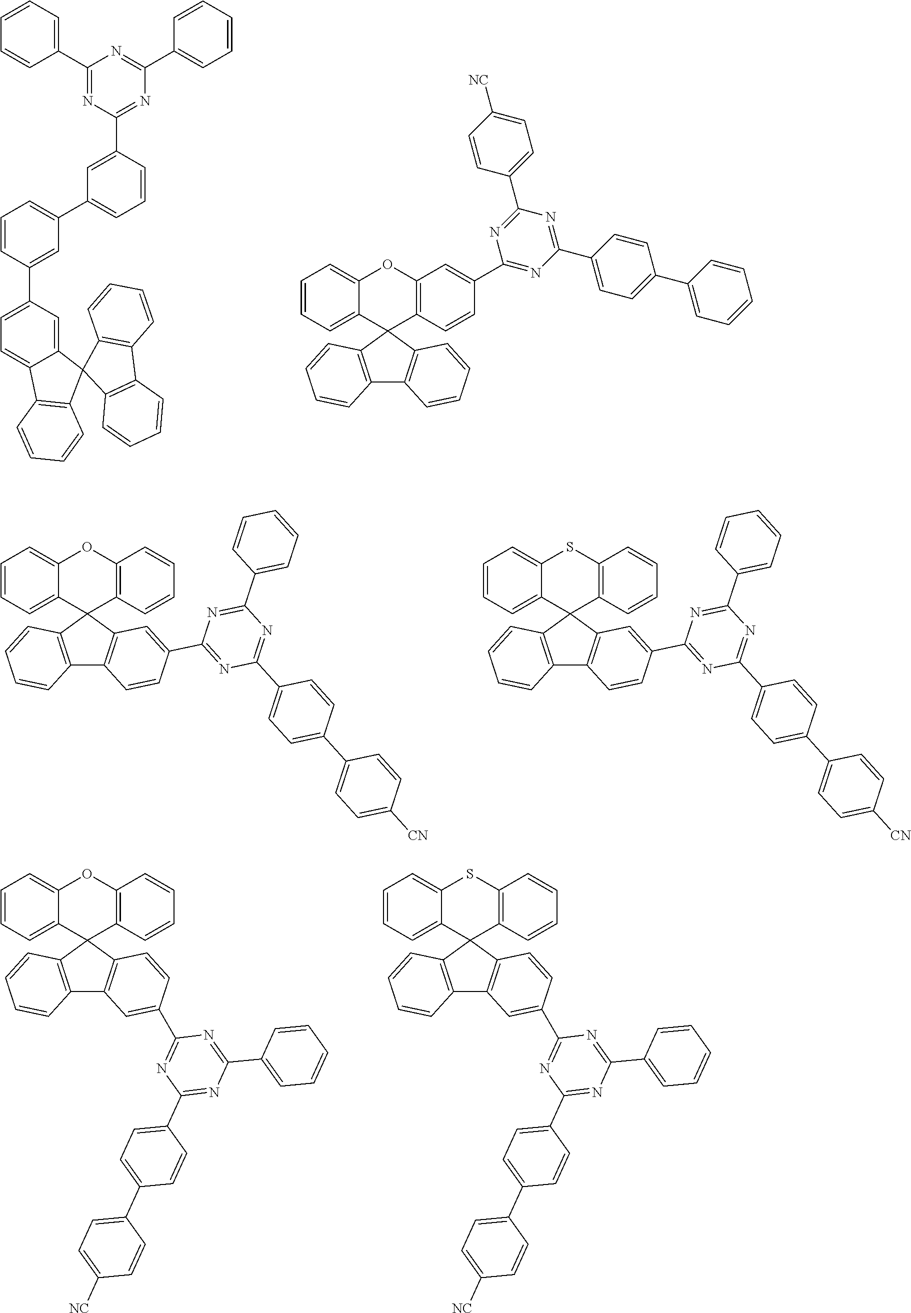

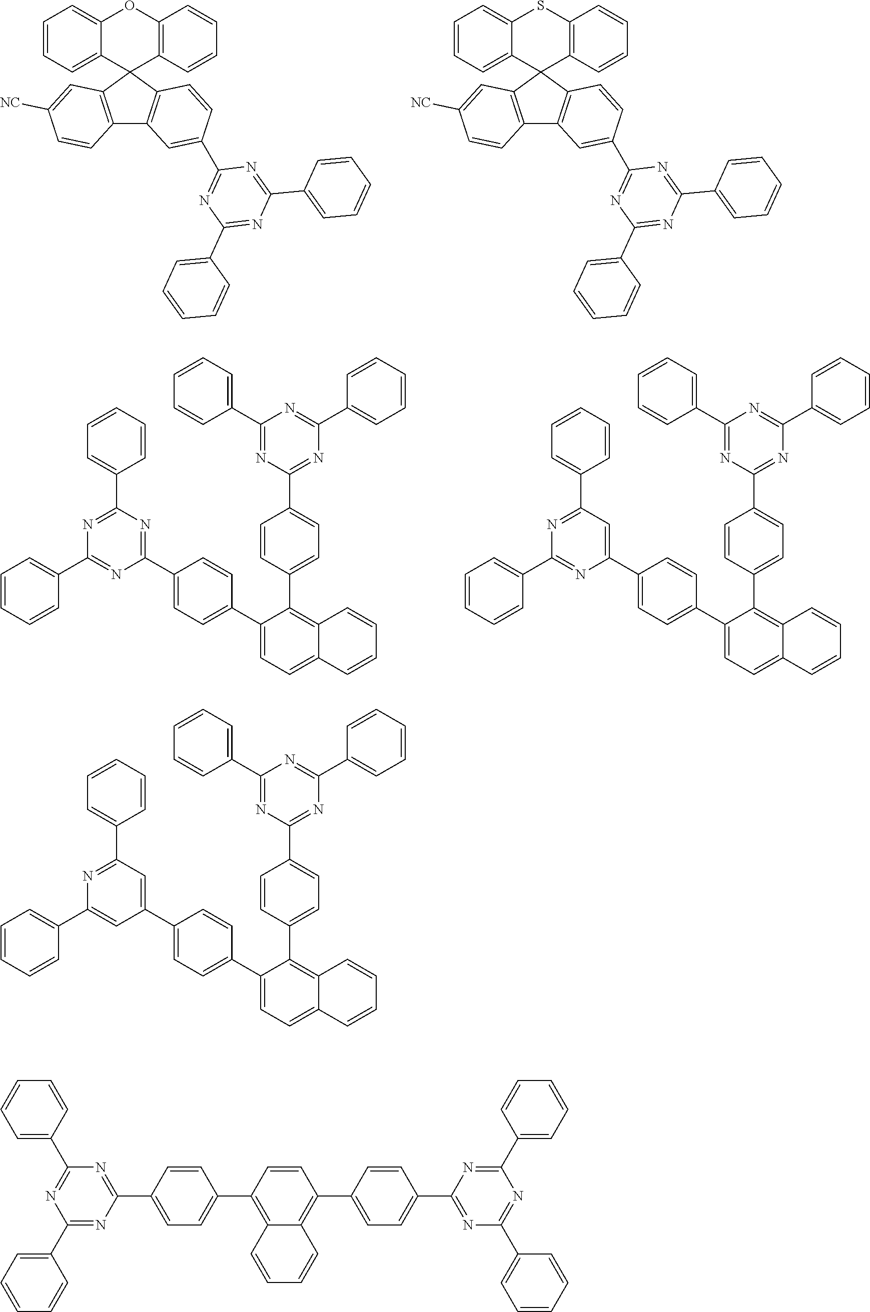

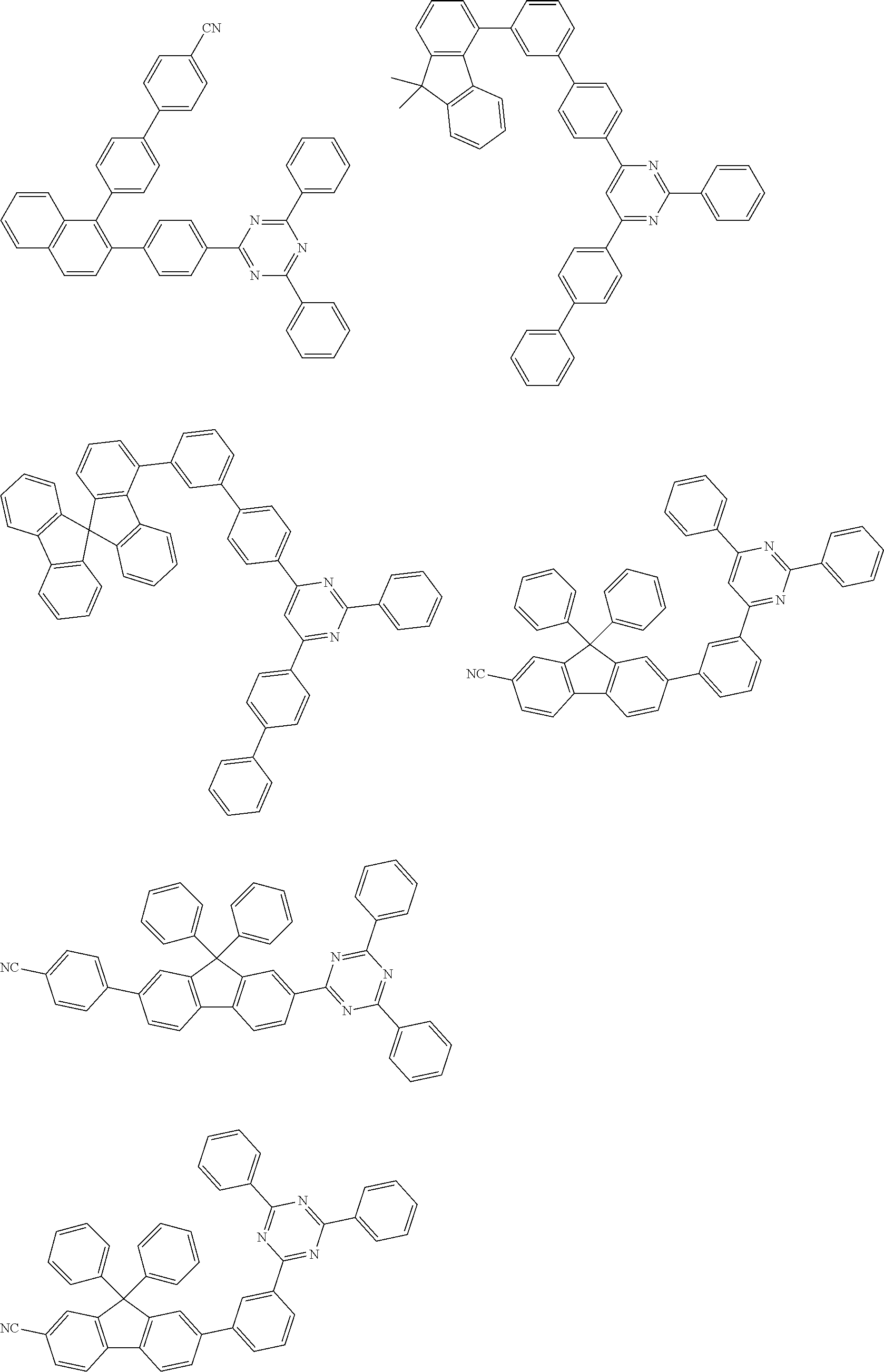

Some example embodiments may be an organic light emitting diode including the light emitting layer as the organic layer.

Some example embodiments may be an organic light emitting diode including a light emitting layer and a hole transport region as the organic layer.

Some example embodiments may be an organic light emitting diode including a light emitting layer and an electron transport region as the organic layer.

An organic light emitting diode according to an implementation includes a hole transport region 140 and an electron transport region 150 in addition to the light emitting layer 130 as the organic layer 105, as shown in the FIGURE.

In an implementation, an organic light emitting diode may further include an electron injection layer a hole injection layer, or the like, in addition to the light emitting layer as the organic layer.

The organic light emitting diodes 100 may be manufactured by forming an anode or a cathode on a substrate, and then forming an organic layer by a dry film method, e.g., vacuum deposition, sputtering, plasma plating, or ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by known methods.

### Preparation of Compound for Organic Optoelectronic Device

### Synthesis Example 1: Synthesis of Intermediate I-1

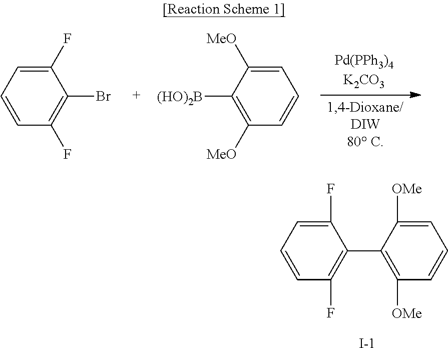 [SMILES: Fc1cccc(F)c1Br ; COc1cccc(OC)c1B(O)O ; COc1cccc(OC)c1-c1c(F)cccc1F ; [I-]]

In a nitrogen environment, 2-bromo-1,3-difluorobenzene (50 g, 259 mmol) and (2,6-dimethoxyphenyl) boronic acid (49.5 g, 272 mmol) were dissolved by adding 800 m1 of 1,4-dioxane thereto, and 250 ml of an aqueous solution prepared by dissolving potassium carbonate (89.5 g, 648 mmol) was added thereto and then stirred. Subsequently, tetrakis(triphenylphosphine)palladium (15 g, 13 mmol) was added thereto and then heated under reflux at 80° C. for 12 hours. When a reaction was completed, after adding water to the reaction solution, the mixture was extracted with dichloromethane, treated with magnesium sulfate anhydrous to remove moisture, filtered, and concentrated under a reduced pressure. The obtained residue therefrom was separated and purified through flash column chromatography to obtain Intermediate I-1 (30 g, 46%).

HRMS (70 eV, EI+): m/z calcd for C14H12F2O2: 250.0855, found: 250.

Elemental Analysis: C, 67%; H, 5%

### Synthesis Example 2: Synthesis of Intermediate I-2

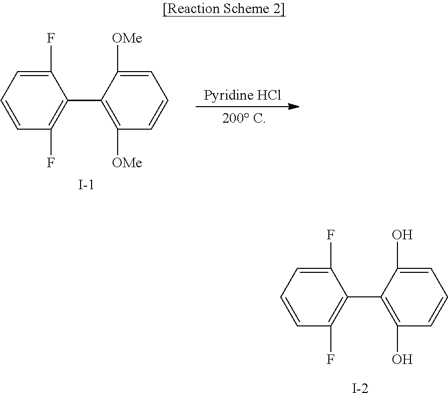

In a nitrogen environment, Intermediate I-1 (30 g, 120 mmol) and pyridine hydrochloride (70 g, 600 mmol) were added and then stirred under reflux by heating at 200° C. for 12 hours. When a reaction was completed, after adding water to the reaction solution, the mixture was extracted with dichloromethane, treated with magnesium sulfate anhydrous to remove moisture, filtered, and concentrated under a reduced pressure. The obtained residue was separated and purified through flash column chromatography to obtain Intermediate I-2 (20 g, 75%).

HRMS (70 eV, EI+): m/z calcd for C14H8F2O2: 222.0492, found: 222.

Elemental Analysis: C, 65%; H, 4%

### Synthesis Example 3: Synthesis of Intermediate I-3

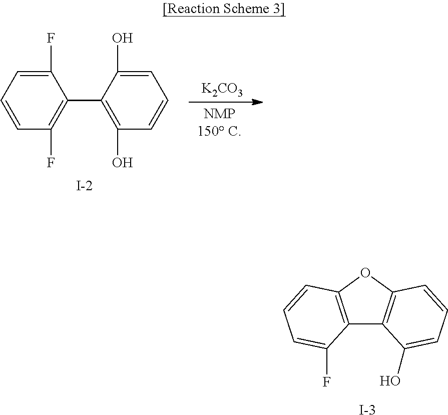

In a nitrogen environment, Intermediate I-2 (20 g, 90 mmol) and potassium carbonate (15 g, 108 mmol) were dissolved by adding 60 m1 of N-methyl-2-pyrrolidine thereto and then stirred under reflux by heating 150° C. for 12 hours. When a reaction was completed, after adding water to the reaction solution, the mixture was extracted with dichloromethane, treated with magnesium sulfate anhydrous to remove moisture, filtered, and concentrated under a reduced pressure. The obtained residue was separated and purified through flash column chromatography to obtain Intermediate I-3 (15 g, 82%).

HRMS (70 eV, EI+): m/z calcd for C12H7FO2: 202.0430, found: 202.

Elemental Analysis: C, 71%; H, 3%

### Synthesis Example 4: Synthesis of Intermediate I-4

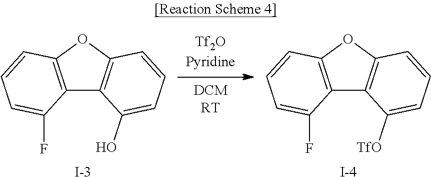

In a nitrogen environment, Intermediate I-3 (15 g, 74 mmol) and pyridine (15 ml, 89 mmol) were dissolved by adding 50 m1 of dichloromethane thereto and then stirred for 30 minutes. After decreasing the temperature to 0° C., trifluoromethanesulfonic anhydride (9 ml, 111 mmol) was slowly added thereto and then stirred for 12 hours. When a reaction was completed, after adding water to the reaction solution, the mixture was extracted with dichloromethane, treated with magnesium sulfate anhydrous to remove moisture, filtered, and concentrated under a reduced pressure. The obtained residue therefrom was separated and purified through flash column chromatography to obtain Intermediate I-4 (20 g, 81%).

HRMS (70 eV, EI+): m/z calcd for C13H6F4O4S: 333.9923, found: 333.

Elemental Analysis: C, 45%; H, 2%

### Synthesis Example 5: Synthesis of Intermediate I-5

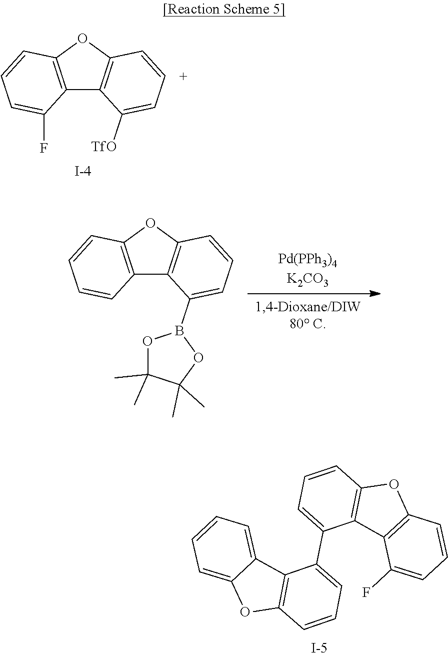

Intermediate I-5 (15 g, 71%) was obtained in the same manner as in Synthesis Example 1 except that Intermediate I-4 (20 g, 60 mmol) and 2-(dibenzo[b,d]furan-1-yl)-4,4,5,5-tetramethyl-1,3,2-dioxaborolane (21 g, 72 mmol) were used.

HRMS (70 eV, EI+): m/z calcd for C24H13FO2: 352.0900, found: 352.

Elemental Analysis: C, 82%; H, 4%

### Synthesis Example 6: Synthesis of Compound 1

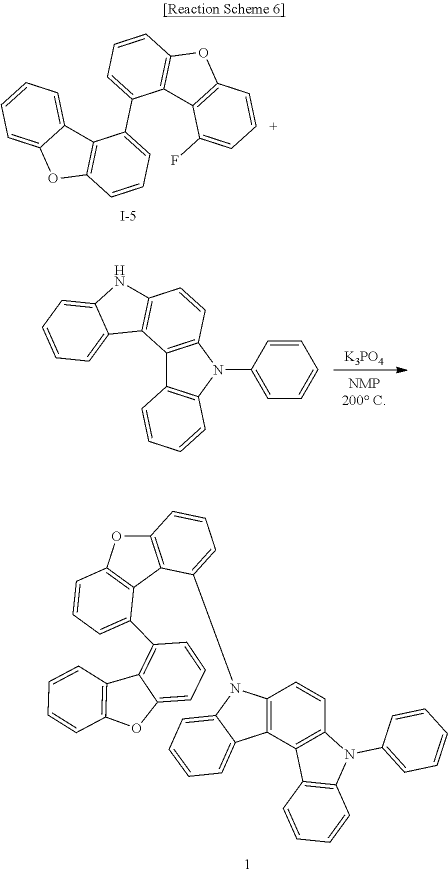

In a nitrogen environment, Intermediate I-5 (15 g, 57 mmol), 5-phenyl-5,8-dihydroindolo[2,3-c]carbazole (28 g, 85 mmol), and potassium phosphate (48 g, 227 mmol) were dissolved by slowly adding 180 m1 of N-methyl-2-pyrrolidine and then stirred under reflux by heating at 200° C. for 12 hours. When a reaction was completed, after adding water to the reaction solution, the mixture was extracted with dichloromethane, treated with magnesium sulfate anhydrous to remove moisture, filtered, and concentrated under a reduced pressure

The obtained residue therefrom was separated and purified through flash column chromatography to obtain Compound 1 (20 g, 53%).

HRMS (70 eV, EI+): m/z calcd for C48H28N2O2: 664.2151, found: 664.

Elemental Analysis: C, 87%; H, 4%

### Synthesis Example 7: Synthesis of Intermediate I-6

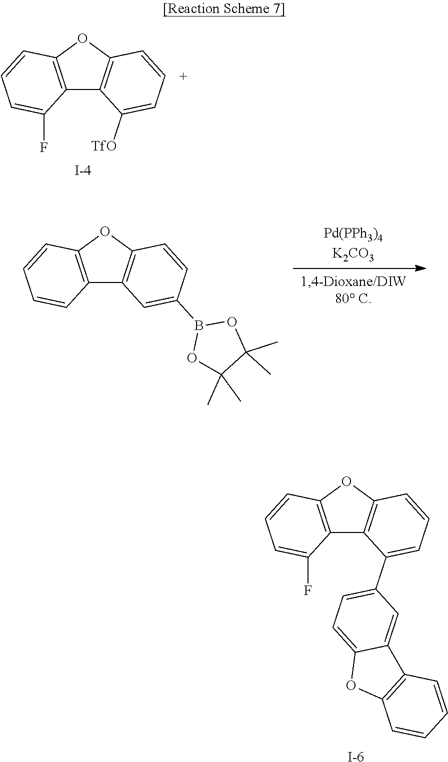

Intermediate I-6 (15 g, 71%) was obtained in the same manner as in Synthesis Example 1 except that Intermediate I-4 (20 g, 60 mmol) and 2-(dibenzo[b,d]furan-2-yl)-4,4,5,5-tetramethyl-1,3,2-dioxaborolane (21 g, 72 mmol) were used.

HRMS (70 eV, EI+): m/z calcd for C24H13FO2: 352.0900, found: 352.

Elemental Analysis: C, 82%; H, 4%

### Synthesis Example 8: Synthesis of Compound 36

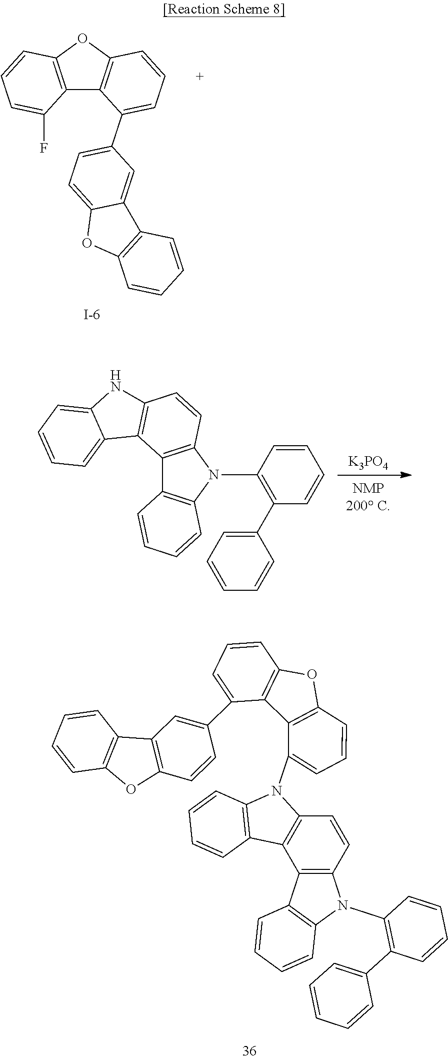

Compound 36 (20 g, 63%) was obtained in the same manner as in Synthesis Example 6 except that Intermediate I-6 (15 g, 42 mmol), 5-([1,1′-biphenyl]-2-yl)-5,8-dihydroindolo[2,3-c]carbazole (21 g, 64 mmol), and potassium phosphate (36 g, 170 mmol) were used.

HRMS (70 eV, EI+): m/z calcd for C48H28N2O2: 664.2151, found: 664.

Elemental Analysis: C, 87%; H, 4%

### Synthesis Example 9: Synthesis of Intermediate I-7

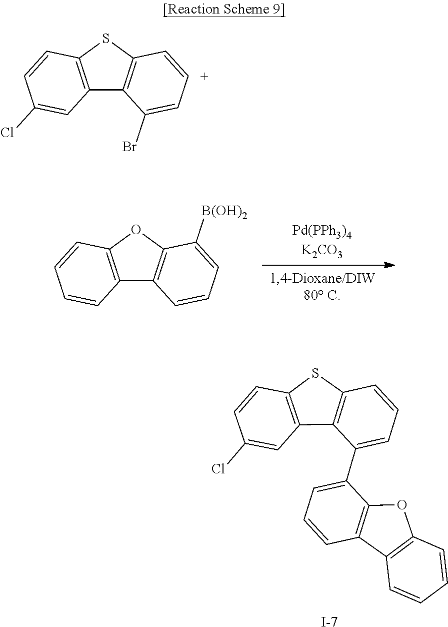

Intermediate I-7 (20 g, 77%) was obtained in the same manner as in Synthesis Example 1 except that 1-bromo-8-chlorodibenzo[b,d]thiophene (20 g, 67 mmol) and dibenzo[b,d]furan-4-ylboronic acid (15 g, 70 mmol) were used.

HRMS (70 eV, EI+): m/z calcd for C24H13ClOS: 384.0376, found: 384.

Elemental Analysis: C, 75%; H, 3%

### Synthesis Example 10: Synthesis of Compound 52

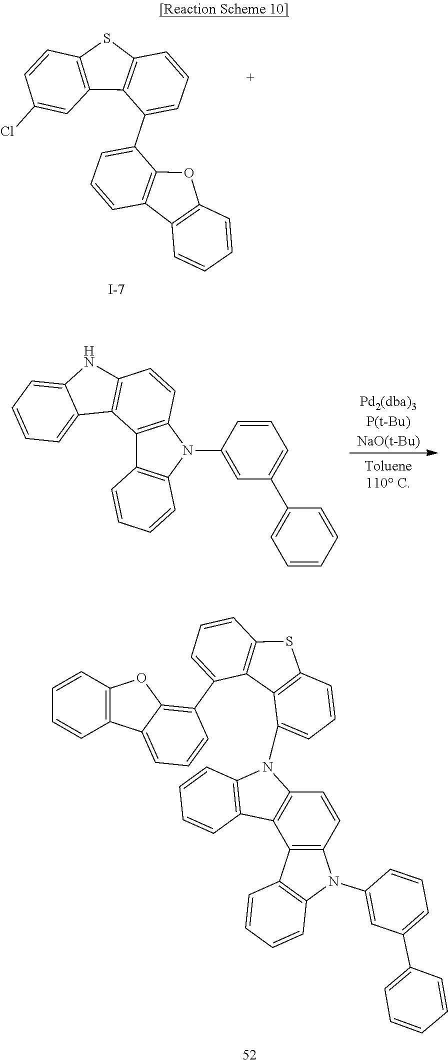

In a nitrogen environment, Intermediate I-7 (20 g, 52 mmol) was dissolved in 500 m1 of toluene, and 5-([1,1′-biphenyl]-3-yl)-5,8-dihydroindolo[2,3-c]carbazole (25 g, 62 mmol), tris(dibenzylideneacetone) dipalladium (1.4 g, 1.5 mmol), tris-tert butylphosphine (3.8 ml, 7.8 mmol), and sodium tert-butoxide (6 g, 62 mmol) were added thereto and then refluxed by heating at 100° C. for 12 hours. When a reaction was completed, after adding water to the reaction solution to extract an organic layer therefrom, the organic layer was treated with magnesium sulfate anhydrous to remove moisture, filtered, and concentrated under a reduced pressure. The obtained residue was separated and purified through flash column chromatography to obtain Compound 52 (20 g, 51%).

HRMS (70 eV, EI+): m/z calcd for C54H32N2OS: 756.2235, found: 756.

Elemental Analysis: C, 86%; H, 4%

### Synthesis Example 11: Synthesis of Intermediate I-8

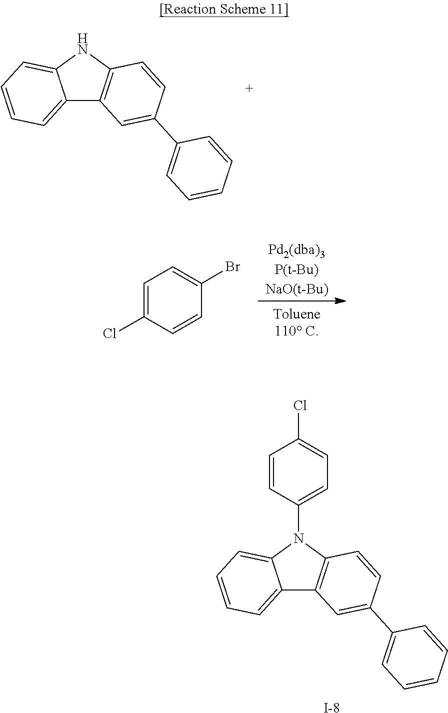

Intermediate I-8 (25 g, 86%) was obtained in the same manner as in Synthesis Example 10 except that 3-phenyl-9H-carbazole (20 g, 82 mmol) and 1-bromo-4-chlorobenzene (16 g, 86 mmol) were used.

HRMS (70 eV, EI+): m/z calcd for C24H16ClN: 353.0971, found: 353.

Elemental Analysis: C, 81%; H, 5%

### Synthesis Example 12: Synthesis of Intermediate I-9

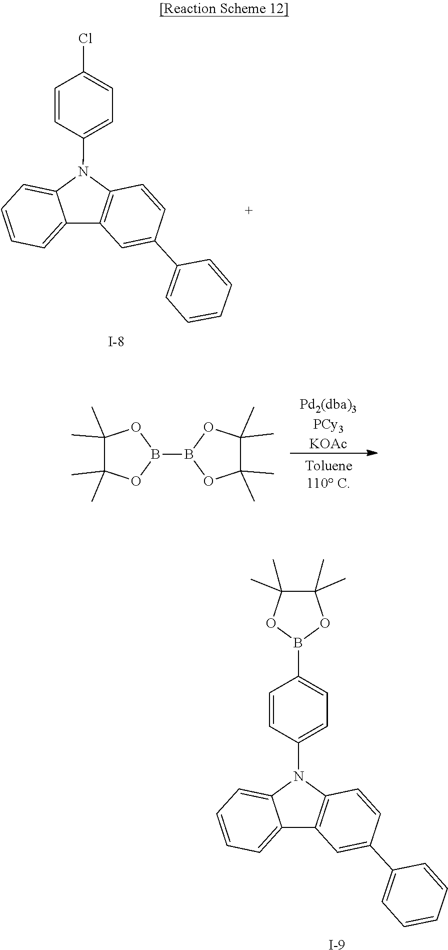

In a nitrogen environment, Intermediate I-8 (25 g, 70 mmol) was dissolved in 700 ml of toluene, and 4,4,4′,4′,5,5,5′,5′-octamethyl-2,2′-bi (1,3,2-dioxaborolane) (22 g, 85 mmol), tris(dibenzylideneacetone) dipalladium (1.9 g, 2.1 mmol), tricyclohexyl phosphine (4.76 g, 17 mmol), and potassium acetate (17 g, 177 mmol) were added thereto and then refluxed by heating 110° C. for 12 hours. When a reaction was completed, after adding water to the reaction solution, the mixture was extracted with dichloromethane, treated with magnesium sulfate anhydrous to remove moisture, filtered, and concentrated under a reduced pressure. The obtained residue therefrom was separated and purified through flash column chromatography to obtain Intermediate I-9 (20 g, 64%).

HRMS (70 eV, EI+): m/z calcd for C30H8BNO2: 445.2213, found: 445.

Elemental Analysis: C, 81%; H, 6%

### Synthesis Example 13: Synthesis of Compound A-30

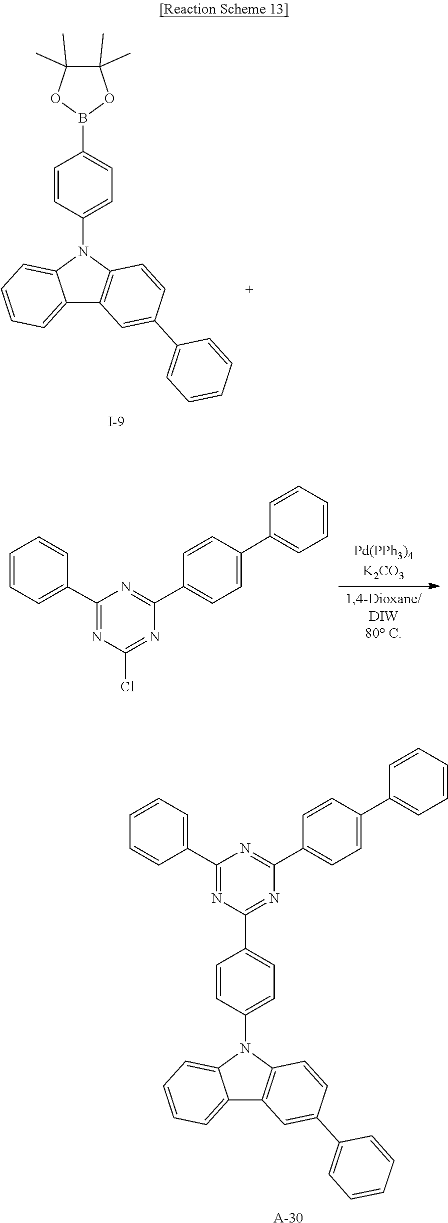

Compound A-30 (20 g, 71%) was obtained in the same manner as in Synthesis Example 1 except that Intermediate I-9 (20 g, 45 mmol) and 2-([1,1′-biphenyl]-4-yl)-4-chloro-6-phenyl-1,3,5-triazine (18.5 g, 54 mmol) were used.

HRMS (70 eV, EI+): m/z calcd for C45H30N4: 626.2470, found: 626.

Elemental Analysis: C, 86%; H, 5%

### Synthesis of Comparative Compound

### Synthesis Example 14: Synthesis of Intermediate I-10

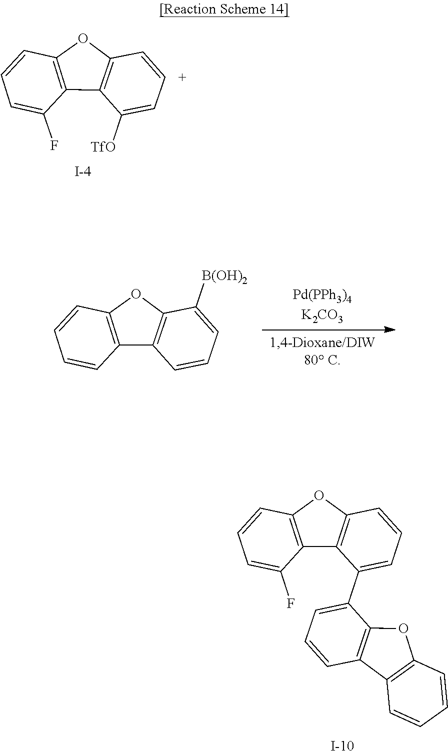

Intermediate I-10 (20 g, 95%) was obtained in the same manner as in Synthesis Example 1 except that Intermediate I-4 (20 g, 60 mmol) and dibenzo[b,d]furan-4-ylboronic acid (15 g, 72 mmol) were used.

HRMS (70 eV, EI+): m/z calcd for C24H13FO2: 352.0900, found: 352.

Elemental Analysis: C, 82%; H, 4%

### Synthesis Example 15: Synthesis of Compound R-1

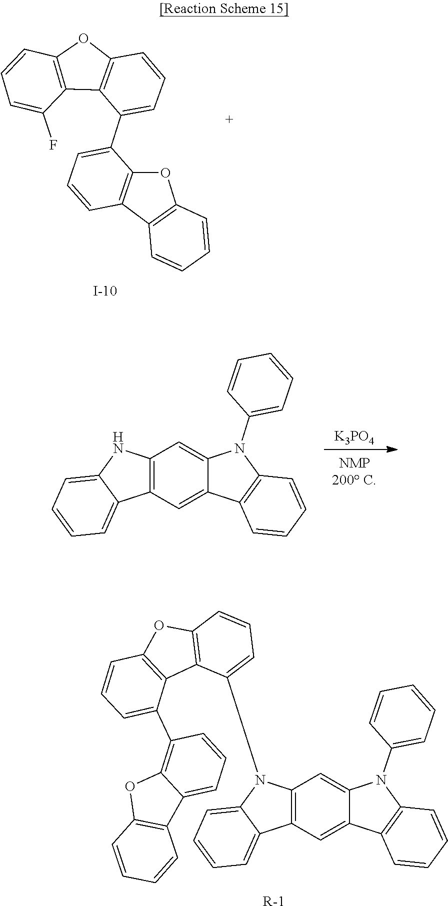

Compound R-1 (20 g, 63%) was obtained in the same manner as in Synthesis Example 6 except that Intermediate I-10 (20 g, 57 mmol), 5-phenyl-5,7-dihydroindolo[2,3-b]carbazole (28 g, 85 mmol), and potassium phosphate (48 g, 227 mmol) were used.

HRMS (70 eV, EI+): m/z calcd for C48H28N2O2: 664.2151, found: 664.

Elemental Analysis: C, 87%; H, 4%

### Synthesis Example 16: Synthesis of Intermediate I-11

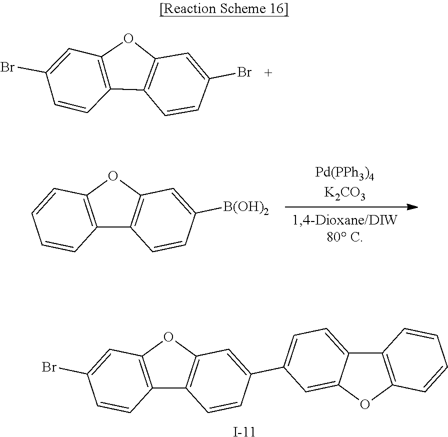

Intermediate I-11 (20 g, 79%) was obtained in the same manner as in Synthesis Example 1 except that 3,7-dibromodibenzo[b,d]furan (20 g, 61 mmol) and dibenzo[b,d]furan-3-yl-boronic acid (14 g, 64 mmol) were used.

HRMS (70 eV, EI+): m/z calcd for C24H13BrO2: 412.0099, found: 412.

Elemental Analysis: C, 70%; H, 3%

### Synthesis Example 17: Synthesis of Comparative Compound R-2

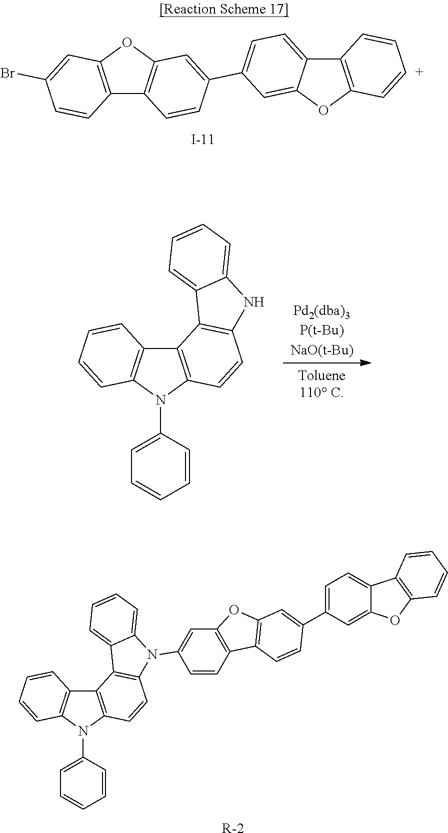

Compound R-2 (30 g, 80%) was obtained in the same manner as in Synthesis Example 10 except that Intermediate I-11 (20 g, 57 mmol) and 5-phenyl-5,8-dihydroindolo[2,3-c]carbazole (28 g, 86 mmol) were used.

HRMS (70 eV, EI+): m/z calcd for C48H28N2O2: 664.2151, found: 664.

Elemental Analysis: C, 87%; H, 4%

### Synthesis Example 18: Synthesis of Comparative Compound R-3

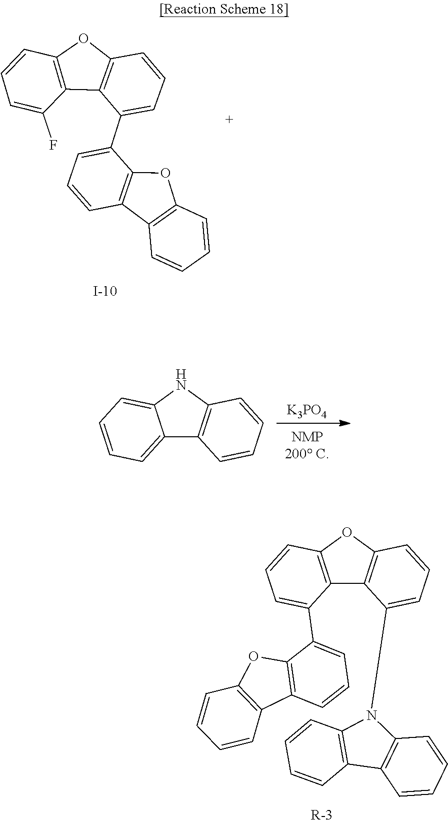

Compound R-3 (20 g, 71%) was obtained in the same manner as in Synthesis Example 6 except that Intermediate I-10 (20 g, 57 mmol), 9H-carbazole (14 g, 85 mmol), and potassium phosphate (48 g, 227 mmol) were used.

HRMS (70 eV, EI+): m/z calcd for C36H21NO2: 499.1572, found: 499.

Elemental Analysis: C, 87%; H, 4%

### Example 1

A glass substrate coated with a thin film of ITO (indium tin oxide) was ultrasonically cleaned with distilled water. After washing with the distilled water, the glass substrate was ultrasonically washed with isopropyl alcohol, acetone, or methanol, and dried and then moved to a plasma cleaner, cleaned by using oxygen plasma for 10 minutes, and moved to a vacuum depositor. This prepared ITO transparent electrode was used as an anode, and Compound A doped with 3% NDP-9 (commercially available from Novaled) was vacuum-deposited on the ITO substrate to form a 100 Å-thick hole injection layer, and a 1,350 Å-thick hole transport layer was formed thereon by depositing Compound A. Compound B was deposited on the hole transport layer to a thickness of 350 Å to form a hole transport auxiliary layer, and Compound 1 synthesized in Synthesis Example 6 was used as a host on the hole transport auxiliary layer and PhGD was doped as a dopant at 7 wt % to form a 400 Å-thick light emitting layer by vacuum deposition. The ratios are described separately for Examples and Comparative Examples. Subsequently, Compound C was deposited on the light emitting layer to a thickness of 50 Å to form an electron transport auxiliary layer, and Compound D and LiQ were simultaneously vacuum-deposited at a weight ratio of 1:1 to form a 300 Å-thick electron transport layer. An organic light emitting diode was manufactured by sequentially vacuum-depositing 15 Å of LiQ and 1,200 Å of Al on the electron transport layer to form a cathode.

The organic light emitting diode was manufactured with the structure of ITO/Compound A (3% NDP-9 doping, 100 Å)/Compound A (1,350 Å)/Compound B (350 Å)/EML [Compound 1 (93 wt %):PhGD (7 wt %)] (400 Å)/Compound C (50 Å)/Compound D: LiQ (300 Å)/LiQ (15 Å)/Al (1,200 Å).
Compound A: N-(biphenyl-4-yl)-9,9-dimethyl-N-(4-(9-phenyl-9H-carbazol-3-yl)phenyl)-9H-fluoren-2-amine
Compound B: N-[4-(4-Dibenzofuranyl)phenyl]-N-[4-(9-phenyl-9H-fluoren-9-yl)phenyl][1,1′-biphenyl]-4-amine
Compound C: 2,4-Diphenyl-6-(4′,5′,6′-triphenyl[1,1′: 2′,1″: 3″,1′″:3′″,1′″-quinquephenyl]-3′″-yl)-1,3,5-triazine
compound D: 2-(1,1′-Biphenyl-4-yl)-4-(9,9-diphenylfluoren-4-yl)-6-phenyl-1,3,5-triazine

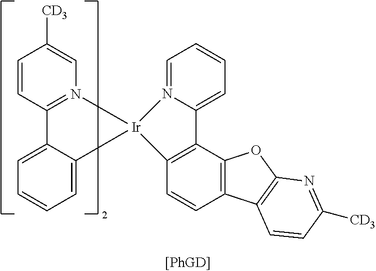

### Examples 2 and 3 and Comparative Examples 1 to 3

Each organic light emitting diode was manufactured in the same manner as in Example 1 except that the composition was changed as described in Table 1.

### Example 4

A glass substrate coated with a thin film of ITO (indium tin oxide) was ultrasonically cleaned with distilled water. After washing with the distilled water, the glass substrate was ultrasonically washed with isopropyl alcohol, acetone, or methanol, and dried and then moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to form a 1,350 Å-thick hole transport layer. Compound E was deposited on the hole transport layer to a thickness of 350 Å to form a hole transport auxiliary layer. Compound 1 synthesized in Synthesis Example 6 and Compound A-30 synthesized in Synthesis Example 13 were simultaneously used as hosts on the hole transport auxiliary layer, and PhGD was doped at 10 wt % as a dopant to form a 400 Å-thick light emitting layer by vacuum deposition. Here, Compound 1 and Compound A-30 were used in a weight ratio of 6:4. Subsequently, Compound F was deposited on the light emitting layer to form a 50 Å-thick electron transport auxiliary layer, and compound G and LiQ were simultaneously vacuum deposited at a weight ratio of 1:1 to form a 300 Å-thick electron transport layer. On the electron transport layer, a cathode was formed by sequentially vacuum-depositing 15 Å of LiQ and 1,200 Å of Al, manufacturing an organic light emitting diode.

The organic light emitting diode was manufactured to have a structure of ITO/Compound A (3% NDP-9 doping, 100 Å)/Compound A (1,350 Å)/Compound E (350 Å)/EML [Compound 1: Compound A-30:PhGD=54:36:10 wt %)] (400 Å)/Compound F (50 Å)/Compound G:LiQ (300 Å)/LiQ (15 Å)/Al (1,200 Å).
Compound E: N,N-bis(9,9-dimethyl-9H-fluoren-4-yl)-9,9-spirobi (fluorene)-2-amine
Compound F: 2-[3′-(9,9-Dimethyl-9H-fluoren-2-yl) [1,1′-biphenyl]-3-yl]-4,6-diphenyl-1,3,5-triazine
Compound G: 2-[4-[4-(4′-Cyano-1,1′-biphenyl-4-yl)-1-naphthyl]phenyl]-4,6-diphenyl-1,3,5-triazine

### Examples 5 and 6 and Comparative Examples 4 to 6

Each organic light emitting diode was manufactured in the same manner as in Example 4 except that the composition was changed as described in Table 2.

### Examples 7 to 9

Organic light emitting diodes according to Examples 7 to 9 were manufactured in the same manner as in Examples 4 to 6, except that the mixing ratio of the host was changed from 6:4 to 7:3.

### Evaluation

The driving voltage, luminous efficiency, and life-span characteristics of the organic light emitting diodes according to Examples 1 to 9 and Comparative Examples 1 to 6 were evaluated.

The specific measurement method is as follows, and the results are as shown in Tables 1 and 2.

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Luminous efficiency (cd/A) at the same current density (10 mA/cm²) was calculated by using the luminance and current density from (1) and (2) above and voltage.

The luminous efficiency values of Example 1 to 3 and Comparative Examples 1 to 3 were calculated as relative values based on Comparative Example 1 and are listed in Table 1.

The luminous efficiency values of Examples 4 to 9 and Comparative Examples 4 to 6 were calculated as relative values based on Comparative Example 4 and are listed in Table 2.

### (4) Measurement of Life-Span

The results were obtained by maintaining the luminance (cd/m²) at 24,000 cd/m² and measuring the time for the current efficiency (cd/A) to decrease to 95%.

The life-span measurement values of Examples 1 to 3 and Comparative Examples 1 to 3 were calculated as relative values based on Comparative Example 1 and are listed in Table 1.

The life-span measurement values of Examples 4 to 9 and Comparative Examples 4 to 6 were calculated as relative values based on Comparative Example 4 and are listed in Table 2.

### (5) Measurement of Driving Voltage

The results were obtained by measuring the driving voltage of each device at 15 mA/cm² using a current-voltage meter (Keithley 2400).

The driving voltages of Examples 1 to 3 and Comparative Examples 1 to 3 were calculated as relative values based on Comparative Example 1 and are listed in Table 1.

The driving voltages of Examples 4 to 9 and Comparative Examples 4 to 6 were calculated as relative values based on Comparative Example 4 and are listed in Table 2.

TABLE 1

| | | Driving voltage | Efficiency | T95 life-span |
|---|---|---|---|---|
| No. | Single host | (%) | (%) | (%) |
| Example 1 | Compound 1 | 92% | 118% | 119% |
| Example 2 | Compound 36 | 94% | 107% | 113% |
| Example 3 | Compound 52 | 91% | 107% | 125% |
| Comparative | R-1 | 100% | 100% | 100% |
| Example 1 | | | | |
| Comparative | R-2 | 98% | 93% | 94% |
| Example 2 | | | | |
| Comparative | R-3 | 104% | 79% | 75% |
| Example 3 | | | | |

TABLE 2

| | | Driving | | T95 |
|---|---|---|---|---|
| | | voltage | Efficiency | life-span |
| No. | Mixed host | (%) | (%) | (%) |
| Example 4 | Compound 1/A-30 | 92% | 112% | 156% |
| Example 5 | Compound 36/A-30 | 95% | 111% | 138% |
| Example 6 | Compound 52/A-52 | 93% | 111% | 163% |
| Example 7 | Compound 1/A-30 | 92% | 116% | 120% |
| Example 8 | Compound 36/A-30 | 94% | 106% | 125% |
| Example 9 | Compound 52/A-52 | 91% | 106% | 125% |
| Comparative | Compound R-1/A-30 | 100% | 100% | 100% |
| Example 4 | | | | |
| Comparative | Compound R-2/A-30 | 97% | 94% | 94% |
| Example 5 | | | | |
| Comparative | Compound R-3/A-30 | 104% | 81% | 75% |
| Example 6 | | | | |

Referring to Table 1 and Table 2, the organic light emitting diodes according to according to Examples 1 to 9 exhibited significantly improved driving voltage, luminous efficiency, and life-span characteristics compared to the organic light emitting diodes according to Comparative Examples 1 to 6.

By way of summation and review, some example embodiments may provide a compound for an organic optoelectronic device capable of implementing an organic optoelectronic device with low-driving, high efficiency, and long life-span.

Some example embodiments may provide a composition for an organic optoelectronic device including the compound for an organic optoelectronic device.

Some example embodiments may provide an organic optoelectronic device including the compound for an organic optoelectronic device or the composition for an organic optoelectronic device.

Some example embodiments may provide a display device including the organic optoelectronic device.

High-efficiency, long life-span organic optoelectronic devices can be realized while lowering the operating voltage

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1 or Chemical Formula 2: 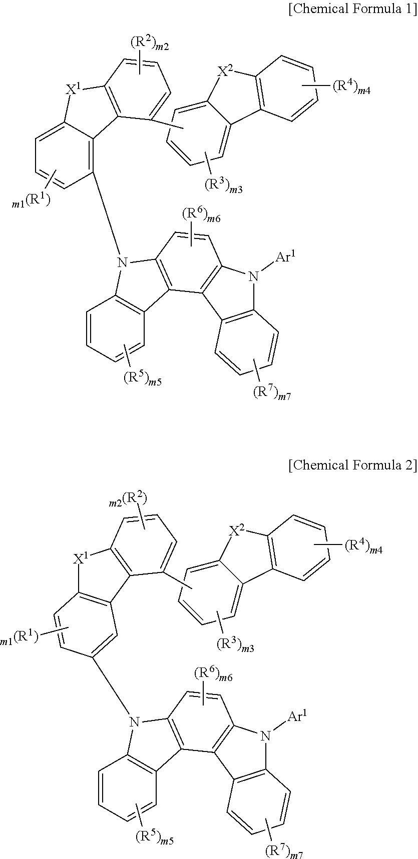
wherein, in Chemical Formula 1 and Chemical Formula 2,
X¹ and X² are each independently O or S,
R¹ to R⁷ are each independently hydrogen, deuterium, a cyano group, a substituted or unsubstituted C1 to C10 alkyl group, a substituted or unsubstituted C1 to C10 alkylsilyl group, a substituted or unsubstituted C3 to C20 cycloalkyl group, or a substituted or unsubstituted C6 to C20 aryl group,
Ar¹ is a substituted or unsubstituted C6 to C20 aryl group,
m1 to m3 are each independently an integer of 1 to 3,
m4, m5, and m7 are each independently an integer of 1 to 4, and
m6 is an integer of 1 or 2.

2. The compound for an organic optoelectronic device as claimed in claim 1, wherein:
the compound is represented by Chemical Formula 1,
Chemical Formula 1 is represented by one of Chemical Formula 1A to Chemical Formula 1D: 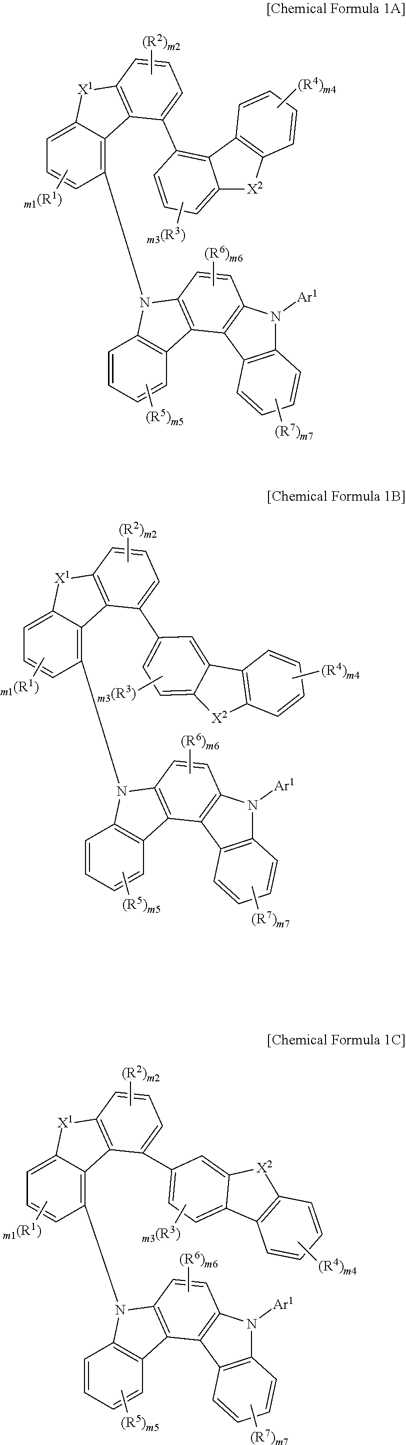 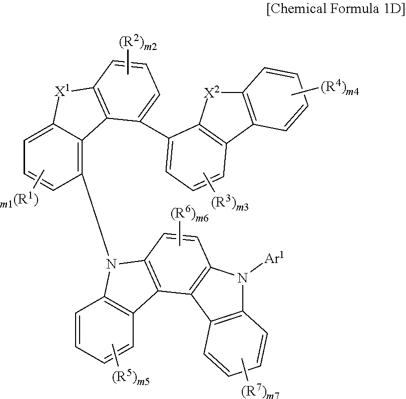
in Chemical Formula 1A to Chemical Formula 1D, X¹ and X², R¹ to R⁷, Ar¹, and m1 to m7 are defined the same as those of Chemical Formula 1.

3. The compound for an organic optoelectronic device as claimed in claim 2, wherein Chemical Formula 1 is represented by one of Chemical Formula 1A, Chemical Formula 1B, and Chemical Formula 1D.

4. The compound for an organic optoelectronic device as claimed in claim 1, wherein:
the compound is represented by Chemical Formula 2,
Chemical Formula 2 is represented by one of Chemical Formula 2A to Chemical Formula 2D: 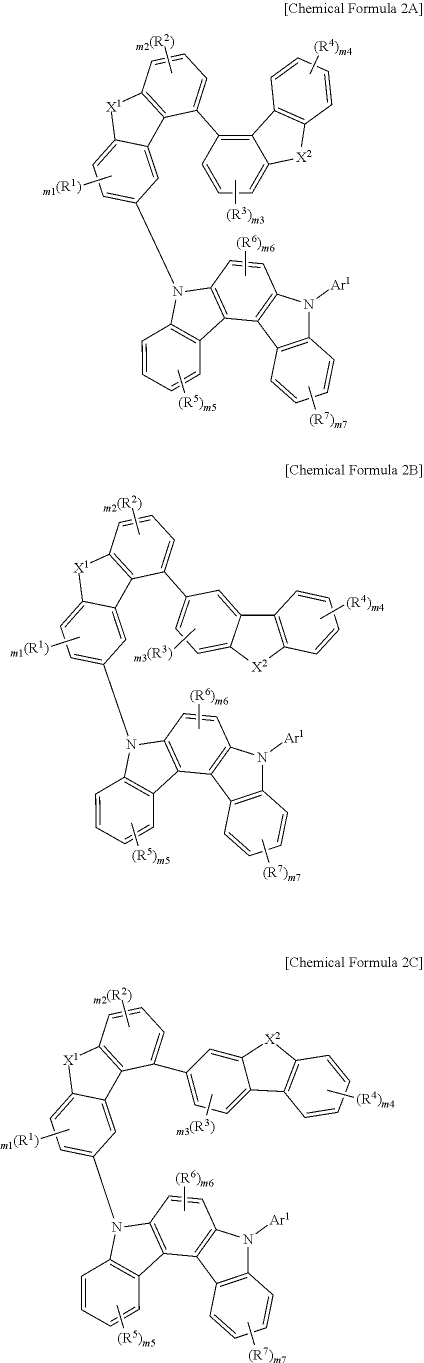 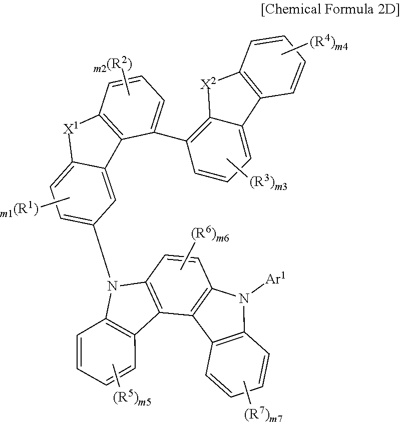
Chemical Formula 2A to Chemical Formula 2D, X¹ and X², R¹ to R⁷, Ar¹, and m1 to m7 are defined the same as those of Chemical Formula 2.

5. The compound for an organic optoelectronic device as claimed in claim 1, wherein Ar¹ is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted fluorenyl group, or a substituted or unsubstituted triphenylene group.

6. The compound for an organic optoelectronic device as claimed in claim 1, wherein the compound is a compound of Group 1, Group 1-1, or Group 1-2:

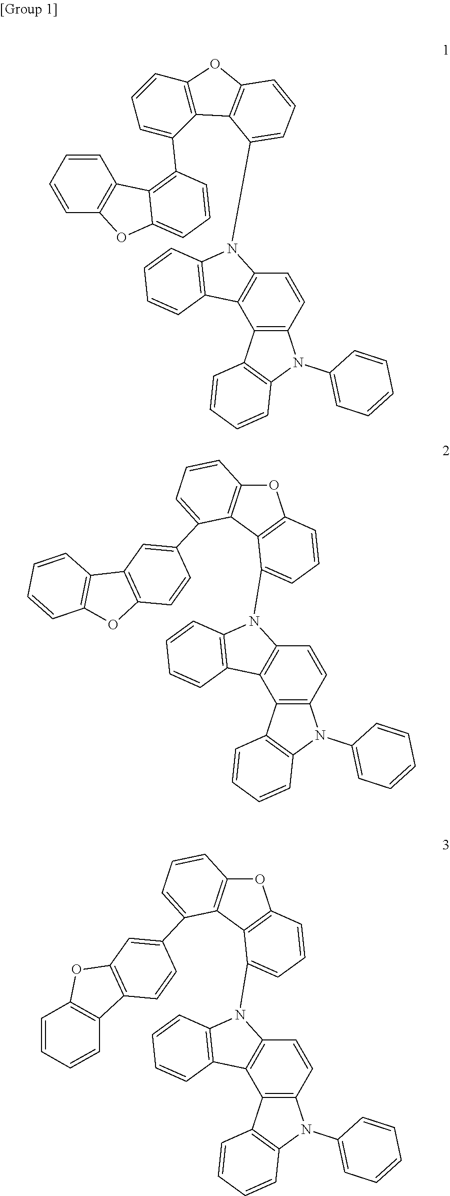

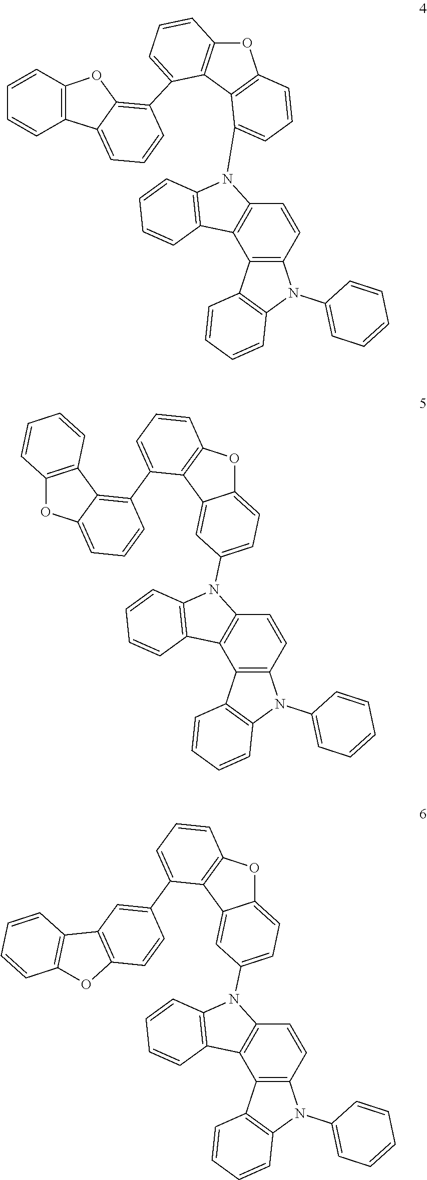 [SMILES: c1ccc(-n2c3ccccc3c3c4c5ccccc5n(-c5cccc6oc7cccc(-c8cccc9c8oc8ccccc89)c7c56)c4ccc32)cc1 ; c1ccc(-n2c3ccccc3c3c4c5ccccc5n(-c5ccc6oc7cccc(-c8cccc9oc%10ccccc%10c89)c7c6c5)c4ccc32)cc1 ; c1ccc(-n2c3ccccc3c3c4c5ccccc5n(-c5ccc6oc7cccc(-c8ccc9oc%10ccccc%10c9c8)c7c6c5)c4ccc32)cc1]

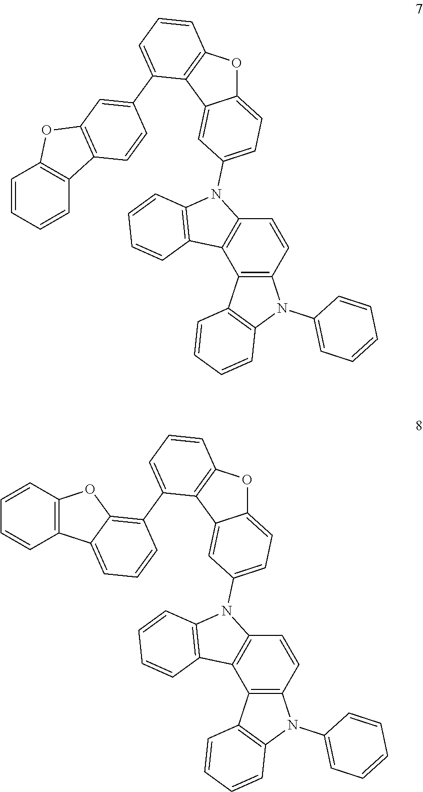 [SMILES: c1ccc(-n2c3ccccc3c3c4c5ccccc5n(-c5ccc6oc7cccc(-c8ccc9c(c8)oc8ccccc89)c7c6c5)c4ccc32)cc1 ; c1ccc(-n2c3ccccc3c3c4c5ccccc5n(-c5ccc6oc7cccc(-c8cccc9c8oc8ccccc89)c7c6c5)c4ccc32)cc1]

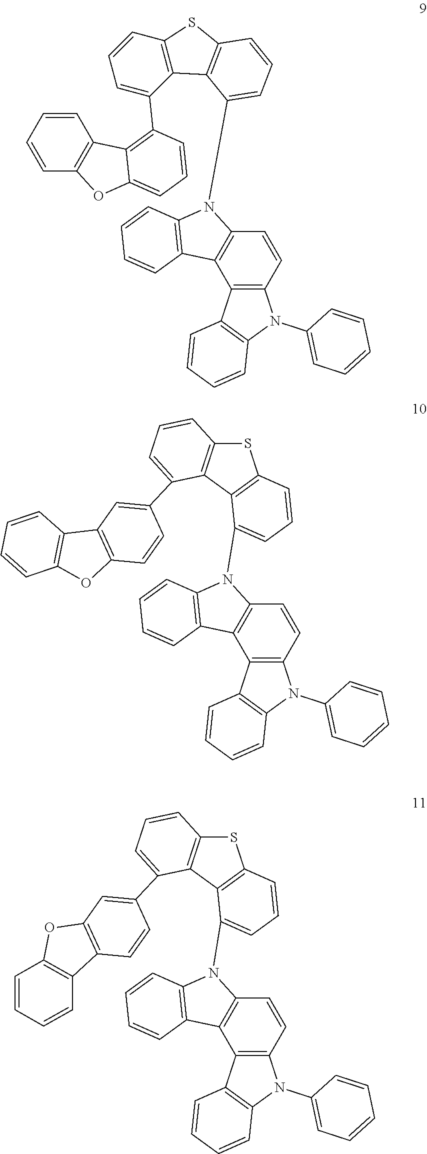

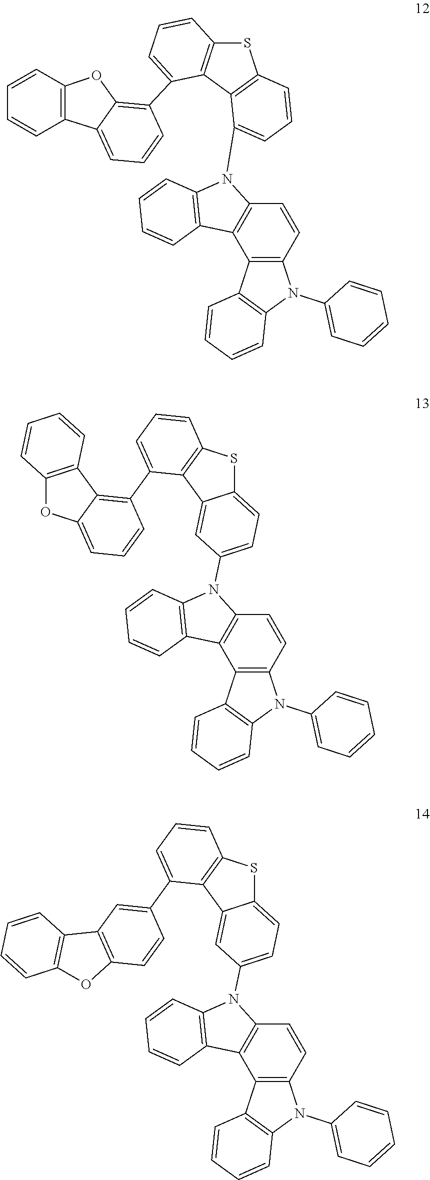 [SMILES: c1ccc(-n2c3ccccc3c3c4c5ccccc5n(-c5cccc6sc7cccc(-c8cccc9c8oc8ccccc89)c7c56)c4ccc32)cc1 ; c1ccc(-n2c3ccccc3c3c4c5ccccc5n(-c5ccc6sc7cccc(-c8cccc9oc%10ccccc%10c89)c7c6c5)c4ccc32)cc1 ; c1ccc(-n2c3ccccc3c3c4c5ccccc5n(-c5ccc6sc7cccc(-c8ccc9oc%10ccccc%10c9c8)c7c6c5)c4ccc32)cc1]

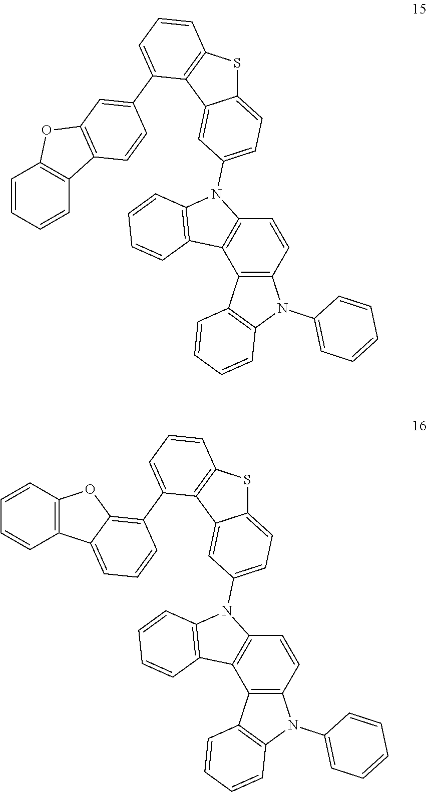 [SMILES: c1ccc(-n2c3ccccc3c3c4c5ccccc5n(-c5ccc6sc7cccc(-c8ccc9c(c8)oc8ccccc89)c7c6c5)c4ccc32)cc1 ; c1ccc(-n2c3ccccc3c3c4c5ccccc5n(-c5ccc6sc7cccc(-c8cccc9c8oc8ccccc89)c7c6c5)c4ccc32)cc1]

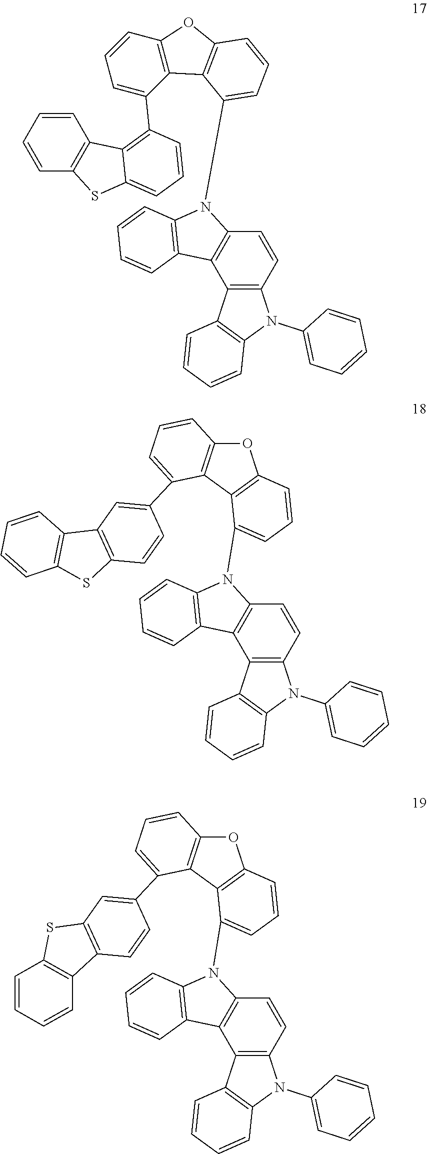

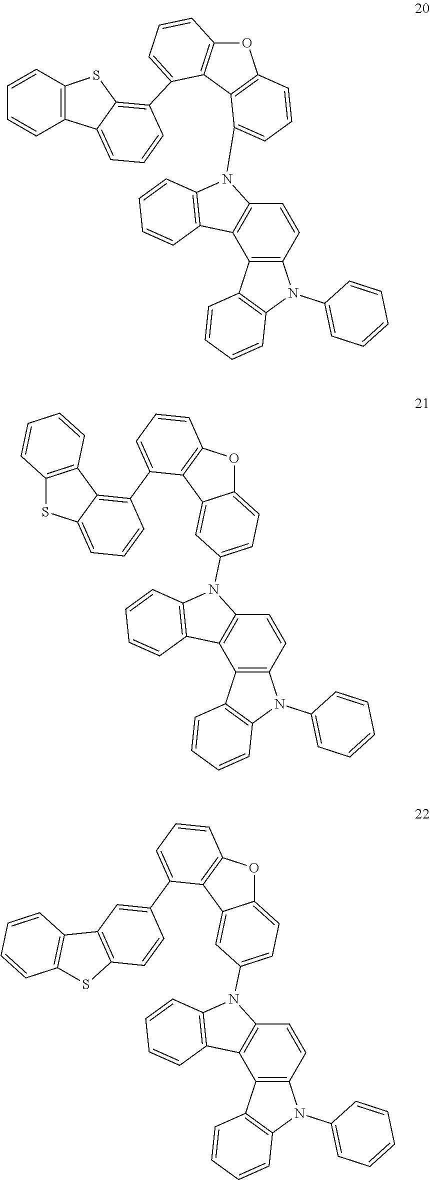 [SMILES: c1ccc(-n2c3ccccc3c3c4c5ccccc5n(-c5cccc6oc7cccc(-c8cccc9c8sc8ccccc89)c7c56)c4ccc32)cc1 ; c1ccc(-n2c3ccccc3c3c4c5ccccc5n(-c5ccc6oc7cccc(-c8cccc9sc%10ccccc%10c89)c7c6c5)c4ccc32)cc1 ; c1ccc(-n2c3ccccc3c3c4c5ccccc5n(-c5ccc6oc7cccc(-c8ccc9sc%10ccccc%10c9c8)c7c6c5)c4ccc32)cc1]

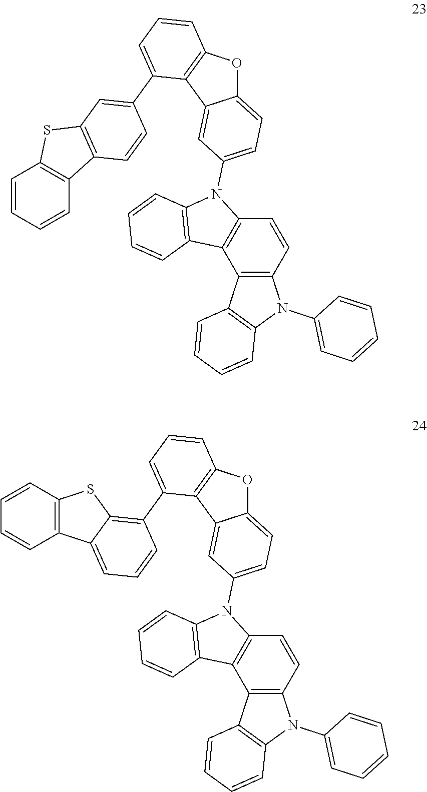 [SMILES: c1ccc(-n2c3ccccc3c3c4c5ccccc5n(-c5ccc6oc7cccc(-c8ccc9c(c8)sc8ccccc89)c7c6c5)c4ccc32)cc1 ; c1ccc(-n2c3ccccc3c3c4c5ccccc5n(-c5ccc6oc7cccc(-c8cccc9c8sc8ccccc89)c7c6c5)c4ccc32)cc1]

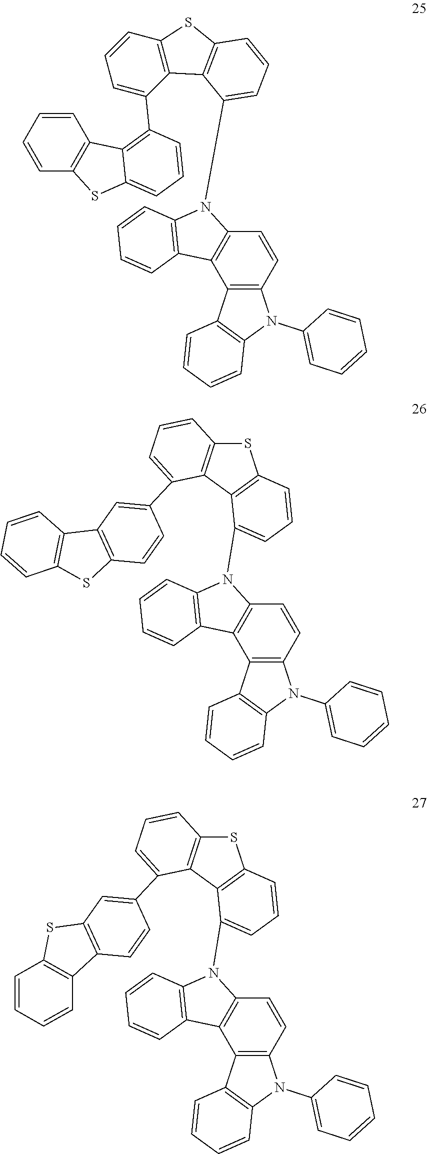

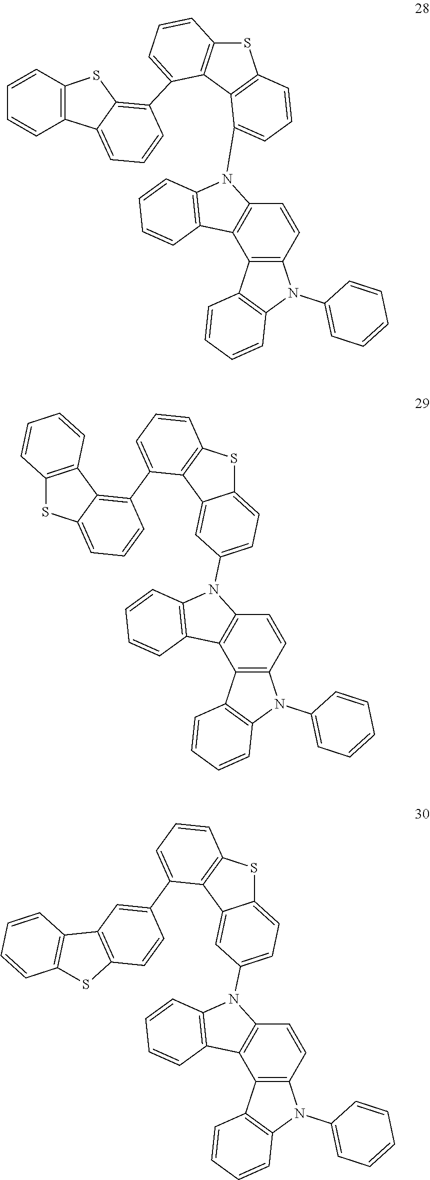 [SMILES: c1ccc(-n2c3ccccc3c3c4c5ccccc5n(-c5cccc6sc7cccc(-c8cccc9c8sc8ccccc89)c7c56)c4ccc32)cc1 ; c1ccc(-n2c3ccccc3c3c4c5ccccc5n(-c5ccc6sc7cccc(-c8cccc9sc%10ccccc%10c89)c7c6c5)c4ccc32)cc1 ; c1ccc(-n2c3ccccc3c3c4c5ccccc5n(-c5ccc6sc7cccc(-c8ccc9sc%10ccccc%10c9c8)c7c6c5)c4ccc32)cc1]

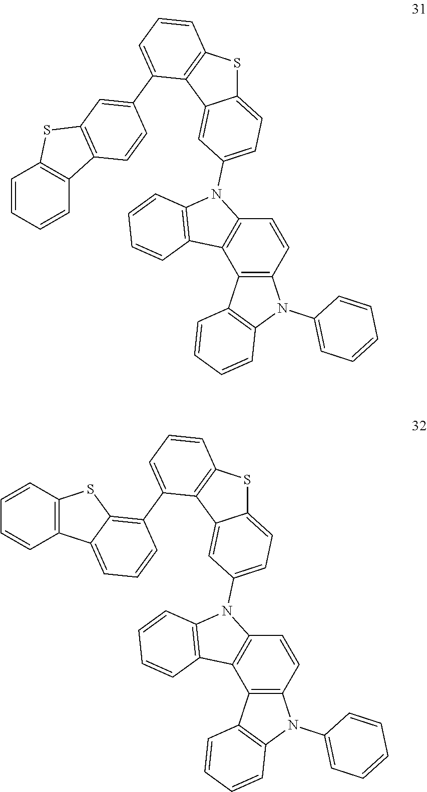 [SMILES: c1ccc(-n2c3ccccc3c3c4c5ccccc5n(-c5ccc6sc7cccc(-c8ccc9c(c8)sc8ccccc89)c7c6c5)c4ccc32)cc1 ; c1ccc(-n2c3ccccc3c3c4c5ccccc5n(-c5ccc6sc7cccc(-c8cccc9c8sc8ccccc89)c7c6c5)c4ccc32)cc1]

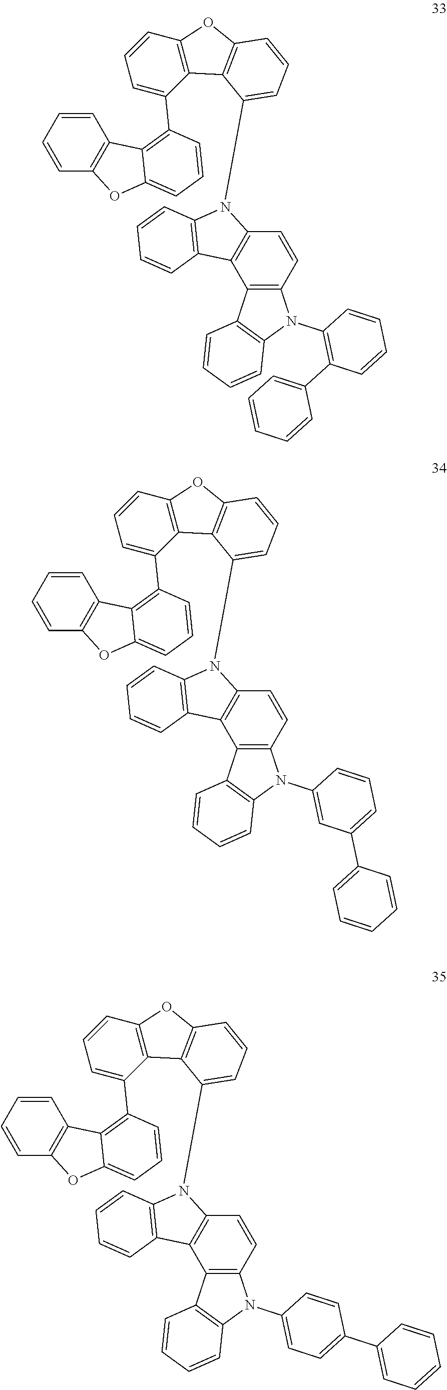 [SMILES: c1ccc(-c2ccccc2-n2c3ccccc3c3c4c5ccccc5n(-c5cccc6oc7cccc(-c8cccc9oc%10ccccc%10c89)c7c56)c4ccc32)cc1 ; c1ccc(-c2cccc(-n3c4ccccc4c4c5c6ccccc6n(-c6cccc7oc8cccc(-c9cccc%10oc%11ccccc%11c9%10)c8c67)c5ccc43)c2)cc1 ; c1ccc(-c2ccc(-n3c4ccccc4c4c5c6ccccc6n(-c6cccc7oc8cccc(-c9cccc%10oc%11ccccc%11c9%10)c8c67)c5ccc43)cc2)cc1]

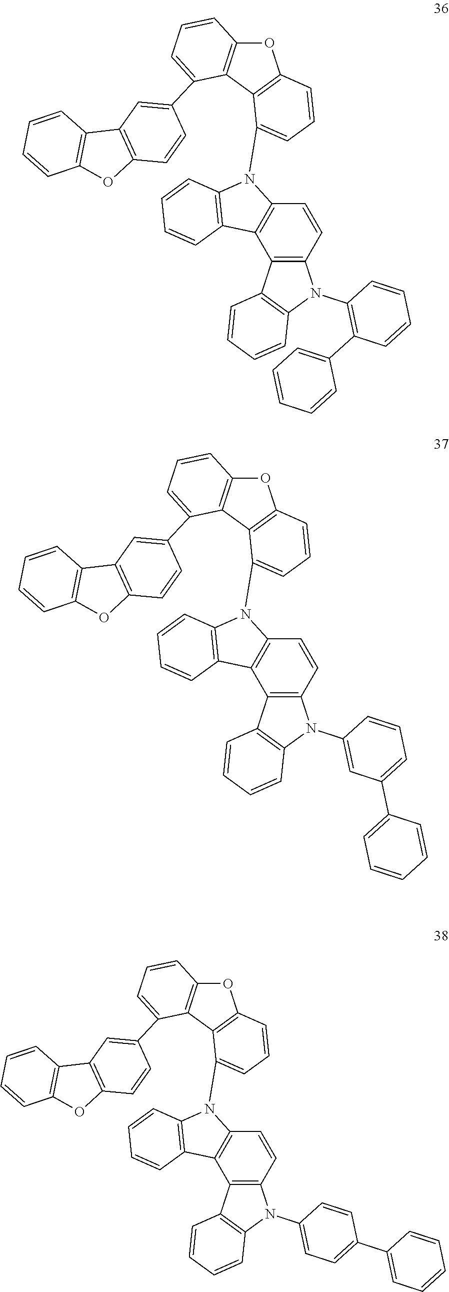 [SMILES: c1ccc(-c2ccccc2-n2c3ccccc3c3c4c5ccccc5n(-c5cccc6oc7cccc(-c8ccc9oc%10ccccc%10c9c8)c7c56)c4ccc32)cc1 ; c1ccc(-c2cccc(-n3c4ccccc4c4c5c6ccccc6n(-c6cccc7oc8cccc(-c9ccc%10oc%11ccccc%11c%10c9)c8c67)c5ccc43)c2)cc1 ; c1ccc(-c2ccc(-n3c4ccccc4c4c5c6ccccc6n(-c6cccc7oc8cccc(-c9ccc%10oc%11ccccc%11c%10c9)c8c67)c5ccc43)cc2)cc1]

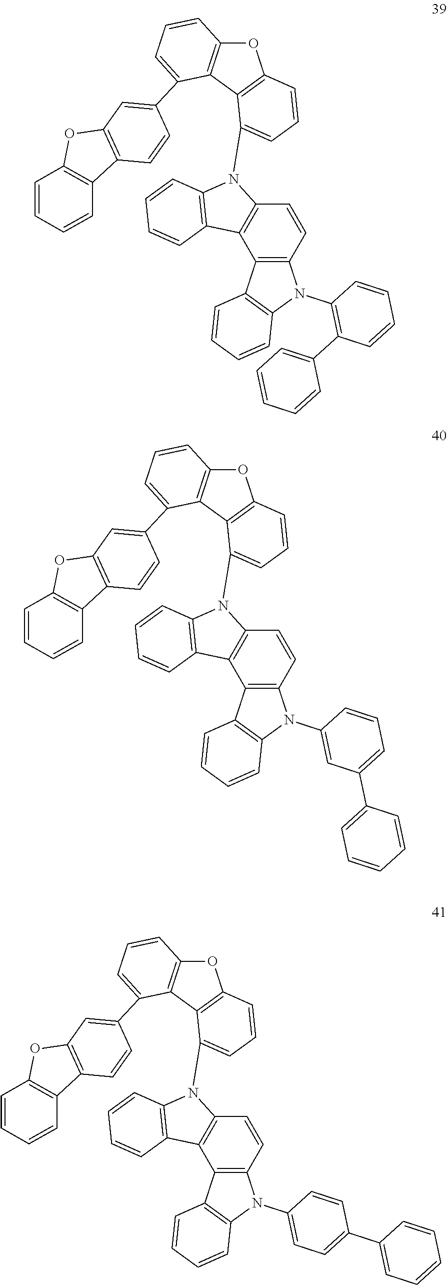 [SMILES: c1ccc(-c2ccccc2-n2c3ccccc3c3c4c5ccccc5n(-c5cccc6oc7cccc(-c8ccc9c(c8)oc8ccccc89)c7c56)c4ccc32)cc1 ; c1ccc(-c2cccc(-n3c4ccccc4c4c5c6ccccc6n(-c6cccc7oc8cccc(-c9ccc%10c(c9)oc9ccccc9%10)c8c67)c5ccc43)c2)cc1 ; c1ccc(-c2ccc(-n3c4ccccc4c4c5c6ccccc6n(-c6cccc7oc8cccc(-c9ccc%10c(c9)oc9ccccc9%10)c8c67)c5ccc43)cc2)cc1]

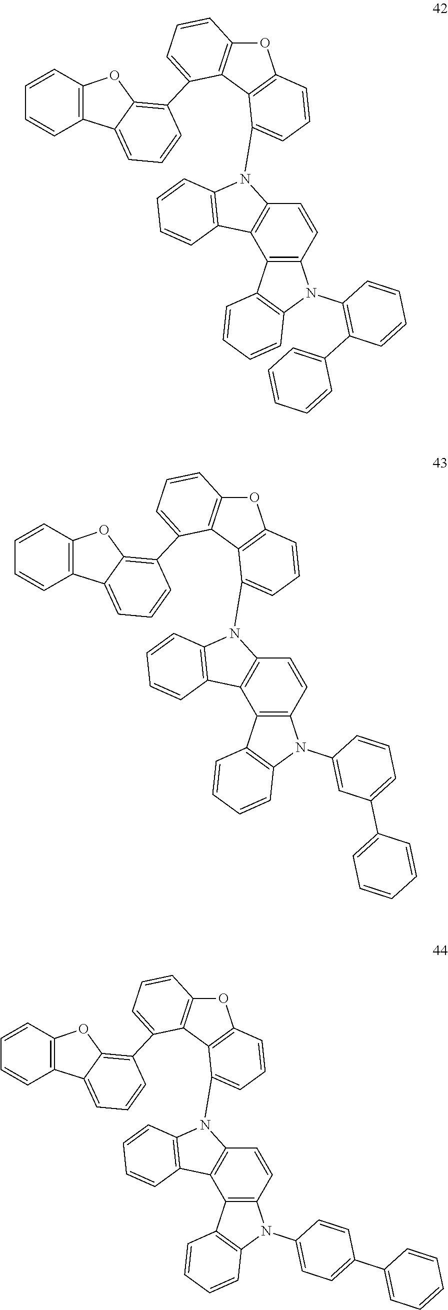 [SMILES: c1ccc(-c2ccccc2-n2c3ccccc3c3c4c5ccccc5n(-c5cccc6oc7cccc(-c8cccc9c8oc8ccccc89)c7c56)c4ccc32)cc1 ; c1ccc(-c2cccc(-n3c4ccccc4c4c5c6ccccc6n(-c6cccc7oc8cccc(-c9cccc%10c9oc9ccccc9%10)c8c67)c5ccc43)c2)cc1 ; c1ccc(-c2ccc(-n3c4ccccc4c4c5c6ccccc6n(-c6cccc7oc8cccc(-c9cccc%10c9oc9ccccc9%10)c8c67)c5ccc43)cc2)cc1]

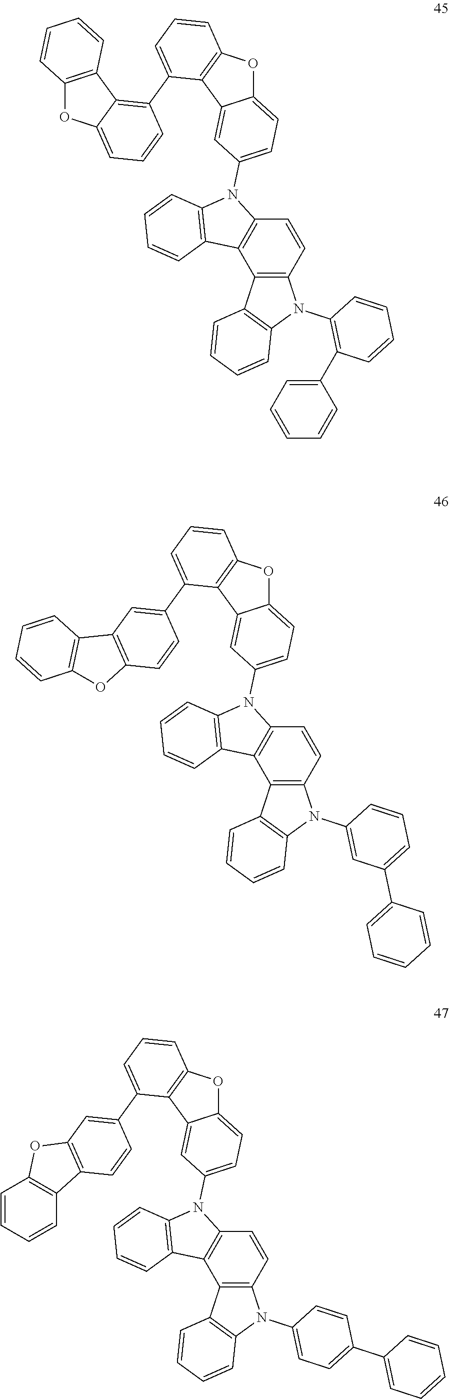 [SMILES: c1ccc(-c2ccccc2-n2c3ccccc3c3c4c5ccccc5n(-c5ccc6oc7cccc(-c8cccc9oc%10ccccc%10c89)c7c6c5)c4ccc32)cc1 ; c1ccc(-c2cccc(-n3c4ccccc4c4c5c6ccccc6n(-c6ccc7oc8cccc(-c9ccc%10oc%11ccccc%11c%10c9)c8c7c6)c5ccc43)c2)cc1 ; c1ccc(-c2ccc(-n3c4ccccc4c4c5c6ccccc6n(-c6ccc7oc8cccc(-c9ccc%10c(c9)oc9ccccc9%10)c8c7c6)c5ccc43)cc2)cc1]

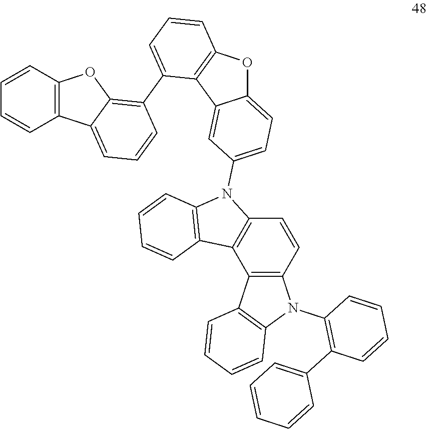 [SMILES: c1ccc(-c2ccccc2-n2c3ccccc3c3c4c5ccccc5n(-c5ccc6oc7cccc(-c8cccc9c8oc8ccccc89)c7c6c5)c4ccc32)cc1]

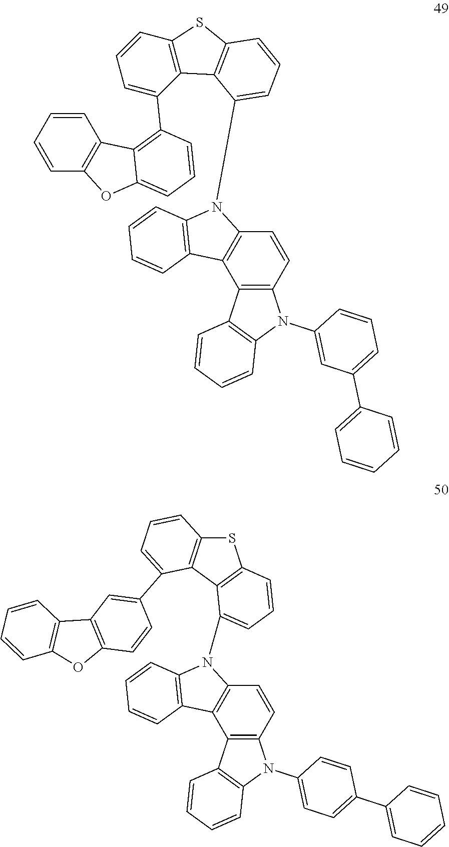 [SMILES: c1ccc(-c2cccc(-n3c4ccccc4c4c5c6ccccc6n(-c6cccc7sc8cccc(-c9cccc%10oc%11ccccc%11c9%10)c8c67)c5ccc43)c2)cc1 ; c1ccc(-c2ccc(-n3c4ccccc4c4c5c6ccccc6n(-c6cccc7sc8cccc(-c9ccc%10oc%11ccccc%11c%10c9)c8c67)c5ccc43)cc2)cc1]

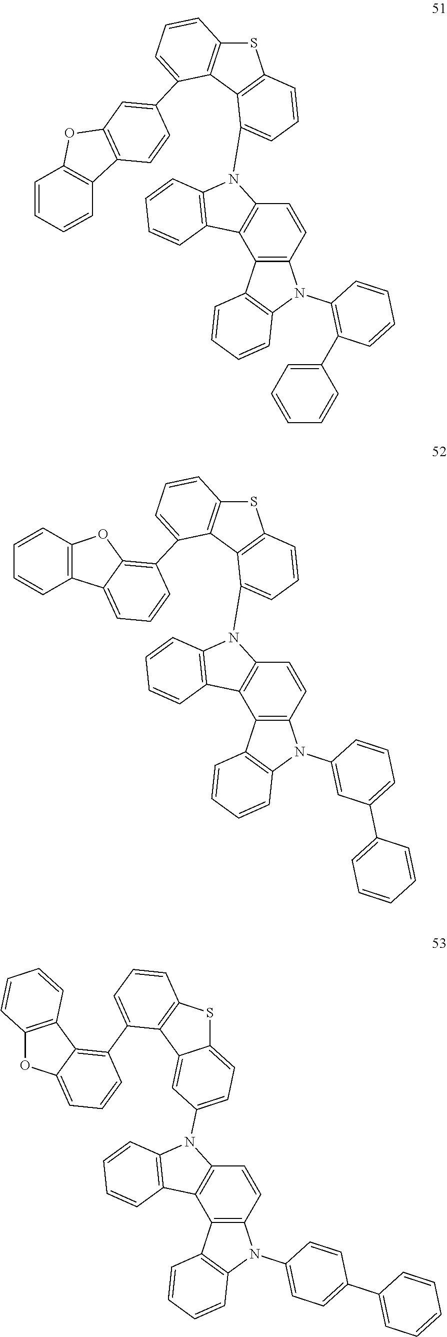 [SMILES: c1ccc(-c2ccccc2-n2c3ccccc3c3c4c5ccccc5n(-c5cccc6sc7cccc(-c8ccc9c(c8)oc8ccccc89)c7c56)c4ccc32)cc1 ; c1ccc(-c2cccc(-n3c4ccccc4c4c5c6ccccc6n(-c6cccc7sc8cccc(-c9cccc%10c9oc9ccccc9%10)c8c67)c5ccc43)c2)cc1 ; c1ccc(-c2ccc(-n3c4ccccc4c4c5c6ccccc6n(-c6ccc7sc8cccc(-c9cccc%10oc%11ccccc%11c9%10)c8c7c6)c5ccc43)cc2)cc1]

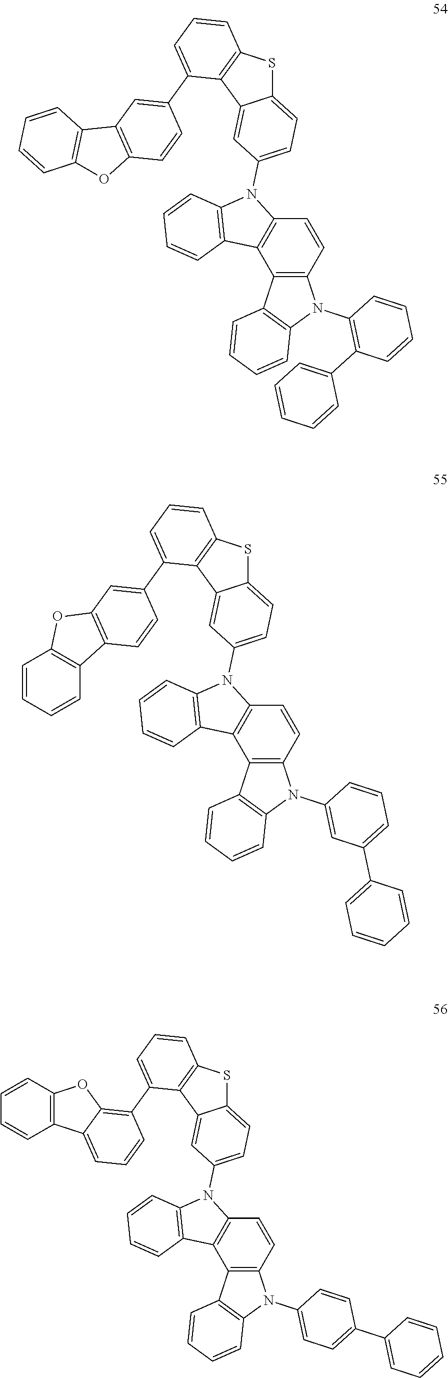 [SMILES: c1ccc(-c2ccccc2-n2c3ccccc3c3c4c5ccccc5n(-c5ccc6sc7cccc(-c8ccc9oc%10ccccc%10c9c8)c7c6c5)c4ccc32)cc1 ; c1ccc(-c2cccc(-n3c4ccccc4c4c5c6ccccc6n(-c6ccc7sc8cccc(-c9ccc%10c(c9)oc9ccccc9%10)c8c7c6)c5ccc43)c2)cc1 ; c1ccc(-c2ccc(-n3c4ccccc4c4c5c6ccccc6n(-c6ccc7sc8cccc(-c9cccc%10c9oc9ccccc9%10)c8c7c6)c5ccc43)cc2)cc1]

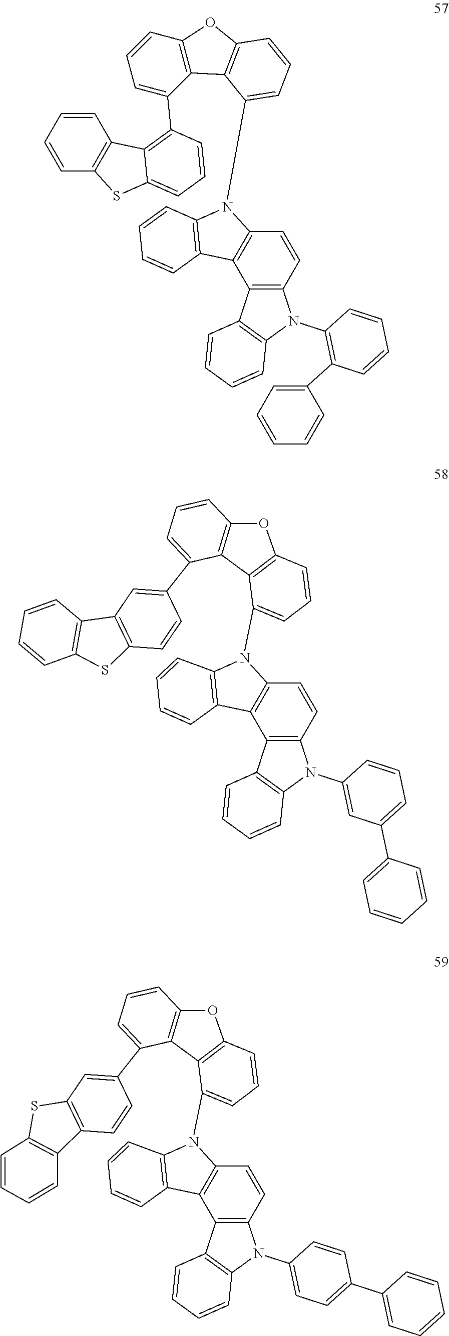 [SMILES: c1ccc(-c2ccccc2-n2c3ccccc3c3c4c5ccccc5n(-c5cccc6oc7cccc(-c8cccc9sc%10ccccc%10c89)c7c56)c4ccc32)cc1 ; c1ccc(-c2cccc(-n3c4ccccc4c4c5c6ccccc6n(-c6cccc7oc8cccc(-c9ccc%10sc%11ccccc%11c%10c9)c8c67)c5ccc43)c2)cc1 ; c1ccc(-c2ccc(-n3c4ccccc4c4c5c6ccccc6n(-c6cccc7oc8cccc(-c9ccc%10c(c9)sc9ccccc9%10)c8c67)c5ccc43)cc2)cc1]

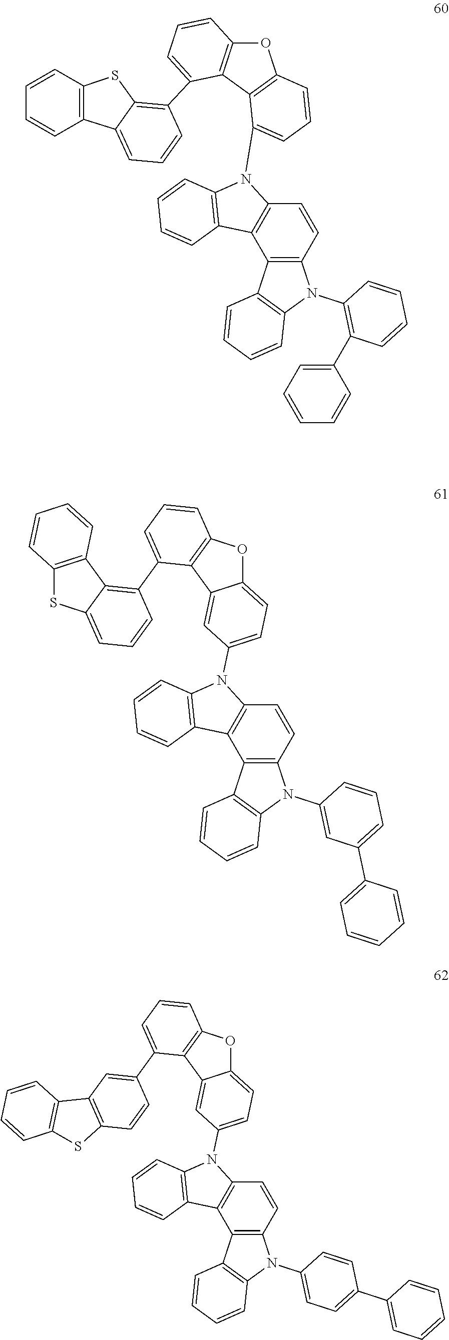 [SMILES: c1ccc(-c2ccccc2-n2c3ccccc3c3c4c5ccccc5n(-c5cccc6oc7cccc(-c8cccc9c8sc8ccccc89)c7c56)c4ccc32)cc1 ; c1ccc(-c2cccc(-n3c4ccccc4c4c5c6ccccc6n(-c6ccc7oc8cccc(-c9cccc%10sc%11ccccc%11c9%10)c8c7c6)c5ccc43)c2)cc1 ; c1ccc(-c2ccc(-n3c4ccccc4c4c5c6ccccc6n(-c6ccc7oc8cccc(-c9ccc%10sc%11ccccc%11c%10c9)c8c7c6)c5ccc43)cc2)cc1]

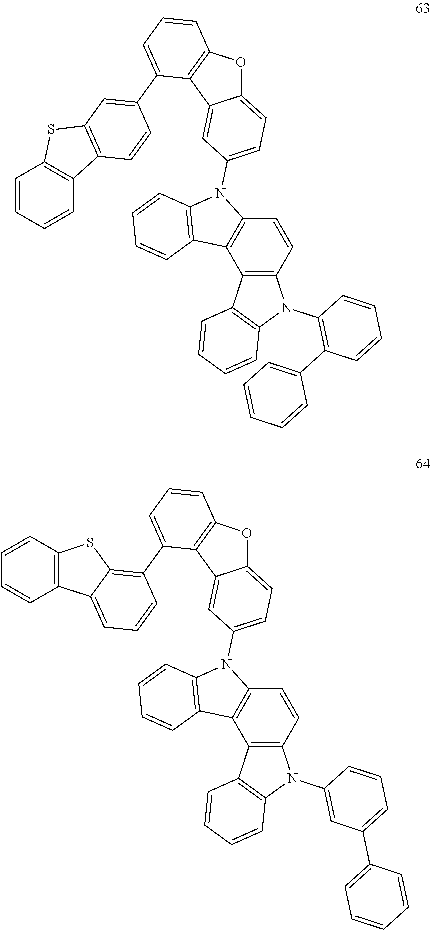 [SMILES: c1ccc(-c2ccccc2-n2c3ccccc3c3c4c5ccccc5n(-c5ccc6oc7cccc(-c8ccc9c(c8)sc8ccccc89)c7c6c5)c4ccc32)cc1 ; c1ccc(-c2cccc(-n3c4ccccc4c4c5c6ccccc6n(-c6ccc7oc8cccc(-c9cccc%10c9sc9ccccc9%10)c8c7c6)c5ccc43)c2)cc1]

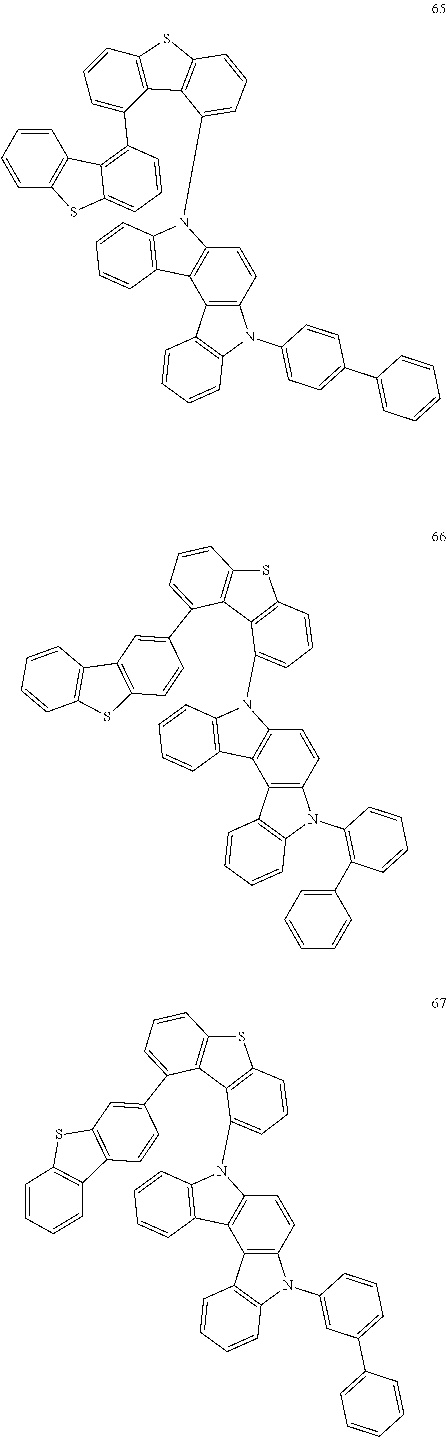 [SMILES: c1ccc(-c2ccc(-n3c4ccccc4c4c5c6ccccc6n(-c6cccc7sc8cccc(-c9cccc%10sc%11ccccc%11c9%10)c8c67)c5ccc43)cc2)cc1 ; c1ccc(-c2ccccc2-n2c3ccccc3c3c4c5ccccc5n(-c5cccc6sc7cccc(-c8ccc9sc%10ccccc%10c9c8)c7c56)c4ccc32)cc1 ; c1ccc(-c2cccc(-n3c4ccccc4c4c5c6ccccc6n(-c6cccc7sc8cccc(-c9ccc%10c(c9)sc9ccccc9%10)c8c67)c5ccc43)c2)cc1]

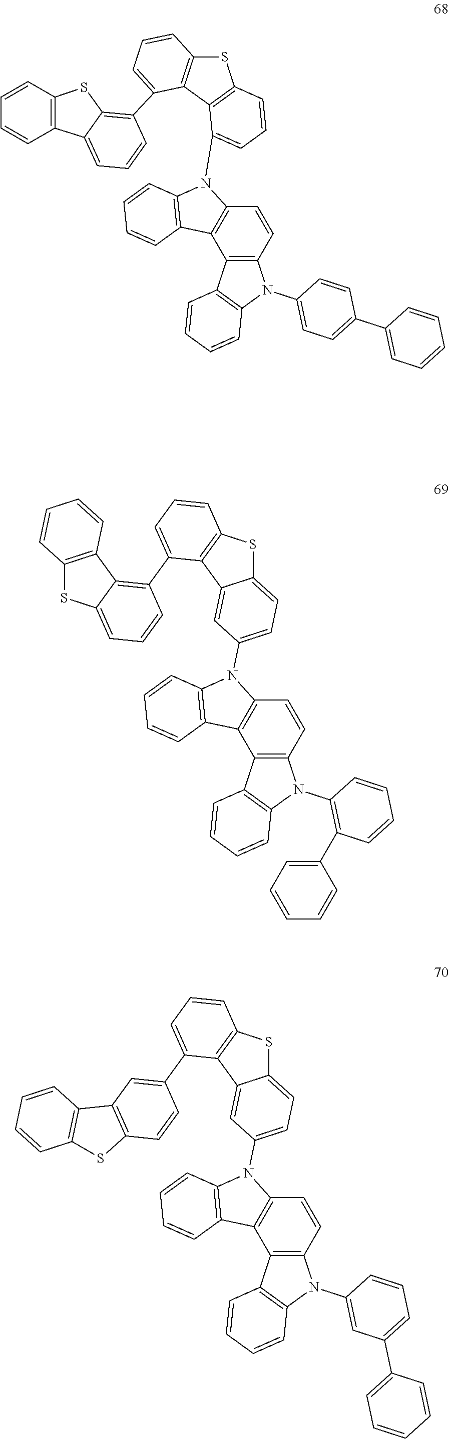 [SMILES: c1ccc(-c2ccc(-n3c4ccccc4c4c5c6ccccc6n(-c6cccc7sc8cccc(-c9cccc%10c9sc9ccccc9%10)c8c67)c5ccc43)cc2)cc1 ; c1ccc(-c2ccccc2-n2c3ccccc3c3c4c5ccccc5n(-c5ccc6sc7cccc(-c8cccc9sc%10ccccc%10c89)c7c6c5)c4ccc32)cc1 ; c1ccc(-c2cccc(-n3c4ccccc4c4c5c6ccccc6n(-c6ccc7sc8cccc(-c9ccc%10sc%11ccccc%11c%10c9)c8c7c6)c5ccc43)c2)cc1]

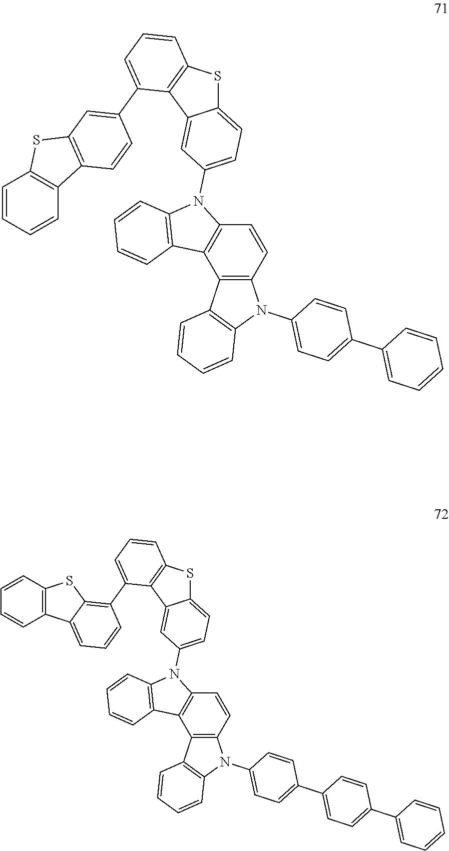 [SMILES: c1ccc(-c2ccc(-n3c4ccccc4c4c5c6ccccc6n(-c6ccc7sc8cccc(-c9ccc%10c(c9)sc9ccccc9%10)c8c7c6)c5ccc43)cc2)cc1 ; c1ccc(-c2ccc(-c3ccc(-n4c5ccccc5c5c6c7ccccc7n(-c7ccc8sc9cccc(-c%10cccc%11c%10sc%10ccccc%10%11)c9c8c7)c6ccc54)cc3)cc2)cc1]

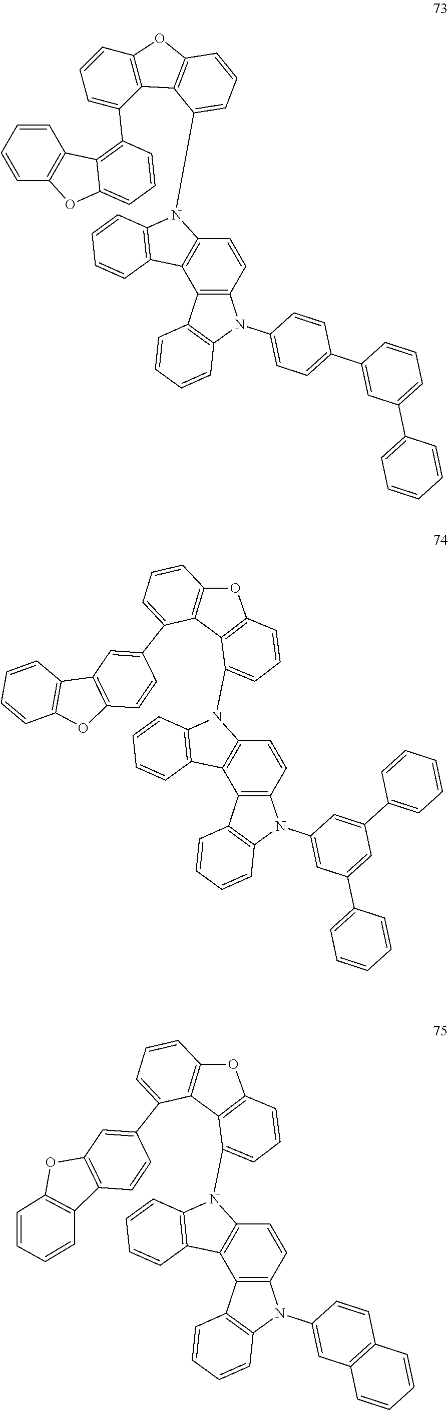 [SMILES: c1ccc(-c2cccc(-c3ccc(-n4c5ccccc5c5c6c7ccccc7n(-c7cccc8oc9cccc(-c%10cccc%11oc%12ccccc%12c%10%11)c9c78)c6ccc54)cc3)c2)cc1 ; c1ccc(-c2cc(-c3ccccc3)cc(-n3c4ccccc4c4c5c6ccccc6n(-c6cccc7oc8cccc(-c9ccc%10oc%11ccccc%11c%10c9)c8c67)c5ccc43)c2)cc1 ; c1ccc2cc(-n3c4ccccc4c4c5c6ccccc6n(-c6cccc7oc8cccc(-c9ccc%10c(c9)oc9ccccc9%10)c8c67)c5ccc43)ccc2c1]

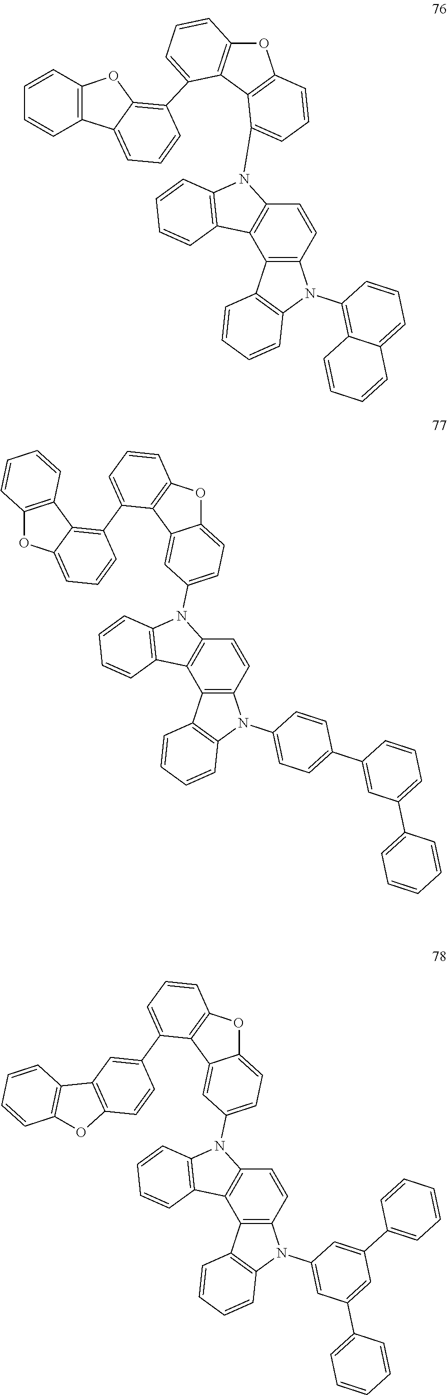 [SMILES: c1ccc2c(-n3c4ccccc4c4c5c6ccccc6n(-c6cccc7oc8cccc(-c9cccc%10c9oc9ccccc9%10)c8c67)c5ccc43)cccc2c1 ; c1ccc(-c2cccc(-c3ccc(-n4c5ccccc5c5c6c7ccccc7n(-c7ccc8oc9cccc(-c%10cccc%11oc%12ccccc%12c%10%11)c9c8c7)c6ccc54)cc3)c2)cc1 ; c1ccc(-c2cc(-c3ccccc3)cc(-n3c4ccccc4c4c5c6ccccc6n(-c6ccc7oc8cccc(-c9ccc%10oc%11ccccc%11c%10c9)c8c7c6)c5ccc43)c2)cc1]

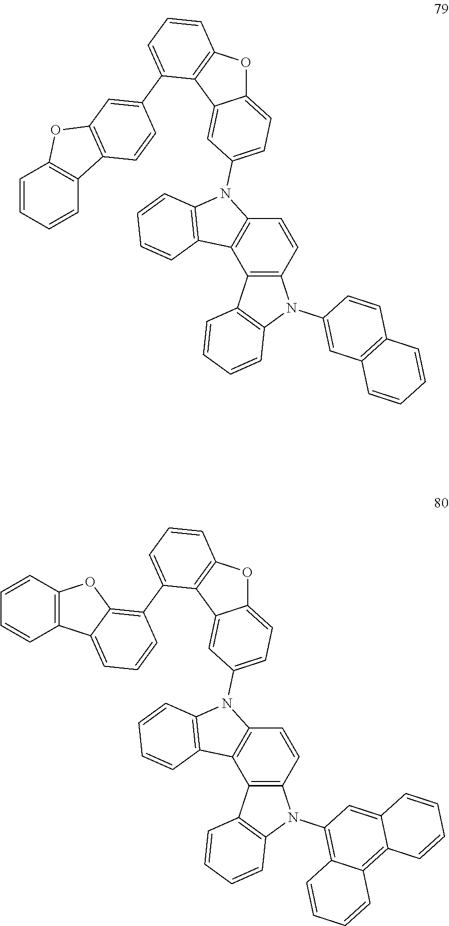 [SMILES: c1ccc2cc(-n3c4ccccc4c4c5c6ccccc6n(-c6ccc7oc8cccc(-c9ccc%10c(c9)oc9ccccc9%10)c8c7c6)c5ccc43)ccc2c1 ; c1ccc2c(c1)cc(-n1c3ccccc3c3c4c5ccccc5n(-c5ccc6oc7cccc(-c8cccc9c8oc8ccccc89)c7c6c5)c4ccc31)c1ccccc12]

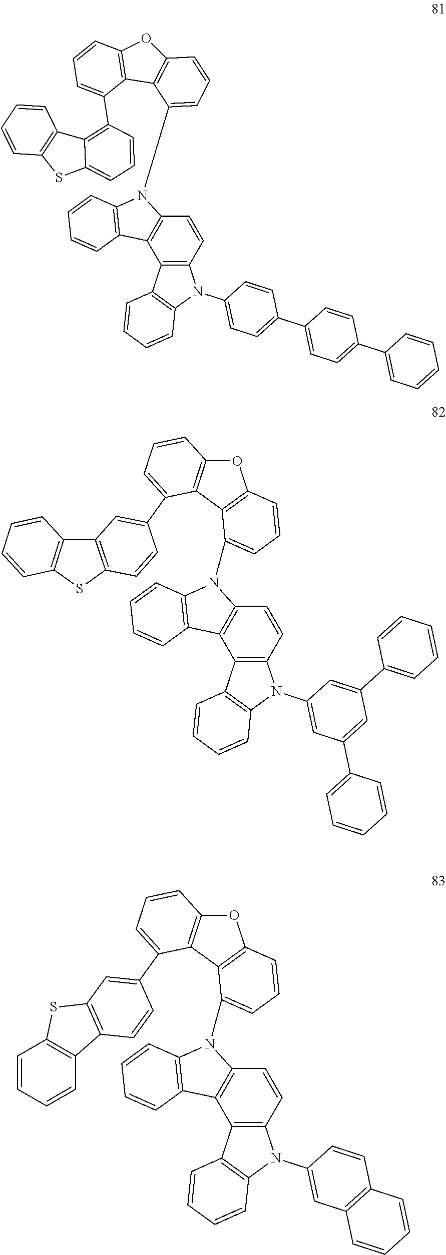 [SMILES: c1ccc(-c2ccc(-c3ccc(-n4c5ccccc5c5c6c7ccccc7n(-c7cccc8oc9cccc(-c%10cccc%11sc%12ccccc%12c%10%11)c9c78)c6ccc54)cc3)cc2)cc1 ; c1ccc(-c2cc(-c3ccccc3)cc(-n3c4ccccc4c4c5c6ccccc6n(-c6cccc7oc8cccc(-c9ccc%10sc%11ccccc%11c%10c9)c8c67)c5ccc43)c2)cc1 ; c1ccc2cc(-n3c4ccccc4c4c5c6ccccc6n(-c6cccc7oc8cccc(-c9ccc%10c(c9)sc9ccccc9%10)c8c67)c5ccc43)ccc2c1]

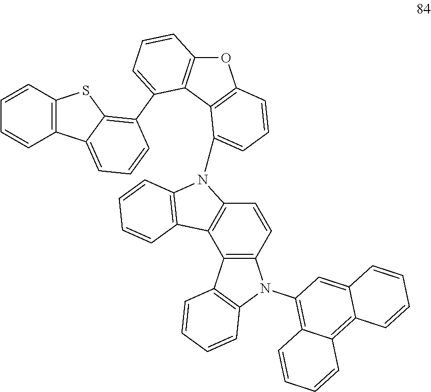 [SMILES: c1ccc2c(c1)cc(-n1c3ccccc3c3c4c5ccccc5n(-c5cccc6oc7cccc(-c8cccc9c8sc8ccccc89)c7c56)c4ccc31)c1ccccc12]

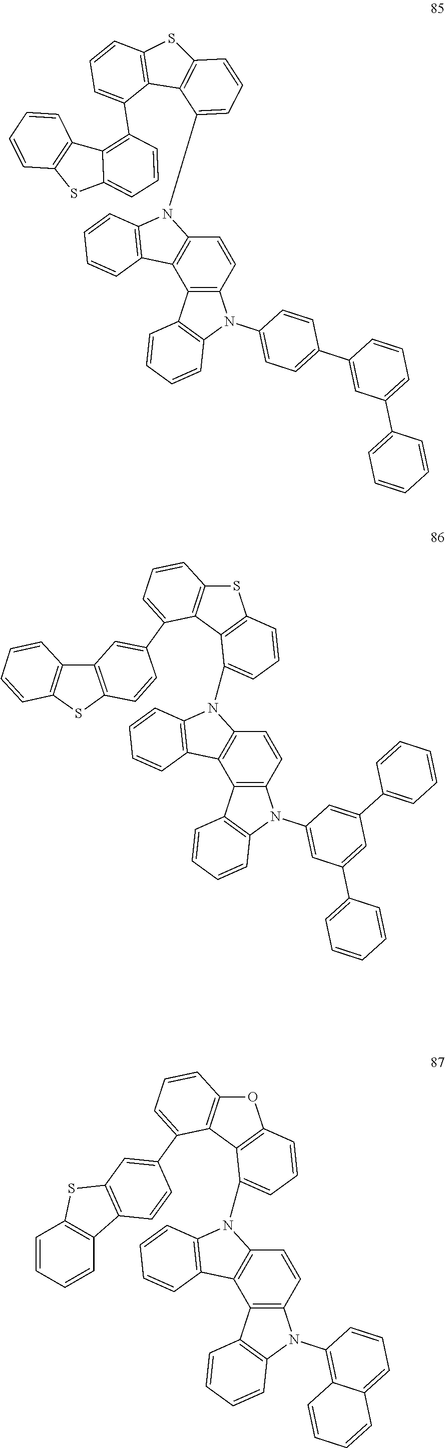 [SMILES: c1ccc(-c2cccc(-c3ccc(-n4c5ccccc5c5c6c7ccccc7n(-c7cccc8sc9cccc(-c%10cccc%11sc%12ccccc%12c%10%11)c9c78)c6ccc54)cc3)c2)cc1 ; c1ccc(-c2cc(-c3ccccc3)cc(-n3c4ccccc4c4c5c6ccccc6n(-c6cccc7sc8cccc(-c9ccc%10sc%11ccccc%11c%10c9)c8c67)c5ccc43)c2)cc1 ; c1ccc2c(-n3c4ccccc4c4c5c6ccccc6n(-c6cccc7oc8cccc(-c9ccc%10c(c9)sc9ccccc9%10)c8c67)c5ccc43)cccc2c1]

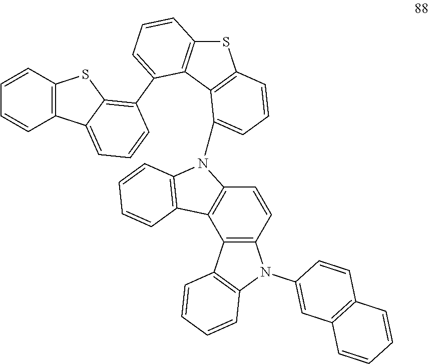 [SMILES: c1ccc2cc(-n3c4ccccc4c4c5c6ccccc6n(-c6cccc7sc8cccc(-c9cccc%10c9sc9ccccc9%10)c8c67)c5ccc43)ccc2c1]

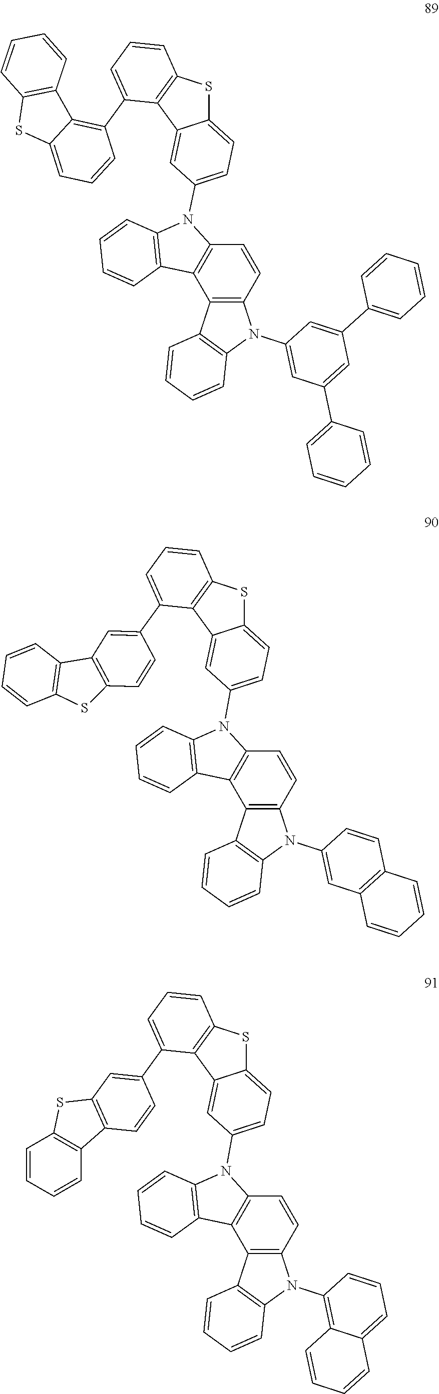 [SMILES: c1ccc(-c2cc(-c3ccccc3)cc(-n3c4ccccc4c4c5c6ccccc6n(-c6ccc7sc8cccc(-c9cccc%10sc%11ccccc%11c9%10)c8c7c6)c5ccc43)c2)cc1 ; c1ccc2cc(-n3c4ccccc4c4c5c6ccccc6n(-c6ccc7sc8cccc(-c9ccc%10sc%11ccccc%11c%10c9)c8c7c6)c5ccc43)ccc2c1 ; c1ccc2c(-n3c4ccccc4c4c5c6ccccc6n(-c6ccc7sc8cccc(-c9ccc%10c(c9)sc9ccccc9%10)c8c7c6)c5ccc43)cccc2c1]

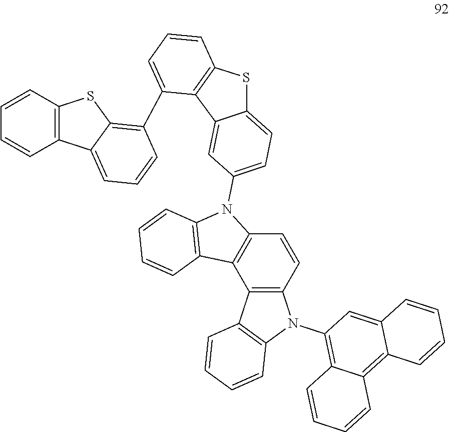 [SMILES: c1ccc2c(c1)cc(-n1c3ccccc3c3c4c5ccccc5n(-c5ccc6sc7cccc(-c8cccc9c8sc8ccccc89)c7c6c5)c4ccc31)c1ccccc12]

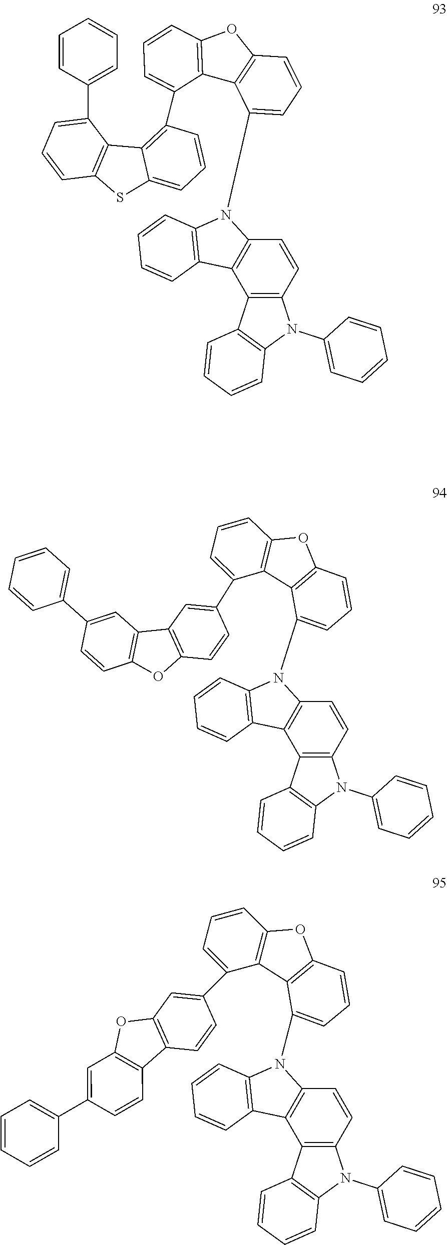 [SMILES: c1ccc(-c2cccc3sc4cccc(-c5cccc6oc7cccc(-n8c9ccccc9c9c%10c%11ccccc%11n(-c%11ccccc%11)c%10ccc98)c7c56)c4c23)cc1 ; c1ccc(-c2ccc3oc4ccc(-c5cccc6oc7cccc(-n8c9ccccc9c9c%10c%11ccccc%11n(-c%11ccccc%11)c%10ccc98)c7c56)cc4c3c2)cc1 ; c1ccc(-c2ccc3c(c2)oc2cc(-c4cccc5oc6cccc(-n7c8ccccc8c8c9c%10ccccc%10n(-c%10ccccc%10)c9ccc87)c6c45)ccc23)cc1]

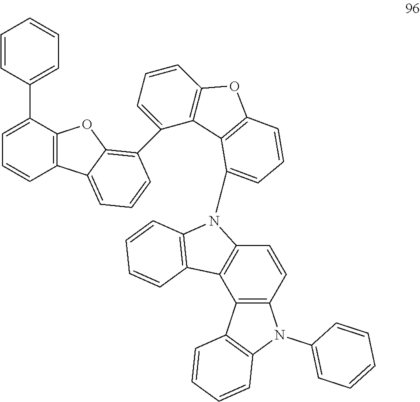 [SMILES: c1ccc(-c2cccc3c2oc2c(-c4cccc5oc6cccc(-n7c8ccccc8c8c9c%10ccccc%10n(-c%10ccccc%10)c9ccc87)c6c45)cccc23)cc1]

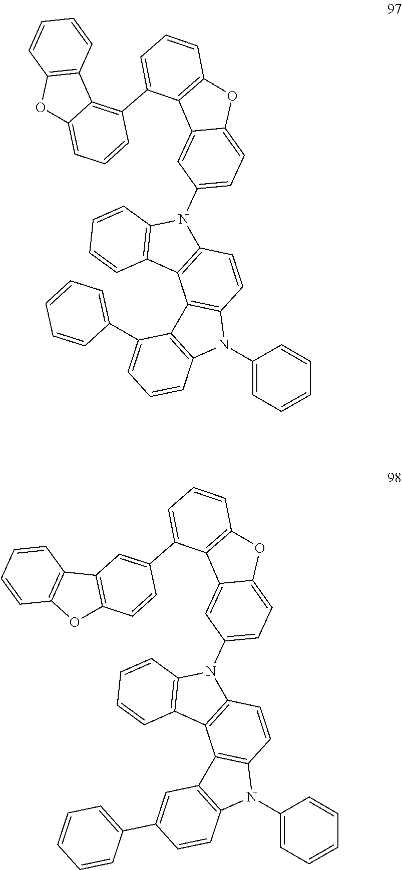 [SMILES: c1ccc(-c2cccc3c2c2c4c5ccccc5n(-c5ccc6oc7cccc(-c8cccc9oc%10ccccc%10c89)c7c6c5)c4ccc2n3-c2ccccc2)cc1 ; c1ccc(-c2ccc3c(c2)c2c4c5ccccc5n(-c5ccc6oc7cccc(-c8ccc9oc%10ccccc%10c9c8)c7c6c5)c4ccc2n3-c2ccccc2)cc1]

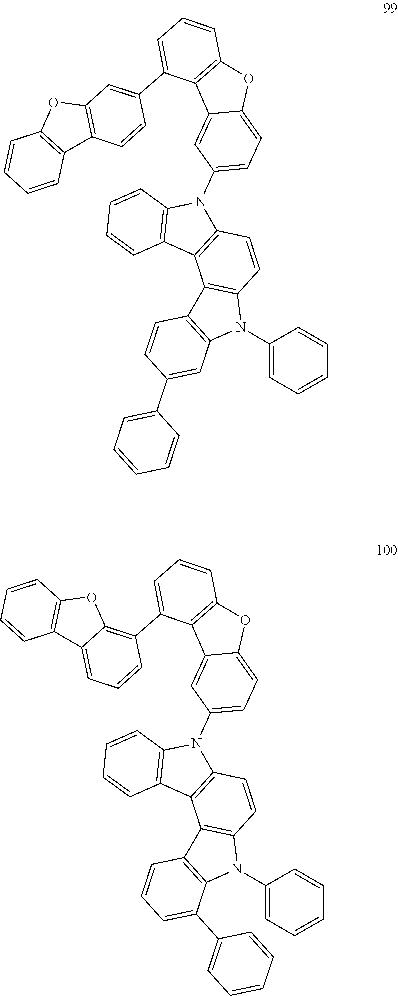 [SMILES: c1ccc(-c2ccc3c4c5c6ccccc6n(-c6ccc7oc8cccc(-c9ccc%10c(c9)oc9ccccc9%10)c8c7c6)c5ccc4n(-c4ccccc4)c3c2)cc1 ; c1ccc(-c2cccc3c4c5c6ccccc6n(-c6ccc7oc8cccc(-c9cccc%10c9oc9ccccc9%10)c8c7c6)c5ccc4n(-c4ccccc4)c23)cc1]

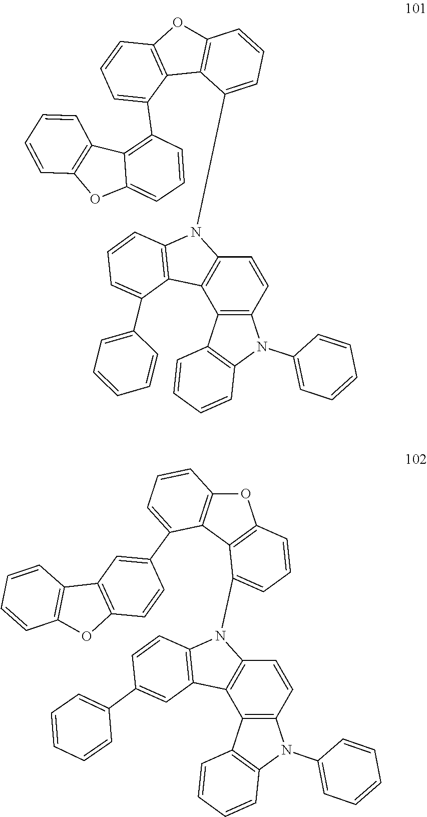 [SMILES: c1ccc(-c2cccc3c2c2c4c5ccccc5n(-c5ccccc5)c4ccc2n3-c2cccc3oc4cccc(-c5cccc6oc7ccccc7c56)c4c23)cc1 ; c1ccc(-c2ccc3c(c2)c2c4c5ccccc5n(-c5ccccc5)c4ccc2n3-c2cccc3oc4cccc(-c5ccc6oc7ccccc7c6c5)c4c23)cc1]

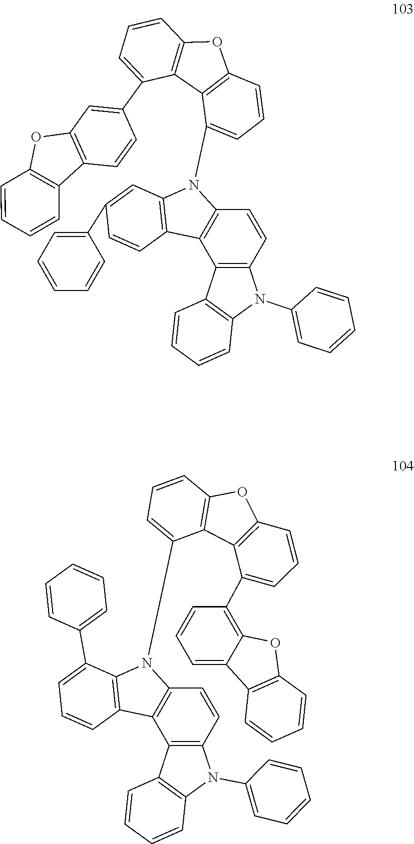 [SMILES: c1ccc(-c2ccc3c4c5c6ccccc6n(-c6ccccc6)c5ccc4n(-c4cccc5oc6cccc(-c7ccc8c(c7)oc7ccccc78)c6c45)c3c2)cc1 ; c1ccc(-c2cccc3c4c5c6ccccc6n(-c6ccccc6)c5ccc4n(-c4cccc5oc6cccc(-c7cccc8c7oc7ccccc78)c6c45)c23)cc1]

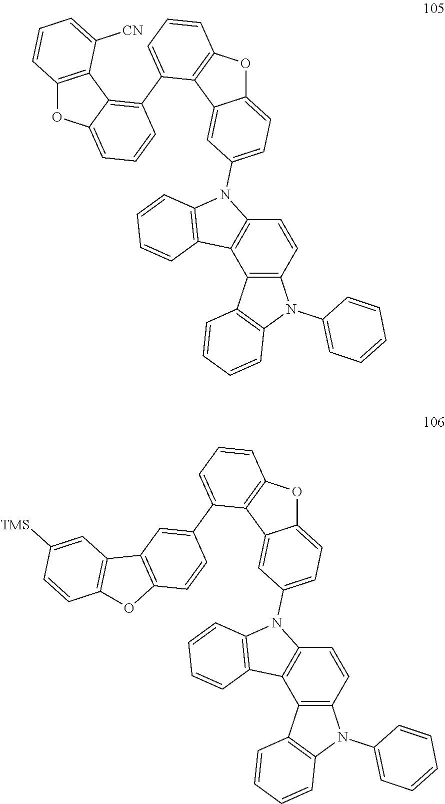 [SMILES: N#Cc1cccc2oc3cccc(-c4cccc5oc6ccc(-n7c8ccccc8c8c9c%10ccccc%10n(-c%10ccccc%10)c9ccc87)cc6c45)c3c12 ; C[Si](C)(C)c1ccc2oc3ccc(-c4cccc5oc6ccc(-n7c8ccccc8c8c9c%10ccccc%10n(-c%10ccccc%10)c9ccc87)cc6c45)cc3c2c1]

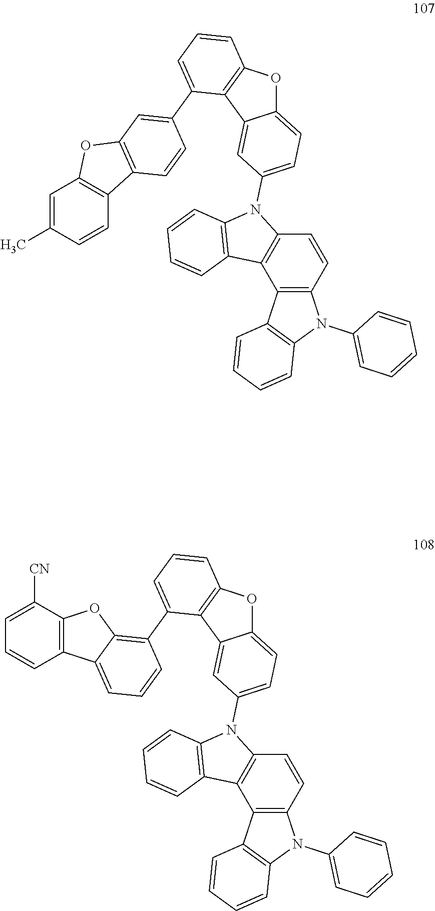 [SMILES: Cc1ccc2c(c1)oc1cc(-c3cccc4oc5ccc(-n6c7ccccc7c7c8c9ccccc9n(-c9ccccc9)c8ccc76)cc5c34)ccc12 ; N#Cc1cccc2c1oc1c(-c3cccc4oc5ccc(-n6c7ccccc7c7c8c9ccccc9n(-c9ccccc9)c8ccc76)cc5c34)cccc12]

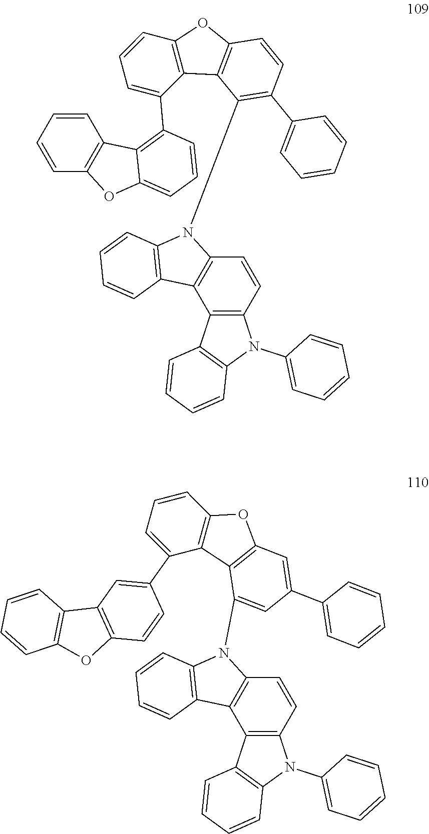 [SMILES: c1ccc(-c2ccc3oc4cccc(-c5cccc6oc7ccccc7c56)c4c3c2-n2c3ccccc3c3c4c5ccccc5n(-c5ccccc5)c4ccc32)cc1 ; c1ccc(-c2cc(-n3c4ccccc4c4c5c6ccccc6n(-c6ccccc6)c5ccc43)c3c(c2)oc2cccc(-c4ccc5oc6ccccc6c5c4)c23)cc1]

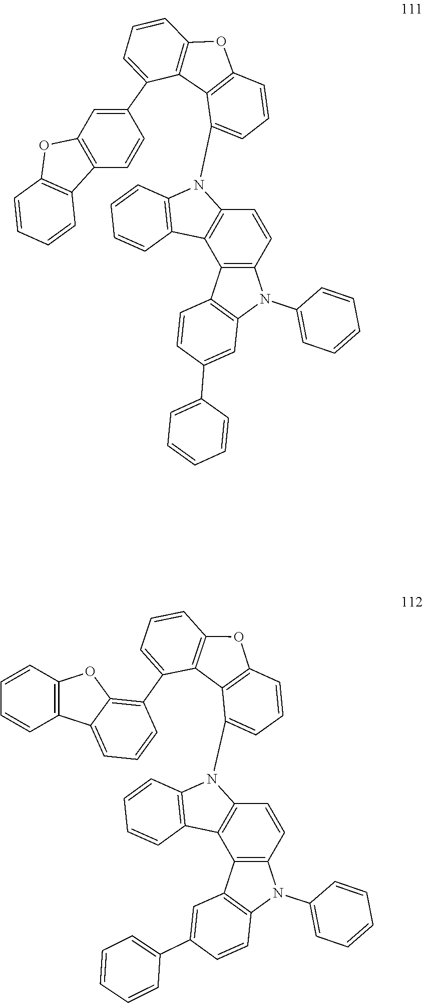 [SMILES: c1ccc(-c2ccc3c4c5c6ccccc6n(-c6cccc7oc8cccc(-c9ccc%10c(c9)oc9ccccc9%10)c8c67)c5ccc4n(-c4ccccc4)c3c2)cc1 ; c1ccc(-c2ccc3c(c2)c2c4c5ccccc5n(-c5cccc6oc7cccc(-c8cccc9c8oc8ccccc89)c7c56)c4ccc2n3-c2ccccc2)cc1]

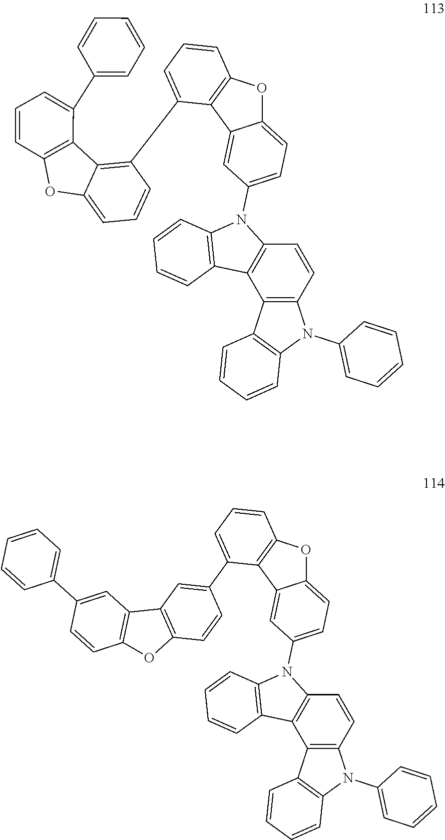 [SMILES: c1ccc(-c2cccc3oc4cccc(-c5cccc6oc7ccc(-n8c9ccccc9c9c%10c%11ccccc%11n(-c%11ccccc%11)c%10ccc98)cc7c56)c4c23)cc1 ; c1ccc(-c2ccc3oc4ccc(-c5cccc6oc7ccc(-n8c9ccccc9c9c%10c%11ccccc%11n(-c%11ccccc%11)c%10ccc98)cc7c56)cc4c3c2)cc1]

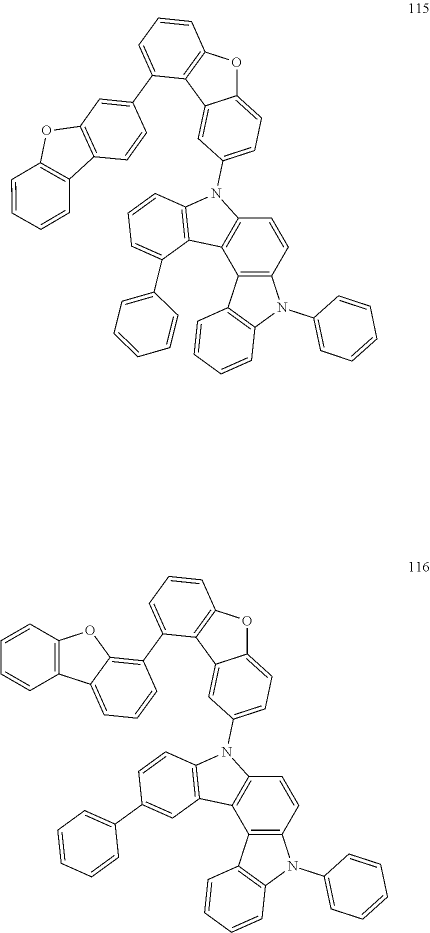 [SMILES: c1ccc(-c2cccc3c2c2c4c5ccccc5n(-c5ccccc5)c4ccc2n3-c2ccc3oc4cccc(-c5ccc6c(c5)oc5ccccc56)c4c3c2)cc1 ; c1ccc(-c2ccc3c(c2)c2c4c5ccccc5n(-c5ccccc5)c4ccc2n3-c2ccc3oc4cccc(-c5cccc6c5oc5ccccc56)c4c3c2)cc1]

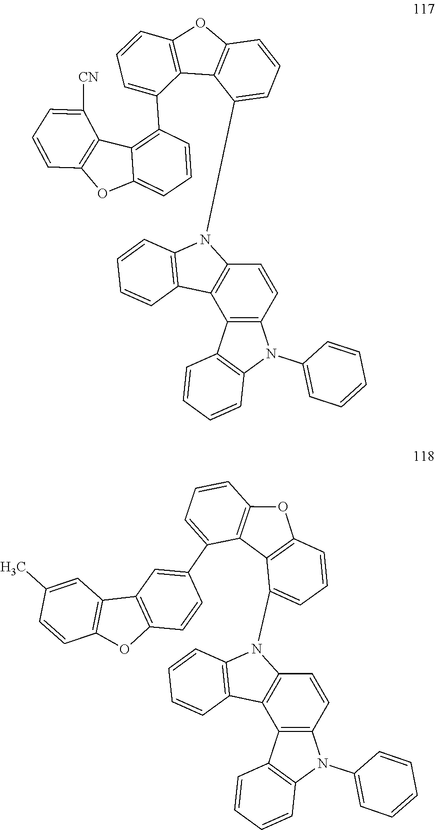 [SMILES: N#Cc1cccc2oc3cccc(-c4cccc5oc6cccc(-n7c8ccccc8c8c9c%10ccccc%10n(-c%10ccccc%10)c9ccc87)c6c45)c3c12 ; Cc1ccc2oc3ccc(-c4cccc5oc6cccc(-n7c8ccccc8c8c9c%10ccccc%10n(-c%10ccccc%10)c9ccc87)c6c45)cc3c2c1]

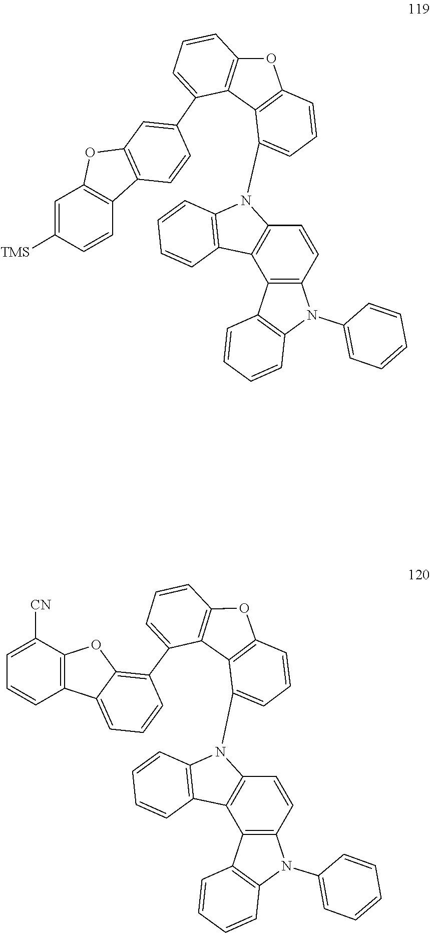 [SMILES: C[Si](C)(C)c1ccc2c(c1)oc1cc(-c3cccc4oc5cccc(-n6c7ccccc7c7c8c9ccccc9n(-c9ccccc9)c8ccc76)c5c34)ccc12 ; N#Cc1cccc2c1oc1c(-c3cccc4oc5cccc(-n6c7ccccc7c7c8c9ccccc9n(-c9ccccc9)c8ccc76)c5c34)cccc12]

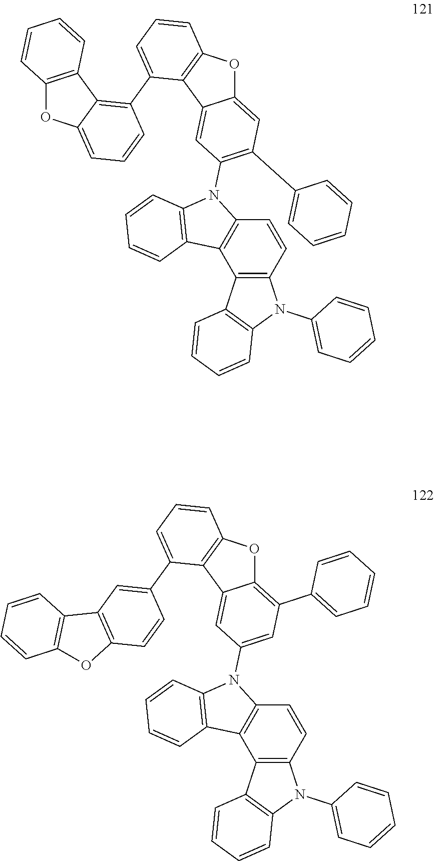 [SMILES: c1ccc(-c2cc3oc4cccc(-c5cccc6oc7ccccc7c56)c4c3cc2-n2c3ccccc3c3c4c5ccccc5n(-c5ccccc5)c4ccc32)cc1 ; c1ccc(-c2cc(-n3c4ccccc4c4c5c6ccccc6n(-c6ccccc6)c5ccc43)cc3c2oc2cccc(-c4ccc5oc6ccccc6c5c4)c23)cc1]

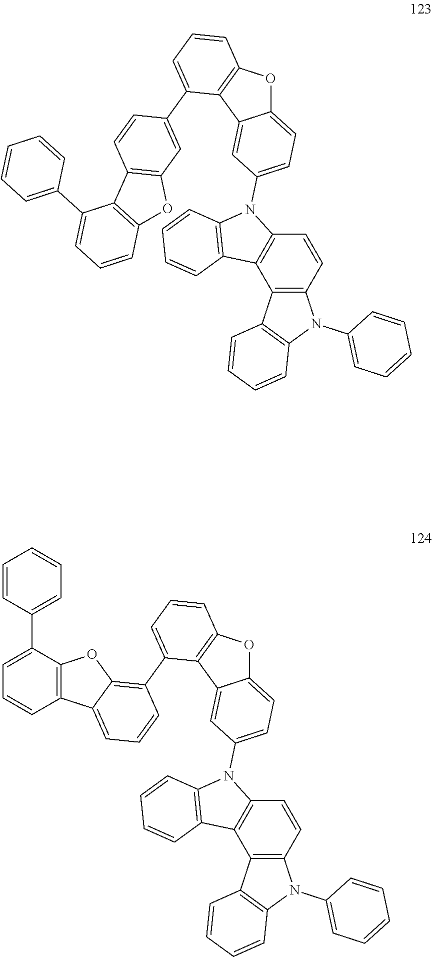 [SMILES: c1ccc(-c2cccc3oc4cc(-c5cccc6oc7ccc(-n8c9ccccc9c9c%10c%11ccccc%11n(-c%11ccccc%11)c%10ccc98)cc7c56)ccc4c23)cc1 ; c1ccc(-c2cccc3c2oc2c(-c4cccc5oc6ccc(-n7c8ccccc8c8c9c%10ccccc%10n(-c%10ccccc%10)c9ccc87)cc6c45)cccc23)cc1]

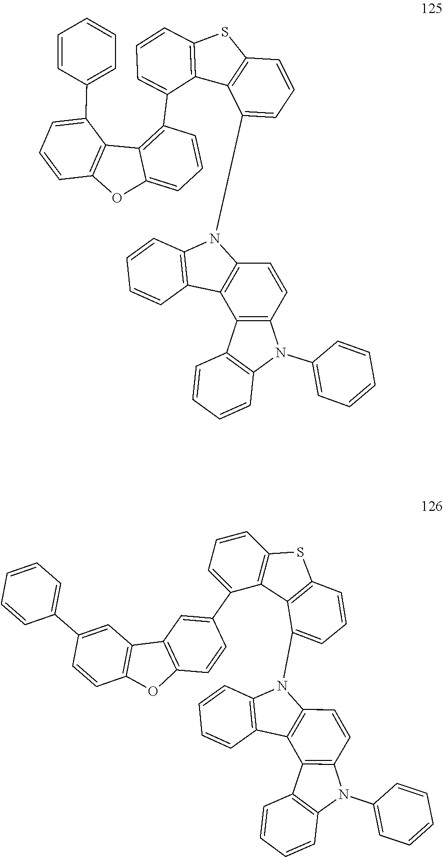 [SMILES: c1ccc(-c2cccc3oc4cccc(-c5cccc6sc7cccc(-n8c9ccccc9c9c%10c%11ccccc%11n(-c%11ccccc%11)c%10ccc98)c7c56)c4c23)cc1 ; c1ccc(-c2ccc3oc4ccc(-c5cccc6sc7cccc(-n8c9ccccc9c9c%10c%11ccccc%11n(-c%11ccccc%11)c%10ccc98)c7c56)cc4c3c2)cc1]

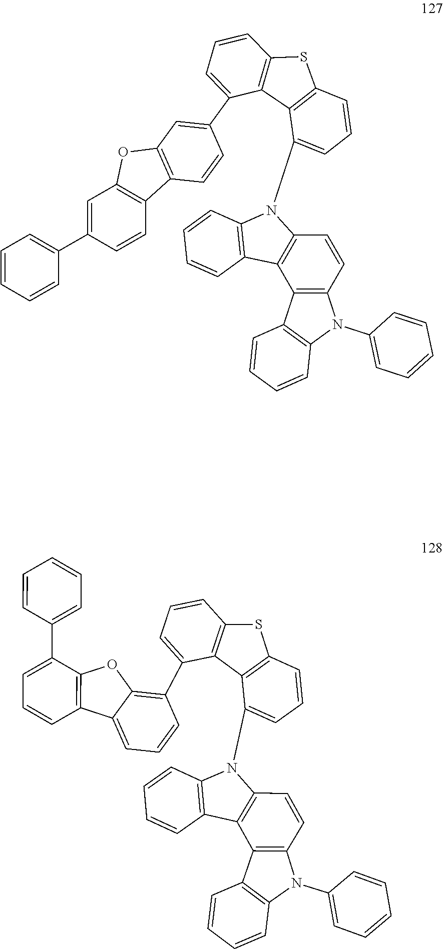 [SMILES: c1ccc(-c2ccc3c(c2)oc2cc(-c4cccc5sc6cccc(-n7c8ccccc8c8c9c%10ccccc%10n(-c%10ccccc%10)c9ccc87)c6c45)ccc23)cc1 ; c1ccc(-c2cccc3c2oc2c(-c4cccc5sc6cccc(-n7c8ccccc8c8c9c%10ccccc%10n(-c%10ccccc%10)c9ccc87)c6c45)cccc23)cc1]

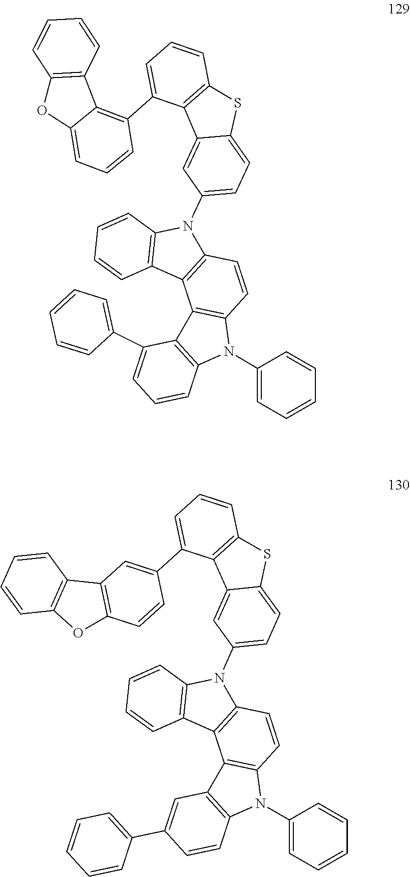 [SMILES: c1ccc(-c2cccc3c2c2c4c5ccccc5n(-c5ccc6sc7cccc(-c8cccc9oc%10ccccc%10c89)c7c6c5)c4ccc2n3-c2ccccc2)cc1 ; c1ccc(-c2ccc3c(c2)c2c4c5ccccc5n(-c5ccc6sc7cccc(-c8ccc9oc%10ccccc%10c9c8)c7c6c5)c4ccc2n3-c2ccccc2)cc1]

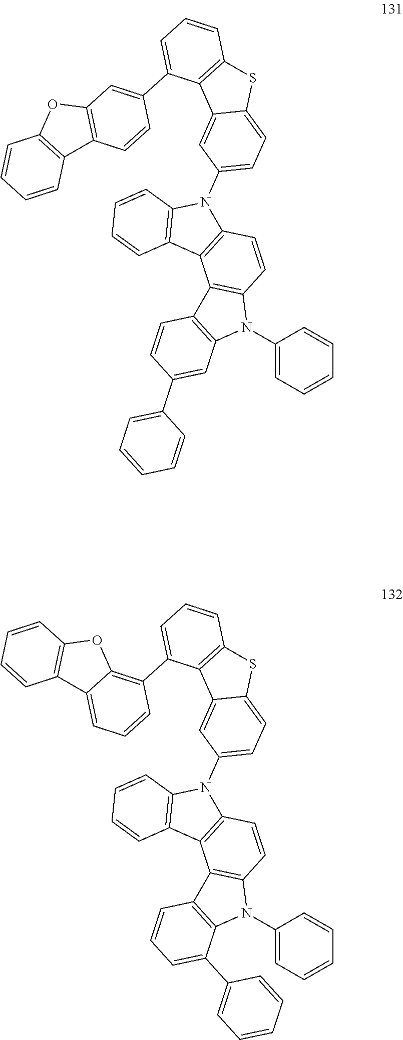 [SMILES: c1ccc(-c2ccc3c4c5c6ccccc6n(-c6ccc7sc8cccc(-c9ccc%10c(c9)oc9ccccc9%10)c8c7c6)c5ccc4n(-c4ccccc4)c3c2)cc1 ; c1ccc(-c2cccc3c4c5c6ccccc6n(-c6ccc7sc8cccc(-c9cccc%10c9oc9ccccc9%10)c8c7c6)c5ccc4n(-c4ccccc4)c23)cc1]

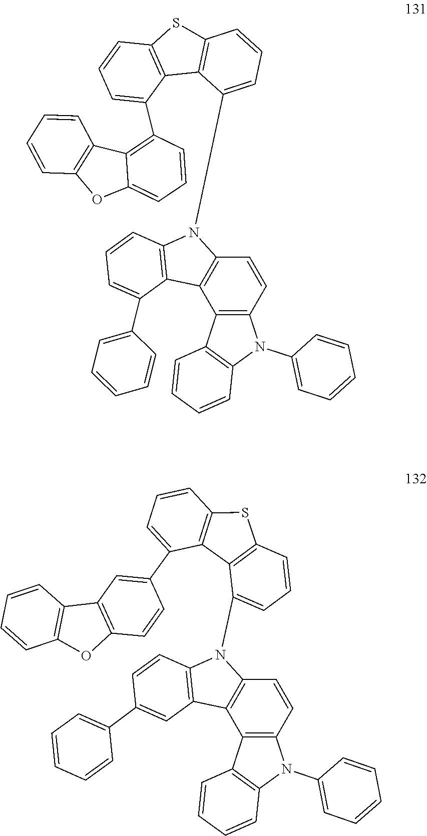 [SMILES: c1ccc(-c2cccc3c2c2c4c5ccccc5n(-c5ccccc5)c4ccc2n3-c2cccc3sc4cccc(-c5cccc6oc7ccccc7c56)c4c23)cc1 ; c1ccc(-c2ccc3c(c2)c2c4c5ccccc5n(-c5ccccc5)c4ccc2n3-c2cccc3sc4cccc(-c5ccc6oc7ccccc7c6c5)c4c23)cc1]

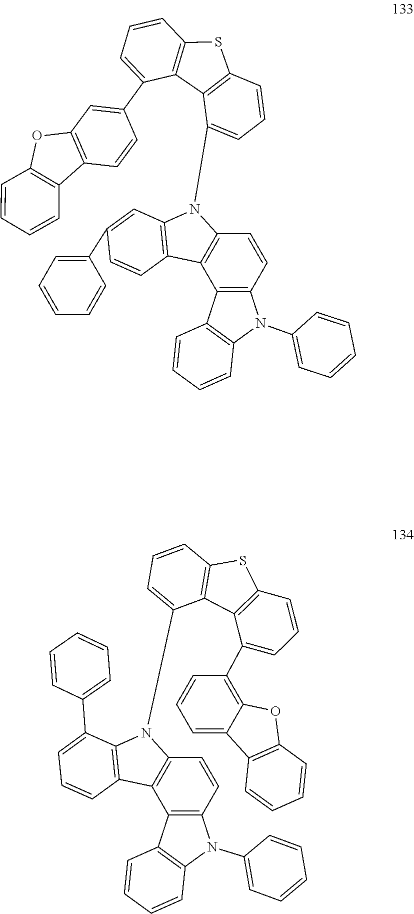 [SMILES: c1ccc(-c2ccc3c4c5c6ccccc6n(-c6ccccc6)c5ccc4n(-c4cccc5sc6cccc(-c7ccc8c(c7)oc7ccccc78)c6c45)c3c2)cc1 ; c1ccc(-c2cccc3c4c5c6ccccc6n(-c6ccccc6)c5ccc4n(-c4cccc5sc6cccc(-c7cccc8c7oc7ccccc78)c6c45)c23)cc1]

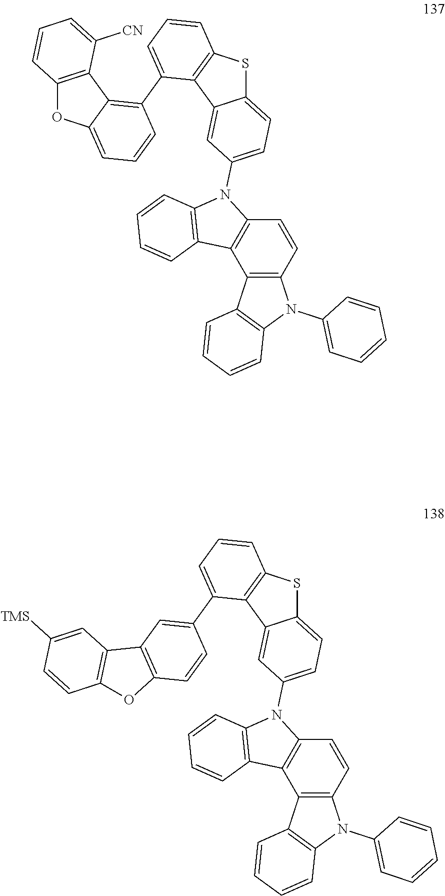 [SMILES: N#Cc1cccc2oc3cccc(-c4cccc5sc6ccc(-n7c8ccccc8c8c9c%10ccccc%10n(-c%10ccccc%10)c9ccc87)cc6c45)c3c12 ; C[Si](C)(C)c1ccc2oc3ccc(-c4cccc5sc6ccc(-n7c8ccccc8c8c9c%10ccccc%10n(-c%10ccccc%10)c9ccc87)cc6c45)cc3c2c1]

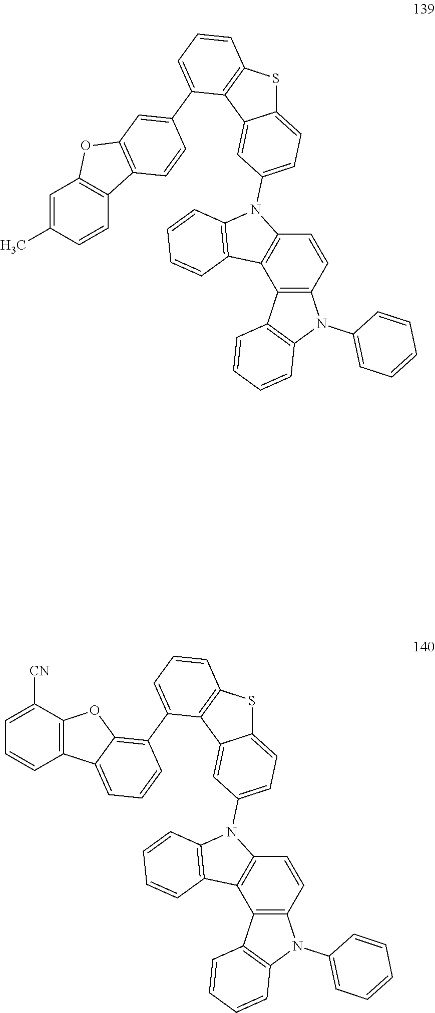 [SMILES: Cc1ccc2c(c1)oc1cc(-c3cccc4sc5ccc(-n6c7ccccc7c7c8c9ccccc9n(-c9ccccc9)c8ccc76)cc5c34)ccc12 ; N#Cc1cccc2c1oc1c(-c3cccc4sc5ccc(-n6c7ccccc7c7c8c9ccccc9n(-c9ccccc9)c8ccc76)cc5c34)cccc12]

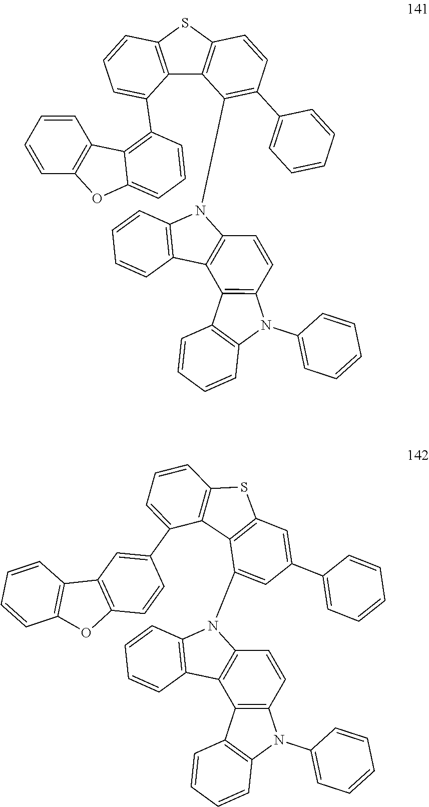 [SMILES: c1ccc(-c2ccc3sc4cccc(-c5cccc6oc7ccccc7c56)c4c3c2-n2c3ccccc3c3c4c5ccccc5n(-c5ccccc5)c4ccc32)cc1 ; c1ccc(-c2cc(-n3c4ccccc4c4c5c6ccccc6n(-c6ccccc6)c5ccc43)c3c(c2)sc2cccc(-c4ccc5oc6ccccc6c5c4)c23)cc1]

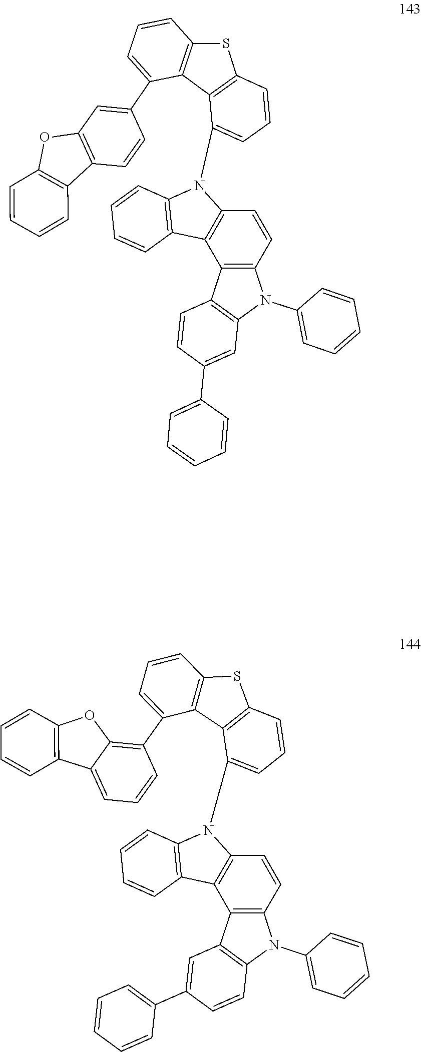 [SMILES: c1ccc(-c2ccc3c4c5c6ccccc6n(-c6cccc7sc8cccc(-c9ccc%10c(c9)oc9ccccc9%10)c8c67)c5ccc4n(-c4ccccc4)c3c2)cc1 ; c1ccc(-c2ccc3c(c2)c2c4c5ccccc5n(-c5cccc6sc7cccc(-c8cccc9c8oc8ccccc89)c7c56)c4ccc2n3-c2ccccc2)cc1]

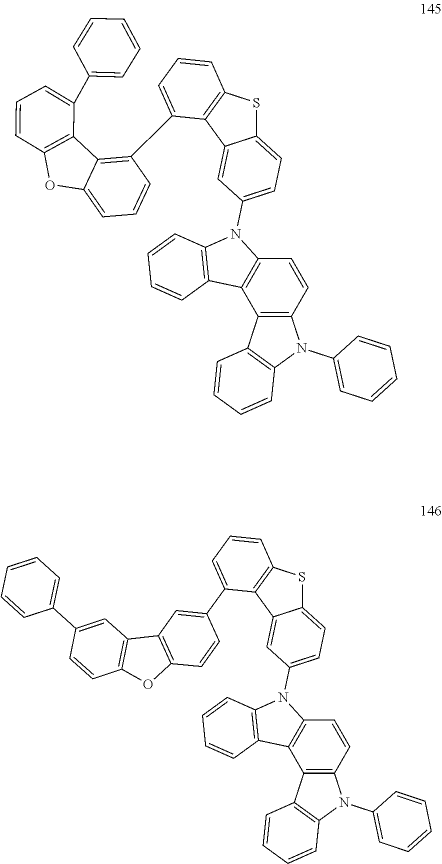 [SMILES: c1ccc(-c2cccc3oc4cccc(-c5cccc6sc7ccc(-n8c9ccccc9c9c%10c%11ccccc%11n(-c%11ccccc%11)c%10ccc98)cc7c56)c4c23)cc1 ; c1ccc(-c2ccc3oc4ccc(-c5cccc6sc7ccc(-n8c9ccccc9c9c%10c%11ccccc%11n(-c%11ccccc%11)c%10ccc98)cc7c56)cc4c3c2)cc1]

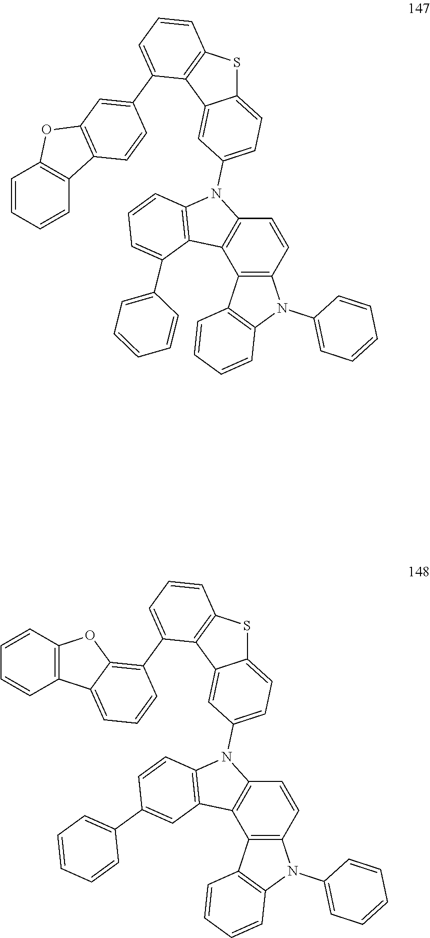 [SMILES: c1ccc(-c2cccc3c2c2c4c5ccccc5n(-c5ccccc5)c4ccc2n3-c2ccc3sc4cccc(-c5ccc6c(c5)oc5ccccc56)c4c3c2)cc1 ; c1ccc(-c2ccc3c(c2)c2c4c5ccccc5n(-c5ccccc5)c4ccc2n3-c2ccc3sc4cccc(-c5cccc6c5oc5ccccc56)c4c3c2)cc1]

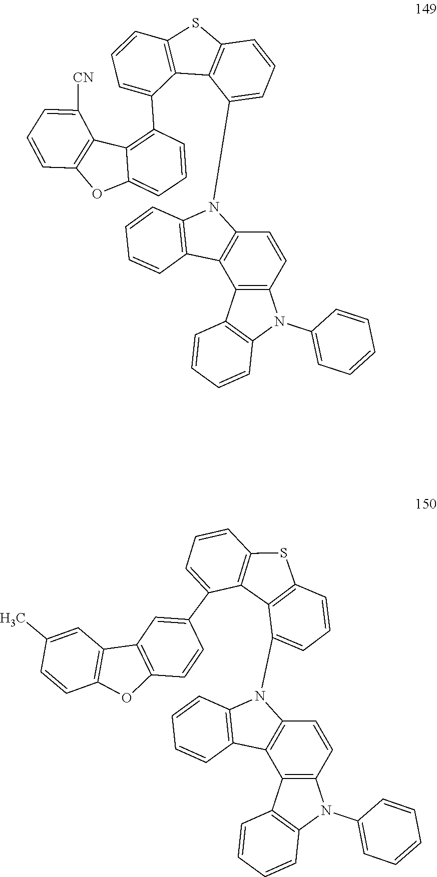 [SMILES: N#Cc1cccc2oc3cccc(-c4cccc5sc6cccc(-n7c8ccccc8c8c9c%10ccccc%10n(-c%10ccccc%10)c9ccc87)c6c45)c3c12 ; Cc1ccc2oc3ccc(-c4cccc5sc6cccc(-n7c8ccccc8c8c9c%10ccccc%10n(-c%10ccccc%10)c9ccc87)c6c45)cc3c2c1]

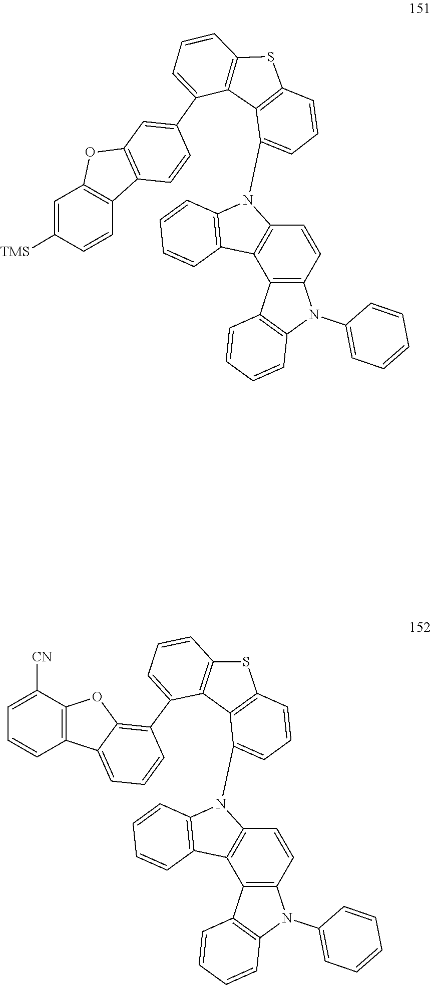 [SMILES: C[Si](C)(C)c1ccc2c(c1)oc1cc(-c3cccc4sc5cccc(-n6c7ccccc7c7c8c9ccccc9n(-c9ccccc9)c8ccc76)c5c34)ccc12 ; N#Cc1cccc2c1oc1c(-c3cccc4sc5cccc(-n6c7ccccc7c7c8c9ccccc9n(-c9ccccc9)c8ccc76)c5c34)cccc12]

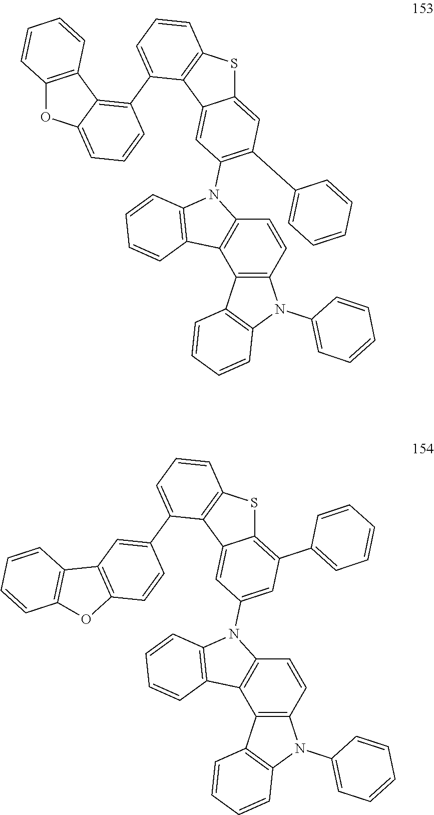 [SMILES: c1ccc(-c2cc3sc4cccc(-c5cccc6oc7ccccc7c56)c4c3cc2-n2c3ccccc3c3c4c5ccccc5n(-c5ccccc5)c4ccc32)cc1 ; c1ccc(-c2cc(-n3c4ccccc4c4c5c6ccccc6n(-c6ccccc6)c5ccc43)cc3c2sc2cccc(-c4ccc5oc6ccccc6c5c4)c23)cc1]

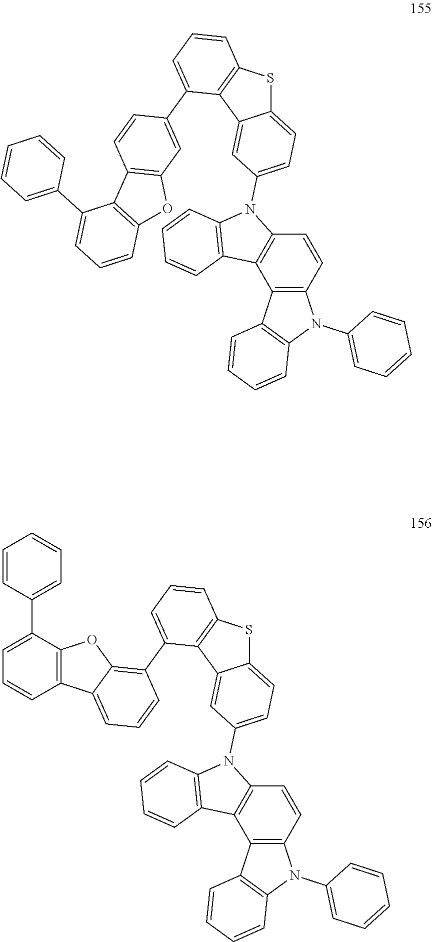 [SMILES: c1ccc(-c2cccc3oc4cc(-c5cccc6sc7ccc(-n8c9ccccc9c9c%10c%11ccccc%11n(-c%11ccccc%11)c%10ccc98)cc7c56)ccc4c23)cc1 ; c1ccc(-c2cccc3c2oc2c(-c4cccc5sc6ccc(-n7c8ccccc8c8c9c%10ccccc%10n(-c%10ccccc%10)c9ccc87)cc6c45)cccc23)cc1]

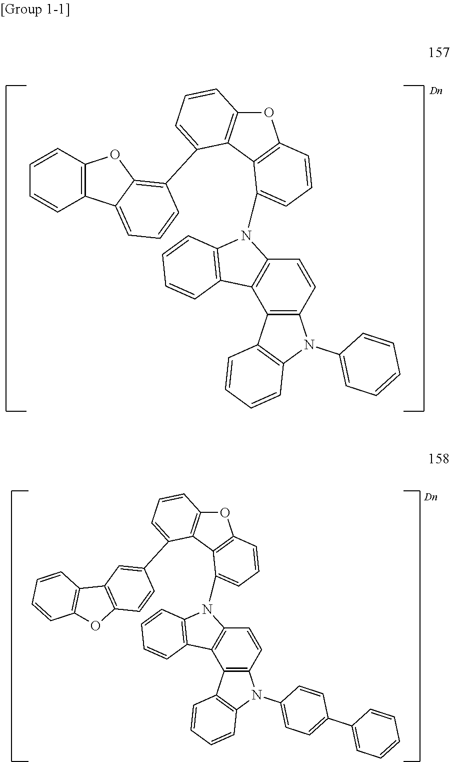

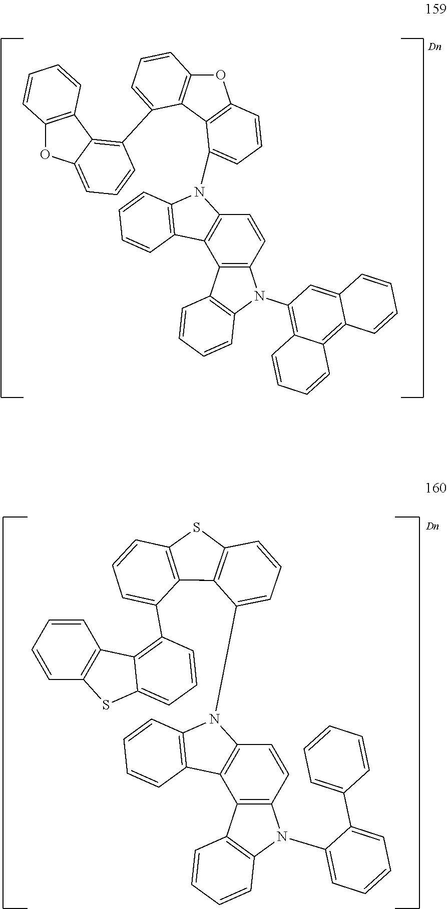

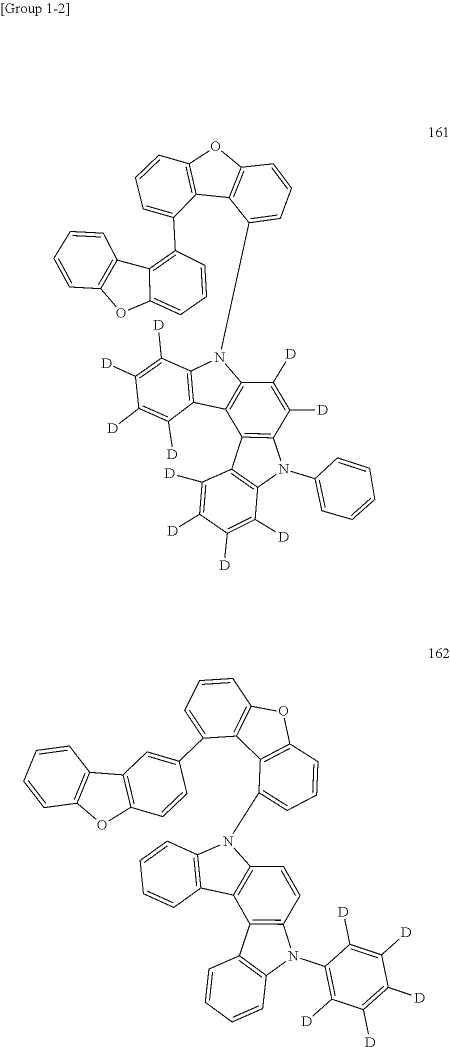 [SMILES: [2H]c1c([2H])c([2H])c2c(c1[2H])c1c3c4c([2H])c([2H])c([2H])c([2H])c4n(-c4cccc5oc6cccc(-c7cccc8oc9ccccc9c78)c6c45)c3c([2H])c([2H])c1n2-c1ccccc1 ; [2H]c1c([2H])c([2H])c(-n2c3ccccc3c3c4c5ccccc5n(-c5cccc6oc7cccc(-c8ccc9oc%10ccccc%10c9c8)c7c56)c4ccc32)c([2H])c1[2H]]

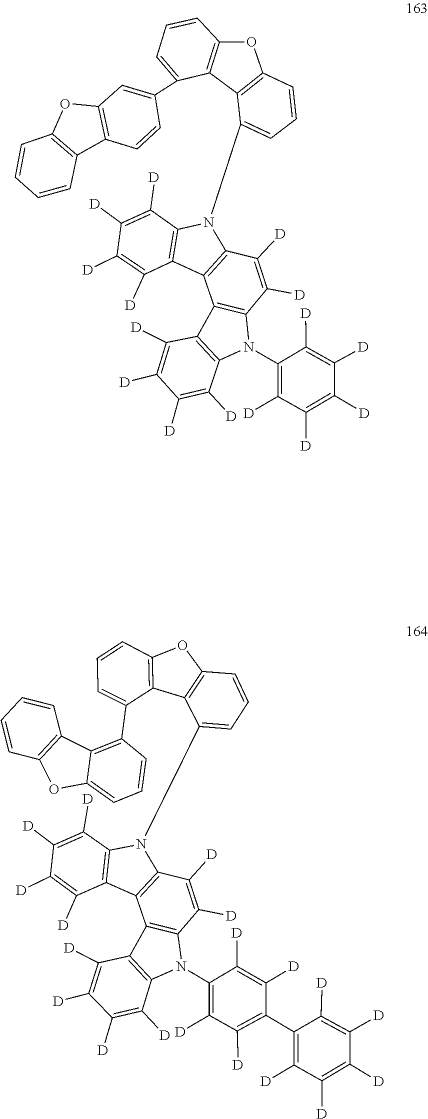 [SMILES: [2H]c1c([2H])c([2H])c(-n2c3c([2H])c([2H])c([2H])c([2H])c3c3c4c5c([2H])c([2H])c([2H])c([2H])c5n(-c5cccc6oc7cccc(-c8ccc9c(c8)oc8ccccc89)c7c56)c4c([2H])c([2H])c32)c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6cccc7oc8cccc(-c9cccc%10oc%11ccccc%11c9%10)c8c67)c5c([2H])c([2H])c43)c([2H])c2[2H])c([2H])c1[2H]]

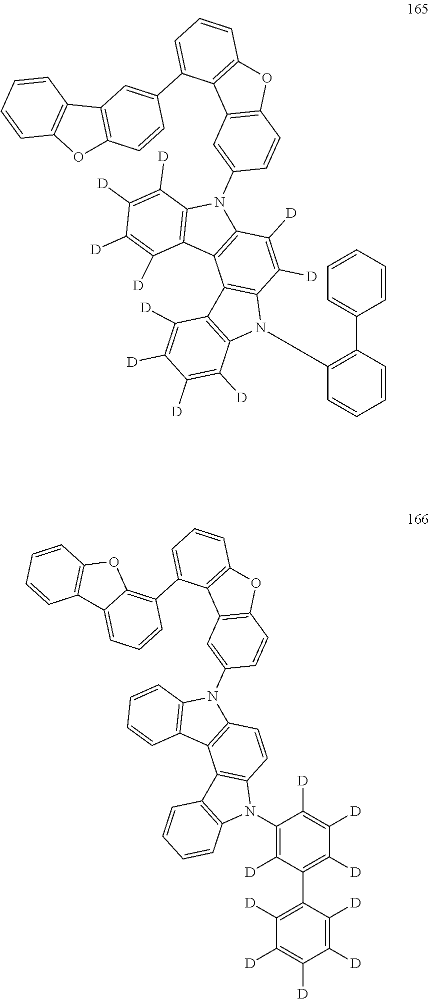 [SMILES: [2H]c1c([2H])c([2H])c2c(c1[2H])c1c3c4c([2H])c([2H])c([2H])c([2H])c4n(-c4ccccc4-c4ccccc4)c3c([2H])c([2H])c1n2-c1ccc2oc3cccc(-c4ccc5oc6ccccc6c5c4)c3c2c1 ; [2H]c1c([2H])c([2H])c(-c2c([2H])c([2H])c([2H])c(-n3c4ccccc4c4c5c6ccccc6n(-c6ccc7oc8cccc(-c9cccc%10c9oc9ccccc9%10)c8c7c6)c5ccc43)c2[2H])c([2H])c1[2H]]

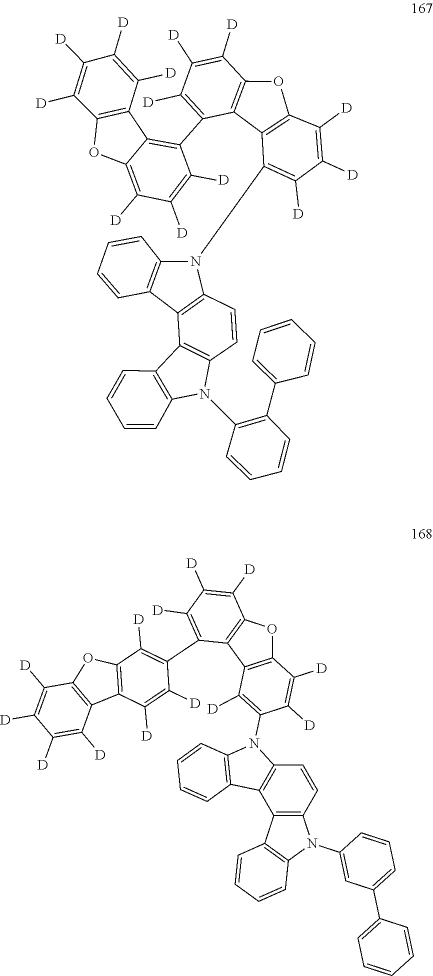 [SMILES: [2H]c1c([2H])c([2H])c2c(oc3c([2H])c([2H])c([2H])c(-c4c([2H])c([2H])c([2H])c5oc6c([2H])c([2H])c([2H])c(-n7c8ccccc8c8c9c%10ccccc%10n(-c%10ccccc%10-c%10ccccc%10)c9ccc87)c6c45)c32)c1[2H] ; [2H]c1c([2H])c([2H])c2c(oc3c([2H])c(-c4c([2H])c([2H])c([2H])c5oc6c([2H])c([2H])c(-n7c8ccccc8c8c9c%10ccccc%10n(-c%10cccc(-c%11ccccc%11)c%10)c9ccc87)c([2H])c6c45)c([2H])c([2H])c32)c1[2H]]

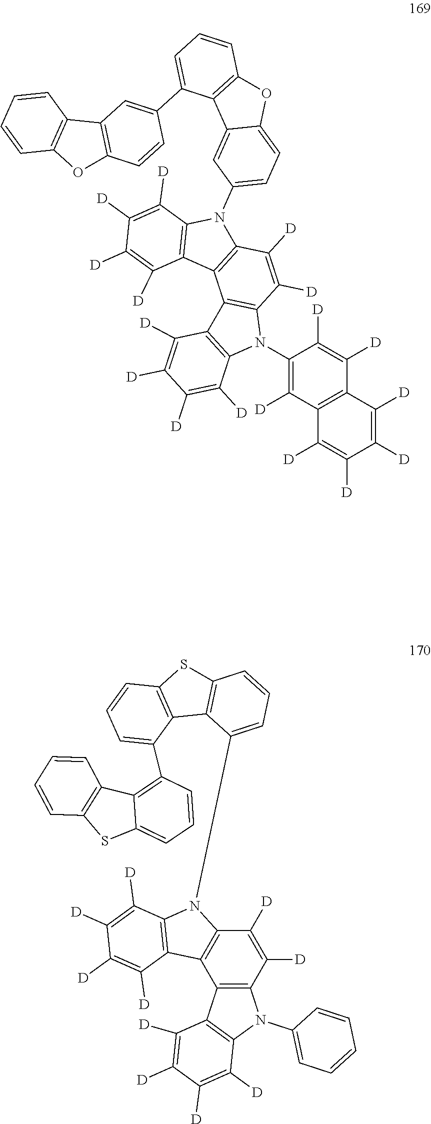 [SMILES: [2H]c1c([2H])c([2H])c2c([2H])c(-n3c4c([2H])c([2H])c([2H])c([2H])c4c4c5c6c([2H])c([2H])c([2H])c([2H])c6n(-c6ccc7oc8cccc(-c9ccc%10oc%11ccccc%11c%10c9)c8c7c6)c5c([2H])c([2H])c43)c([2H])c([2H])c2c1[2H] ; [2H]c1c([2H])c([2H])c2c(c1[2H])c1c3c4c([2H])c([2H])c([2H])c([2H])c4n(-c4cccc5sc6cccc(-c7cccc8sc9ccccc9c78)c6c45)c3c([2H])c([2H])c1n2-c1ccccc1]

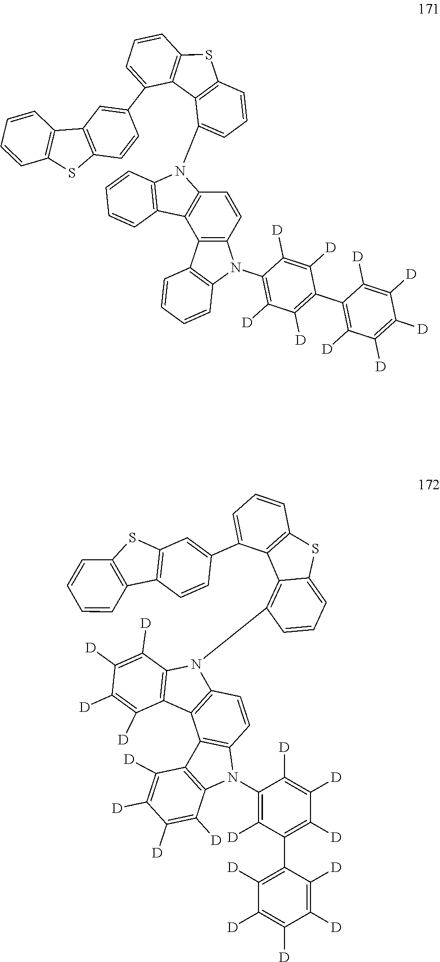 [SMILES: [2H]c1c([2H])c([2H])c(-c2c([2H])c([2H])c(-n3c4ccccc4c4c5c6ccccc6n(-c6cccc7sc8cccc(-c9ccc%10sc%11ccccc%11c%10c9)c8c67)c5ccc43)c([2H])c2[2H])c([2H])c1[2H] ; [2H]c1c([2H])c([2H])c(-c2c([2H])c([2H])c([2H])c(-n3c4ccc5c(c6c([2H])c([2H])c([2H])c([2H])c6n5-c5cccc6sc7cccc(-c8ccc9c(c8)sc8ccccc89)c7c56)c4c4c([2H])c([2H])c([2H])c([2H])c43)c2[2H])c([2H])c1[2H]]

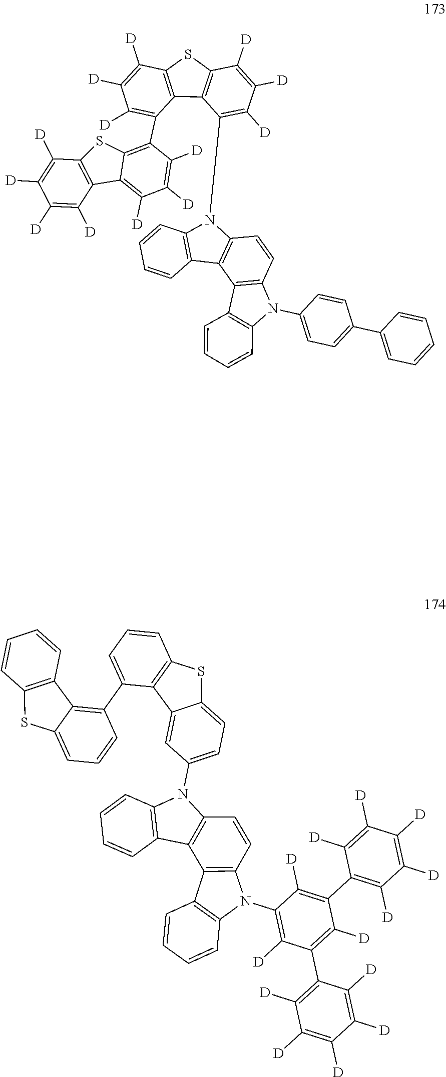 [SMILES: [2H]c1c([2H])c([2H])c2c(sc3c(-c4c([2H])c([2H])c([2H])c5sc6c([2H])c([2H])c([2H])c(-n7c8ccccc8c8c9c%10ccccc%10n(-c%10ccc(-c%11ccccc%11)cc%10)c9ccc87)c6c45)c([2H])c([2H])c([2H])c32)c1[2H] ; [2H]c1c([2H])c([2H])c(-c2c([2H])c(-c3c([2H])c([2H])c([2H])c([2H])c3[2H])c([2H])c(-n3c4ccccc4c4c5c6ccccc6n(-c6ccc7sc8cccc(-c9cccc%10sc%11ccccc%11c9%10)c8c7c6)c5ccc43)c2[2H])c([2H])c1[2H]]

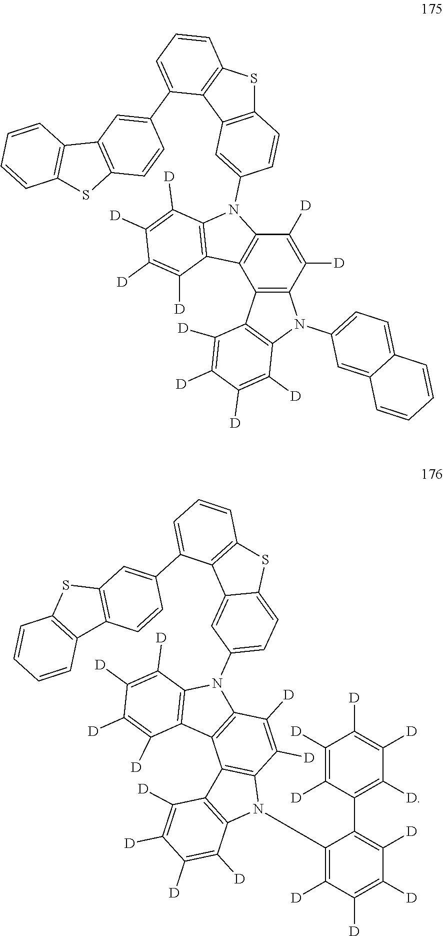

7. A composition for an organic optoelectronic device, the composition comprising:
a first compound; and
a second compound,
wherein:
the first compound is the compound for an organic optoelectronic device as claimed in claim 1, and
the second compound is represented by Chemical Formula 3: 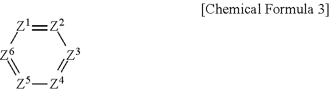
in Chemical Formula 3,
Z¹ to Z⁶ are each independently N or C-L^{a}-R^{a}, provided that at least two of Z¹ to Z⁶ are N,
each L^{a} is independently a single bond, a substituted or unsubstituted C6 to C20 arylene group, a substituted or unsubstituted C2 to C20 heterocyclic group, or a combination thereof,
each R^{a} is independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, and
each R^{a} is separately present or adjacent groups thereof are linked to each other to form a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring.

8. The composition for an organic optoelectronic device as claimed in claim 7, wherein:
Chemical Formula 3 is represented by one of Chemical Formula 3A to Chemical Formula 3C: 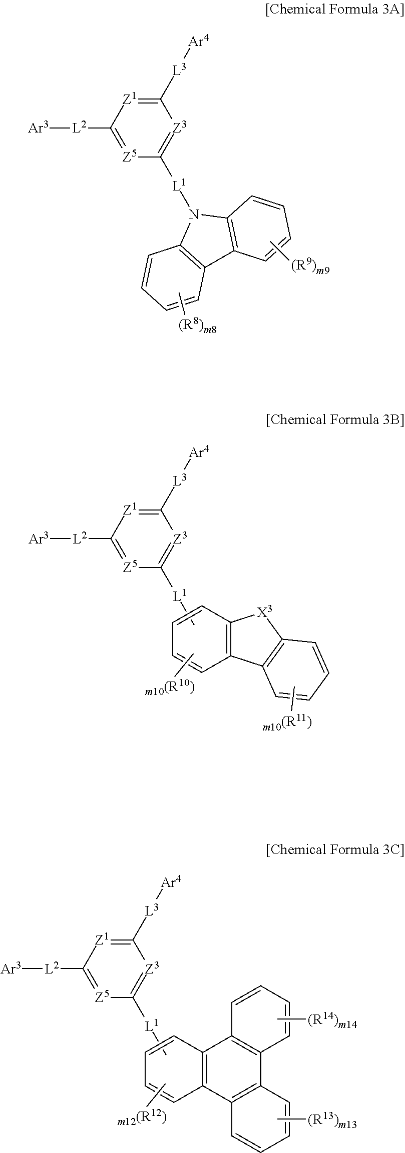
in Chemical Formulas 3A to 3C,
Z¹, Z³, and Z⁵ are each independently N or C-L^{a}-R^{a}, provided that at least two of Z¹, Z³, and Z⁵ are N,
X³ is O, S, or NR^{b},
L^{a} and L¹ to L³ are each independently a single bond, a substituted or unsubstituted C6 to C20 arylene group, a substituted or unsubstituted C2 to C20 heterocyclic group, or a combination thereof,
R^{a}, R^{b}, and R⁸ to R¹⁴ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof,
R⁸ and R⁹ are each separately present or adjacent groups thereof are linked to each other to form a substituted or unsubstituted aromatic monocyclic or polycyclic ring,
Ar³ and Ar⁴ are each independently a substituted or unsubstituted C6 to C30 aryl group, a substituted or unsubstituted C2 to C30 heterocyclic group, or a combination thereof,
R^{a}, Ar³, and Ar⁴ are each separately present, or adjacent groups of R^{a}, Ar³, and Ar⁴ are linked to each other to form a substituted or unsubstituted aromatic monocyclic or polycyclic ring or a substituted or unsubstituted heteroaromatic monocyclic or polycyclic ring,
m8, m9, m11, m13, and m14 are each independently an integer of 1 to 4, and
m10 and m12 are each independently an integer of 1 to 3.

9. The composition for an organic optoelectronic device as claimed in claim 7, wherein:
Chemical Formula 3 is represented by Chemical Formula 3A-I: 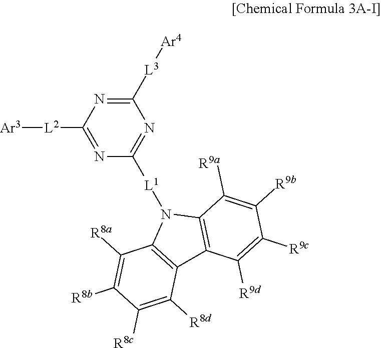
in Chemical Formula 3A-I,
L¹ to L³ are each independently a single bond, a substituted or unsubstituted phenylene group, a substituted or unsubstituted biphenylene group, a substituted or unsubstituted naphthylene group, a substituted or unsubstituted dibenzofuranylene group, or a substituted or unsubstituted dibenzothiophenylene group,
Ar³ and Ar⁴ are each independently a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted carbazolyl group, a substituted or unsubstituted dibenzofuranyl group, or a substituted or unsubstituted dibenzothiophenyl group, and
R^{8a}, R^{8b}, R^{8c}, R^{8d}, R^{9a}, R^{9b}, R^{9c}, and R^{9d} are each independently hydrogen, deuterium, a substituted or unsubstituted phenyl group, a substituted or unsubstituted dibenzofuranyl group, or a substituted or unsubstituted dibenzothiophenyl group.

10. An organic optoelectronic device, comprising:
an anode and a cathode facing each other, and
at least one organic layer between the anode and the cathode,
wherein the at least one organic layer includes the compound for an organic optoelectronic device as claimed in claim 1.

11. The organic optoelectronic device as claimed in claim 10, wherein:
the at least one organic layer includes a light emitting layer, and
the light emitting layer includes the compound for an organic optoelectronic device.

12. A display device comprising the organic optoelectronic device as claimed in claim 10.

13. An organic optoelectronic device, comprising:
an anode and a cathode facing each other, and
at least one organic layer between the anode and the cathode,
wherein the at least one organic layer includes the composition for an organic optoelectronic device as claimed in claim 7.

14. The organic optoelectronic device as claimed in claim 13, wherein:
the at least one organic layer includes a light emitting layer, and
the light emitting layer includes the composition for an organic optoelectronic device.

15. A display device comprising the organic optoelectronic device as claimed in claim 13.
